(19) United States
(12) Patent Application Publication
(10) Pub. No.: US 2026/0276637 A1
(43) Pub. Date: Sep. 17, 2026

(54) ASSAYS FOR CHARACTERIZATION OF EXTRACELLULAR VESICLES AND METHODS OF USING THE SAME

(71) Applicants: The Translational Genomics Research Institute, Phoenix, AZ (US); City of Hope, Duarte, CA (US); The General Hospital Corporation, Boston, MA (US)

(72) Inventors: Tijana Talisman, Duarte, CA (US); Kendall Jensen, Phoenix, AZ (US); Andras Saftics, Duarte, CA (US); Saumya Das, Boston, MA (US)

(21) Appl. No.: 19/166,704

(22) PCT Filed: Mar. 20, 2024
(86) PCT No.: PCT/US2024/020702

§ 371 Date: Sep. 18, 2025

Related U.S. Application Data
(60) Provisional application No. 63/491,489, filed on Mar. 21, 2023.

(51) Int. Cl. G01N 33/569; G01N 33/58

(52) U.S. Cl. CPC G01N 33/56966; G01N 33/582; G01N 2333/70596

(57) ABSTRACT

Disclosed herein, in certain embodiments, are methods for isolation and characterization of extracellular vesicles (EVs) subpopulations derived from one or more target tissues of interest using a novel assay that combines affinity capture of EVs, super-resolution microscopy, transcriptomic analysis, and proteomic analysis.

### CROSS-REFERENCE TO RELATED APPLICATIONS

The present application is an International Patent Application claiming priority to and benefit from U.S. Provisional Application No. 63/491,489, filed Mar. 21, 2023, the entire contents of which are herein incorporated by reference.

### GOVERNMENT LICENSE RIGHTS

This invention was made with government support under grant UG3/UH3 TR002878 awarded by the National Institutes of Health (NIH), grant P30CA033572 awarded by the National Cancer Institute of the NIH, and grant 1R35HL150807-01 awarded by the National Heart, Lung, and Blood Institute of the NIH. The government has certain rights in the invention.

### BACKGROUND

Extracellular vesicles (EVs) are membrane-encapsulated particles released from all cells and capable of mediating intercellular communication through their cargo, including nucleic acids, metabolites, lipids, and proteins. EVs have diverse biophysical characteristics and a broad size distribution (~30 nm-10 μm). Conventional isolation methods group EVs into fractions based on size, charge, and/or density. Further selectivity can be achieved by targeting specific biomolecules located on EV membranes. For example, most EVs contain common tetraspanin proteins such as CD9, CD63, and CD81. Thus, selective antibodies against tetraspanins can be used to affinity isolate a wide range of EVs. Moreover, EVs carry specific membrane proteins from cells of their origin. These proteins can be enriched in certain tissue types or diseases and used as handles to select for the specific subpopulations of EVs. This is especially relevant for the diagnostic applications of EVs, as assaying cargo of tissue-specific EVs (TSEVs) may improve their biomarker potential.

EVs isolated from easily accessible biofluids hold diagnostic promise, as they contain cargo molecules from their target tissues of origin. When cells undergo phenotypic changes (e.g., as occurs in disease states), EVs reflect departure from homeostasis and can rapidly report on the disease status. However, biofluids contain a complex mixture of EVs released from cells across the entire body, and only a small fraction of EVs come from phenotypically altered cells. The ability to isolate and characterize EV subpopulations with high sensitivity remains challenging.

### SUMMARY OF THE DISCLOSURE

Disclosed herein, in certain embodiments, are methods for isolation and characterization of extracellular vesicles (EVs) subpopulations derived from one or more target tissues of interest using a novel assay that combines affinity capture of EVs, super-resolution microscopy, transcriptomic analysis, and proteomic analysis.

Disclosed herein, in certain embodiments, is a multiplexed method for characterizing a population of tissue-specific extracellular vesicles (TSEVs) in a biofluid and/or tissue sample, comprising a mixed population of EVs (MEVs), the method comprising: (a) immobilizing the population of TSEVs in the sample on a surface functionalized with an affinity capture agent that specifically binds to a biomarker present on the plasma membrane of TSEVs; and (b) analyzing individual TSEVs within the population TSEVs using super-resolution microscopy to produce a profile of the population of TSEVs; wherein the profile of the population of TSEVs includes information on at least: (i) membrane protein composition of individual TSEVs; and (ii) intra-vesicular cargo composition of individual TSEVs. In certain embodiments, the method further comprises, prior to step (a), identifying a biomarker as a tissue-specific biomarker in the population of TSEVs on the basis that the biomarker: (a) is a plasma membrane protein; (b) exhibits greater than 5 transcripts per million (TPM) RNA expression in a tissue of origin; and (c) exhibits less than 0.1 TPM RNA expression in tissues other than the tissue of origin. In certain embodiments, the method further comprises, prior to step (a), identifying a biomarker as a tissue-specific biomarker in the population of TSEVs on the basis of a tissue enrichment score, tau (C). In certain embodiments, t is calculated according to the following formula:

$$τ = \frac{{\sum }_{i = 1}^{n} ( 1 - {\hat{x}}_{i} )}{n - 1} ; {\hat{x}}_{i} = \frac{{x}_{i}}{{max}_{1 \leq i \leq n} ( {x}_{i} )} ,$$

wherein n is the number of tissues; xᵢ corresponds to expression of the gene in a given tissue; and \hat{x}₁ is the expression profile component normalized by the maximal component value. In certain embodiments, τ is greater than or equal to 0.9. In certain embodiments, τ is greater than 0.9. In certain embodiments, the method further comprises, prior to step (a), isolating the population of MEVs from the sample, thereby producing an enriched population of MEVs. In certain embodiments, the method further comprises isolating the population of TSEVs from the enriched population of MEVs, wherein the isolated population of TSEVs is used for immobilizing in step (a). In certain embodiments, isolating the population of MEVs from the sample is performed via affinity capture, size exclusion chromatography (SEC), ultracentrifugation, ultrafiltration, flow field-flow fractionation, hydrostatic filtration dialysis, enzyme-linked immunosorbent assay (ELISA) density gradient, immunoprecipitation, polyethylene glycol (PEG) precipitation, PEG/dextran aqueous two phase system (ATPS) isolation, lectin-induced agglutination, acoustic nanofilter, and/or a microfluidic separation. In certain embodiments, isolating the population of MEVs is performed using an affinity capture method selected from the group consisting of affinity chromatography, flow-based affinity immunoassay, affinity pulldown, affinity bead capture, affinity resin capture, microfluidic affinity capture, ELISA, magneto-immunoprecipitation, mixed-mode chromatography (MMC), and membrane-affinity spin column (MASC). In certain embodiments, isolating the population of MEVs is performed using an affinity capture agent that specifically binds to one or more EV-specific biomarkers selected from the group consisting of CD9, CD63, and CD81. In certain embodiments, the method does not comprise, prior to step (a), isolating the population of MEVs from the sample. In certain embodiments, the biomarker present on the TSEVs of step (a) is a tissue-specific biomarker or an EV-specific biomarker. In certain embodiments, the EV-specific biomarker is a protein selected from the group consisting of CD9, CD63, and CD81. In certain embodiments, the MEVs and TSEVs do not exhibit substantial expression of a negative selection marker selected from the group consisting of Apolipoprotein A1 (ApoA1), Apolipoprotein A2 (ApoA2), Apolipoprotein B (ApoB), albumin (ALB), cytochrome C (CYC), fibronectin (FN), and nuclear RNA (nRNA). In certain embodiments, the super-resolution microscopy comprises Single Extracellular Vesicle Nanoscopy (SEVEN). In certain embodiments, SEVEN comprises use of single-molecule localization microscopy (SMLM). In certain embodiments, the SMLM is quantitative SMLM (qSMLM). In certain embodiments, the qSMLM comprises use of a surface assay for molecular isolation (SAMI-qSMLM). In certain embodiments, the SMLM is photoactivated localization microscopy (PALM). In certain embodiments, the SMLM is stochastic optical reconstruction microscopy (STORM). In certain embodiments, the STORM is direct STORM (dSTORM). In certain embodiments, the SMLM is point accumulation in nanoscale topography (PAINT). In certain embodiments, the SMLM has a single-molecule localization precision of 6-10 nm (e.g., 6 nm, 7 nm, 8 nm, 9 nm, or 10 nm). In certain embodiments, the super-resolution microscopy comprises super-resolution radial fluctuations (SRRF) imaging. In certain embodiments, the super resolution microscopy comprises total internal reflection fluorescence (TIRF) illumination. In certain embodiments, the super resolution microscopy comprises SRRF imaging and TIRF illumination. In certain embodiments, the super resolution microscopy comprises wide field illumination. In certain embodiments, the super resolution microscopy comprises SRRF imaging and wide field illumination. In certain embodiments, the super resolution microscopy comprises confocal illumination. In certain embodiments, the super resolution microscopy comprises SRRF imaging and confocal illumination. In certain embodiments, the profile of the population of TSEVs further includes information on one or more of the following: (i) size of TSEVs; (ii) shape of TSEVs; (ii) quantity or concentration of TSEVs; and (iii) heterogeneity of TSEVs. In certain embodiments, the information on the shape of TSEVs comprises information on the circularity and/or eccentricity of the TSEVs. In certain embodiments, the tissue-specific biomarker is from a tissue selected from the group consisting of cardiac tissue, neural tissue, pancreatic tissue, immune tissue, and cancer tissue. In certain embodiments, the EV-specific biomarker and/or the tissue-specific biomarker is a membrane protein, cytoplasmic protein, glycan, nucleic acid, lipid, or a combination thereof. In certain embodiments, the affinity capture agent of step (a) is a protein, peptide, aptamer, carbohydrate, or a combination thereof. In certain embodiments, the protein is lactadherin. In certain embodiments, the carbohydrate is a lectin. In certain embodiments, the protein or peptide is selected from the group consisting of a primary antibody, secondary antibody. Fab, F(ab′), F(ab′)2, single chain variable fragment (scFv), Fd, minibody, variable heavy domain, variable light domain, variable NAR domain, single chain binding polypeptide, dAb fragment, nanobody, VHH, and toxin. In certain embodiments, the method further comprises labeling the TSEVs with a labeling agent. In certain embodiments, the labeling agent a fluorescent reporter or a binding agent conjugated to a fluorescent reporter. In certain embodiments, the fluorescent reporter is a photoswitchable fluorescent reporter, photoactivatable fluorescent reporter, photoconvertible fluorescent reporter, spontaneously blinking fluorescent reporter, or temporarily binding fluorescent reporter. In certain embodiments, the fluorescent reporter is selected from the group consisting of AF532. AF488, AF532, AF555, AF568, AF594, AF647, AF680, AF700, AF750, Atto488, Atto532, Atto647N, Atto680, Atto700, CF532, CF555, CF568, CF647, CF660C, CF680, CF750, CF488A, CF583R, CF597R, CF680R, CF535ST, Cy3, Cy3b, Cy5, DY-634, DyLight650, Dronpa, JF549, JF646. JFX549, JFX554, JFX646, JFX650, mIrisFP, mMaple, mMaple 3, PAGFP, PAmCherry, PATagRFP, PAmKate, PS-CFP2, and quantum dots. In certain embodiments, the functionalized surface of step (a) is a coverslip. In certain embodiments, the functionalized surface of step (a) is coated with a coupling agent. In certain embodiments, the coupling agent is attached to the functionalized surface by way of a linker moiety. In certain embodiments, the coupling agent is selected from the group consisting of MCP2, MCP4, p-aminophenyltrimethoxysilane (APTMS), and aminotrimethoxy silane (ATMS). In certain embodiments, the intra-vesicular cargo comprises a protein, nucleic acid, lipid, or carbohydrate. In certain embodiments, the nucleic acid is an RNA or a DNA. In certain embodiments, the RNA is a messenger RNA (mRNA), microRNA (miRNA), long non-coding RNA (lncRNA), transfer RNA (tRNA), tRNA-derived small RNA (tsRNA), ribosomal RNA (rRNA), small rDNA-derived RNA (srRNA), small nucleolar RNA (snoRNA), Piwi-interacting RNA (piRNA). In certain embodiments, the membrane protein composition and/or the intra-vesicular cargo composition of individual TSEVs is further assayed using proteomic analysis to generate a proteomic profile of the population of TSEVs. In certain embodiments, the proteomic analysis comprises an immunoassay, mass spectrometry (MS), high performance liquid chromatography (HPLC), reversed-phase chromatography, dot blot analysis, two-dimensional gel electrophoresis, Edman sequencing, protein microarray analysis, structural proteomic analysis, functional proteomic analysis, protein-protein interaction analysis, proteome mining, and post-translational modification analysis. In certain embodiments, the intra-vesicular cargo composition of individual TSEVs is further analyzed using transcriptomic analysis to generate a transcriptomic profile of the population of TSEVs. In certain embodiments, the transcriptomic analysis comprises RNA sequencing (RNA-Seq). In certain embodiments, the transcriptomic analysis comprises analysis of lncRNAs. In certain embodiments, the sample is a biofluid sample. In certain embodiments, the sample is a tissue sample. In certain embodiments, the biofluid sample is: (1) a biofluid sample obtained from a subject; or (2) a cell culture medium. In certain embodiments, the biofluid sample obtained from a subject is selected from the group consisting of whole blood, plasma, serum, cerebrospinal fluid, saliva, sputum, nasal secretion, ocular secretion, cystic fluid, synovial fluid, bronchoalveolar lavage fluid, amniotic fluid, bone marrow aspirate, bile, milk, stool, swab, smear, semen, or urine. In certain embodiments, the biofluid sample has a volume between 0.08 μL and 400 μL. In certain embodiments, the biofluid sample has a volume no greater than 1 μL. In certain embodiments, the tissue sample is a formalin-fixed paraffin-embedded (FFPE) tissue block, fixed tissue, fresh tissue, or frozen tissue. In certain embodiments, the method is performed in accord with Minimal Information for Studies of Extracellular Vesicles (MISEV) guidelines. In certain embodiments, the method identifies one or more biomarkers present on the plasma membrane or in the lumen of TSEVs as being associated with a disease or disorder.

Disclosed herein, in certain embodiments, is a method of identifying one or more biomarkers associated with a disease or disorder from an population of TSEVs in a sample, comprising: (i) performing the method of any one of the foregoing aspects and embodiments, thereby obtaining a level of expression of one or more tissue-specific biomarkers from an enriched population of disease-associated TSEVs in the sample; (ii) calculating a difference score for the one or more tissue-specific biomarkers by comparing a level of expression of the one or more tissue-specific biomarkers from the population of disease-associated TSEVs to a level of expression of the one or more tissue-specific biomarkers from a reference population of TSEVs; wherein a difference score above a cutoff value indicates that the one more tissue-specific biomarkers are associated with a disease or disorder. In certain embodiments, the cutoff value is at a 50th percentile, 60th percentile. 70th percentile, 80th percentile, 90th percentile, or greater of the difference score in the reference population of TSEVs. In certain embodiments, the sample is: (1) a fluid sample obtained from a subject having the disease or disorder; or (2) a cell culture medium comprising a population of cells that model the disease or disorder.

Disclosed herein, in certain embodiments, is a method of diagnosing a subject as having or at risk of developing a disease or disorder, comprising: (i) performing the method of any one of the foregoing aspects and embodiments, on a sample obtained from the subject thereby obtaining a level of expression of one or more tissue-specific biomarkers associated with the disease or disorder; (ii) calculating a difference score for the subject by comparing a level of expression of the one or more tissue-specific biomarkers in the subject to a level of expression of the one or more tissue-specific biomarkers from a reference population of subjects without the disease or disorder; wherein a difference score above a cutoff value identifies the subject as having or at risk of developing the disease or disorder. In certain embodiments, the cutoff value is at a 50th percentile, 60th percentile. 70th percentile, 80th percentile, 90th percentile, or greater of the difference score in the reference population of subjects without the disease or disorder.

Disclosed herein, in certain embodiments, is a method of assessing therapeutic efficacy of a therapeutic agent in a subject having or at risk of developing a disease or disorder, comprising: (i) performing the method of any one of any one of the foregoing aspects and embodiments on a sample obtained from the subject prior to treatment with the therapeutic agent thereby obtaining a pre-treatment level of expression of one or more tissue-specific biomarkers characterized as having altered expression associated with the disease or disorder; (ii) administering an amount of the therapeutic agent to the subject; (iii) performing the method of any one of the foregoing aspects and embodiments on a sample obtained from the subject following treatment with the therapeutic agent thereby obtaining a post-treatment level of expression of the one or more tissue-specific biomarkers; (iv) calculating a difference score for the therapeutic agent by comparing the pre-treatment level of expression of the one or more tissue-specific biomarkers to the post-treatment level of expression of the one or more tissue-specific biomarkers; wherein a difference score above a cutoff value indicates that the therapeutic agent is effective at treating the disease or disorder. In certain embodiments, the cutoff value is at a 50th percentile, 60th percentile, 70th percentile, 80th percentile, 90th percentile, or greater of the difference score in the pretreatment level of expression of the one or more tissue-specific biomarkers. In certain embodiments, the method comprises characterizing the one or more tissue-specific biomarkers as having altered expression associated with the disease or disorder prior to performing step (i).

Disclosed herein, in certain embodiments, is a method of selecting a therapeutic agent for the treatment of a subject having or at risk of developing a disease or disorder, comprising: (i) performing the method of any one of the foregoing aspects and embodiments, thereby obtaining a level of expression of one or more tissue-specific biomarkers from an enriched population of disease-associated TSEVs; (ii) calculating a difference score for the one or more tissue-specific biomarkers by comparing a level of expression of the one or more tissue-specific biomarkers from the population of disease-associated TSEVs to a level of expression of the one or more tissue-specific biomarkers from a reference population of TSEVs, wherein a difference score above a cutoff value indicates that the one more tissue-specific biomarkers are associated with a disease or disorder: (iii) identifying a common biological signaling pathway associated with the one or more disease-associated tissue-specific biomarkers; and (iv) selecting a therapeutic agent that modulates activity of the common biological signaling pathway. In certain embodiments, the method further comprises (v) administering an effective amount of the therapeutic agent to the subject having or at risk of developing the disease or disorder. In certain embodiments, the cutoff value is at a 50th percentile, 60th percentile, 70th percentile, 80th percentile, 90th percentile, or greater of the difference score in the reference population of TSEVs. In certain embodiments, the level of expression is a mean or median level of expression. In certain embodiments, the method further comprises obtaining the sample from the subject having or at risk of developing the disease or disorder prior to performing step (i). In certain embodiments, the method further comprises obtaining the sample from the subject having or at risk of developing the disease or disorder prior to performing step (i). In certain embodiments, the disease or disorder is selected from the group consisting of a cancer, cardiovascular disease or disorder, neurological disease or disorder, and autoimmune disease or disorder. In certain embodiments, the subject is a human, non-human primate, or rodent.

### Definitions

Unless defined otherwise, all technical and scientific terms used herein have the same meaning as is commonly understood by one of skill in the art to which the claimed subject matter belongs. Generally, nomenclatures utilized in connection with, and techniques of, immunology, oncology, cell and tissue culture, molecular biology, and protein and oligo- or polynucleotide chemistry and hybridization described herein are those well-known and commonly used in the art. It is to be understood that the foregoing general description and the following detailed description are exemplary and explanatory only and are not restrictive of any subject matter claimed. The section headings used herein are for organizational purposes only and are not to be construed as limiting the subject matter described.

As used herein, singular forms “a,” “and,” and “the” include plural referents unless the context clearly indicates otherwise. Thus, e.g., reference to “an antibody” includes a plurality of antibodies and reference to “an antibody” in some embodiments includes multiple antibodies, and so forth.

As used herein, all numerical values or numerical ranges include whole integers within or encompassing such ranges and fractions of the values or the integers within or encompassing ranges unless the context clearly indicates otherwise. Thus, e.g., reference to a range of 90-100%, includes 91%, 92%, 93%, 94%, 95%, 95%, 97%, etc., as well as 91.1%, 91.2%, 91.3%, 91.4%, 91.5%, etc., 92.1%, 92.2%, 92.3%, 92.4%, 92.5%, etc., and so forth. In another example, reference to a range of 1-5,000 fold includes 1, 2, 3, 4, 5, 6, 7, 8, 9, 10, 11, 12, 13, 14, 15, 16, 17, 18, 19, 20-fold, etc., as well as 1.1, 1.2, 1.3, 1.4, 1.5-fold, etc., 2.1, 2.2, 2.3, 2.4, 2.5-fold, etc., and so forth.

“About” a number, as used herein, refers to range including the number and ranging from 10% below that number to 10% above that number. “About” a range refers to 10% below the lower limit of the range, spanning to 10% above the upper limit of the range.

As used herein, the term “functionalized surface” refers to a surface, typically a coverslip, coated with a coupling agent (e.g., a linker moiety) that is used to covalently link (i.e., “functionalize”) the surface to binding agents, including but not limited to proteins or peptides. e.g., antibodies.

As used herein, the terms “isolated,” “purified,” and variants thereof generally refer to isolation of a substance (e.g., an EV or a population of EVs) such that the substance comprises a significant percent (e.g., greater than 1%, greater than 2%, greater than 5%, greater than 10%, greater than 20%, greater than 50%, or more, usually up to about 90%-100%) of the sample in which it resides. In certain embodiments, a substantially purified component comprises at least 50%, 80%-85%, or 90%-95% of the sample. Techniques for purifying EVs of interest are well-known in the art and include those methods disclosed herein, e.g., without limitation, ultracentrifugation (e.g., differential ultracentrifugation and density gradient ultracentrifugation), hydrostatic filtration dialysis, flow field fractionation, exosome isolation kit, sequential filtration, ultrafiltration, size exclusion chromatography, immunocapture, ELISA, PEG-induced precipitation, lectin-induced precipitation, acoustic nanofilter, and immune-based microfluidic methods. Generally, a substance is purified when it exists in a sample in an amount, relative to other components of the sample, that is more than as it is found naturally.

As used herein, the term “labeling agent” refers to any agent capable of producing a detectable signal when bound to a substance it is intended to label. Non-limiting examples of a labeling agent include fluorescent reporters, such as a photoswitchable fluorescent reporter, photoactivatable fluorescent reporter, photoconvertible fluorescent reporter, spontaneously blinking fluorescent reporter, or temporarily binding fluorescent reporter. In certain embodiments, the labeling agent is a small molecule, protein, or nucleic acid.

As used herein, the terms “mixed population of extracellular vesicles” or “MEVs” refer to extracellular vesicles (EVs) from two or more (e.g., 2, 3, 4, 5, 6, 7, 8, 9, 10, or more) tissues of origin that are present in a single sample, such as a biofluid sample or tissue sample obtained from a subject.

As used herein, the terms “plasma membrane protein” and “protein present on plasma membrane” refer to transmembrane proteins of EVs and proteins found on the EV corona (e.g., proteins present on the extracellular surface of the EV).

The terms “recipient,” “individual,” “subject,” “host,” and “patient,” are used interchangeably herein and refer to any mammalian subject for whom diagnosis, treatment, or therapy is desired, particularly humans. “Mammal” for purposes of treatment refers to any animal classified as a mammal, including humans, domestic and farm animals, and laboratory, zoo, sports, or pet animals, such as dogs, horses, cats, cows, sheep, goats, pigs, mice, rats, rabbits, guinea pigs, monkeys, etc. In some embodiments, the mammal is a human. None of the terms require the supervision of a medical professional.

As used herein, the term “proteomic profile” refers to a representation of the expression pattern of a plurality of proteins in a biological sample, e.g., a biological fluid or tissue at a given time. In certain embodiments, “proteomic profile” refers to a representation of a pattern of protein expression as measured from EVs derived from one or more target cells of origin.

As used herein, the terms “tissue-specific extracellular vesicles” and “TSEVs” refer to EVs secreted from a specific target tissue (e.g., cardiac tissue, neural tissue, pancreatic tissue, immune tissue, cancer tissue, etc.) and containing in its membrane or lumen a biomarker (e.g., a protein, peptide, lipid, nucleic acid, and/or carbohydrate) that is selectively present in the target tissue. In certain embodiments, TSEVs are identified as being tissue specific if they contain a “tissue-specific biomarker” (e.g., transmembrane biomarker or luminal biomarker). As used herein, a “tissue-specific biomarker” can be defined on the basis that the biomarker is (a) a plasma membrane protein; (b) exhibits greater than 5 transcripts per million (TPM) RNA expression in a tissue of origin; and (c) exhibits less than 0.1 TPM RNA expression in tissues other than the tissue of origin.

As used herein, the terms “treatment,” “treating,” and the like, in some cases, refer to administering an agent, or carrying out a procedure, for the purposes of obtaining an effect. The effect may be prophylactic in terms of completely or partially preventing a disease or symptom thereof and/or is therapeutic in terms of effecting a partial or complete cure for a disease and/or symptoms of the disease. “Treatment,” as used herein, includes treatment of a disease or disorder in a mammal, particularly in a human, and includes: (a) preventing the disease or a symptom of a disease from occurring in a subject which is predisposed to the disease but has not yet been diagnosed as having it (e.g., including diseases associated with or caused by a primary disease; (b) inhibiting the disease, i.e., arresting its development; and (c) relieving the disease. i.e., causing regression of the disease. The term treating includes any indicia of success in the treatment, amelioration, or prevention of a disease or disorder, including any objective or subjective parameter such as abatement, remission, diminishing of symptoms or making the disease condition more tolerable to the patient, slowing in the rate of degeneration or decline, or making the final point of degeneration less debilitating. The treatment or amelioration of symptoms is based on one or more objective or subjective parameters, including the results of an examination by a physician. Accordingly, the term “treating” includes the administration of the agents or compositions of the present disclosure to prevent or delay, to alleviate, or to arrest or inhibit development of the symptoms or conditions associated with diseases. The term “therapeutic effect” refers to the reduction, elimination, or prevention of the disease, symptoms of the disease, or side effects of the disease in the subject. A subject is “treated” for a disease or disorder if, after receiving a therapeutic amount of a therapeutic agent or composition of the present disclosure, the patient shows observable and/or measurable change in a parameter or symptom of the disease or disorder.

### BRIEF DESCRIPTION OF THE DRAWINGS

FIGS. 1A-1E show an overview of the isolation and characterization of extracellular vesicles (EVs) from pooled human plasma. FIG. 1A and FIG. 1B show dot blots evaluating size exclusion chromatography (SEC) fractions of isolated extracellular vesicles (EVs) from pooled human plasma. Dot blots of total protein concentration of fractions (FIG. 1A). Dot blots showing EV markers and impurities across different fractions (F1-F6: FIG. 1B). “VV” indicates void volume. FIG. 1C shows a schematic for the experimental workflow for isolating and characterizing EVs using the disclosed assays. The pooled human plasma was isolated using a 70 nM SEC column, and collected fractions were assessed by dot blots. Tetraspanins (TSPAN) CD9, CD63, and CD81 served as positive markers and ApoA as a negative marker. Fractions F1-F5 were selected for downstream applications. FIG. 1D (left panel) shows a representative transmission electron microscopy (TEM) image of combined EV fractions F1-F5. FIG. 1D (right panel) shows dots blots of combined fractions F1-F5, assessing the CD9, CD63, CD81, and syntenin content (EV markers): as well as the cytochrome C (CytC) and ApoA content (confounding proteins co-isolated with EVs). FIG. 1E shows a size distribution of the combined fractions F1-F5 acquired using nanoparticle tracking analysis (NTA). The data represent mean±SEM values of 3 runs. The EV concentration was determined as 5.17×109±4.89×107 particle/mL.

FIGS. 2A-2K are plots and images demonstrating characterization of EVs using an assay disclosed herein and named Single Extracellular Vesicle Nanoscopy (SEVEN). FIG. 2A and FIG. 2B show photophysical properties of anti-TSPAN-AF647 probes (mixture of anti-CD9, anti-CD63, and anti-CD81 antibodies labeled with AF647 that was used for staining). Data is based on 15 regions of interest (ROIs). FIG. 2A shows EV density as a function of dark time (s). The maximum observed dark time was 250 s. FIG. 2B shows average number of localizations per fluorescent probe. Average number of localizations/probe was 14. FIG. 2C shows estimation oflocalization precision. SMLM ROIs used to assess tetraspanin-enriched EVs were evaluated for localization precision using coordinate-based localization precision estimator (CBLPE) and Nikon's NIS Elements software. The data represent at least 14 analyzed ROIs per condition, with mean z SEM values indicated. FIG. 2D shows a schematic of affinity isolated EV stained with fluorescent antibodies. Anti-TSPAN antibodies (anti-CD9, anti-CD63, and anti-CD81 antibodies) were covalently immobilized on the polymer-coated glass coverslip surface. After affinity capture, EVs were stained using a mixture of fluorescently labeled anti-TSPAN antibodies. FIG. 2E shows a fluorescence image with an anti-TSPAN Abs-coated spot and capped polymer surface. A clear border is visible between the Ab-labeled dot and the capped polymer surface. FIG. 2F shows Voronoi tessellation-based detection of EVs in a SMLM image. FIG. 2G shows raw SMLM image of TSPAN-enriched EVs. FIG. 2H shows TSPAN-enriched EVs are imaged in SMLM (dark gray dots, signal from fluorescent anti-TSPAN Abs) and TIRF (light gray pixels, signal from GFP). (Middle) Tessellation polygons (white lines) are outlining SMLM localization (dark dots within EV); EV is outlined in white dashed line using the Voronoi tessellation algorithm. FIG. 2I shows number of detected EVs per ROI for spot coated with either a mixture of anti-TSPAN antibodies or anti-rabbit IgG control. FIG. 2J shows EV diameter detected with SMLM imaging and tessellation analysis (SMLMT); SMLM imaging and EVSCAN analysis (SMLME); TEM and segmentation analysis; and NTA. FIG. 2K shows EV circularity detected with SMLMT. SMLME, and TEM. Box plots indicate interquartile range (box), median (center line), mean (cross); the hollow dots (for TEM and SMLM) indicate EVs detected beyond 1.5-times the interquartile range (marked by the Whisker lines under and over the box). Error bars. SEM; *** indicates p<0.001. Numerical values and p-values are provided in Table 1 and Table 2.

FIGS. 3A-3F show application of the SEVEN assay for characterization of SEC-enriched EVs from pooled human plasma. FIG. 3A shows (left) tetraspanin-enriched EVs detected using CF568-maleimide (labels membrane available cysteine residues; white dots) and AF647 labeled anti-TSPAN Abs (labels membrane tetraspanins, gray dots); and (right) 2D distribution of relative centroid shifts for two-color EVs (centroid of the CF568 channel is assigned position at 0,0). Full width half maximum values for Gaussian fittings of EV counts are shown: n=3. FIG. 3B shows the number of detected TSPAN-enriched EVs per ROI. The x-axis represents EV concentrations normalized to the highest applied EV concentration (undiluted SEC-enriched EVs). Mean z SEM; minimum n=4, 20 ROIs; R2=0.9986. Inset, Raw SMLM image of TSPAN-enriched EVs. The arrow represents the dilution at which the SMLM image was acquired. FIG. 3C shows the number of detected EVs per ROI in the CD9-, CD63-, and CD81-enriched EV subpopulations and controls: anti-rabbit IgG and anti-cytochrome C Abs were immobilized onto surfaces; anti-TSPAN Abs were immobilized onto surfaces and EVs were lysed with Triton X-100; surfaces were not immobilized with Abs. Mean±SEM; n=4, 20 ROIs. FIG. 3D shows raw SMLM images of CD9-, CD63-, and CD81-enriched EVs. FIG. 3E shows raw SMLM images of control surfaces for SEC-enriched EVs from pooled human plasma, including anti-rabbit IgG immobilized onto surfaces; anti-cytochrome C Ab immobilized onto surfaces; anti-TSPAN Abs immobilized onto surfaces and EVs were lysed with Triton X-100; and no Ab immobilized onto surfaces. FIG. 3F shows 2D histograms and corresponding box plots for CD9-, CD63-, and CD81-enriched EVs. Each dot represents a single EV with a corresponding diameter (x-axis) and the number of detected TSPAN molecules (y-axis). Box plots: interquartile range (box), median (center line), mean (cross); the hollow dots indicate EVs detected beyond 1.5-times the interquartile range (marked by the Whisker lines under and over the box), n=4, 20 ROIs. ** Indicates p<0.01; *** indicates p<0.001. Numerical values and p-values are provided in Table 1 and Table 2.

FIGS. 4A-4D show box and whisker plots indicating circularity of EVs from human plasma. FIG. 4A shows shape of CD9-, CD63-, and CD81-enriched EVs from SEC-enriched pooled human plasma. FIG. 4B shows shape of CD9-, CD63-, and CD81-enriched EVs from crude plasma. FIG. 4C shows shape of TSPAN-enriched EVs isolated via SEC or from crude plasma. FIG. 4D shows shape of CD9-enriched EVs from crude pooled healthy control plasma (blue) and PDAC patient plasma (red color palette, P1-P4).

FIGS. 5A-5F show plots and images demonstrating application of the SEVEN assay for characterization of EVs from crude pooled human plasma. FIG. 5A shows the number of detected TSPAN-enriched EVs per ROI. The x-axis represents EV concentrations normalized to the highest applied EV concentration (1:20 dilution of crude plasma). Mean±SEM; n=3, 15 ROIs; R2=0.9947. Inset, Raw SMLM image of TSPAN-enriched EVs. The arrow represents the dilution at which the SMLM image was acquired. FIG. 5B shows 2D histograms and corresponding box plots for TSPAN-enriched EVs from either SEC-enriched or crude plasma samples. Each dot represents a single EV with a corresponding diameter (x-axis) and the number of detected TSPAN molecules (y-axis), n=5, 25 ROIs. FIG. 5C shows the number of detected EVs per ROI in the CD9-, CD63-, and CD81-enriched EV subpopulations and controls: anti-rabbit IgG and anti-cytochrome C antibodies were immobilized onto surfaces: anti-TSPAN antibodies were immobilized onto surfaces and EVs were lysed with Triton X-100; surfaces were not immobilized with antibodies. Mean±SEM; n=4, 20 ROIs. FIG. 5D shows raw SMLM images of CD9-, CD63-, and CD81-enriched EVs. FIG. 5E shows 2D histograms and corresponding box plots for CD9-, CD63-, and CD81-enriched EVs isolated directly from crude plasma. Each dot represents a single EV with a corresponding diameter (x-axis) and the number of detected TSPAN molecules (y-axis). n=4, 20 ROIs. Box plots in FIG. 5B and FIG. 5E: interquartile range (box), median (center line), mean (cross); the hollow dots indicate EVs detected beyond 1.5-times the interquartile range (marked by the Whisker lines under and over the box). *** Indicates p<0.001. Numerical values and p-values are provided in Tables 1-3. FIG. 5F shows raw SMLM images of control surfaces for EVs from 1:100 diluted crude pooled human plasma, including (from left to right): anti-rabbit IgG immobilized onto surfaces; anti-cytochrome C antibodies immobilized onto surfaces; anti-TSPAN antibodies immobilized onto surfaces and EVs lysed with a detergent; and no antibody immobilized onto surfaces.

FIGS. 6A-6C are images and plots showing lactadherin-isolated EVs. FIG. 6A shows a raw SMLM image of EVs. FIG. 6B shows 2D histograms and corresponding box plots for lactadherin-enriched EVs isolated directly from crude plasma. Each dot represents a single EV with a corresponding diameter (x-axis) and the number of detected TSPAN molecules (y-axis). FIG. 6C shows a box plot indicating EV circularity: interquartile range (box), median (center line), mean (cross): the hollow dots indicate EVs detected beyond 1.5-times the interquartile range (marked by the Whisker lines under and over the box); n=3, 15 ROIs.

FIGS. 7A-7D show use of the SEVEN assay for characterization of EVs from plasma of pancreatic ductal adenocarcinoma (PDAC) patients. 2D histograms and corresponding box plots for CD9-enriched EVs (FIG. 7A) and IGF1R-enriched EVs (FIG. 7B) isolated from healthy control plasma and PDAC patient plasma (P1-P4). Each dot represents a single EV with a corresponding diameter (x-axis) and the number of detected TSPAN molecules (y-axis). n=3, 15 ROIs. FIG. 7C shows box plots indicating circularities of IGF1R-enriched EVs isolated from healthy control plasma and PDAC patient plasma (P1-P4). FIG. 7D For IGF1R-enriched EVs, a unique population was identified (indicated by the dashed grey gating polygon in FIG. 7B) with a larger diameter and higher TSPAN content. The number of gated EVs for healthy control and PDAC patients is shown. Box plots in FIGS. 7A-7C: interquartile range (box), median (center line), mean (cross); the hollow dots indicate EVs detected beyond 1.5-times the interquartile range (marked by the Whisker lines under and over the box). n=3, 15 ROIs. Significance tests were performed against healthy control. ** Indicates p<0.01; *** indicates p<0.001. Numerical values and p-values are provided in Table 7 and Table 8.

FIGS. 8A-8G shows plots demonstrating ExoView data analysis. FIGS. 8A-8D show CD9-, CD63-, CD81-, and CD41a-enriched EVs detected on the Ab-coated spots. FIG. 8E shows control IgG spot used as the negative control. FIG. 8F shows CD41a-enriched EVs positive for CD9 used to evaluate platelet-derived EVs. EV count per spot was determined from fluorescence channel signals. The data represent 4 independent repeats per spot for pooled healthy plasma and 3 independent repeats per spot for PDAC patient plasma with mean±SEM values indicated. Each point represents the average of the number of EVs per spot in a single repeat (1-3 spots were evaluated per repeat). EV counts were normalized to a spot size of 150 μm. FIG. 8G shows diameters of CD9-, CD63-, and CD81-enriched EVs for healthy pooled human plasma as determined from interferometric microscopy (IM) channel. Box plots: interquartile range (box), median (center line), mean (cross): the hollow dots indicate EVs detected beyond 1.5-times the interquartile range (marked by the Whisker lines under and over the box). 4 independent repeats. ** indicates p<0.01; *** indicates p<0.001.

FIGS. 9A-9C shows characterization of EVs from plasma using transmission electron microscopy (TEM), microfluidic resistive pulse sensing (MRPS), and ExoView. FIG. 9A shows TEM images of SEC-enriched EVs from PDAC patient plasma. FIG. 9B shows MRPS measurement of EV diameters of SEC-enriched EVs from plasma. FIG. 9C shows ExoView size measurement of EV diameters of CD9-enriched EVs from crude plasma. (P1-P3 had significantly smaller EV sizes compared to healthy, but differences were not significant between healthy and P4).

FIG. 10 shows ExoView images for crude plasma samples from healthy pooled plasma and PDAC patient plasma. Full field of view is on top, and zoomed-in region on the bottom. EVs isolated on anti-CD81 Ab coated spot and stained with Abs against CD9, CD63, and CD81; single, double, and triple positive EVs can be seen.

FIG. 11 shows raw SMLM images of CD9- and IGF1R-enriched EVs from healthy pooled plasma and PDAC patient plasma.

FIGS. 12A-12F show data indicating differential expression of EV long RNA cargo in healthy control patients and patients with heart failure (HF). (FIG. 12A) Unsupervised hierarchical clustering of all long non-coding RNAs (lncRNAs) in the discovery cohort (Ctrl=9, HFpEF=7, HFrEF=7). V1 and V2 indicate admission (during decompensation) and discharge (after therapy), respectively. (FIG. 12B) Volcano plots showing the significantly differentially expressed genes from the long RNA sequencing analysis in HFpEF versus Control and HFrEF versus Control indicated as HFpEF and HFrEF, respectively. (FIG. 12C) Bar plot showing up- and down-regulated genes in HFpEF versus Control and HFrEF versus Control indicated as HFpEF and HFrEF respectively. (FIG. 12D) Commonly expressed mRNAs and lncRNAs between the two analyses (HFpEF versus Control and HFrEF versus Control indicated as HFpEF and HFrEF respectively). (FIG. 12E) KEGG Pathway enrichment analysis for HFrEF vs Ctrl and (FIG. 12F) HFpEF vs Ctrl. ExRNA indicates Extracellular RNA: Ctrl, Control; HFpEF, Heart Failure with preserved Ejection Fraction; HFrEF, Heart Failure with reduced Ejection Fraction; V1, at Admission (decompensation); V2, at Discharge (after therapy); mRNA, messenger RNA; lncRNA, long non-coding RNA.

FIGS. 13A-13G show plots images and plots indicating that differentially expressed lncRNA cargo of EVs are often distinctively fragmented. (FIG. 13A) EV visualization using Transmission Electron Microscope (TEM) imaging, (FIG. 13B) EV quantification using microfluidic resistive pulse sensing (MRPS) analysis, and (FIG. 13C) westem blot for EV markers including Alix, CD63, CD81. and Syntenin, as well as “negative” marker 58k Golgi protein reflecting the degree of EV preparation purity. (FIG. 13D) Analysis of differentially expressed targets from the RNA-sequencing analysis examined by digital PCR in various RNA compartments of pooled human plasma from both heart failure and control samples, namely, EV compartments composed of Exoeasy kit and SEC-AFC; Ago2-associated RNA compartment obtained by immunocapture using Ago2 antibody; Lipoprotein-associated, LDL and HDL RNA compartments obtained through ultracentrifugation. Validation of the EV compartment using SEC-AFC. (FIGS. 13E-13G) Two lncRNA fragments of the same lncRNA LINC00989 show differential expression patterns in heart failure patient plasma vs. control in the discovery cohort, as seen in the coverage plots as well as in the validation cohort evaluated using RT-qPCR. The x-axis shows the full length of the transcript. Y axis represents sample expression. Box plots are obtained using RT-qPCR demonstrated as 2-dct values. dCT values are calculated using a Spike-in normalizer. Significance is indicated by * p<0.01 ** p<0.001 ***p<0.0001, where p is calculated using the Kruskal-Wallis test. lncRNAs indicates long non-coding RNA, EV, Extracellular vesicles; RT-qPCR, Real-Time quantitative PCR; Frag 1, Fragment 1; Frag 2, Fragment 2; Ctrl, Control; HFpEF, Heart Failure with preserved Ejection Fraction; HFrEF, Heart Failure with reduced Ejection Fraction; SEC-AFC, Size Exclusion Chromatography using Automated Fraction Collector: AGO2 IC, Immunocapture using Ago2 antibody; LDL, Low Density Lipoprotein; HDL, High Density Lipoprotein; MRPS, microfluidic resistive pulse sensing.

FIGS. 14A-14J show plots indicating EV-derived transcripts were differentially expressed between control and acute decompensated heart failure patients in the validation cohort. (FIGS. 14A-14H) Box plots showing some of the differentially expressed targets that were experimentally confirmed on the validation cohort (Ctrl=24, HFpEF=86, HFrEF=72) using RT-qPCR demonstrated as 2^{-dCt} values. dCT values are calculated using a Spike-in normalizer. Significance is indicated by * p<0.01 ** p<0.001 ***p<0.0001, where p is calculated using the Kruskal-Wallis test. (FIG. 141) Forest plot of all the experimentally validated differentially expressed EV cargo. The coefficient estimate represents the numerical differences between each HF subtype and control adjusted for age and sex. Negative values represent decreasing dCT values or increased plasma EV-RNA expression. (FIG. 14J) Diagnostic receiver-operating characteristic (ROC) curves for the five topmost validated targets between Control and Heart Failure Subtypes at Acute decompensated state. EV indicates Extracellular vesicles; RT-qPCR, Real-Time quantitative PCR; I, Fragment 1; II, Fragment 2; Ctrl, Control; HFpEF, Heart Failure with preserved Ejection Fraction; HFrEF, Heart Failure with reduced Ejection Fraction; dCT, delta CT value.

FIGS. 15A-15K are a series of plots showing EV-derived lncRNAs or their fragments change dynamically and significantly between acute congested to decongested states. (FIG. 15A) Box plots of the differentially expressed targets that were experimentally confirmed on the validation cohort between admission and discharge using RT-qPCR. Significance expressed as * p<0.01 ** p<0.001 ***p<0.0001 where p is calculated using the Kruskal-Wallis test. EV and cellular RNA expression of AC092656.1, lnc-CALML5-7, RMRP, and LINC00989 upon hypoxia and nutrient deprivation (Glucose serum deprivation) in Induced Pluripotent Stem Cell-derived cardiomyocytes (iPSC-CMs; FIGS. 15B-15E) and Human Cardiac Fibroblasts (HCF; FIGS. 15F-15I) assessed using qRT-PCR. Fold change is calculated using the 2-AACt method relative to 5s rRNA in EVs and BACT in cells. Significance is expressed as * p<0.01 ** p<0.001 ***p<0.0001 where p is calculated using ANOVA test. (FIG. 15J) UMAP plot of LINC00989 from single nuclear transcriptomic data showing expression in pericytes. (FIG. 15K) Expression of LINC00989 in normal, DCM, and HCM pericytes from single nuclear transcriptomic data; Expression of LINC00989 in pericyte EVs and pericyte cells subjected to stressors obtained from qRT-PCR as mentioned above. EV indicates Extracellular vesicles: RT-qPCR, Real-Time quantitative PCR; Ctrl, Control; HFpEF, Heart Failure with preserved Ejection Fraction; HFrEF, Heart Failure with reduced Ejection Fraction; HF, Heart Failure; dCT, delta CT values; Hyp, Hypoxia; GSD, Glucose Serum Deprivation.

FIGS. 16A-16D are as a series of schematics and plots showing molecular characterization of EVs isolated from conditioned cell culture media of four types of HER2-positive breast cancer cell lines (BT-474, BT-474R, SK-BR-3, and JIMT-1). Top of FIG. 16A shows the experimental scheme for SEVEN: tetraspanin (TSPAN)-enriched EVs were detected with SEVEN using fluorescently labeled antibodies against TSPANs. The average numbers of detected EVs per region of interest (ROI) per 1 μL of media are reported on bottom. n=3 (15 ROIs); Error bars, SEM; ** p<0.01; *** p<0.001; **** p<0.0001. FIG. 16B shows graphs illustrating diameter and shape (eccentricity) of HER2-enriched and TSPAN-enriched EVs; EVs were detected using fluorescently labeled antibodies against TSPANs. FIG. 16C shows detected molecular densities of TSPANs and HER2 on TSPAN-enriched EVs detected using either fluorescently labeled antibodies against TSPANs (left) or fluorescently labeled trastuzumab (right). For FIGS. 16B and 16C, boxes indicate interquartile ranges, center lines indicate medians, crosses indicate means, and the dots indicate EVs beyond 1.5-times the interquartile range. n=3 (15 ROIs); **** p<0.0001. FIG. 16D shows a plot illustrating the average numbers of detected EVs per ROI detected with SEVEN using fluorescently labeled antibodies against TSPANs. Different EV isolation methods were tested (capto core resin (CC) 700 and 400, PEG precipitation, thrombin treatment, and dilution) using plasma from HER2-positive breast cancer patient, TNBC patient, and healthy pool. n=3 technical replicates (15 ROIs); Error bars, SEM; * p<0.05; ** p<0.01; **** p<0.001: **** p<0.0001.

FIGS. 17A-17B are plots showing molecular characterization of heart-enriched EVs from healthy patients and myocardial infarction (MI) patients using the SEVEN assay. Tetraspanin (TSPAN)- and Nicotinic Acetylcholine Receptor, Epsilon Subunit (CHRNE)-enriched EVs from five healthy patients and five myocardial infarction (MI) patients were isolated and characterized according to the SEVEN assay described herein. For each individual. 10 ROIs were obtained in two independent experiments for both CHRNE and TSPAN captures. FIG. 17A shows plots illustrating differences in EV counts per ROI (left panel), average detected TSPAN content/EV per ROI (middle panel), and average EV diameter per ROI(right panel) of CHRNE- and TSPAN-enriched EVs obtained from healthy and MI patients. FIG. 17B is a plot showing counts of CHRNE-enriched complex EVs per ROI in healthy and MI patients.

FIGS. 18A-18C are schematics, images, and plots showing molecular characterization of brain-enriched EVs using the SEVEN assay and transcriptomic analysis. FIG. 18A (top panel) shows a schematic illustrating the experimental design for capture of Myelin Oligodendrocyte Glycoprotein (MOG)-enriched EVs using coverslips coated with anti-MOG antibodies and untethered, AF647-conjugated anti-TSPAN antibodies. The bottom panel of FIG. 18A shows an SMLM image of two MOG-enriched EVs detected with fluorescent anti-TSPAN antibodies. FIG. 18B shows plots illustrating EV counts per ROI for TSPAN-enriched and MOG-enriched EVs (left panel), diameter of MOG-enriched EVs (middle panel), and detected TSPAN content per EV of MOG-enriched EVs (right panel). FIG. 18C is a scatter plot showing transcriptomic analysis of EVs pulled down with anti-MOG antibodies. A cut-off value of tau>0.9 was used to assess tissue enrichment. Transcripts with fold-change from input >2 and high levels of detection are shown by name, including Syntrophin Gamma 1 (SNTG1), Gamma-Aminobutyric Acid Type A Receptor Subunit Gamma 2 (GABRG2), DLX6 Antisense RNA 1 (DLX6-AS1), Myelin Transcription Factor 1 (MYT1), G-Protein Coupled Receptor 2 (GPR2), Regulator of G Protein Signaling 7 (RGS7), Myelin Transcription Factor 1 Like (MYTIL). Inc-KLHL32-4, and lnc-RWDD3-6.

FIGS. 19A-19B are images and plots showing characterization of EVs using a liver-on-chip culture model in steady state or upon treatment with fatty acids and the SEVEN assay. FIG. 19A shows SMLM images of EVs from hepatocyte (Hep) and non-parenchymal cell (NPC) effluents (scale bar=200 nm). FIG. 19B shows plots of EV count per ROI, average EV diameter per ROI, and average detected TSPAN content per EV per ROI isolated from effluents in steady state or after treatment with fatty acids (FA). Unconditioned control media is included (p<0.0001 between media and all effluents). Error bars show SEM. n=3 chips in technical duplicates.

FIG. 20 shows plots illustrating molecular profiles of HER2-enriched EVs from plasma of breast cancer patients with elevated HER2 expression (HER2; n=4) and breast cancer patients that were triple negative at diagnosis (TNBC; n=3). Number of gated EVs with unique characteristics (size and detected TSPAN content/EV) for the to groups and healthy pooled plasma (n=1) is shown.

FIGS. 21A-21F are images and plots illustrating the application of super-resolution radial fluctuation (SRRF) imaging and data analysis for EV characterization. FIG. 21A shows a processed image with overlap between SMLM (light gray dots in the center of semi-circles) and optimized EV-SRRF in total internal reflection fluorescence (TIRF) illumination mode (large gray and white semi-circles). FIG. 21B is a scatter plot showing a significant positive correlation in EV diameter measured using the SMLM and EV-SRRF modalities. FIG. 21C is a scatter plot showing a significant positive correlation in TSPAN molecule count using the SMLM and EV-SRRF modalities. FIG. 21D shows the distribution of EV sizes and detected TSPAN content/EV between SMLM and EV-SRRF for plasma EVs. FIG. 21E shows comparison of mean values per ROI (diameter, EV counts, TSPAN/EV content) for SMLM and EV-SRRF for plasma EVs. FIG. 21F shows fluorescence images of recombinant EVs (rEVs) imaged in two illumination modes (TIRF or wide field) with two microscopes.

FIGS. 22A-22J are schematics and plots showing analysis of induced pluripotent stem cell (iPSC)-derived cardiomyocytes (iPSC-CMs) and their EVs, highlighting the enrichment of POPDC2 and CHNRE in iPSC-CM-derived EVs. (FIG. 22A) LC/MS (Liquid Chromatography-based Mass Spectrometric) analysis workflow confirmed the presence of POPDC2 and CHRNE in iPSC-CM-derived EVs, identifying them as markers of cardiomyocytes. (FIG. 22B) Bar graph showing relative protein abundance of POPDC2 obtained using proteomic analysis of iPSC-CMs (“Cell”) and their derived EVs (“EV”). (FIG. 22C) Western blot assays verified the enrichment of POPDC2 and CHRNE in both iPSC-CMs and EVs. (FIG. 22D) UMAP (Uniform Manifold Approximation and Projection) legend. (FIG. 22E) Plot showing tissue-wise POPDC2 enrichment data from Genotype Tissue Expression (GTEx) tissue bank. (FIG. 22F) UMAP from single-nuclear RNA (snRNA) sequencing atlas from patient dataset demonstrating POPDC2 expression across tissues and cellular levels in the heart, respectively. (FIG. 22G) Plot showing tissue-wise CHRNE enrichment. (FIG. 22H) UMAP of CHRNE expression across tissues and cellular levels in the heart, respectively. (FIG. 22I-22J) Plots showing flow cytometry data demonstrating co-localization of CHRNE and POPDC2 in double-positive populations of iPSC-CM—(FIG. 22H) and human plasma-derived EVs (FIG. 22I).

FIGS. 23A-23C are schematics and plots showing cardiac-specificity of POPDC2 and CHRNE expression using a cardiac-specific Cre-driven EXOMAP mouse model. (FIG. 23A) Schematic illustrating the creation of a transgenic exomap1 transgenic mouse model expressing HsCD81mNG (humanized CD81 fused with mNeonGreen) in a Cre-recombinase-dependent manner. (FIG. 23B) Schematic illustrating an overview of the process of using biotin-streptavidin affinity and Streptavidin Magnetic Beads for EV immunocapture (ExoCapture-MSB method) of cardiac-tissue specific EVs derived from αMHC-Cre ExomapI mouse. (FIG. 23C) Western blot (WB) assay confirming the enrichment of CHRNE and Troponin, a cardiac-specific protein, in the HsCD81-positive EVs derived from the heart captured using the ExoCapture-MSB method from the Cardiac-Specific EV EXOMAP mouse model.

FIGS. 24A-24M are schematics and plots showing plasma transcriptomic analysis of POPDC2 and CHRNE EVs (cardiovesicles) using ExoCapture-MSB. (FIG. 24A) Schematic illustrating an overview of the ExoCapture-MSB method for isolation of cardiac-derived extracellular vesicles (Cardiovesicles) from 500 μL of human plasma. (FIG. 24B) Western blot analysis of POPDC2 immunocapture of Cardiovesicles showing enrichment for cardiac protein such as Troponin. (FIG. 24C) Scatter plot showing transcriptomic enrichment of heart-specific transcripts (prioritized by tau score) in the EVs immunocaptured with biotinylated POPDC2 antibody. (FIG. 24D) Representative box plots showing the transcriptomic enrichment of individual transcripts immunocaptured with CD81 and POPDC2, confirming the specific capture and analysis of Cardiovesicles. (FIG. 24E) Western blot analysis of CHRNE immunocapture of Cardiovesicles showing enrichment for cardiac protein such as Troponin. (FIG. 24F) Scatter plot showing transcriptomic enrichment of heart-specific transcripts (prioritized by tau score) in the EVs immunocaptured with biotinylated CHRNE antibody. (FIG. 24G) Representative box plots showing the transcriptomic enrichment of individual transcripts immunocaptured with CD81 and CHRNE, confirming the specific capture and analysis of Cardiovesicles. (FIGS. 24H-24K) Plots showing tissue-wise enrichment of BMP10 (FIG. 2411), FBXO40 (FIG. 24I), LRRC10 (FIG. 24J), and TNNI3 (FIG. 24K). (FIGS. 24L, 24M) UMAP analysis (from snRNA sequencing) showing BMP10, FBXO40 (FIG. 24L), LRRC10, and TNNI3 (FIG. 24M) as highly cardiac-enriched transcripts within Cardiovesicles, confirming their cardiac origin.

FIGS. 25A-25I are schematics and plots illustrating heart enrichment of transcripts from Cardiovesicles using a single cell atlas. (FIG. 25A) Schematic plan to isolate and analyze the plasma Cardiovesicles from cardiovascular cohorts (90 samples) using ExoCapture-MSB, Cardiovesicle exRNA sequencing, snRNA sequence mapping, and differential gene expression analysis from control patients, patients with heart failure (HF), and myocardial infarction patients (MI). The most consistently abundant transcripts immunocaptured using POPDC2 and CHRNE from control patients were identified and mapped onto the multiorgan single cell transcriptomic atlas dataset (Tabula Sapiens) (FIG. 25D and FIG. 25G, respectively; UMAP legend in FIG. 25B) and a single-nuclear dataset of human dilated and hypertrophic cardiomyopathy (FIGS. 25E, 25F, 25H, and 25I). Summary dotplots shown in FIGS. 25E, 25H and individual target UMAPs shown in FIGS. 25F, 25I (UMAP legend in FIG. 25C).

FIGS. 26A-26L are plots showing analysis of cardiovascular transcripts immunocaptured with POPDC2 and CHRNE. (FIGS. 26A-26F) Plots showing transcriptomic analysis of Cardiovesicles from heart failure patients, including POPDC2-captured Cardiovesicles (FIGS. 26A-26C) and CHRNE-captured Cardiovesicles (FIGS. 26D-26F). (FIGS. 26G-26L) Plots showing transcriptomic analysis of Cardiovesicles from myocardial infarction patients, including POPDC2-captured Cardiovesicles (FIGS. 26G-261) and CHRNE-captured Cardiovesicles (FIGS. 26J-26L).

FIGS. 27A-27L are plots and heatmaps showing differentially expressed genes in Cardiovesicles from heart failure and myocardial infarction patients as compared to control patients. (FIG. 27A) Heatmap and principle component analysis (PCA) showing gene expression levels in POPDC2 Cardiovesicles from heart failure patients. (FIG. 27B) Scatter plot showing principle component 2 (PC2) plotted against principle component 1 (PC1) between control and heart failure patients. (FIG. 27C) Box plots showing differentially expressed transcripts in POPDC2 Cardiovesicles from control and heart failure patients. (FIG. 27D) Heatmap and PCA showing gene expression levels in CHRNE Cardiovesicles from heart failure patients. (FIG. 27E) Scatter plot showing PC2 plotted against PC1 between control and heart failure patients. (FIG. 27F) Box plots showing differentially expressed transcripts in CHRNE Cardiovesicles from control and heart failure patients. (FIG. 27G) Heatmap and PCA showing gene expression levels in POPDC2 Cardiovesicles from myocardial infarction patients. (FIG. 27H) Scatter plot showing PC2 plotted against PC1 between control and myocardial infarction patients. (FIG. 271) Box plots showing differentially expressed transcripts in POPDC2 Cardiovesicles from control and myocardial infarction patients. (FIG. 27J) Heatmap and PCA showing gene expression levels in CHRNE Cardiovesicles from myocardial infarction patients. (FIG. 27K) Scatter plot showing PC2 plotted against PC1 between control and myocardial infarction patients. (FIG. 27L) Box plots showing differentially expressed transcripts in CHRNE Cardiovesicles from control and myocardial infarction patients. Together, these data are consistent with the enrichment of cardiomyocyte and cardiac specific transcripts in cardiovesicles derived with POPDC2 and CHRNE immuno-pulldown in control. MI or heart failure patients. Specific transcripts differentiate these different cardiovascular disease categories and support the claim that cardiovesicles provide a high-fidelity representation akin to a liquid biopsy for cardiovascular diseases.

### DETAILED DESCRIPTION

Disclosed herein, in certain embodiments, are methods for isolation, detection, and analysis of extracellular vesicles (EVs) derived from a target tissue of origin using a combination of affinity capture, super-resolution microscopy, proteomic analysis, and sequencing analysis. Such methods are advantageous for the production of proteomic and transcriptomic profiles of target tissues of origin, and use of such profiles to diagnose or prognose a variety of human diseases. The disclosed methods are useful for identification of one or more biomarkers associated with a disease or disorder in a subject (e.g., a human), assessment of therapeutic efficacy of therapeutic agents in the treatment of a disease or a disorder, and for selection of a therapeutic agent for the treatment of a particular disease or disorder. Furthermore, the disclosed methods provide additional advantages for isolation, detection, and analysis of extracellular vesicles, including use of very small sample volumes (e.g., as little as 0.08 μL of a biofluid sample), high sensitivity, high signal-to-noise ratio, capacity to count individual molecules (e.g., proteins) on the surface of EVs, elimination of highly invasive biopsy procedures to isolate EVs from a target tissue of origin, ease of imaging via functionalization and staining of EVs directly on coverslips, and elimination of requirement for EV enrichment, among others.

### Extracellular Vesicles (EVs)

Extracellular vesicles (EVs) are small (e.g., 20 nm to 10 μm in diameter) lipid-bilayer particles that are naturally released by all known cell types. EVs are generally categorized by size and route of generation. For example, exosomes are produced in the endosomal compartment and range in size from about 20 nm to 150 nm in diameter. Microvesicles are released from the cell membrane and typically range from 30 nm to 1 μm in diameter. Apoptotic bodies are produced during final stages of apoptosis, are generally the some of largest EVs, and include phosphatidylserine in the outer bilayer of the vesicle. Large oncosomes (Los) are micron-sized EVs that are produced by cancer cells and may be as large as 20 μm in diameter. Furthermore, EVs have been associated with a variety of biological functions, including, without limitation, disposal of cellular waste, intercellular communication via transfer of intravesicular cargo between cells, molecular recycling, formation of metastatic niche, cellular pathfinding, quorum sensing, among others.

EVs have molecular cargo that may include proteins, lipids, nucleic acids, metabolites, and/or organelles that reflect their cellular origin. Changes of these molecules in the cells of origin may reflect disease etiology or status (e.g., progression, remission, etc.) or response to therapy. Such changes in EV content can be monitored by measuring their levels in biofluids that are obtained using minimally invasive methods (e.g., liquid biopsy of whole blood, plasma, serum, cerebrospinal fluid, saliva, sputum, nasal secretion, ocular secretion, cystic fluid, synovial fluid, bronchoalveolar lavage fluid, amniotic fluid, bone marrow aspirate, bile, milk, stool, swab, smear, semen, or urine). This feature of EVs poises them to serve as powerful diagnostic, prognostic, and/or theranostic tools. Common methods for assaying EVs include population-level analysis, such as measurement of concentration of cargo of a particular type within a fraction of EVs. Single particle analysis commonly involves methods that study individual EVs on the basis of, e.g., size, surface protein expression, cargo content, zeta potential, etc. Various guidelines have been established for the isolation, detection and analysis of EVs, including, e.g., Minimal Information for Studies of Extracellular Vesicles (MISEV).

One major drawback of currently available methods for analysis of EV biomarkers is the lack of tractable tools and reagents to measure cell or tissue-specific biomarkers in EVs. The present disclosure provides methods and reagents for analysis of EVs, such methods embodied in an assay that combines affinity isolation of EVs on functionalized coversheets, super-resolution microscopy, molecular counting, proteomic analysis, and transcriptomic analysis. The sections that follow describe the assay in greater detail.

### Single Extracellular Vesicle Nanoscopy (SEVEN) Assay

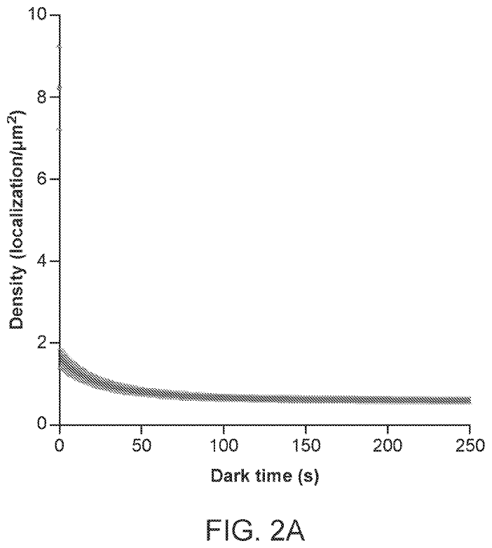

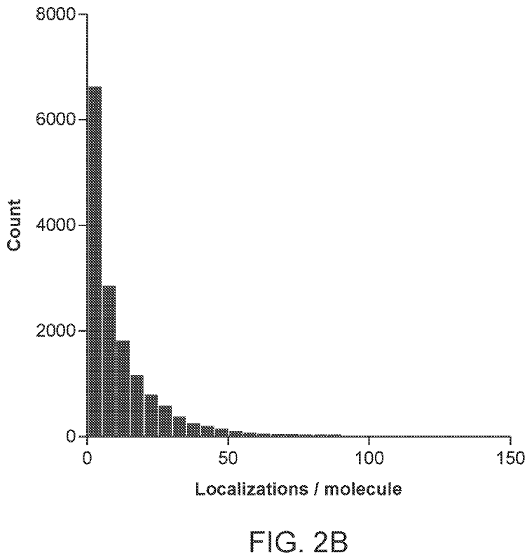

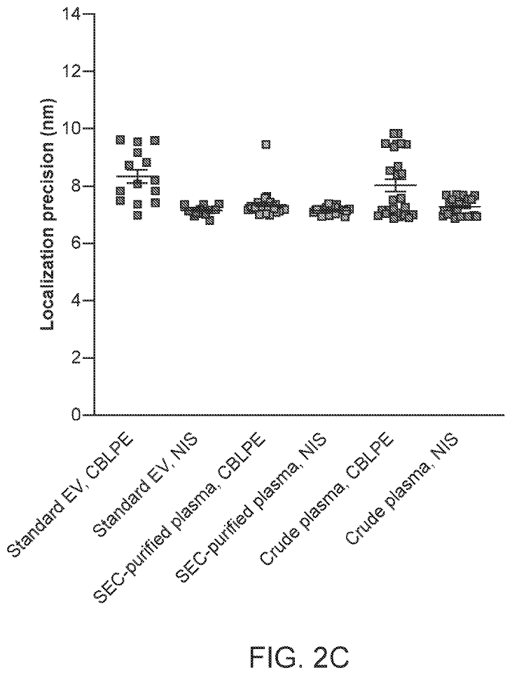

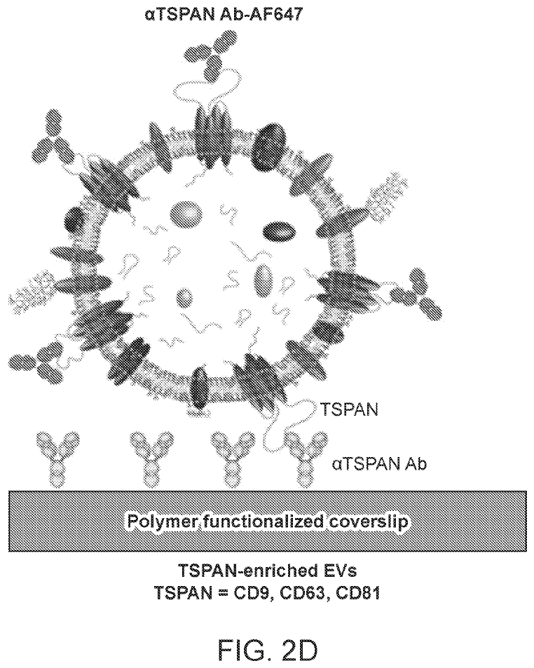

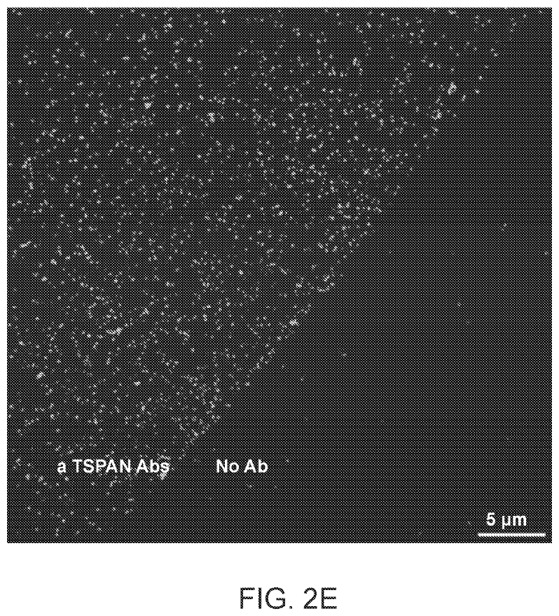

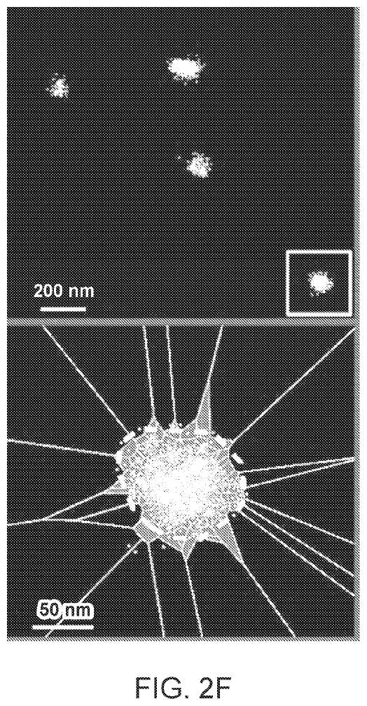

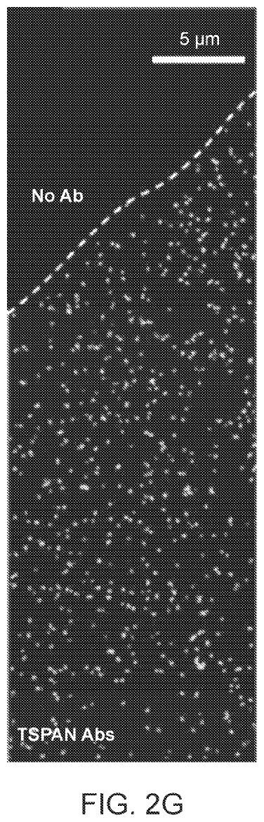

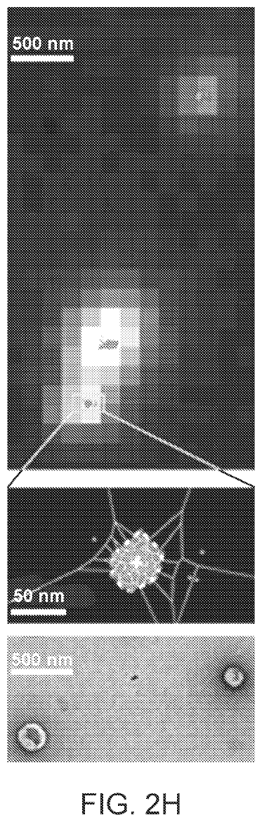

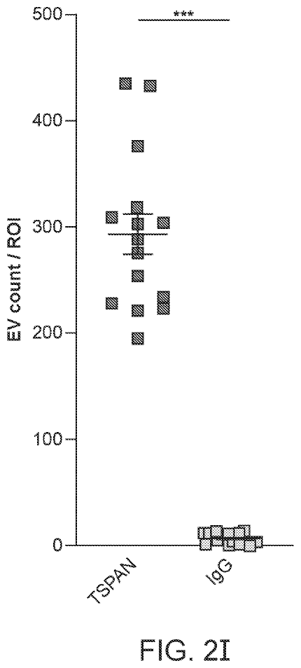

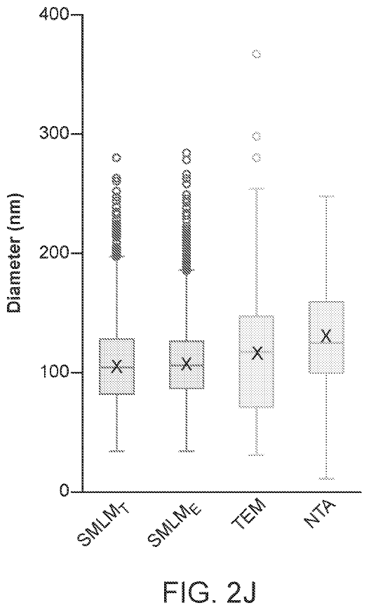

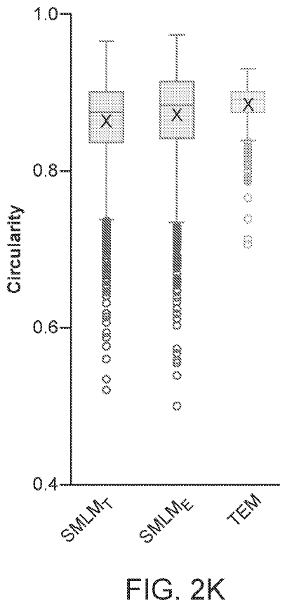

The present invention is based, in part, on the development of a highly sensitive assay for EV detection and characterization. This assay, named Single Extracellular Vesicle Nanoscopy (SEVEN), includes seven steps, namely: (1) Photophysical characterization of EVs using fluorescent probes used for EV staining, which allows efficient molecular counting; (2) functionalization (e.g., coating) of coverslips with a polymer having dense N-hydroxysuccinimide (NHS) groups: (3) use of a mixture of anti-tetraspanin antibodies (Abs: e.g., anti-CD9, anti-CD63, and anti-CD81 antibodies: anti-TSPAN) to covalently attach to polymer-coated coverslips and capping unreacted NHS groups: (4) affinity capture of EVs onto the functionalized coverslips; (5) staining of affinity captured EVs with a mixture of fluorescently labeled anti-TSPAN antibodies (see FIG. 2C); (6) imaging of EVs using super-resolution microscopy methods, including but not limited to single-molecule localization microscopy (SMLM) and super-resolution radial fluctuations (SRRF) imaging; and (7) data analysis using Voronoi tessellation-based algorithm.

### Photophysical Characterization of Fluorescent Reporters

Disclosed herein, in certain embodiments, are methods for detection of biomarkers (e.g., proteins, lipids, nucleic acids, and/or carbohydrates) expressed on EVs using fluorescent probes, which exhibit photophysical properties that are advantageous for specific application in single-molecule detection experiments. In certain embodiments, the biomarker is a protein. In certain embodiments, the biomarker is a lipid. In certain embodiments, the biomarker is a nucleic acid. In certain embodiments, the biomarker is a carbohydrate. Photophysical properties of fluorescent probes may include, without limitation, average number of localizations per individual fluorescent probe and maximum dark time. These parameters can be assessed using known methods, such as surface assay for molecular isolation (SAMI), which can be used in conjunction with quantitative single molecule localization microscopy (qSMLM), qSMLM relies on total internal reflection illumination to excite photosensitive molecules. Images generated with qSMLM require rigorous quantitative analysis to assess protein organization and molecular density. Common fluorescent reporters used with qSMLM include optical highlighter proteins and photoswitchable dyes, which exhibit complex photophysical characteristics. These reporters oscillate between dark and fluorescent states in a pattern that is highly dependent on the molecular structure of the fluorophore, its stoichiometry with the binding agent to which it is conjugated (e.g., a binding polypeptide, such as an antibody), and imaging conditions.

In a typical SAMI protocol, fluorescent reports are isolated on a functionalized surface (e.g., coverslip), e.g., by covalently and sparsely attaching target proteins to the surface, and affinity labeling the target proteins with fluorescent reporters. In certain embodiments, the fluorescent reporter is an antibody conjugated to a fluorescent reporter. In certain antibodies, the antibody is an anti-tetraspanin (anti-TSPAN) antibody, including but not limited to anti-CD9, anti-CD63, and/or anti-CD81 antibodies. In certain embodiments, the fluorescent reporter is selected from the group consisting of AF532, AF488, AF532, AF555, AF568, AF594, AF647, AF680, AF700, AF750, Atto488, Atto532, Atto647N, Atto680, Atto700, CF532, CF555, CF568, CF647, CF660C, CF680, CF750, CF488A, CF583R, CF597R, CF680R, CF535ST, Cy3, Cy3b, Cy5, DY-634, DyLight650, Dronpa, JF549, JF646, JFX549, JFX554. JFX646, JFX650, mIrisFP, mMaple, mMaple 3. PAGFP, PamCherry, PATagRFP. PamKate, PS-CFP2, and quantum dots. Photophysical characterization of fluorescent reporters can be summarized using known methods, e.g., by plotting localization density as a function of dark time, measuring average number of localizations, measuring photoactivation efficiency, etc.

### Functionalization of Surfaces for EV Binding

As discussed herein, the SEVEN assay is based, in part, on isolation, detection, and analysis of EVs from a sample (e.g., a biofluid or tissue sample) obtained from a subject (e.g., a human) on a surface (e.g., glass coverslip) functionalized with covalently-linked binding agents (e.g., proteins, such as antibodies). In certain embodiments, functionalization of a coverslip includes, without limitation: (1) preparation of the coverslip surface for functionalization via attachment of a coupling agent (e.g., a linker moiety) to the coverslip; and (2) attachment of a binding agent, such as a binding protein (e.g., one or more antibodies), to the linker-covered surface.

In certain embodiments, preparation of a coverslip includes, without limitation: (1) providing a clean glass coverslip; (2) activating the coverslip (e.g., via treatment with concentrated hydrochloric acid (HCl)) for a time sufficient to activate the coverslip (e.g., about 10 minutes): (3) washing the activated coverslips, e.g., with deionized water, to remove residual HCl; (4) treating the surface with a solution containing a coupling agent (e.g., a linker moiety); (5) washing the linker-coated coverslip, e.g., with deionized water, to remove residual solution containing the coupling agent (e.g., a linker moiety): (6) curing or drying the linker-coated coverslips at a suitable temperature (e.g., between 80° C. and 90° C.) and for a sufficient time (e.g., about 15 minutes) and allowing the cured/dried coverslips to cool to room temperature; and, optionally, (7) storing the coverslips at a suitable temperature (e.g., about −80° C.) under conditions that minimize exposure to moisture. In certain embodiments, the coupling agent comprises one or more N-hydroxysuccinimide (NHS)-ester residues. In certain embodiments, the linker agent is selected from the group consisting of MCP2, MCP4, p-aminophenyltrimethoxysilane (APTMS), and aminotrimethoxy silane (ATMS).

In certain embodiments, functionalization of the linker-covered surface (e.g. coverslip) with a binding agent includes, without limitation, the following steps: (1) spotting a solution containing the binding agent (e.g., one or more antibodies) and a diluent (e.g., 1% glycerol solution in NaCl solution) on the coverslip in a suitable volume (e.g., 0.5 μL) and incubating the spotted coverslip under suitable conditions (e.g., room temperature) and for a sufficient time (e.g., 4 hours); (2) washing the spotted coverslip following incubation; and (3) deactivating NHS-ester residues with amine-containing blocking solution and blocking free surface areas with a blocking buffer. In certain embodiments, the binding agent is an antibody. In certain embodiments, the antibody is an anti-TSPAN antibody selected from the group consisting of anti-CD9, anti-CD63, and anti-CD81 antibodies.

### EV-Specific Affinity Capture Agents

The disclosed methods provide various affinity capture agents that are suitable for use in in conjunction with the disclosed assays (e.g., for binding to EVs). Non-limiting examples of affinity capture agents of the disclosure include proteins, peptides, aptamers, carbohydrates, and combinations thereof. In certain embodiments, the affinity capture agent is a protein. In certain embodiments, the affinity capture agent is a peptide. In certain embodiments, the affinity capture agent is an aptamer. In certain embodiments, the affinity capture agent is a carbohydrate. In certain embodiments, the carbohydrate is a lectin. In certain embodiments, the protein or peptide is selected from the group consisting of a primary antibody, secondary antibody, Fab, F(ab′), F(ab′)2, single chain variable fragment (scFv), Fd, minibody, variable heavy domain, variable light domain, variable NAR domain, single chain binding polypeptide, dAb fragment, nanobody, VHH, and toxin. In certain embodiments, the EV-specific capture agent is an anti-TSPAN antibody selected from the group consisting of anti-CD9, anti-CD63, and anti-CD81 antibodies. In certain embodiments, the EV-specific capture agent is an antibody that specifically binds to a tissue-specific biomarker present on the surface of the EV. In certain embodiments, the affinity capture agent comprises (e.g., is conjugated to) a fluorescent reporter.

### Affinity Capture of EVs on Functionalized Surfaces

Upon functionalization of a coverslip according to the methods described above, the surface can be used for binding to EVs. In certain embodiments, EVs are obtained from a sample (e.g., a biofluid or tissue sample) using conventional methods. In certain embodiments, the biofluid sample is selected from the group consisting of whole blood, plasma, serum, cerebrospinal fluid, saliva, sputum, nasal secretion, ocular secretion, cystic fluid, synovial fluid, bronchoalveolar lavage fluid, amniotic fluid, bone marrow aspirate, bile, milk, stool, swab, smear, semen, or urine. In certain embodiments, the tissue sample is selected from the group consisting of formalin-fixed paraffin-embedded (FFPE) tissue block, fixed tissue, fresh tissue, or frozen tissue. In certain embodiments, the sample is obtained from a control subject (e.g., a healthy subject). In certain embodiments, the sample is obtained from a test subject (e.g., a subject having or at risk of developing a disease or disorder.

In certain embodiments, the method includes isolating a mixed population of EVs (MEVs) from a crude sample prior to being bound to the functionalized surface in order to produce an enriched population of MEVs. In certain embodiments, the MEVs are isolated from the crude sample using affinity capture, size exclusion chromatography (SEC), ultracentrifugation, ultrafiltration, flow field-flow fractionation, hydrostatic filtration dialysis, enzyme-linked immunosorbent assay (ELISA) density gradient, immunoprecipitation, polyethylene glycol (PEG) precipitation, PEG/dextran aqueous two phase system (ATPS) isolation, lectin-induced agglutination, acoustic nanofilter, and/or a microfluidic separation. In certain embodiments, the MEVs are isolated from the crude sample using SEC (e.g., using a 70 nm SEC column). In certain embodiments, the MEVs are isolated from a crude sample using an affinity capture method selected from the group consisting of affinity chromatography, flow-based affinity immunoassay, affinity pulldown, affinity bead capture, affinity resin capture, microfluidic affinity capture, ELISA, magneto-immunoprecipitation, mixed-mode chromatography (MMC), and membrane-affinity spin column (MASC). In certain embodiments, isolation of MEVs from the crude sample is performed using an affinity capture agent that specifically binds to one or more EV-specific biomarkers selected from the group consisting of CD9, CD63, and CD81. In certain embodiments, the method further includes isolating a population of tissue-specific EVs (TSEVs) from the MEVs prior to binding the TSEVs to the functionalized coverslip. In certain embodiments, the method does not include producing the enriched population of MEVs. In certain embodiments, the coverslip is functionalized with an affinity capture agent that specifically binds to an EV-specific biomarker (e.g., one or more antibodies selected from an anti-CD9, anti-CD63, and anti-CD81 antibody) or an affinity capture agent that specifically binds to a tissue-specific biomarker (e.g., an antibody that specifically binds to the tissue-specific biomarker).

In certain embodiments, binding of isolated EVs (e.g., MEVs and/or TSEVs) to the functionalized coverslips includes, without limitation, the following steps: (1) incubation of the EVs on the coverslip under suitable conditions (e.g., room temperature, rocking shaker) and for a sufficient time (e.g., 14-17 hours); and (2) washing the coverslip following EV incubation.

### EV Staining

In certain embodiments, the disclosed methods further include a step of labeling EVs bound to functionalized coverslips, e.g., with a reporter molecule. In certain embodiments, the reporter molecule is a fluorescent reporter. In certain embodiments, the fluorescent reporter is conjugated to an antibody. In certain embodiments, the antibody is selected from the group consisting of an anti-CD9, anti-CD63, and anti-CD81 antibody. In certain embodiments, the antibody is an antibody that specifically binds to a tissue-specific biomarker. In certain embodiments, the fluorescent reporter is a photoswitchable fluorescent reporter, photoactivatable fluorescent reporter, photoconvertible fluorescent reporter, spontaneously blinking fluorescent reporter, or temporarily binding fluorescent reporter. In certain embodiments, the fluorescent reporter is selected from the group consisting of AF532, AF488. AF532, AF555, AF568. AF594. AF647, AF680, AF700, AF750. Atto488. Atto532, Atto647N, Atto680, Atto700, CF532, CF555, CF568, CF647, CF660C, CF680, CF750, CF488A, CF583R, CF597R, CF680R, CF535ST, Cy3, Cy3b, Cy5, DY-634, DyLight650, Dronpa, JF549, JF646, JFX549, JFX554, JFX646, JFX650, mIrisFP, mMaple, mMaple 3, PAGFP, PamCherry. PATagRFP, PamKate. PS-CFP2, and quantum dots. In certain embodiments, the functionalized coverslip bound to the EVs is washed subsequent to labeling. In certain embodiments, the functionalized coverslip bound to the EVs is treated with a fixative (e.g., 4% paraformaldehyde and 0.2% glutaraldehyde).

### Super-Resolution Microscopy

As is discussed herein, the SEVEN assay is based, in part, on combining affinity capture with super-resolution microscopy for the isolation, detection, and analysis of EVs using the disclosed methods. Super-resolution microscopy generally refers to a collection of optical microscopy techniques designed to surpass the diffraction limit, which is a limit in being able to resolve features of an imaged object that are less than a few hundred nanometers in size because such features become comparable or smaller than the physical wavelength of the light used to illuminate the object. When this occurs, features of an object cannot be resolved due to the diffraction of light when it passes through a small aperture or is focused to a tiny spot. Formally defined, the diffraction limit is the distance that two point-source objects have to be separated to be able to distinguish the objects from one another. According to the definition of the diffraction limit set forth by Ernst Abbe, the diffraction limit is equal to 0.5k/NA, where, is the wavelength of light and NA is the numerical aperture of the object lens that collects light. Some super-resolution microscopy techniques involve moving higher spatial frequencies of light that may be unresolvable to lower spatial frequencies that may be resolved.

Certain super-resolution microscopy techniques can generate images having a resolution that surpasses the diffraction limit using fluorescent probes that can be activated and de-activated. By selectively, or randomly, activating targeted probes and detecting their fluorescence, these super-resolution techniques can be configured to distinguish emissions from two molecules that are located within a diffraction-limited range. Generally described, these super-resolution microscopy methods involve switching fluorophores between light and dark states, combined with spatial illumination schemes to isolate the switching behaviors in sub-diffraction areas. The disclosed methods allow for capturing one or more images of the light and localizing the light-emitting particles using one or more single molecule microscopic methods.

Non-limiting examples of super-resolution microscopy techniques suitable for use in conjunction with the disclosed methods include single-molecule localization microscopy (SMLM), such as quantitative SMLM (qSMLM), qSMLM is a super-resolution fluorescence microscopy approach that can achieve single-molecule sensitivity, nanoscale resolution, and robust molecular counting. Notably, compared to other techniques for single EV characterization, qSMLM has superior sensitivity; this property is essential for the detection of=EV populations that are in low abundance. In certain embodiments, the SMLM is photo-activated localization microscopy (PALM). In certain embodiments, the SMLM is stochastic optical reconstruction microscopy (STORM). In certain embodiments, the STORM is direct STORM (dSTORM). STORM microscopy employs a photochemical switching mechanism to induce on/off transitions. For example, STORM microscopy is a type of super-resolution optical microscopy technique that is based on stochastic switching of single-molecule fluorescence signals. STORM utilizes fluorescent probes that can switch between fluorescent and dark states and the microscopy system can excite an optically resolvable fraction of the fluorophores. Because only a fraction of the fluorophores is excited, the microscopy system can determine the positions of the fluorophores with relatively high precision based on the center positions of the detected fluorescent signals. With multiple snapshots of the sample, each capturing a subset of the fluorophores based on the patterned illumination described herein, a final super-resolution image can be reconstructed from the accumulated positions. In certain embodiments, the SMLM is point accumulation in nanoscale topography (PAINT). PAINT is a super-resolution microscopy technique that uses fast and transient dyes to capture multiple fluorescence points simultaneously by relying on stochastic binding properties of a fluorescent probe.

In certain embodiments, the disclosed super-resolution microscopy methods include super-resolution radial fluctuations (SRRF) analysis. SRRF is an analytical approach that analyzes a sequence of images to produce a super-resolution image without the need for fluorophore detection and localization. SRRF is based on the analysis of fluctuation in radial symmetry throughout image frames, using the assumption that the point spread function (PSF) of fluorescent probes possesses higher radial symmetry as compared to the background. Exemplary applications of these methods include, without limitation, those that perform optical detection of molecular probes that interact with targets. e.g., antibodies, such as antibodies functionalized on a coverslip for use in capturing EVs obtained from a sample (e.g., biofluid or tissue sample).

In certain embodiments, the devices, methods, and systems used for super-resolution imaging may be any suitable imager including but not limited to a charge coupled device (CCD), electron multiplying charge coupled device (EMCCD), camera, and complementary metal-oxide-semiconductor (CMOS) imager. In certain embodiments, the devices, methods, and systems of the disclosure employ light-emitting diode (LED)-based imaging systems. In certain embodiments, the devices, methods, and systems of the disclosure employ laser-based imaging systems. In certain embodiments, the devices, methods, and systems of the disclosure employ wide-field microscopy. In certain embodiments, the devices, methods, and systems of the disclosure employ confocal microscopy. In certain embodiments, the devices, methods, and systems of the disclosure may be any suitable spectral filtering element including but not limited to a dispersive element, transmission grating, grating, band-pass filter or prism. In certain embodiments, the devices, methods, and systems used for super-resolution imaging may use any light source suitable for spectroscopic super-resolution microscopic imaging, including but not limited to a laser, laser diode, visible light source, ultraviolet light source or infrared light source, super-luminescent diodes, continuous wave lasers or ultrashort pulsed lasers.

Generally, the wavelength range of one or more beams of light may range from about 500 nm to about 620 nm. In certain embodiments, the wavelength may range between 200 nm to 600 nm. In certain embodiments, the wavelength may range between 300 to 900 nm. In certain embodiments, the wavelength may range between 500 nm to 1200 nm. In certain embodiments, the wavelength may range between 500 nm to 800 nm. In certain embodiments, the wavelength range of the one or more beams of light may have wavelengths at or around 500 nm. 510 nm, 520 nm. 530 nm, 540 nm, 550 nm, 560 nm, 570 nm. 580 nm, 590 nm, 600 nm, 610 nm, and 620 nm. Generally, the wavelength range of the one or more beams of light may range from 200 nm to 1500 nm. In certain embodiments, the wavelength range of the one or more beams of light may range from 200 nm to 1500 nm. The wavelength range of the one or more beams of light may range from 300 nm to 1500 nm. The wavelength range of the one or more beams of light may range from 400 nm to 1500 nm. The wavelength range of the one or more beams of light may range from 500 nm to 1500 nm. The wavelength range of the one or more beams of light may range from 600 nm to 1500 nm. The wavelength range of the one or more beams of light may range from 700 nm to 1500 nm. The wavelength range of the one or more beams of light may range from 800 nm to 1500 nm. The wavelength range of the one or more beams of light may range from 900 nm to 1500 nm. The wavelength range of the one or more beams of light may range from 1000 nm to 1500 nm. The wavelength range of the one or more beams of light may range from 1100 nm to 1500 nm. The wavelength range of the one or more beams of light may range from 1200 nm to 1500 nm. The wavelength range of the one or more beams of light may range from 1300 nm to 1500 nm. The wavelength range of the one or more beams of light may range from 1300 nm to 1500 nm. In certain embodiments, spectroscopic super-resolution microscopic imaging devices, methods, and systems of the present disclosure include two or more beams of light with wavelengths in the visible light spectrum or the near infrared (NIR) light spectrum. In certain embodiments, spectroscopic super-resolution microscopic imaging includes beams of light with wavelengths in the visible light spectrum, ultraviolet (UV) or the NIR spectrum. Those of skill in the art will appreciate that the wavelength of light may fall within any range bounded by any of these values (e.g., from about 200 nm beam to about 1500 nm).

In certain embodiments, spectroscopic super-resolution microscopic imaging may include multi-band scanning. In certain embodiments, a band may include one or more wavelength ranges containing continuous wavelengths of light within a bounded range. In certain embodiments, a band may include one or more wavelength ranges containing continuous group of wavelengths of light with an upper limit of wavelengths and a lower limit of wavelengths. In certain embodiments, the bounded ranges within a band may include the wavelength ranges described herein. In certain embodiments, spectroscopic super-resolution microscopic imaging may include bands that overlap. In certain embodiments, spectroscopic super-resolution microscopic imaging may include bands that are substantially separated. In certain embodiments, bands may partially overlap. In certain embodiments, spectroscopic super-resolution microscopic may include one or more bands ranging from 1 band to 100 bands. In certain embodiments, the number of bands may include 1-5 bands. In certain embodiments, the number of bands may include 5-10 bands. In certain embodiments, the number of bands may include 10-50 bands. In certain embodiments, the number of bands may include 25-75 bands. In certain embodiments, the number of bands may include 25-100 bands. Those of skill in the art will appreciate that the number of bands of light may fall within any range bounded by any of these values (e.g., from about 1 band to about 100 bands). In certain embodiments, a frequency of light of one or more beams of light, or bands used in spectroscopic super-resolution microscopic imaging may be chosen based on the absorption-emission bands known for a target. In certain embodiments, a wavelength or wavelengths of light may be chosen such that those wavelengths are within the primary absorption-emission bands known or thought to be known for a particular target.

In certain embodiments, spectroscopic super-resolution microscopic may be performed with a range of 1-100,000,000 images generated for resolving one or more one or more non-diffraction limited images. In certain embodiments, n images generated may range from 100-100,000.000. In certain embodiments, n images generated may range from 1000-100,000,000. In certain embodiments, n images generated may range from 1-100,000,000. In certain embodiments, n images generated may range from 100,000-100,000,000. In certain embodiments, n images generated may range from 1,000,000-100,000,000. In certain embodiments, n images generated may range from 10,000,000-100,000,000. In certain embodiments, n images generated may range from 1-100,000. In certain embodiments, n images generated may range from 1-20,000. In certain embodiments, n images generated may range from 1,000-10,000. In certain embodiments, n images generated may range from 50,000-100,000. In certain embodiments, n images generated may range from 100,000-5,000.000. In certain embodiments, n images generated may range from 1,000,000-100,000,000. In certain embodiments, n images generated may range from 10,000,000-50,00,000. In certain embodiments, n images generated may be at least about 1, 1000, 10,000, 20,000, 50,000, 100,0000, 1,000,000, 10,000,000, or 100,000,000. In certain embodiments, n images generated may be at most about 1, 1000, 10,000, 20,000, 50,000, 100,0000, 1,000,000, 10,000,000, or 100,000,000. Those of skill in the art will appreciate that n images generated may range from 1-100,000,000 images.

### Data Analysis

Disclosed herein, in certain embodiments, are methods for data processing and image analysis for data generated using the aforementioned super-resolution microscopy techniques. Various techniques are known for analysis of super-resolution images. The disclosed analyses may be used to assess the size, shape, spatiotemporal distribution, internal cargo, surface proteins, as well as other phenotypic features of EVs. Furthermore, the disclosed analyses may be used to characterize photophysical properties of fluorescent probes utilized in the methods of the disclosure, including but not limited to the (average) number of localizations of fluorescent signals, peak shape, peak width, and maximum dark time, and other attributes of spectral information. In certain embodiments, single molecule localization methods may be chosen based on the density of the spacing of the data obtained. In certain embodiments, emission spots may be located through the method of iteratively fitting multiple point spread functions (PSFs) to regions of image data which appear to contain overlapping signals. In certain embodiments, the emission spots may be located using compressed sensing. An example of compressed sensing includes: extracting emission spot co-ordinates from potentially overlapping image data by first calculating the expected image from each possible emission spot position; and determining the emission spot positions that give rise to real signals in light of this complete prior knowledge. In certain embodiments, emission spots may be identified if their diameters match the diameter of the expected PSF of the collection optics. The expected PSF may be calculated or may be determined by experiment. Spots may be determined to have diameters that match the expected PSF if they are equal to the expected diameter or vary from the expected diameter by less than a threshold value. The threshold value may be based on the expected standard deviation of the PSF. The threshold value may be adjusted iteratively. Identification of emission spots may further include selecting an axial focus of the images by suitably selecting the PSF diameter and/or threshold value.

Non-limiting examples of further useful techniques include Voronoi tessellation, Extracellular Vesicle Spatial Clustering of Applications with Noise (EVSCAN), density-based spatial clustering of applications (DBSCAN), pair-correlation cluster analysis, and clustering methods, which are routinely used for analysis of super-resolution images. Microscopy images are also amenable to unsupervised analyses using machine learning algorithms. In some embodiments, the machine learning algorithm comprises an unsupervised machine learning algorithm. In some embodiments, the unsupervised machine learning algorithm comprises an artificial neural network, an association rule learning algorithm, a hierarchical clustering algorithm, a cluster analysis algorithm, a matrix factorization approach, a dimensionality reduction approach, or any combination thereof. In some embodiments, the unsupervised machine learning algorithm is an artificial neural network comprising an autoencoder, a stacked autoencoder, a denoising autoencoder, a variational autoencoder, or any combination thereof. In some embodiments, the autoencoder, stacked autoencoder, denoising autoencoder, variational autoencoder, or any combination thereof, is used to determine a set of one or more latent variables that comprise a compressed representation of one or more key cell attributes. In some embodiments, the autoencoder, stacked autoencoder, denoising autoencoder, variational autoencoder, or any combination thereof, is used to perform generative modeling to predict a change in one or more cell phenotypic traits of EVs based on a change in one or more latent variables.

### Further EV Analysis Methods

i. Transmission Electron Microscopy (TEM)

In certain embodiments, the methods disclosed here further comprise use of transmission electron microscopy (TEM) methods for analysis and characterization of individual EVs in a sample. In certain embodiments, TEM is combined with negative staining for imaging and analysis of EVs. In TEM, a stationary, spread electron beam with energy between 60 and 300 keV irradiates a sample that is thinner (often much thinner) than 0.5 μm. The sample modifies the phase and amplitude of the transmitted electrons, so that the resulting image contains information about the sample. In certain embodiments,

ii. Nanoparticle Tracking Analysis (NTA)

In certain embodiments, the methods disclosed herein further comprise use of nanoparticle tracking analysis (NTA) for measuring EV concentration and size in a sample. NTA visualizes and measures EVs in solution based on the relationship between the rate of Brownian motion and EV size. NTA methods permit analysis of the size distribution of EVs having a diameter between 10 nm and 1 μm. Generally. NTA relies on the use of a microscope in combination with a laser to illuminate EVs in a liquid suspension. Scattering of light by EVs across multiple image frames facilitates tracking the motion of each particle between frames.

iii. Dot Blots

In certain embodiments, provided herein are methods for analyzing EVs using a dot blot. Dot blotting is a molecular technique used for detecting the presence of specific proteins in a sample. Unlike western blots, dot blots do not require electrophoretic separation of proteins, and can be used to detect the presence of specific proteins in a sample (e.g., on the surface or in the lumen of EVs) by applying the sample on a membrane in an individual spot and performing the blotting thereon. In certain embodiments, dot blots are used to assess the purity of a sample containing EVs. In certain embodiments, dot blots are used to assess EV biomarker content. In certain embodiments, dot blots are used to confirm EV particle identity. e.g., by labeling with anti-TSPAN antibodies (e.g., anti-CD9, anti-CD63, and/or anti-CD81).

### Transcriptomic Analysis of EVs

Disclosed herein, in certain embodiments, are methods for generating a transcriptomic profile of a cell of a target tissue of origin using EVs obtained from a sample (e.g., a biofluid sample or a tissue sample). Transcriptomics is a collection of methods for quantitatively analyzing RNA molecules produced by cells and transferred across different subcellular and extracellular compartments. The determination of the transcriptomic content of a cell or tissue (i.e., “RNA or gene expression profiling”) provides a method for the functional analysis of normal and diseased cells or tissues, including characterization of the functional state of the cell(s) or tissue(s).

In certain embodiments, it may desirable to analyze the messenger RNA (mRNA) or non-coding RNA (ncRNA) content of a target cell, such as a cell that expresses a particular gene(s) of interest. Such analysis can be facilitated using existing tools for single-cell transcriptome sequencing, including microarrays, 96-well based methods, and microfluidic instruments. These tools can be used to prepare whole transcriptome and target libraries. Furthermore, analysis of sequencing data may be performed, in certain embodiments, using several known analytical approaches for transcript profiling, including but not limited to: microarray-based approaches (cDNAs or oligonucleotides); sequencing-based approaches, such as serial analysis of gene expression (SAGE) or massively parallel signature sequencing (MPSS); and differential-display-based approaches, such as arbitrarily primed (AP) PCR and cDNA-amplified fragment length polymorphism (AFLP).

In certain embodiments, the methods disclosed herein include analysis of RNA transcript sequences present in EVs isolated from a sample (e.g., biofluid sample or tissue sample) obtained from a subject (e.g., a human) according to the present disclosure. In certain embodiments, analysis of RNA transcript sequences present in EVs includes isolation and purification of RNA present in isolated EVs. In certain embodiments, the isolated and purified RNA includes a mixed population of RNAs of two or more different types. In certain embodiments, the mixed population of RNAs includes coding RNA and non-coding RNA. In certain embodiments, the mixed population of RNAs includes short RNAs and long RNAs. In certain embodiments, short RNAs include no more than 200 nucleotides (nt)(e.g., no more than 200, 190, 180, 170, 160, 150, 140, 130, 120, 110, 100, 90, 80, 70, 60, 50, 40, 30, 20, 10, 9, 8, 7, 6, or 5 nt). In certain embodiments, the long RNAs include at least 200 nt (e.g., at least 200, 250, 300, 350, 400, 450, 500, 600, 700, 800, 900, 1,000, 1,200, 1,400, 1,600, 1,800, 2,000, 2,500, 3,000, 3,500, 4,000, 4.500, 5,000, 6.000, 7,000, 8,000, 9,000, 10.000, 15.000, 20,000, 30,000, 40,000, 50,000, 100,000, 200.000, 300,000, 400,000, 500,000, 750,000, 1,000,000, 1.500,000, 2,000.000, 2,500,000 nt, or more). In certain embodiments, the short RNAs include, without limitation, microRNA (miRNA), transfer RNA, (tRNA), tRNA-derived small RNA (tsRNA), ribosomal RNA (rRNA), small rDNA-derived RNA (srRNA), small nucleolar RNA (snoRNA), Piwi-interacting RNA (piRNA), or any combination thereof. In certain embodiments, the long RNAs include, without limitation, mRNA, long ncRNA (lncRNA) such as Y RNA, pseudogenes, or a combination thereof. In certain embodiments, the RNA is linear RNA. In certain embodiments, the RNA is circular RNA.

In certain embodiments, analysis of RNA transcript sequences present in EVs includes preparation of a polynucleotide library from a single EVs. In certain embodiments, analysis of RNA transcript sequences present in EVs includes preparation of a polynucleotide library from a plurality (e.g., 2 or more) of EVs. In certain embodiments, the polynucleotide library includes a cDNA library. In certain embodiments, the cDNA library is analyzed for the present of target sequences of interest. In certain embodiments, the cDNA library is analyzed for the presence of target sequences of interest using PCR (e.g., RT-PCR, such as quantitative RT-PCR) or sequencing (e.g., Sanger sequencing or next generation sequencing (NGS)). In certain embodiments, analysis of RNA transcript sequences includes use of high-throughput sequencing technology.

In certain embodiments, analysis of RNA transcript sequences present in EVs includes use of emulsion-based methods. For example, EVs from a sample are encapsulated in droplets using microfluidic emulsion-based technology, in certain embodiments. In certain embodiments, the droplets containing EVs are attached to specific barcodes to target polynucleotides within the droplets to facilitate high-throughput genetic and/or expression analysis of single EVs contained within the droplets. In certain embodiments, the barcodes are present initially as single molecule DNA templates with a randomized central sequence portion flanked by known primer sites. The templates are, in certain embodiments, reverse transcribed to generate an amplicon and/or PCR amplified to yield one or more amplicons within the droplets and attached to EV-derived nucleic acids by sequence overlap. In certain embodiments, an EV-derived nucleic acid is amplified using a target-specific PCR primer to amplify a target RNA(s) of interest. In certain embodiments, amplification of several target RNAs provides information about various features of the cells from which an EV is derived, such as the phenotype, functional state, or other feature of the cell.

In certain embodiments, a reaction to amplify an EV-derived nucleic acid is carried out in a one-pot reaction that performs cell lysis, target RNA reverse transcription, molecular barcoding of cDNA, PCR amplification of a droplet-specific barcode, and attachment of a copy of the barcode to each cDNA. In certain embodiments, the products are recovered and sequenced, such as using any of a variety of sequencing platforms. For example, an Illumina MiSeq platform can be used using 325×300 bp to sequence the entire length of each product. In certain embodiments, the droplet barcodes allow identification of all products from each single EV. In certain embodiments, the molecular barcodes allow expression quantification for each EV and, in some cases, elimination of sequencing and RT-PCR errors. Emulsion-based methods, PCR, molecular barcoding, adaptor ligation, and sequencing methods are all well-known in the art.

In certain embodiments, the disclosed methods facilitate sampling of a large number of EVs. Using similarity of expression patterns, a map of cells of origin from which the analyzed EVs are derived is constructed, in certain embodiments. This map can be used to distinguish cell types in silico, by detecting clusters of closely related cells. This method allows for access to expression data from every distinct cell type represented by the EVs present in the sample without the need for purification of these distinct EV subtypes. In certain embodiments, use of known markers can facilitate in silico delineation of EVs from defined cell types of origin.

In certain embodiments, a polynucleotide library is created from a plurality of EVs by releasing RNA from each single EV to provide a plurality of individual samples, wherein the RNA in each individual sample is from a single EV, synthesizing a first strand of cDNA (amplicon) from the RNA in each individual RNA sample, and incorporating a nucleotide barcode into the cDNA amplicon to provide a plurality of barcoded cDNA samples, wherein each cDNA sample is complementary to an RNA from a single EV, pooling the barcoded cDNA samples, and amplifying the pooled cDNA samples to generate a cDNA library comprising barcoded cDNA. In certain embodiments, the barcoded double-stranded cDNA is denatured to generate barcoded single-stranded cDNA to facilitate addition of an adaptor for sequencing.

In certain embodiments, the generated cDNA libraries are suitable for analysis of RNA expression profiles of single EVs by direct sequencing. In certain embodiments, the RNA expression profile is indicative of a state of a target cell of origin from which an EV is derived, such as, e.g., phase of cell cycle, cell stress, cell activation, etc. In certain embodiments, the RNA expression profile is indicative of a disease state in a target cell of origin from which a single EV is derived. For example, the RNA expression profile may indicate an increase or a decrease in the expression of an RNA, wherein the increase or decrease in expression of RNA is associated with disease etiology, progression, remission, etc. In certain embodiments, the RNA expression profile may indicate responsiveness (or lack thereof) to one or more therapeutic agents administered to a subject from which the sample containing the isolated EVs is obtained. In certain embodiments, the RNA expression profile may indicate abundance and/or distribution of RNA(s) of interest within or across tissues of interest. In certain embodiments, the RNA expression profile indicates enrichment of a particular RNA within a cell type and/or tissue type. In certain embodiments, the tissue of interest is selected from the group consisting of neural tissue (e.g., brain or spinal cord tissue, including neuronal, glial, neurovascular, neuroimmune, or other neural tissues), thyroid tissue, parathyroid tissue, adrenal gland, nasopharyngeal tissue, bronchus, lung, oral mucosa, salivary gland, esophagus, stomach, duodenum, small intestine, colon, rectum, liver, gallbladder, pancreas, kidney, urinary bladder, testis, epididymis, seminal vesicle, prostate, vagina, ovary, fallopian tube, endometrium, cervix, placenta, breast, cardiac tissue, smooth muscle, skeletal muscle, soft tissue, adipose tissue, skin, appendix, spleen, lymph node, tonsil, bone marrow, and thymus.

### Proteomic Analysis of EVs

Disclosed herein, in certain embodiments, are methods for generating a proteomic profile of a cell of a target tissue of origin using EVs obtained from a sample (e.g., a biofluid sample or a tissue sample). The proteome is the complete set of proteins produced or post-translationally modified by an organism or system. Proteomics is a systematic study of protein composition, structure, function, modifications, and interactions.

In certain embodiments, proteomics refers to experimental analysis of proteins that have been isolated (e.g., purified) and analyzed using, without limitation, 2D gel electrophoresis, Warburg-Christian method, Lowry assay, Bradford assay, spectrometry, antibody-dependent methods (e.g., ELISA, immunoprecipitation, immune-electrophoresis, western blot, immuno-staining, etc.), mass spectrometry (MS), X-ray crystallography, protein NMR, cryo-electron microscopy, small-angle X-ray scattering, circular dichroism, protein footprinting, two-hybrid system, protein-fragment complementation assay, co-immunoprecipitation, proximity ligation proximity labeling, ChIP-on-chip. ChIP-sequencing, DamID, microscale thermophoresis, toeprinting assay, TCP-seq, molecular dynamics, protein structure prediction, protein sequence alignment, protein structural alignment, protein ontology, hydrogen-deuterium exchange, protein sequencing, ligand-binding assay, isotopic labeling, etc.

In certain embodiments, the proteomic profile can, for example, be represented as a mass spectrum, but other representations based on any physicochemical or biochemical properties of the proteins may also be used. Thus, the proteomic profile may e.g., be based on differences in the electrophoretic properties of proteins, as determined by 2D gel electrophoresis and can be represented, e.g., as a plurality of spots in a two-dimensional electrophoresis gel. Differential expression profiles may have important diagnostic value, even in the absence of specifically identified proteins. Single protein spots can then be detected, for example, by immunoblotting multiple spots or proteins using protein microarrays. The proteomic profile typically represents or contains information that could range from a few peaks to a complex profile representing 50 or more peaks. Thus, for example, the proteomic profile may contain or represent at least 2, or at least 5 or at least 10 or at least 15, or at least 20, or at least 25 or at least 30 or at least 35, or at least 40, or at least 45, or at least 50 proteins. In certain embodiments, proteomic analysis of EVs includes lysis of EVs to release proteins contained therein. In certain embodiments, proteomic analysis of EVs includes proteolytic degradation (e.g., enzymatic digestion) of proteins into protein fragments.

Typically, protein patterns (e.g., proteome maps) of samples from different sources, such as healthy (e.g., control) biofluids and a test biological biofluid (test sample), are compared to detect proteins that are up- or down-regulated in a disease. These proteins can then be excised for identification and full characterization, e.g., using peptide-mass fingerprinting and/or mass spectrometry and sequencing methods. In certain embodiments, the healthy and/or disease-specific proteome map can be used directly for the diagnosis of the disease of interest, or to confirm the presence or absence of the disease. In comparative analysis, it is important to treat the healthy and test samples identically in order to correctly represent the relative abundance of proteins and obtain accurate results. The required amount of total protein will depend on the analytical technique used, and can be readily determined using known methods. Proteins present in biological samples (e.g., biofluids or tissue samples) are typically separated by two-dimensional gel electrophoresis (2-DE) according to their isoelectric point (pI) and molecular weight (MW). Proteins are first separated by charge using isoelectric focusing (one-dimensional gel electrophoresis). In certain embodiments, this step is carried out using commercially available immobilized pH-gradient (IPG) strips. The second dimension is a healthy SDS-PAGE analysis, in which the focused IPG strip is used as the sample. After 2-DE separation, proteins are visualized with conventional dyes like Coomassie Blue or silver staining, and imaged using known techniques and equipment, in certain embodiments. Individual spots are then cut from the gel, de-stained, and subjected to tryptic digestion. The peptide mixtures can be analyzed by mass spectrometry (MS). Alternatively, the peptides can be separated, for example by capillary high pressure liquid chromatography (HPLC) and can be analyzed by MS either individually, or in pools.

Mass spectrometers consist of an ion source, mass analyzer, ion detector, and data acquisition unit. First, the peptides are ionized in the ion source. Then the ionized peptides are separated according to their mass-to-charge ratio in the mass analyzer and the separate ions are detected. MS has been widely used in protein analysis, especially since the advent of matrix-assisted laser-desorption ionization/time-of-flight (MALDI-TOF) and electrospray ionization (ESI) methods. There are several versions of mass analyzer, including, for example MALDI-TOF and triple or quadrupole-TOF, or ion trap mass analyzer coupled to ESI. Thus, for example, a Q-Tof-2 mass spectrometer utilizes an orthogonal time-of-flight analyzer that allows the simultaneous detection of ions across the full mass spectrum range. In certain embodiments, the amino acid sequences of the peptide fragments and eventually the proteins from which they derived can be determined by conventional methods, e.g., mass spectrometry or Edman degradation, among others.

In certain embodiments, the protein expression profile generated by proteomic analysis of EVs is indicative of a state of a target cell of origin from which an EV is derived, such as, e.g., phase of cell cycle, cell stress, cell activation, etc. In certain embodiments, the protein expression profile is indicative of a disease state in a target cell of origin from which a single EV is derived. For example, the protein expression profile may indicate an increase or a decrease in the expression of a protein, wherein the increase or decrease in expression of protein is associated with disease etiology, progression, remission, etc. In certain embodiments, the protein expression profile may indicate responsiveness (or lack thereof) to one or more therapeutic agents administered to a subject from which the sample containing the isolated EVs is obtained. In certain embodiments, the protein expression profile may indicate abundance and/or distribution of protein(s) of interest within or across tissues of interest. In certain embodiments, the protein expression profile indicates enrichment of a particular protein within a cell type and/or tissue type. In certain embodiments, the tissue of interest is selected from the group consisting of neural tissue (e.g., brain or spinal cord tissue, including neuronal, glial, neurovascular, neuroimmune, or other neural tissues), thyroid tissue, parathyroid tissue, adrenal gland, nasopharyngeal tissue, bronchus, lung, oral mucosa, salivary gland, esophagus, stomach, duodenum, small intestine, colon, rectum, liver, gallbladder, pancreas, kidney, urinary bladder, testis, epididymis, seminal vesicle, prostate, vagina, ovary, fallopian tube, endometrium, cervix, placenta, breast, cardiac tissue, smooth muscle, skeletal muscle, soft tissue, adipose tissue, skin, appendix, spleen, lymph node, tonsil, bone marrow, and thymus.

### Discovery and Validation of Biomarkers

Disclosed herein are methods for the discovery and validation of biomarkers of target tissues (e.g., healthy or diseased target tissues) using the disclosed assays for isolation, detection, and analysis of EVs. Many disease therapies, especially those developed in unselected patient populations, have only limited clinical benefits, with many patients not responding to a particular drug or therapy. Predictive biomarkers that define patient populations who are most likely to benefit from a given therapy, are crucial tools in the field of personalized medicine. Notably, likelihood for development and/or progression of certain diseases may be predicted long before presentation of specific symptoms associated with these processes on the basis of changes (e.g., increases or decreases) in expression and/or activity of specific biomarkers or sets of biomarkers. For example, in cancers, some predictive cancer biomarkers have been identified. For example, in colorectal cancer, KRAS is a predictive biomarker where somatic mutations in KRAS are associated with poor response to anti-EGFR directed therapies. Similarly, overexpression of the HER2 gene in breast and gastric cancers predicts response to anti-HER2 agents such as trastuzumab. Therefore, discovery and validation of biomarkers associated with specific disease processes may be especially useful for the selection of therapeutic modalities used to target the disease.

Accordingly, the assays disclosed herein are, in certain embodiments, advantageous for diagnosis, prognosis, and theranosis (i.e., determination of likelihood of effectiveness of specific therapies for the treatment of a disease). Predictive biomarkers have several advantages, including improving patient health and outcome by not administering treatments that are unlikely to provide a benefit to the patient. Further, accurate predictive biomarkers can be used to select patient subgroups that are likely to respond to treatment for clinical trials. Testing only likely responsive patients can decrease the cost of clinical trials since the trial would need fewer patients. Additionally, clinical trials including only patients likely to respond to a given therapeutic may allow therapeutics that previously failed clinical trials to show efficacy. New predictive biomarkers are needed. Many putative biomarkers in the art are identified during retrospective studies where patient samples from a clinical trial are analyzed for the presence or absence of biomarkers that correlate with the drug response observed during the clinical trial. However, discovering biomarkers in retrospective analyses is problematic. For example, the sample set cannot be used to confirm that the biomarker identified is predictive, and these studies often yield a high number of false correlations. Further, confirming putative biomarkers in subsequent clinical trials to rule out random, chance associations and the overfitting of data is costly and time-intensive. Typically, in order to validate the biomarker discovered from retrospective analysis (e.g., analyzing samples from a clinical trial), a new prospective clinical trial will need to be done. Drugs that failed late-stage clinical trials often offer many data points with which to discover potential biomarkers because of the uniform nature of the trial and the large sample sets. However, because these trials will typically require a prospective validating trial they are often not done.

The present disclosure provides methods for identification and validation of candidate biomarkers (e.g., proteins, nucleic acids, lipids, and/or carbohydrates) based on, e.g., differential expression of such biomarkers in EVs obtained from healthy and diseased tissues, e.g., using assays disclosed herein. In certain embodiments, candidate biomarkers are identified on the basis of enrichment or depletion of tissue-specific biomarkers as measured in EVs. In certain embodiments, the biomarker is identified as indicative of a disease process if the biomarker displays enrichment in EVs isolated from diseased tissue that is at least 1.1-fold, 1.2-fold, 1.3-fold, 1.4-fold, 1.5-fold, 2-fold, 3-fold, 4-fold, 5-fold, 6-fold, 7-fold, 8-fold, 9-fold, 10-fold, 15-fold, 20-fold, 30-fold, 40-fold, 50-fold, 60-fold, 70-fold, 80-fold, 90-fold, 100-fold, 120-fold, 140-fold, 160-fold, 180-fold, 200-fold, 250-fold, 300-fold, 350-fold. 400-fold. 450-fold, 500-fold, 600-fold, 700-fold, 800-fold, 900-fold, 1,000-fold greater, or more, as compared to the level of the biomarker in EVs isolated from healthy tissue. In certain embodiments, In certain embodiments, the biomarker is identified as indicative of a disease process if the biomarker displays enrichment in EVs isolated from diseased tissue that is at least 5-fold greater as compared to the level of the biomarker in EVs isolated from healthy tissue. In certain embodiments, the biomarker is identified as indicative of a disease process if the biomarker displays depletion in EVs isolated from diseased tissue that is at least 1.1-fold. 1.2-fold, 1.3-fold, 1.4-fold, 1.5-fold. 2-fold, 3-fold, 4-fold, 5-fold, 6-fold, 7-fold, 8-fold, 9-fold, 10-fold, 15-fold, 20-fold, 30-fold, 40-fold, 50-fold, 60-fold, 70-fold, 80-fold, 90-fold, 100-fold, 120-fold, 140-fold, 160-fold, 180-fold, 200-fold, 250-fold, 300-fold, 350-fold, 400-fold, 450-fold, 500-fold, 600-fold, 700-fold, 800-fold, 900-fold. 1,000-fold or more lower, as compared to the level of the biomarker in EVs isolated from healthy tissue. In certain embodiments, the biomarker candidate is identified using transcriptomic methods (e.g., methods disclosed herein). In certain embodiments, the biomarker candidate is identified using proteomic methods (e.g., methods disclosed herein). In certain embodiments, the candidate biomarker exhibits correlated changes in expression or activity with one or more (e.g., 1, 2, 3, 4, 5, 6, 7, 8, 9, 10, or more) additional biomarkers. In certain embodiments, the method of the disclosure comprises generating a biomarker signature, the biomarker signature comprising information about a change (e.g., increase or decrease) in expression or activity of one or more (e.g., 2, 3, 4, 5, 6, 7, 8, 9, 10, or more) biomarkers. In certain embodiments, the method of the disclosure comprises generating a biomarker signature, the biomarker signature comprising correlated changes in expression or activity of one or more (e.g., 2, 3, 4, 5, 6, 7, 8, 9, 10, or more) biomarkers. In certain embodiments, expression of a biomarker in a target tissue of interest is obtained from a tissue bank or tissue atlas. In certain embodiments, expression of a biomarker in a target tissue of interest is obtained using methods disclosed herein. In certain embodiments, expression of a biomarker in a target tissue of interest is obtained from a tissue atlas. In certain embodiments, expression of a biomarker in a target tissue of interest is obtained using conventional methods.

In certain embodiments, biomarkers present in/on EVs can be indicative of a disease state or susceptibility thereto with or without exhibiting changes in expression and/or activity of the biomarker. For example, in the context of nucleic acid biomarkers (e.g., DNA, pre-mRNA, mRNA, or other RNAs), certain modifications (e.g., one or more modifications) to the nucleic acid sequence are indicative of a disease state or susceptibility thereto. In certain embodiments, the one or more modifications to the nucleic acid sequence of a biomarker include a single nucleotide polymorphism (SNP). In certain embodiments, the one or more modifications to the nucleic acid sequence of a biomarker include deletion of one or more (e.g., 1, 2, 3, 4, 5, 6, 7, 8, 9, 10, 20, 30, 40, 50, 60, 70, 80, 90, 100, 200, 300, 400, 500, 600, 700, 800, 900, 1.000, or more) nucleotides as compared to a wild-type nucleic acid sequence of the biomarker. In certain embodiments, the one or more modifications to the nucleic acid sequence of a biomarker include insertion of one or more (e.g., 1, 2, 3, 4, 5, 6, 7, 8, 9, 10, 20, 30, 40, 50, 60, 70, 80, 90, 100, 200, 300, 400, 500, 600, 700, 800, 900, 1,000, or more) nucleotides as compared to a wild-type nucleic acid sequence of the biomarker. In certain embodiments, the one or more modifications to the nucleic acid sequence of a biomarker include retention of one or more (e.g., 1, 2, 3, or more) cryptic exons as compared to a wild-type nucleic acid sequence of the biomarker.

In the context of protein biomarkers, certain modifications (e.g., one or more modifications, such as one or more post-translational modifications) to the protein relative to its wild-type counterpart are indicative of a disease state or susceptibility thereto. In certain embodiments, the one or more post-translational modifications include phosphorylation, methylation, acetylation, glycosylation, myristoylation, palmitoylation, isoprenylation, prenylation, glypiation, lipoylation, flavination, heme C attachment, phosphopantetheinylation, retinylidene Schiff base formation, diphthamide formation, ethanolamine phosphoglycerol attachment, hypusine formation, beta-Lysine addition, amidation, amide bond formation, butyrylation, gamma-carboxylation, malonylation, hydroxylation, iodination, nucleotide addition, phosphate ester or phosphoramidate formation, propionylation, pyroglutamate formation, S-glutathionylation, S-nitrosylation, S-sulfenylation, S-sulfinvlation, S-sulfonylation, succinylation, sulfation, glycation, carbamylation, carbonylation, isopeptide bond formation, oxidation, ubiquitination, SUMOylation, neddylation, ISGylation, pupylation, formation of disulfide bridges, proteolytic cleavage, isoaspartate formation, and racemization.

Biomarker expression and/or activity in EVs can be measured using a variety of methods. In certain embodiments, biomarker expression and/or activity in EVs is measured using the SEVEN assay disclosed herein. In certain embodiments, biomarker expression and/or activity in EVs is measured using one or more convention methods, including, without limitation, polymerase chain reaction (PCR, e.g., RT-PCR), quantitative real-time PCR (qRT-PCR), an array (e.g., a microarray), a gene chip, pyrosequencing, nanopore sequencing, sequencing by synthesis, sequencing by expansion, single molecule real time technology, sequencing by ligation, microfluidics, infrared fluorescence, next generation sequencing (e.g., RNA-Seq techniques), dot blots, Northern blots, western blots, Southem blots, NanoString nCounter technologies, proteomic techniques, and combinations thereof. In certain embodiments, the aforementioned methods require use of a device capable of performing the assay. The device may be used to detect the level of a given biomarker by specific hybridization between the single-stranded nucleic acid and the biomarker (e.g., an mRNA, genomic DNA, or non-coding RNA), a nucleic acid of the biomarker (e.g., an mRNA), or a complementary nucleic acid thereof. The device may be or include a super-resolution microscope. The device may also include or be used with reagents and materials for next generation sequence (e.g., sequencing by synthesis). The device may also include or be used with NanoString reagents and at least one nCounter cartridge. The device may be or include a protein array, which contains one or more protein binding moieties (e.g., proteins, antibodies, nucleic acids, aptamers, affibodies, lipids, phospholipids, small molecules, labeled variants of any of the above, and any other moieties useful for protein detection as well known in the art) capable of detectably binding to the polypeptide product(s) of one or more biomarkers. The device may also be a cartridge for measuring an amplification product resulting from hybridization between one or more nucleic acid molecules from the patient and at least one single-stranded nucleic acid single-stranded nucleic acid molecules of the device, such as a device for performing qRT-PCR.

### Diagnostic, Prognostic, and Theranostic Methods

Disclosed herein, in certain embodiments, are methods for the diagnosis, prognosis, and/or theranosis of patients in need thereof, such as patients afflicted or at risk of developing a disease or disorder.

For example, the present disclosure provides a method for identifying one or more biomarkers associated with a disease or disorder from a population of EVs (e.g., TSEVs) in a sample (e.g., a biofluid or tissue sample) obtained from a subject. In certain embodiments, the method includes obtaining a level of expression of one or more tissue-specific biomarkers from an enriched population of disease-associated EVs (e.g., TSEVs) in the sample. In certain embodiments, the method includes calculating a difference score for the one or more tissue-specific biomarkers by comparing a level of expression of the one or more tissue-specific biomarkers from the population of disease-associated TSEVs to a level of expression of the one or more tissue-specific biomarkers from a reference population of TSEVs (e.g., a healthy control population), wherein a difference score above a cutoff value indicates that the one more tissue-specific biomarkers are associated with a disease or disorder. In certain embodiments, the cutoff value is at a 50^{th} percentile, 60^{th} percentile, 70^{th} percentile, 80^{th} percentile, 90^{th} percentile, or greater of the difference score in the reference population of TSEVs. In certain embodiments, the sample is: (1) a fluid sample obtained from a subject having the disease or disorder; or (2) a cell culture medium comprising a population of cells that model the disease or disorder.

Disclosed herein, in certain embodiments, is a method of diagnosing a subject as having or at risk of developing a disease or disorder, comprising: (i) performing the method of any one of the foregoing aspects and embodiments, on a sample obtained from the subject thereby obtaining a level of expression of one or more tissue-specific biomarkers associated with the disease or disorder; (ii) calculating a difference score for the subject by comparing a level of expression of the one or more tissue-specific biomarkers in the subject to a level of expression of the one or more tissue-specific biomarkers from a reference population of subjects without the disease or disorder: wherein a difference score above a cutoff value identifies the subject as having or at risk of developing the disease or disorder. In certain embodiments, the cutoff value is at a 50th percentile, 60th percentile, 70th percentile, 80th percentile, 90th percentile, or greater of the difference score in the reference population of subjects without the disease or disorder.

Disclosed herein, in certain embodiments, is a method of assessing therapeutic efficacy of a therapeutic agent in a subject having or at risk of developing a disease or disorder, comprising: (i) performing the method of any one of any one of the foregoing aspects and embodiments on a sample obtained from the subject prior to treatment with the therapeutic agent thereby obtaining a pre-treatment level of expression of one or more tissue-specific biomarkers characterized as having altered expression associated with the disease or disorder; (ii) administering an amount of the therapeutic agent to the subject; (iii) performing the method of any one of the foregoing aspects and embodiments on a sample obtained from the subject following treatment with the therapeutic agent thereby obtaining a post-treatment level of expression of the one or more tissue-specific biomarkers; (iv) calculating a difference score for the therapeutic agent by comparing the pre-treatment level of expression of the one or more tissue-specific biomarkers to the post-treatment level of expression of the one or more tissue-specific biomarkers; wherein a difference score above a cutoff value indicates that the therapeutic agent is effective at treating the disease or disorder. In certain embodiments, the cutoff value is at a 50th percentile, 60th percentile, 70th percentile, 80th percentile, 90th percentile, or greater of the difference score in the pretreatment level of expression of the one or more tissue-specific biomarkers. In certain embodiments, the method comprises characterizing the one or more tissue-specific biomarkers as having altered expression associated with the disease or disorder prior to performing step (i).

Disclosed herein, in certain embodiments, is a method of selecting a therapeutic agent for the treatment of a subject having or at risk of developing a disease or disorder, comprising: (i) performing the method of any one of the foregoing aspects and embodiments, thereby obtaining a level of expression of one or more tissue-specific biomarkers from an enriched population of disease-associated TSEVs: (ii) calculating a difference score for the one or more tissue-specific biomarkers by comparing a level of expression of the one or more tissue-specific biomarkers from the population of disease-associated TSEVs to a level of expression of the one or more tissue-specific biomarkers from a reference population of TSEVs, wherein a difference score above a cutoff value indicates that the one more tissue-specific biomarkers are associated with a disease or disorder; (iii) identifying a common biological signaling pathway associated with the one or more disease-associated tissue-specific biomarkers; and (iv) selecting a therapeutic agent that modulates activity of the common biological signaling pathway. In certain embodiments, the method further comprises (v) administering an effective amount of the therapeutic agent to the subject having or at risk of developing the disease or disorder. In certain embodiments, the cutoff value is at a 50th percentile, 60th percentile, 70th percentile, 80th percentile, 90th percentile, or greater of the difference score in the reference population of TSEVs. In certain embodiments, the level of expression is a mean or median level of expression. In certain embodiments, the method further comprises obtaining the sample from the subject having or at risk of developing the disease or disorder prior to performing step (i). In certain embodiments, the method further comprises obtaining the sample from the subject having or at risk of developing the disease or disorder prior to performing step (i). In certain embodiments, the disease or disorder is selected from the group consisting of a cancer, cardiovascular disease or disorder, neurological disease or disorder, and autoimmune disease or disorder. In certain embodiments, the subject is a human, non-human primate, or rodent.

### EXAMPLES

The following examples are put forth to provide those of ordinary skill in the art with a description of how the compositions and methods described herein may be used, made, and evaluated, and are intended to be purely exemplary of the disclosure and are not intended to limit the scope of what the inventors regard as their invention

### Example 1: SEVEN Assay for Analysis and Characterization of Plasma-Derived Extracellular Vesicles

### Materials and Methods

### Antibodies and Proteins

The following antibodies (Abs) were used in the experiments of this study: anti-CD9, anti-CD63, anti-CD81, anti-IGF1R (ganitumab), anti-cytochrome C (anti-CytC), anti-syntenin (anti-Synt), and anti-APOA1, as well as goat anti-rabbit (anti-rabbit IgG), IRDye 800CW goat anti-mouse, and IRDve 680RD goat anti-rabbit secondary antibodies. For ExoView experiments, fluorescently labeled anti-CD9-CF488A, anti-CD63-CF647, and anti-CD81-CF555 antibodies were used. Human lactadherin (MFG-E8) and aa Leu-24-Cys387 (contains both C1 and C2 domains) were also used antibodies.

Antibody Conjugation with a Fluorophore

Anti-CD9, anti-CD63, and anti-CD81 antibodies were conjugated with Alexa Fluor 647 N-hydroxysuccinimide (NHS) ester dye (AF647) using routine methods. The degree of labeling was assessed by spectrophotometry. Typical degree of labeling was 1.0-1.5.

### Human Plasma Samples and Patient Cohorts

Two types of human plasma samples were used in this study: (1) pooled human plasma (blood derived), which provided whole blood collected from donors in an FDA-approved collection center (used as healthy control); and (2) plasma from four pancreatic ductal adenocarcinoma (PDAC) patients (P1-P4). Control blood collected was in K2 ethylenediaminetetraacetic acid (EDTA)-containing dry blood collection bag, and spun at 5,000×g for 15 min in a refrigerated centrifuge. Plasma was isolated with a plasma extractor, frozen, and shipped on dry ice. Upon receipt, the plasma was stored at −80° C. Blood from PDAC patients was collected in EDTA-containing lavender top blood collection tubes and kept on ice until processed (typically within 1 h or less). The blood was transferred into a 15 mL conical tube and centrifuged at 1200×g for 15 min at room temperature (RT). The plasma fraction was carefully transferred to a 15 mL conical tube and aliquoted into cryovials. The vials were immediately frozen and stored at −80° C. The experimental team involved in this study was blinded to patient characteristics and was unblinded after all data collection and analysis were completed.

Enrichment of EVs from Plasma Using Size Exclusion Chromatography

EVs from pooled human plasma or PDAC patient plasma were isolated using 70 nm size exclusion chromatography (SEC) columns. Following column equilibration with phosphate-buffered saline (PBS), 400 μL of plasma sample was loaded onto the column and 13×500 μL fractions (F) were collected (F1-F13) following the collection of the 3 mL void volume (VV). For further experiments, combined fractions F1-F5 were used.

### Negative Staining Transmission Electron Microscopy (TEM)

4 μL of undiluted EV sample (SEC-enriched EVs from human pooled plasma or PDAC patient plasma) or 1:2 diluted recombinant EV reference material (rEVs) was adsorbed onto glow-discharged, carbon-coated 200 mesh electron microscopy grids for 5 mins. The grids were then sequentially washed in deionized (DI) water three times for 30 seconds and were contrasted with 1% (w/v) uranyl acetate solution three times for 10 seconds. TEM images were acquired on a TEM microscope at an acceleration voltage of 120 kV using LaB6 filament. The images were captured with a 2 k×2 k CCD camera. 4×4 μm, 8-bit, TEM images were converted into 16-bit images and analyzed by outlining individual EVs using a manual editing tool. EV diameter and roundness were determined by an automatic analysis module, and EVs within the size range of 30-400 nm were considered. Samples from three independent repeats were analyzed (in total 928 EVs were detected).

### Nanoparticle Tracking Analysis

The EV concentration and size distribution of rEVs and SEC-enriched pooled human plasma samples were determined by nanoparticle tracking analysis (NTA). The samples were injected into the sample chamber with a sterile syringe until the liquid reached the tip of the nozzle. All measurements were performed at RT. The samples for SEC-enriched pooled human plasma EVs were diluted 1:10 in 1 mL PBS. The samples for rEVs were diluted 1:200 in 1 mL PBS. Using default settings, three measurements were completed, each including a 60-second movie captured at 30 frames per second (fps) with manual shutter and gain adjustments. For statistical analysis of the NTA-determined size of EVs, only rEVs detected in the 30-400 nm size range were considered

### Microfluidic Resistive Pulse Sensing (MRPS)

Microfluidic resistive pulse sensing (MRPS) measurements were performed using a nanoparticle analyzer. The microfluidic system was primed with a solution of 0.1% Tween-20 (v/v) in PBS (PBS-T 0.1%). Instrument parameters were automatically determined by the device, including pressure at each cartridge port and voltage at each bias electrode. Diluent was filtered with syringe filters of 0.02 μm to avoid false-positive counts. Particle size distributions were determined using TS-400 cartridges (65 to 400 nm). Samples were diluted 1:100 in PBS-T 0.1%. For each measurement, 7 μL of diluted sample was applied to the cartridge. All experiments consisted of continuous acquisitions until the standard error reached <2%, and experiments were repeated in triplicate. Upon completion of a measurement, the data were combined, peak filters were applied, and background subtraction was performed.

### Dot Blots

Standard antibody dot blots and protein stain dot blots were performed to assess the EV marker content and purity of the SEC-enriched pooled human plasma samples. The antibody dot blots on SEC-enriched pooled human plasma were performed according to a standard protocol. The following primary antibodies were used: anti-CD9 at 1:500, anti-CD63 at 1:500, anti-CD81 at 1:500, anti-Synt at 1:1000, anti-CytC at 1:1000, and anti-APOA1 at 1:1000 dilution. Imaging was subsequently performed.

### Super-Resolution Microscopy Comprising Single Extracellular Vesicle Nanoscopy Assay

i. Coverslip Functionalization and Antibody Immobilization

25 mm diameter #1.5H glass coverslips were cleaned using known methods and coated with MCP4 solution. 0.5 μL of affinity reagent solution containing 1% glycerol was pipetted on the center of each coverslip. Anti-IGF1R antibody was spotted at 1 mg/mL; a mixture of anti-CD9, anti-CD63, anti-CD81 antibodies (“anti-TSPAN antibodies”) was spotted at a final concentration of 0.17 mg/mL per antibody; lactadherin was spotted at 0.5 mg/mL, and all other antibodies (anti-CD9, anti-CD63, anti-CD81, anti-rabbit IgG, and anti-cytochrome C) were spotted at 0.5 mg/mL. Immediately after spotting, each coverslip was incubated for 4 h at RT in a humidity-controlled dish. Following incubation, the surfaces were washed incubated in 1 mL blocking solution on an orbital shaker for 30 min at RT to cap unreacted sites. The coverslips were washed with a blocking buffer (BB) consisting of 2% (w/v) BSA and 0.025% (v/v) Tween-20 in PBS (PBS-T) followed by 0.025% (v/v) PBS-T antibodies.

ii. EV Capture and Staining

The EV samples (SEC-isolated pooled human plasma, crude pooled human plasma, and crude PDAC patient plasma) were diluted in PBS to a desired concentration. In the case of control experiments with lysed EVs, 0.3% (v/v) Triton X-100 was added to the EV sample, vortexed for 30 seconds, and incubated on a rotator for 30 min at RT. Prior to adding the EV samples onto surfaces, Tween-20 was added to each sample to a final Tween-20 concentration of 0.025% (v/v). The EV samples were incubated on each surface overnight at RT on a rocking shaker in a humidity-controlled dish. In the case of αIGF1R Ab spots, 160 μL of EV sample was added onto each coverslip. In all other cases, 80 μL of EV sample was added onto each coverslip. Depending on the plasma samples and Ab spots, the following EV sample dilutions were used: 1:1-1:64 dilutions (with dilution factor of 2) for SEC-isolated pooled human plasma sample; 1:20, 1:50, 1:100, 1:200, and 1:1000 dilutions for crude pooled human plasma sample: 1:10 (αIGF1R Ab spots) and 1:100 (αCD9 Ab spots) dilutions for PDAC patient plasma and healthy control samples. Following EV incubation, the coverslips were thoroughly washed with 0.025% (v/v) PBS-T.

The surfaces were rinsed three times with BB and the affinity captured EVs were stained with αTSPAN-AF647 fluorescent probe solution: 10 nM αCD9-AF647 Ab, 10 nM αCD63-AF647 Ab, and 10 nM αCD81-AF647 Ab in BB. 150 μL of αTSPAN-AF647 solution was incubated on each surface for 1 h at RT while protected from light. Next, excess fluorescent probes were removed from the surfaces by thoroughly washing with 0.025% (v/v) PBS-T followed by washing with PBS. The captured and stained EVs were fixed by incubating 200 μL of fixative solution (4% (w/v) paraformaldehyde glutaraldehyde on each surface for 30 min at RT. The fixation was terminated by incubating 200 μL of 25 mM glycine in PBS for 10 min at RT. The surfaces were then rinsed with PBS and stored in PBS until imaging. All surfaces were imaged within 1 day. For two-color imaging, available cysteines on EV membranes of SEC-enriched EVs from pooled human plasma were first labeled using 10 nmol of CF568-maleimide. Following the removal of excess dye with 70 nm qEV single column, 150 μL of isolated EVs in 0.025% (v/v) PBS-T were processed as described above.

iii. Imaging and Analysis

In SMLM, tetraspanins were detected with AF647 labeled anti-TSPAN antibodies. Before they photobleach, these fluorescent reporters alternate between dark and fluorescent states (turn “off” and “on”), allowing detection of a subset of non-overlapping localizations in each frame. 25,000 frames are combined to produce a localization map. These maps are analyzed (using Voronoi tessellation) to detect EVs. To enable proper molecular counting, the photophysical properties of the fluorescent probes were determined. In particular the average number of localizations per individual fluorescent probe and maximum dark time were quantified using surface assay for molecular isolation (SAMI). Here, the MCP4-coated surfaces were incubated with a mixture of 1 nM anti-CD9-AF647, 1 nM anti-CD63-AF647, and 1 nM anti-CD81-AF647 antibodies for 2 h. The surfaces were then washed and blocked. After the PBS wash, surfaces were imaged and analyzed. The number of detected localizations was divided by the average number of localization for a single fluorescent reporter to obtain the detected number of molecules.

The prepared surfaces were loaded into cell chambers and imaged by direct stochastic optical reconstruction microscopy (dSTORM) in dSTORM imaging buffer. The images were acquired using a 3D N-STORM super-resolution microscope. The 640 nm laser power used for excitation was 119.5 mW recorded out of the optical fiber. For each image. 25.000 frames were acquired at 10 ms exposure time on a region of interest (ROI) of 41×41 m (256×256 pixels). For two-color imaging, sequential 640 nm and 561 nm acquisitions were collected using known methods (e.g., 25,000 frames and 10 ms exposure time in each channel, using 119.5 mW power in the 647 nm channel and 108.1 mW power in the 561 nm channel).

The acquired images were initially rendered. In this process, single molecule localization coordinates were determined from the detected point spread functions (PSFs) using Gaussian fitting where the following identification settings were applied: 5,000 as minimum and 65,535 as the maximum height of fluorophore PSF; 100 as CCD baseline; 200 nm as the minimum and 400 nm as the maximum PSF peak width; 300 nm as initial fit width; 1.3 as maximum axial ratio; and 0 pixels as maximum pixel displacement. The minimum and maximum photons were set to 1,400 and 10,000, respectively. The raw SMLM images were analyzed using custom code. The following settings were used: 1) images were segmented using Voronoi tessellation: 2) segmentation polygons were grouped into clusters if the area of the polygon was smaller than 600 nm²; 3) the algorithm identified clusters as large EVs if the number of localizations per cluster was within the range of 100 and 15.000 and if the cluster radius was within 75 and 3,000 nm; 4) the algorithm identified the clusters as small EVs if the number of localizations per cluster was within the range of 55 and 2,000 and if the cluster radius was within 15 and 75 nm; 5) a two-step EV structure refining process was performed to improve EV detection. Using a searching algorithm with an 800 nm width square window centered at the EV centroid, EV neighboring polygons were assigned to their corresponding EVs, resulting in an expanded apparent EV size: 6) outlier localizations of the expanded EVs were detected and filtered out from the group of EV localizations. Localization precision was estimated using the coordinate-based localization precision estimator (CBLPE). TIRF imaging was performed with 488 nm laser with an exposure time of 30 ms and 1.6 mW excitation power recorded out of the optical fiber.

The characterization and analysis of rEVs by Extracellular Vesicle Spatial Clustering of Applications with Noise (EVSCAN) was performed in batches across multiple datasets known methods. A search radius of 40 nm (distance to search for neighboring points around each point) with 20 minimum points (number of neighboring points including itself to define the current point as part of a dense cluster or not) and an EV factor of 0.1 (threshold value to include a localization as part of an EV cluster based on the cluster's localization density profile) A ere used for the analysis. The EVSCAN algorithm is a variation of the density-based spatial clustering of applications (DBSCAN) algorithm designed to improve the clustering quality for EV localization data, by profiling each cluster for its density and removing low-density points within each cluster. Two color data were aligned using a Uniform Dual Channel Alignment tool (the program iteratively finds colocalized EV clusters, measures the average distance and direction of overlapping cluster centroids, and adjusts one channel accordingly until the results converge). Double-positive EVs were subsequently defined as two overlapping clusters of localizations from different channels and their centroids were calculated.

After EV detection, the following data were collected for each analyzed ROI: number of EVs per ROI, diameter of each identified single EV, the number of detected TSPAN molecule per each identified single EV (TSPAN/EV), EV eccentricity, and EV circularity. The diameter of a single EV was determined by calculating the diameter of a circle with an area equivalent to the area of the EV1. The TSPAN/EV was calculated by dividing the number of localizations per EV by the determined average number of localizations per an anti-TSPAN-AF647 probe molecule. EVs which had a diameter ≤400 nm and TSPAN/EV≤200 were included in the final data to exclude artifacts. An extremely small number of EVs were excluded using this filter.

### EV Characterization

The size distribution and tetraspanin content of 1:200 diluted crude pooled human plasma and PDAC patient sample samples (50 μL) were analyzed using a chip-based EV analysis platform. The chips were scanned in both the interferometric microscopy (IM) and in the fluorescence channels (with excitation wavelengths of 480 nm, 555 nm, and 640 nm) and the collected data were analyzed. The EV counts per spot data were normalized to a spot size of 150 μm diameter due to size differences between spots

### Statistical Information and Graphics

Mean, standard error of the mean (SEM), and coefficient of variation (CV) values were determined using standard methods. Statistical significance was determined using two-tailed two-sample Student's t-test with unequal variance. Significance levels were determined based on the resulting p-values and indicated as * p≤0.05, ** p≤0.01, *** p≤0.001. Due to the lognormal characteristic of the distribution of EV diameter, molecular tetraspanin content per EV (TSPAN/EV), and circularity, a logarithmic transformation was applied on each data set to approach normal distribution.

### Results

To assess individual SEC-enriched EVs, affinity isolation of tetraspanin-enriched EVs was combined with qSMLM. This approach allowed characterization of a wide range of EVs (with different biogenesis origins and sizes) that contain common EV markers. This newly developed approach, named Single Extracellular VEsicle Nanoscopy (SEVEN) assay, consists of seven steps (details are provided in Methods). (1) Characterization of photophysical properties of fluorescent probes used for EV staining, which allows efficient molecular counting. Because tetraspanins CD9, CD63, and CD81 are typically abundant on EV surfaces, a mixture of Alexa Fluor 647 (AF647)-labeled antibodies against these tetraspanins was used as fluorescent probes for EV staining. FIG. 2A shows maximum dark time and FIG. 2B shows the average number of localizations per individual fluorescent probe for AF647 labeled antibodies against CD9, CD63, and CD81. (2) Coating of coverslips with a polymer having dense NHS groups. This polymer had a minimal background in SMLM and produced excellent a non-fouling surface when capped using blocking solution. (3) was Affinity reagents (e.g., anti-TSPAN antibodies) covalently attached to polymer-coated coverslips via NHS chemistry. Unlabeled anti-TSPAN antibodies were used for rEVs. Unreacted NHS groups were capped with blocking solution. (4) SEC-enriched EVs were affinity captured onto coverslips. (5) Affinity captured EVs were stained with a mixture of fluorescently labeled anti-TSPAN antibodies (scheme shown in FIG. 2D). (6) EVs were imaged using 2D SMLM. FIG. 2E shows a raw SMLM image of rEVs with a clear border (dashed line) between the spot coated with anti-TSPAN antibodies passivated (non-fouling) coverslip surface. The zoomed in image (FIG. 2H) shows an excellent overlap between SMLM signal (AF647 labeled anti-TSPAN antibodies that stained rEVs) and total internal reflection fluorescence (TIRF) microscopy signal (eGFP within rEVs). This method obtained an excellent signal-to-noise ratio that facilitated clear visualization of EVs. (7) Data was analyzed using Voronoi tessellation-based algorithm (FIG. 2F). Details of data analysis are provided in the Methods section. An example of tessellation analysis for an EV is shown in FIG. 2H). The outline of EV (dashed white line in FIG. 2H), indicates a largely circular structure.

To optimize and validate this assay, publicly available recombinant EV reference material (rEVs) was used. In addition to common luminal and membrane-associated EV markers (including tetraspanins CD9, CD63, and CD81), a fraction of these EVs express gag polyprotein fused to enhanced green fluorescent protein (eGFP). SEVEN was first used to assess tetraspanin-enriched rEVs. The number of rEVs (the same lot # of rEVs used for TEM) isolated either on anti-TSPAN Abs coated spot or control anti-rabbit IgG coated spot was evaluated. From 0.33 μL of rEVs, an average approximately 300 tetraspanin-enriched EVs were isolated in each region of interest (ROI) while a negligible number was isolated on control, IgG coated spot, FIG. 2I and Table 1.

TABLE 1
Properties of EVs isolated using SEVEN assay

| | | | Mean | Median | SEM | CV |
|---|---|---|---|---|---|---|
| rEV | TSPAN | EV count/ROI | 293 | 289 | 19 | 0.253 |
| | anti-rabbit | | 7 | 4 | 1.4 | 0.810 |
| | IgG | | | | | |
| | SMLM_{T} | Diameter | 108 nm | 105 nm | 0.52 nm | 0.319 |
| | | Circularity | 0.863 | 0.875 | 0.001 | 0.063 |
| | SMLM_{E} | Diameter | 109 nm | 106 nm | 0.47 nm | 0.289 |
| | TEM | Circularity | 0.866 | 0.879 | 0.001 | 0.068 |
| | | Diameter | 115 nm | 118 nm | 1.59 nm | 0.423 |
| | NTA | Circularity | 0.882 | 0.890 | 0.001 | 0.031 |
| | | Diameter | 132 nm | 125 nm | 0.001 nm | 0.369 |
| SEC-enriched | CD9 | EV count/ROI | 513 | 542 | 24 | 0.206 |
| pooled | CD63 | | 166 | 156 | 8.4 | 0.227 |
| human | CD81 | | 158 | 154 | 7.3 | 0.208 |
| plasma | anti-rabbit | | 8 | 8 | 1.1 | 0.601 |
| | IgG | | | | | |
| | cytochrome C | | 3 | 3 | 0.4 | 0.558 |
| | TSPAN + lysed | | 12 | 12 | 1.0 | 0.360 |
| | no Ab | | 1 | 0 | 0.2 | 1.250 |
| | CD9 | Diameter | 94 nm | 85 nm | 0.38 nm | 0.409 |
| | | TSPAN/EV | 28 | 18 | 0.28 | 1.022 |
| | | Circularity | 0.868 | 0.876 | 0.0005 | 0.057 |
| | CD63 | Diameter | 91 nm | 87 nm | 0.48 nm | 0.304 |
| | | TSPAN/EV | 25 | 18 | 0.41 | 0.943 |
| | | Circularity | 0.869 | 0.878 | 0.0009 | 0.058 |
| | CD81 | Diameter | 85 nm | 83 nm | 0.42 nm | 0.274 |
| | | TSPAN/EV | 21 | 15 | 0.33 | 0.883 |
| | | Circularity | 0.863 | 0.872 | 0.0009 | 0.061 |
| Crude | CD9 | EV count/ROI | 497 | 510 | 17 | 0.155 |
| pooled | CD63 | | 223 | 239 | 14 | 0.291 |
| human | CD81 | | 254 | 261 | 11 | 0.189 |
| plasma | anti-rabbit | | 9 | 9 | 0.8 | 0.410 |
| | IgG | | | | | |
| | cytochrome C | | 9 | 9 | 0.9 | 0.463 |
| | TSPAN + lysed | | 25 | 22 | 3.4 | 0.614 |
| | no Ab | | 1 | 0 | 0.2 | 1.777 |
| | CD9 | Diameter | 92 nm | 85 nm | 0.32 nm | 0.348 |
| | | TSPAN/EV | 21 | 15 | 0.21 | 0.982 |
| | | Circularity | 0.865 | 0.873 | 0.0005 | 0.057 |
| | CD63 | Diameter | 85 nm | 83 nm | 0.36 nm | 0.285 |
| | | TSPAN/EV | 17 | 12 | 0.24 | 0.929 |
| | | Circularity | 0.862 | 0.871 | 0.0007 | 0.058 |
| | CD81 | Diameter | 87 nm | 85 nm | 0.31 nm | 0.253 |
| | | TSPAN/EV | 16 | 12 | 0.18 | 0.781 |
| | | Circularity | 0.862 | 0.869 | 0.0007 | 0.057 |

The average localization precision for tetraspanin-enriched EVs was estimated between 7 and 8 nm (FIG. 2C). TEM data analysis was subsequently used to validate the EV size. There was an agreement for rEV sizes obtained using SMLM imaging followed by Voronoi tessellation analysis (SMLM1); SMLM imaging followed by EVSCAN analysis (SMLME, a modified version of DBSCAN), and TEM with segmentation analysis; nanoparticle tracking analysis (NTA) analysis resulted in a slightly larger EV size (FIG. 2J, Table 1). The shape of rEVs was also evaluated. In particular, circularity or roundness describes how closely a shape of an object approaches that of a perfect circle (1 indicates a perfect circle and 0 represents an increasingly elongated polygon). An agreement was obtained between SMLM and TEM for the circularity measurements (FIG. 2K, Table 1).

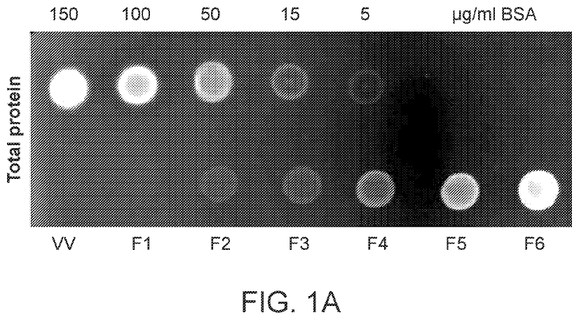

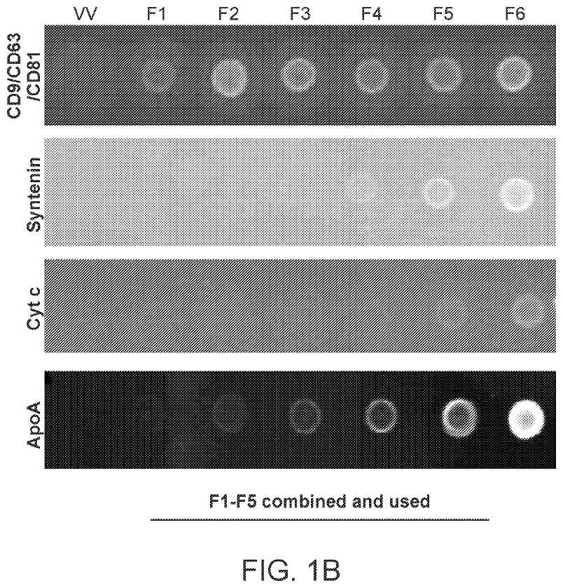

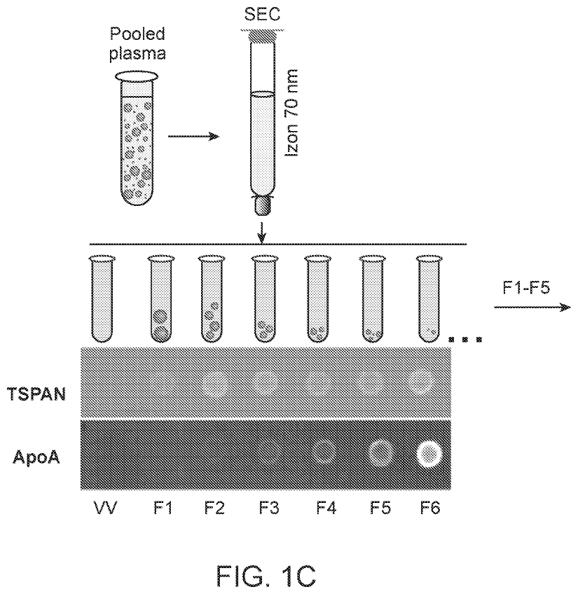

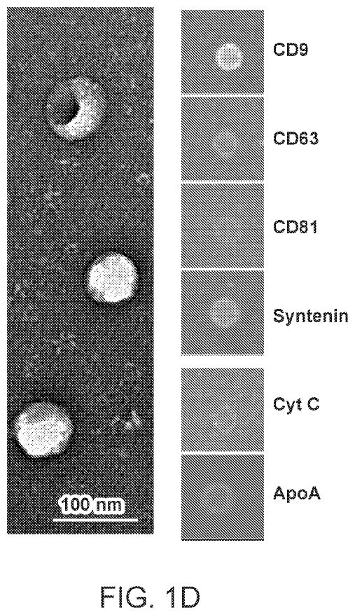

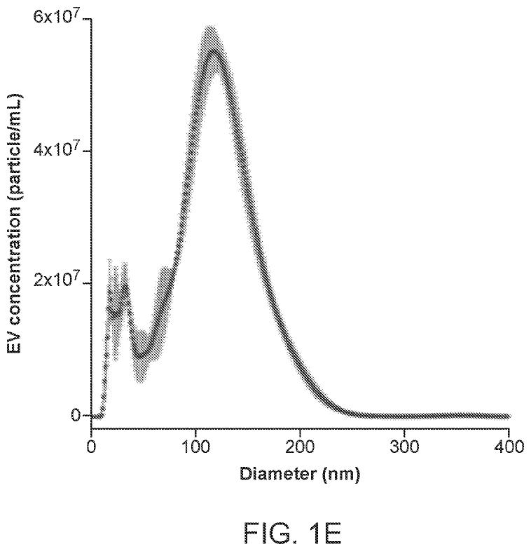

After validation of the method, EVs from pooled human plasma were evaluated. Isolation of EVs using size exclusion chromatography (SEC) is fast and has high recovery, high purity, and high reproducibility. SEC columns were used to enrich for EVs from pooled human plasma (scheme in FIG. 1C, top). Fractions were assessed for total protein content (FIG. 1A). EV markers (CD9, CD63, CD81, syntenin) were evaluated, confounding protein co-isolated with EVs (ApoA) and cytoplasmic marker typically not associated with EVs (cytochrome C), FIG. 1B and FIG. 1C, bottom. Based on this data, fractions F1-F5, which had high EV content and relatively high purity were selected. These fractions were combined for downstream applications. For these SEC-enriched EVs, in line with Minimal Information for Studies of Extracellular Vesicles (MISEV) guidelines, transmission electron microscopy (TEM, FIG. 1D, left) was used to confirm the intact EV morphology. Dot blots (FIG. 1D, right) were used to confirm the presence of canonical EV markers (tetraspanins CD9, CD63, CD81 and luminal EV protein syntenin) and low expression of ApoA and cytochrome C. NTA (FIG. 1E) was used to assess EV concentration and size range.

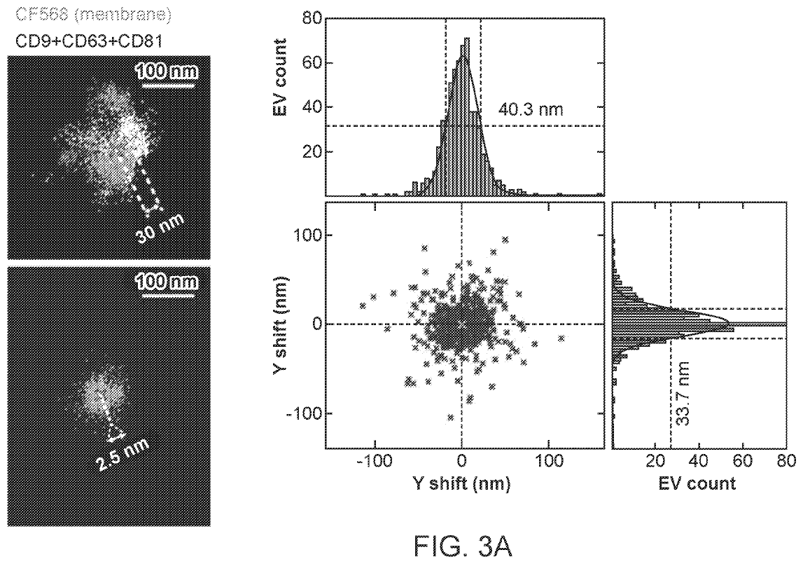

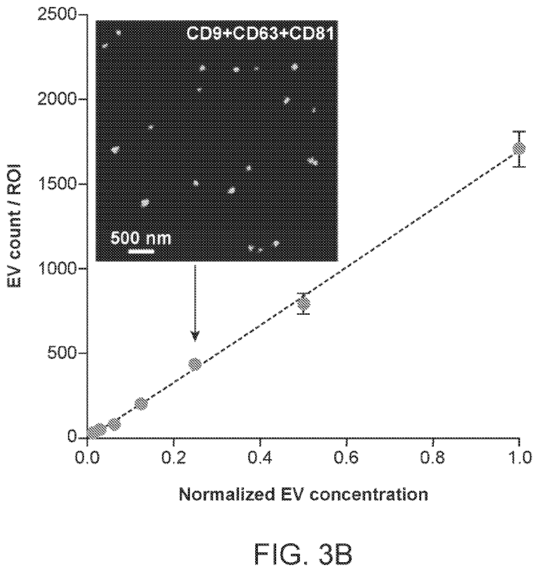

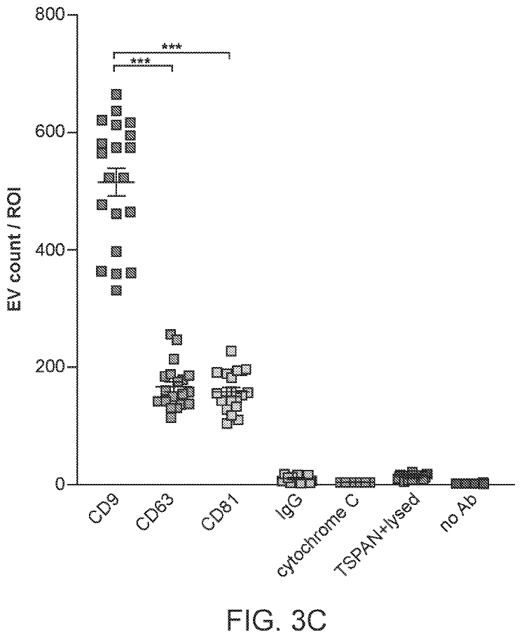

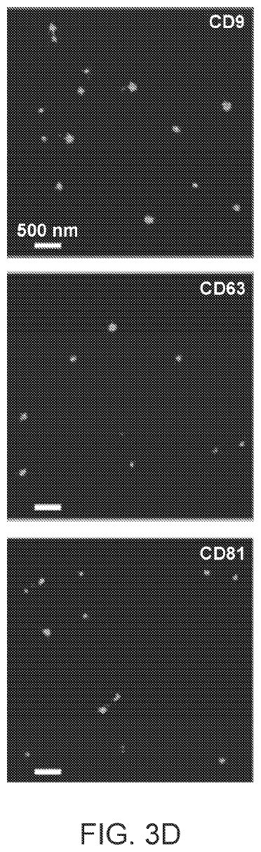

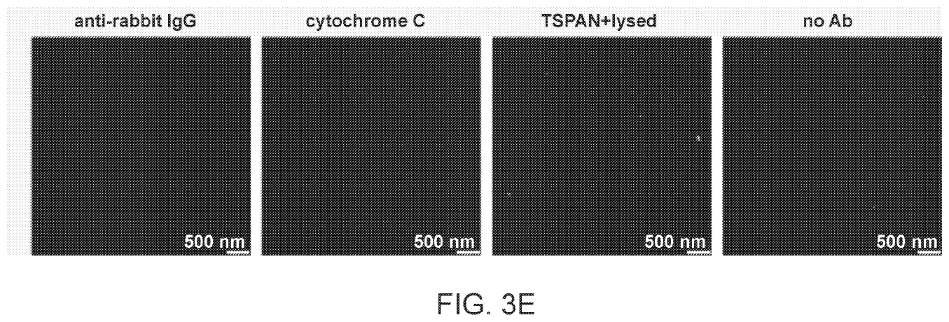

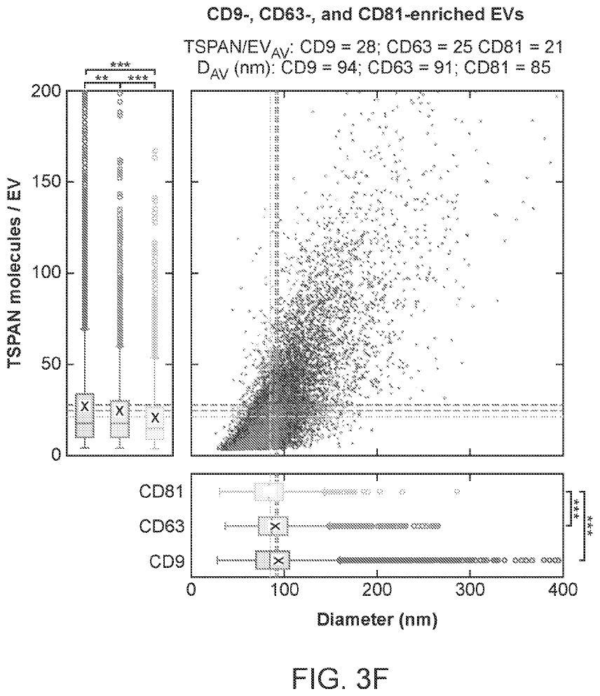

As with rEVs, unlabeled anti-TSPAN antibodies were used to affinity-isolate SEC-enriched EVs from pooled human plasma. To outline the EV surface well, staining was performed with a combination of three antibodies against abundant tetraspanins (CD9, CD63, and CD81) labeled with the same fluorescent dye, AF647. Since the distribution of tetraspanins can be heterogeneous and some EVs may have distinct tetraspanin domains, the overlap between staining of EV membrane (covalent labeling of available cysteines on EV membranes with CF568 maleimide, FIG. 3A, left) and staining of tetraspanins on EV membrane (affinity labeling of CD9, CD63, CD81 using AF647 labeled anti-TSPAN antibodies, FIG. 3A, left) was assessed. For 632 colocalized EVs, centroid shifts (647 channel relative to 568 channel) are shown in FIG. 3A, right. Gaussian distribution of centroid shifts is seen in both x- and y-direction, indicating high overlap for the majority of EVs (e.g., EV shown in FIG. 3A, left bottom). On average, 24.8 nm centroid shift (with SEM of 0.8 nm) was observed, consistent with heterogeneous distribution/clustering of tetraspanins in some EVs (e.g., EV shown in FIG. 3A, left top).

The number of detected tetraspanin-enriched EVs in each imaged region of interest (ROI) was tested for its correlation with the sample concentration. A significant positive correlation (R2=0.9986) was shown in the 64-fold dilution range (FIG. 3B). Density was optimal for 1:4 diluted EVs (FIG. 3B inset) and average localization precision was estimated at approximately 7 nm (FIG. 2C); images at all dilutions had high signal-to-noise ratio. Next, for 1:4 diluted EVs (FIG. 3C, Table 1, and Table 2), the number of detected CD9-, CD63-, and CD91-enriched EVs was counted.

For these experiments, EVs were isolated on surfaces coated with a mixture of fluorescent antibodies against only one type of tetraspanin (e.g., CD9, CD63, or CD81) and stained with AF647 labeled anti-TSPAN antibodies. In agreement with dot blots (FIG. 1D, right), the highest yield for CD9-enriched EVs was obtained. Raw SMLM images in FIG. 3D show clearly defined EVs. A negligible number of EVs was detected with four control conditions: (1) EVs lysed with 0.3% Triton X-100 and incubated onto coverslips coated with anti-TSPAN antibodies; (2) EVs incubated onto coverslips coated with anti-rabbit IgG; (3) EVs incubated onto coverslips coated with anti-cytochrome C antibody; and (4) EVs incubated on coverslips coated with the polymer that was capped (non-fouling surface, no antibody was attached). FIG. 3E shows raw SMLM images for these control surfaces. For all controls, only a EVs. To further characterize individual EVs, for each detected vesicle, the diameter and the number of detected TSPAN molecules per EV were detected. 2D histograms for CD9-, CD63-, and CD81-enriched subpopulations obtained for four independent repeats are shown in FIG. 3F. Each dot represents a detected vesicle and provides its diameter (x-axis) and the number of detected TSPAN molecules (y-axis). For three subpopulations, the mean (cross), median (center line), interquartile range (box), and coefficient of variation (CV) for size and number of detected TSPAN molecules were calculated. P-values are shown in Table 2.

TABLE 2
Summary of SEVEN measurements and p-values

| | | | | | p-value |
|---|---|---|---|---|---|
| rEV | TSPAN | vs | anti-rabbit | EV count/ | 4.72E−10 |
| | | | IgG | ROI | |
| | SMLM_{T} | vs | SMLM_{E} | Diameter | 3.67E−03 |
| | | | | Circularity | 4.60E−06 |
| | SMLM_{T} | vs | TEM | Diameter | 7.11E−01 |
| | | | | Circularity | 2.37E−29 |
| | SMLM_{E} | vs | TEM | Diameter | 4.32E−01 |
| | | | | Circularity | 3.35E−11 |
| SEC-enriched | CD9 | vs | CD63 | EV count/ | 7.48E−13 |
| pooled | | | | ROI | |
| human plasma | | vs | CD81 | | 7.71E−13 |
| | | vs | anti-rabbit | | 9.06E−15 |
| | | | IgG | | |
| | | vs | cytochrome C | | 7.97E−15 |
| | | vs | TSPAN + lysed | | 1.05E−14 |
| | | vs | no Ab | | 7.40E−15 |
| | CD63 | vs | CD81 | EV count/ | 4.84E−01 |
| | | | | ROI | |
| | | vs | anti-rabbit | | 6.36E−14 |
| | | | IgG | | |
| | | vs | cytochrome C | | 5.49E−14 |
| | | vs | TSPAN + lysed | | 1.12E−13 |
| | | vs | no Ab | | 4.46E−14 |
| | CD81 | vs | anti-rabbit | EV count/ | 1.03E−14 |
| | | | IgG | ROI | |
| | | vs | cytochrome C | | 1.06E−14 |
| | | vs | TSPAN + lysed | | 1.99E−14 |
| | | vs | no Ab | | 8.62E−15 |
| | CD9 | vs | CD63 | Diameter | 5.78E−01 |
| | | | | TSPAN/EV | 2.12E−03 |
| | | | | Circularity | 1.42E−01 |
| | CD9 | vs | CD81 | Diameter | 1.82E−29 |
| | | | | TSPAN/EV | 2.23E−32 |
| | | | | Circularity | 1.66E−06 |
| | CD63 | vs | CD81 | Diameter | 2.46E−20 |
| | | | | TSPAN/EV | 2.89E−13 |
| | | | | Circularity | 3.85E−07 |
| Crude | CD9 | vs | CD63 | EV count/ | 1.75E−14 |
| pooled | | | | ROI | |
| human plasma | | vs | CD81 | | 2.66E−13 |
| | | vs | anti-rabbit | | 4.82E−17 |
| | | | IgG | | |
| | | vs | cytochrome C | | 4.67E−17 |
| | | vs | TSPAN + lysed | | 1.91E−17 |
| | | vs | no Ab | | 3.84E−17 |
| | CD63 | vs | CD81 | EV count/ | 9.11E−02 |
| | | | | ROI | |
| | | vs | anti-rabbit | | 6.83E−12 |
| | | | IgG | | |
| | | vs | cytochrome C | | 6.55E−12 |
| | | vs | TSPAN + lysed | | 9.84E−12 |
| | | vs | no Ab | | 3.65E−12 |
| | CD81 | vs | anti-rabbit | EV count/ | 2.37E−15 |
| | | | IgG | ROI | |
| | | vs | cytochrome C | | 2.22E−15 |
| | | vs | TSPAN + lysed | | 4.35E−16 |
| | | vs | no Ab | | 1.55E−15 |
| | CD9 | vs | CD63 | Diameter | 4.80E−36 |
| | | | | TSPAN/EV | 4.60E−45 |
| | | | | Circularity | 4.57E−04 |
| | CD9 | vs | CD81 | Diameter | 6.77E−10 |
| | | | | TSPAN/EV | 1.47E−55 |
| | | | | Circularity | 5.12E−05 |
| | CD63 | vs | CD81 | Diameter | 4.09E−11 |
| | | | | TSPAN/EV | 9.35E−01 |
| | | | | Circularity | 7.53E−01 |

On average (FIG. 3F, Table 3, and Table 4), the EV diameter ranged from 85 nm for CD81-enriched EVs to 94 nm for CD9-enriched EVs. The number of detected TSPAN molecules per EV ranged from 21 for CD81-enriched EVs to 28 for CD9-enriched EVs. According to the CV values, CD81-enriched EVs had the smallest heterogeneity in size, while CD9-enriched EVs had the largest heterogeneity in size and TSPAN content.

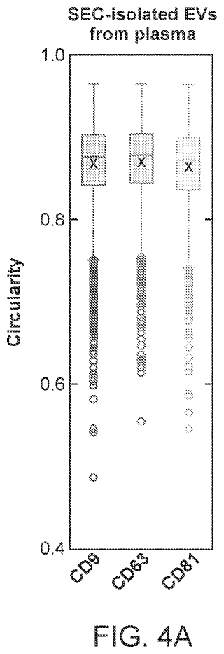

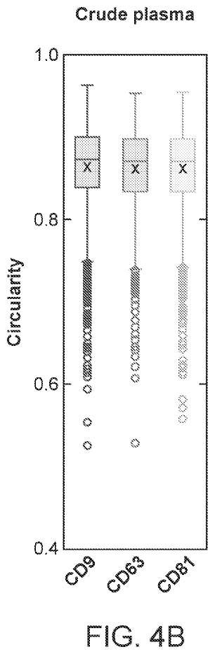

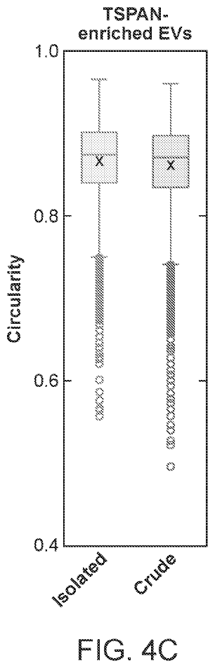

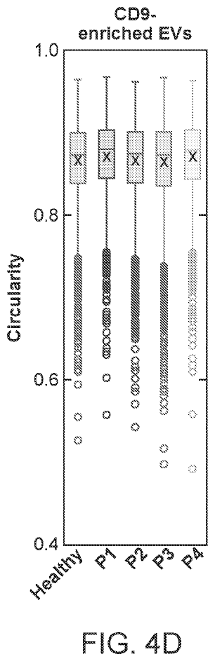

While most EVs are round-shaped, there is some morphological heterogeneity, especially in EVs derived from cancer cells. Thus, the shape of EVs was assessed by calculating the eccentricity and circularity in EV subpopulations. In all subpopulations, EVs were largely round-shaped with circularity values between 0.86 and 0.87 (FIG. 4A, right and Table 3).

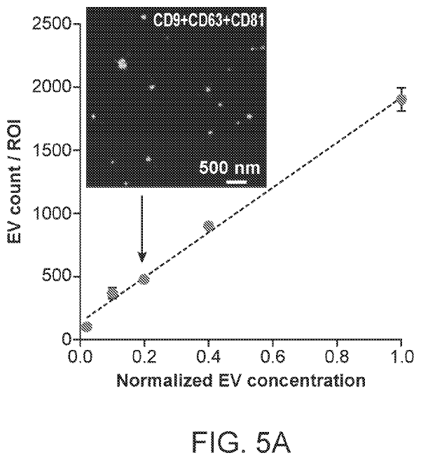

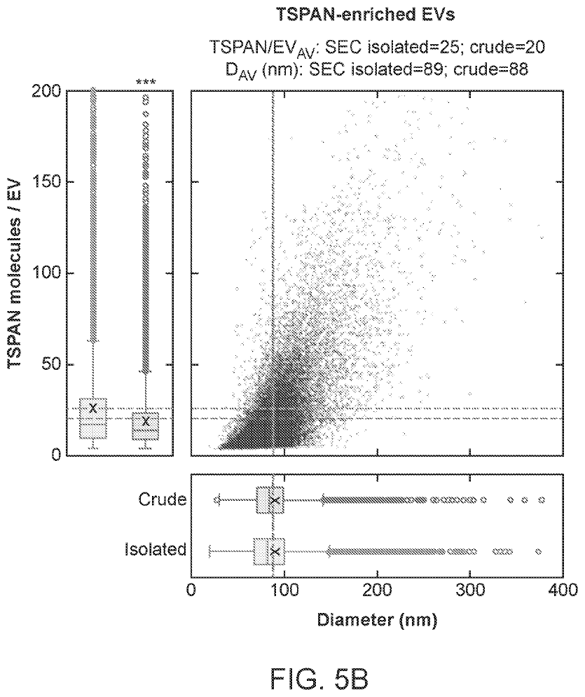

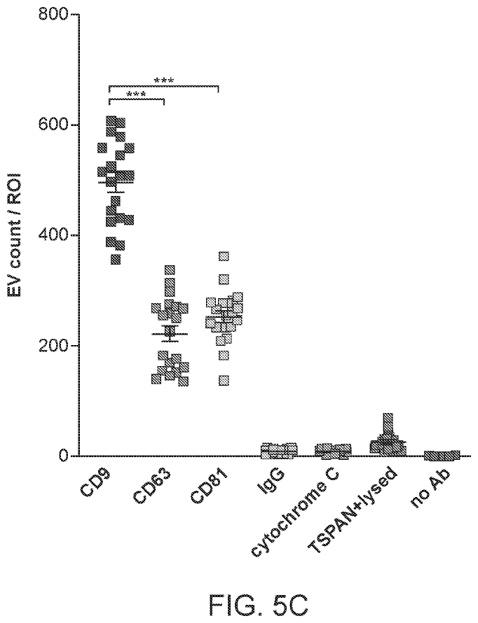

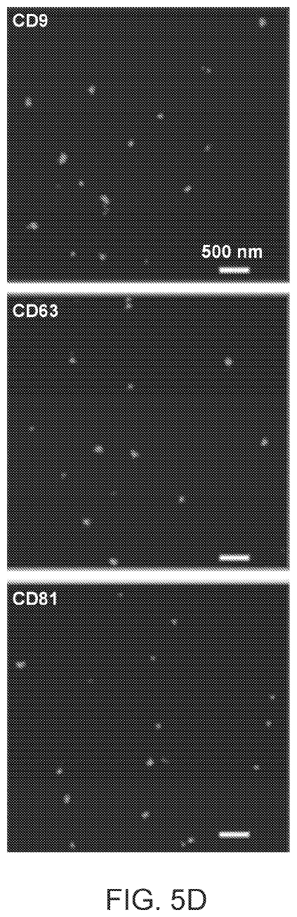

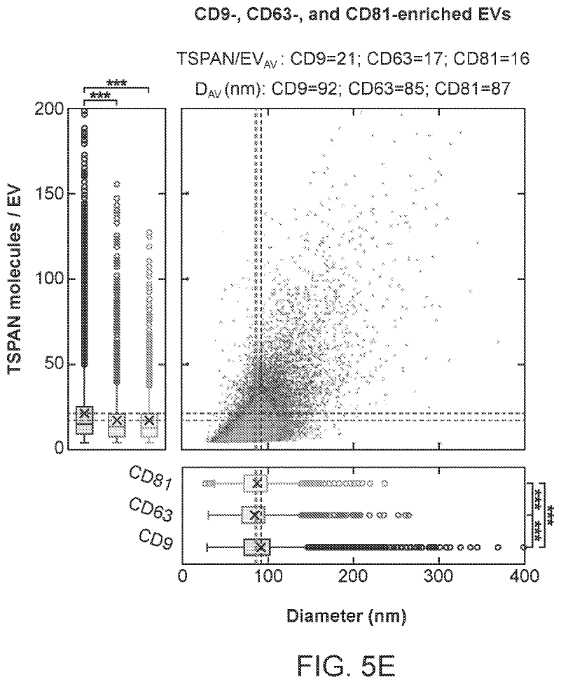

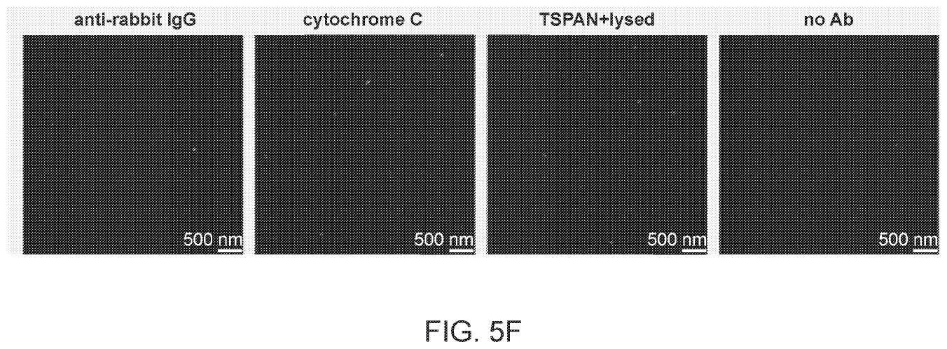

The SEVEN assay was applied to assess EVs directly from crude plasma (no SEC-enrichment). Tetraspanin-enriched EVs were investigated first: EVs were isolated on surfaces coated with unlabeled anti-TSPAN antibodies and stained with AF647 labeled anti-TSPAN antibodies. A clear border between the spot coated with anti-TSPAN Abs and passivated coverslip surface is seen, FIG. 2E. A high signal-to-noise ratio in SMLM was maintained with well-defined EVs (FIG. 2F and inset in FIG. 5A). According to data in FIG. 5A, the number of detected tetraspanin-enriched EVs per ROI positively correlated with sample concentration in 50-fold dilution range with R2=0.9947. Tetraspanin-enriched EVs could be detected from as little as 0.08 μl of plasma. This demonstrates the high sensitivity of the SEVEN assay with low sample volumes. The characteristics oftetraspanin-enriched EVs between SEC-enriched and crude plasma samples were also compared (FIG. 5B and Table 3).

TABLE 3
EV characteristics for tetraspanin-enriched EVs obtained using SEVEN

| | | | Mean | Median | SEM | CV |
|---|---|---|---|---|---|---|
| SEC-enriched | Anti-TSPAN Abs | EV count/ROI | 505 | 491 | 24 | 0.242 |
| | isolated | | | | | |
| Crude | Anti-TSPAN Abs | | 514 | 495 | 16 | 0.156 |
| | isolated | | | | | |
| Crude | Lactadherin-isolated | | 128 | 124 | 6.4 | 0.192 |
| SEC-enriched | Anti-TSPAN Abs | Diameter | 89 nm | 82 nm | 0.29 nm | 0.363 |
| | isolated | TSPAN/EV | 25 | 17 | 0.23 | 1.010 |
| | | Circularity | 0.866 | 0.875 | 0.0005 | 0.058 |
| Crude | Anti-TSPAN Abs | Diameter | 88 nm | 84 nm | 0.25 nm | 0.318 |
| | isolated | TSPAN/EV | 20 | 14 | 0.17 | 0.970 |
| | | Circularity | 0.862 | 0.871 | 0.0005 | 0.060 |
| Crude | Lactadherin-isolated | Diameter | 87 nm | 77 nm | 0.86 nm | 0.434 |
| | | TSPAN/EV | 18 | 12 | 0.45 | 1.089 |
| | | Circularity | 0.849 | 0.861 | 0.0014 | 0.072 |

| | | p-value |
|---|---|---|
| SEC-enriched vs Crude | Diameter | 8.80E−01 |
| (Anti-TSPAN Abs isolated) | TSPAN/EV | 5.63E−87 |
| | Circularity | 5.51E−12 |
| Anti-TSPAN | Diameter | 6.53E−07 |
| Abs vs Lactadherin | TSPAN/EV | 6.30E−11 |
| (Crude plasma) | Circularity | 1.63E−18 |

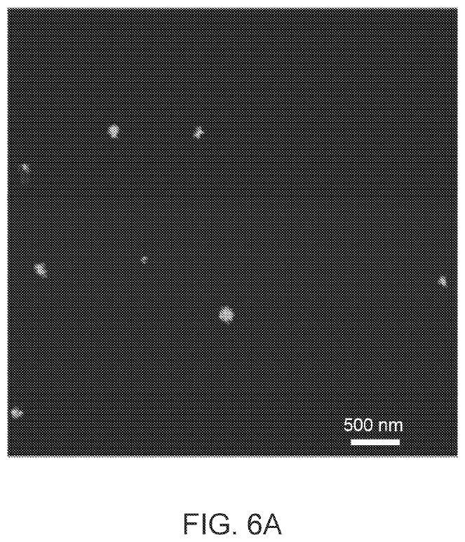

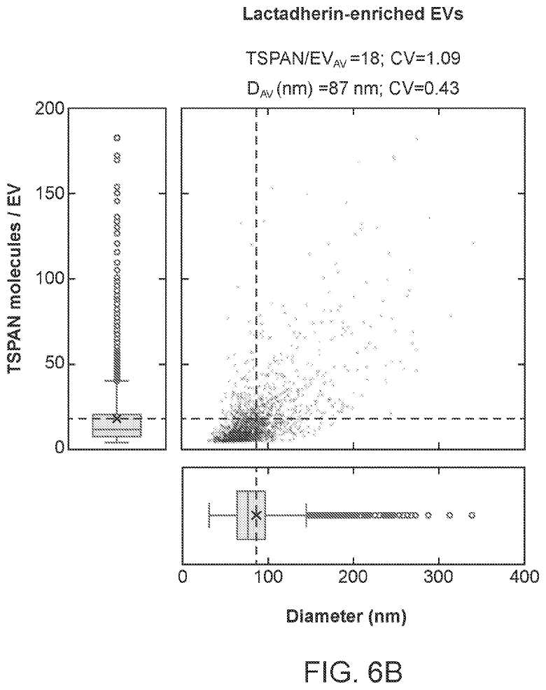

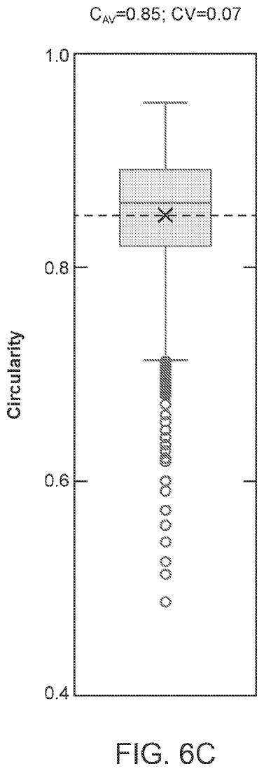

When normalized to sample volume, about 4.1-fold more EVs were obtained directly from crude plasma. While there was no significant difference in EV size and similar values for circularities were observed (FIG. 5B, FIG. 4C, and Table 3), EVs from crude plasma contained fewer detected TSPAN molecules. Tetraspanin-enriched EVs from crude plasma were assessed via isolation on lactadherin-coated surfaces (lactadherin binds to phospholipids that are present on EV membranes) followed by staining with AF647-labelled anti-TSPAN antibodies (FIGS. 6A-6C). Fewer EVs were isolated on lactadherin coated surfaces as compared to anti-TSPAN Abs coated surfaces (Table 3). Lactadherin may pull down other lipid-containing particles present in the plasma. While these particles would not be visualized in SMLM (they do not contain abundant tetraspanins), they would occupy the surface and limit available binding sites. Characteristics of EVs isolated on either anti-TSPAN Abs- or lactadherin-coated surfaces were similar: average values for diameter (88 nm vs 87 nm), circularity (0.86 vs 0.85), and detected tetraspanin content per EV (20 vs 18).

A subpopulation of EVs enriched in specific tetraspanin (EVs were isolated on coverslips coated with one of the three Abs: anti-CD9, anti-CD63, or anti-CD81 and stained with AF647 labeled anti-TSPAN antibodies) were assessed. For 1:100 plasma dilution, a similar distribution of subpopulations was observed between SEC-enriched EVs (FIG. 3C and Table 1) and crude plasma EVs (FIG. 5C and Table 1).

All controls (EVs were incubated onto coverslips coated with anti-rabbit IgG or anti-cvtochrome C Ab, Triton X-100 lysed EVs were incubated onto coverslips coated with anti-TSPAN antibodies, or no Ab) resulted in a low number of detected EVs, FIG. 5C and FIG. 5F. Moreover, raw SMLM images of EV subpopulations (FIG. 5D) indicated that CD9-, CD63-, and CD81-enriched EVs were well-defined.

For CD9-, CD63-, and CD81-enriched EV populations, EV size and detected molecular TSPAN content was defined along with the corresponding heterogeneity values. On average, CD63- and CD81-enriched EVs had similar diameters (85 nm and 87 nm, respectively) and numbers of detected TSPAN molecules per EV (17 and 16, respectively). CD9-enriched EVs had the largest diameter (92 nm), number of detected TSPAN molecules per EV (21), and heterogeneity in size and TSPAN content (FIG. 5E, Table 6, and Table 7). CD9-, CD63-, and CD81-enriched EVs had only small differences in average eccentricities and circularities (FIG. 5E. Table 6, and Table 7), and most were round-shaped.

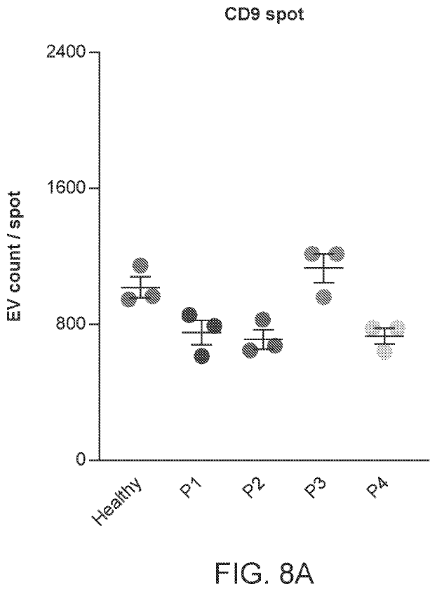

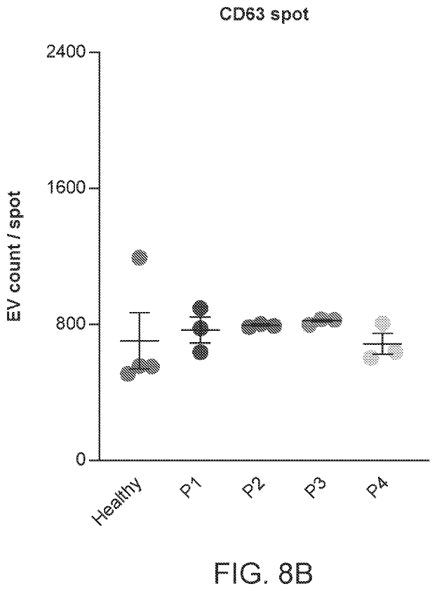

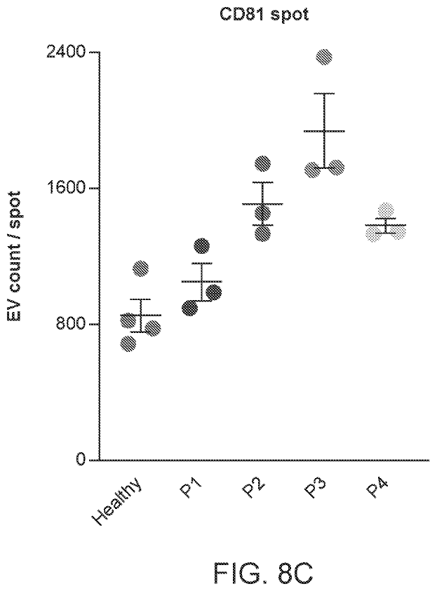

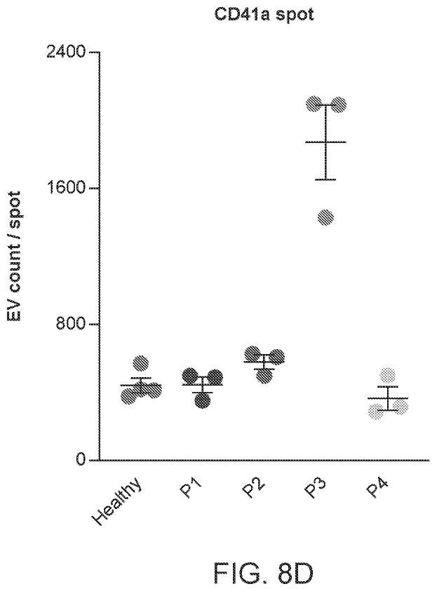

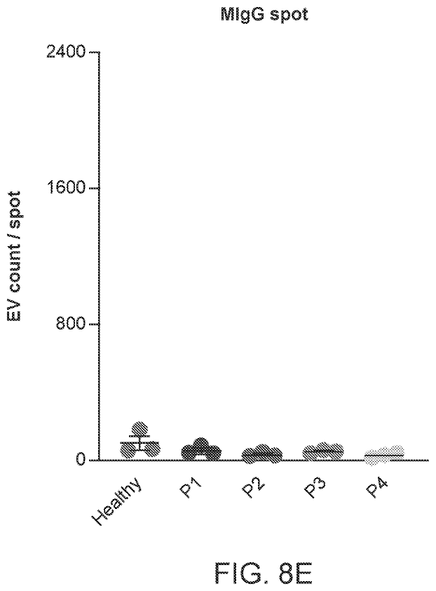

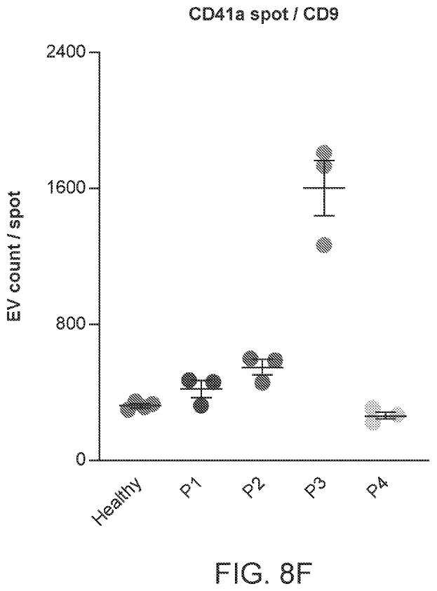

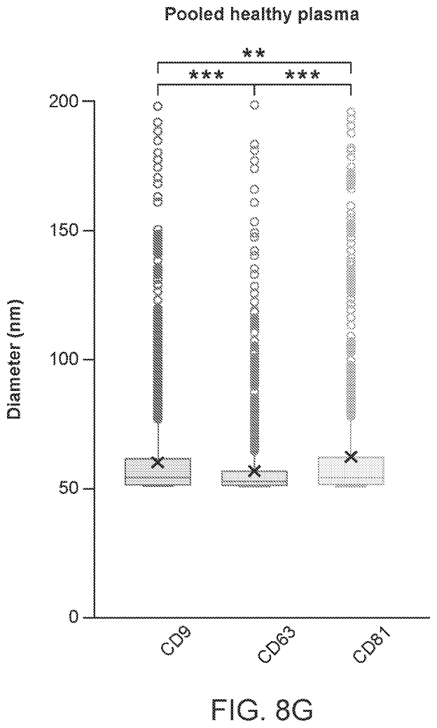

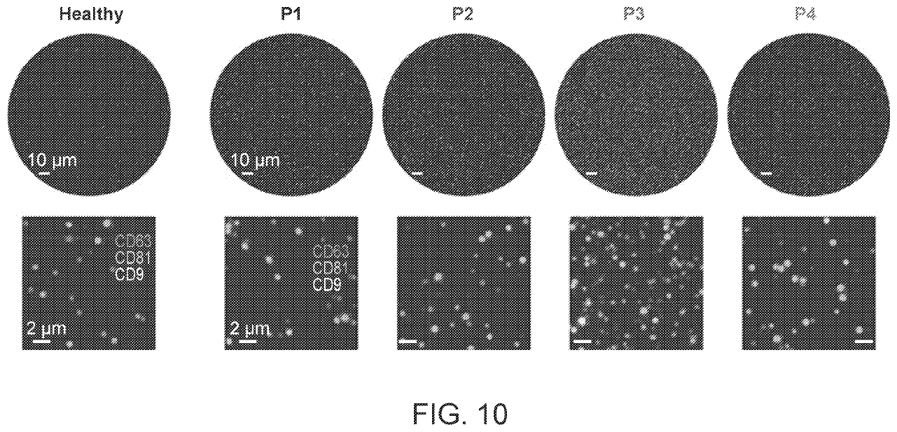

The ExoView assay was used as a complimentary and publicly available affinity-based approach to assess CD9-, CD63-, and CD81-enriched EVs from crude pooled human plasma. This approach provides the size of EVs using interferometry and determines the presence of proteins of interest on individual EVs based on staining with fluorescent antibodies. Fluorescent images of CD81-enriched EVs in three colors (FIG. 10) showed individual EVs that express three different tetraspanin markers (CD9, CD81, and CD63). The abundance of CD9-, CD63-, and CD81-enriched subpopulations (fluorescence channel) and controls are shown in FIGS. 8A-8E, and Table 4.

TABLE 4
Detected EV numbers per spot for
crude pooled human plasma EVs

| | | | Mean | Median | SEM | CV |
|---|---|---|---|---|---|---|
| Healthy | CD9 | EV count/ | 837 | 749 | 142 | 0.340 |
| | CD63 | spot | 708 | 560 | 165 | 0.466 |
| | CD81 | | 856 | 801 | 95 | 0.223 |
| | CD41a | | 443 | 414 | 43 | 0.194 |
| | CD41a/ | | 325 | 324 | 12 | 0.073 |
| | CD9 | | | | | |
| | control IgG | | 26 | 23 | 7 | 0.521 |
| P1 | CD9 | EV count/ | 754 | 790 | 70 | 0.161 |
| | CD63 | spot | 773 | 779 | 76 | 0.170 |
| | CD81 | | 1050 | 993 | 109 | 0.179 |
| | CD41a | | 443 | 483 | 46 | 0.181 |
| | CD41a/ | | | | | |
| | CD9 | | 420 | 464 | 49 | 0.202 |
| | control IgG | | 55 | 42 | 14 | 0.436 |
| P2 | CD9 | EV count/ | 716 | 674 | 57 | 0.139 |
| | CD63 | spot | 802 | 802 | 5 | 0.011 |
| | CD81 | | 1508 | 1450 | 124 | 0.142 |
| | CD41a | | 576 | 606 | 39 | 0.118 |
| | CD41a/ | | 549 | 585 | 43 | 0.135 |
| | CD9 | | | | | |
| | control IgG | | 33 | 31 | 6 | 0.316 |
| P3 | CD9 | EV count/ | 1132 | 1213 | 84 | 0.128 |
| | CD63 | spot | 825 | 834 | 9 | 0.020 |
| | CD81 | | 1934 | 1721 | 219 | 0.196 |
| | CD41a | | 1869 | 2088 | 221 | 0.205 |
| | CD41a/ | | | | | |
| | CD9 | | 1598 | 1727 | 167 | 0.181 |
| | control IgG | | 52 | 53 | 6 | 0.183 |
| P4 | CD9 | EV count/ | 733 | 776 | 44 | 0.105 |
| | CD63 | spot | 688 | 644 | 62 | 0.155 |
| | CD81 | | 1382 | 1350 | 43 | 0.054 |
| | CD41a | | 365 | 313 | 67 | 0.319 |
| | CD41a/ | | 265 | 267 | 21 | 0.136 |
| | CD9 | | | | | |
| | control IgG | | 29 | 30 | 3 | 0.191 |

| | | | | | p-value |
|---|---|---|---|---|---|
| Healthy | CD9 | vs | CD63 | EV count/ | 5.78E−01 |
| | | | CD81 | spot | 9.16E−01 |
| | | | CD41a | | 6.49E−02 |
| | | | control IgG | | 1.06E−02 |
| | CD63 | vs | CD81 | EV count/ | 4.76E−01 |
| | | | CD41a | spot | 2.07E−01 |
| | | | control IgG | | 2.55E−02 |
| | CD81 | vs | CD41a | EV count/ | 1.56E−02 |
| | | | control IgG | spot | 3.10E−03 |
| Healthy | vs | P1 | CD9 | EV count/ | 6.30E−01 |
| | | | CD63 | spot | 7.38E−01 |
| | | | CD81 | | 2.43E−01 |
| | | | CD41a | | 9.95E−01 |
| | | | CD41a/CD9 | | 1.87E−01 |
| | vs | P2 | CD9 | EV count/ | 4.76E−01 |
| | | | CD63 | spot | 6.11E−01 |
| | | | CD81 | | 1.31E−02 |
| | | | CD41a | | 7.20E−02 |
| | | | CD41a/CD9 | | 2.72E−02 |
| | vs | P3 | CD9 | EV count/ | 1.39E−01 |
| | | | CD63 | spot | 5.31E−01 |
| | | | CD81 | | 2.40E−02 |
| | | | CD41a | | 2.00E−02 |
| | | | CD41a/CD9 | | 1.64E−02 |
| | vs | P4 | CD9 | EV count/ | 5.28E−01 |
| | | | CD63 | spot | 9.16E−01 |
| | | | CD81 | | 6.89E−03 |
| | | | CD41a | | 3.88E−01 |
| | | | CD41a/CD9 | | 8.13E−02 |

| | | | SEVEN | Exo View |
|---|---|---|---|---|
| control IgG | vs | CD9 | 1.8% | 3.1% |
| | vs | CD63 | 4.1% | 3.6% |
| | vs | CD81 | 3.6% | 3.0% |
| cytochrome C | vs | CD9 | 1.8% | — |
| | vs | CD63 | 3.9% | — |
| | vs | CD81 | 3.5% | — |
| no Ab | vs | CD9 | 0.10% | — |
| | vs | CD63 | 0.22% | — |
| | vs | CD81 | 0.20% | — |
| TSPAN + lysed | vs | TSPAN | 4.8% | — |

| | SEVEN | Exo View |
|---|---|---|
| | EV density/μL | EV density/μL of |
| | of crude plasma | crude plasma |
| CD9 | 3.70E−01 μm⁻² | 1.89E−01 μm⁻² |
| CD63 | 1.66E−01 μm⁻² | 1.60E−01 μm⁻² |
| CD81 | 1.89E−01 μm⁻² | 1.94E−01 μm⁻² |

Additionally, in the fluorescence channel, CD41a/CD9 double-positive EVs that may be associated with platelets were evaluated. While these EVs were present, their number was lower compared to CD81-enriched subpopulations (CD81-positive EVs are not associated with platelets) based on EDTA-plasma sample origin (FIG. 8F and Table 4). Capturing efficiency and fraction of EVs isolated on control surfaces were compared for SEVEN and ExoView assays (normalized to sample volume and detection area). An agreement was observed (Table 4, bottom). Finally, the size of CD9-, CD63-, and CD81-enriched EVs from pooled human plasma was evaluated (FIG. 8G and Table 5). Compared to SEVEN, ExoView detected a smaller average size for all three subpopulations, consistent with its more limited size range (50-200 nm) and interferometric EV detection.

TABLE 5
EV diameter for crude pooled human
plasma EVs obtained using Exo View

| | | Mean | Median | SEM | CV |
|---|---|---|---|---|---|
| CD9 | Diameter | 60 nm | 54 nm | 0.29 nm | 0.280 |
| CD63 | | 57 nm | 52 nm | 0.26 nm | 0.233 |
| CD81 | | 62 nm | 54 nm | 0.56 nm | 0.360 |
| CD41a | | 62 nm | 56 nm | 0.40 nm | 0.300 |

| | | | | p-value |
|---|---|---|---|---|
| CD9 | vs | CD63 | Diameter | 2.32E−27 |
| | vs | CD81 | | 5.49E−03 |
| | vs | CD41a | | 2.78E−07 |
| CD63 | vs | CD81 | | 7.92E−24 |
| | vs | CD41a | | 1.29E−44 |
| CD81 | vs | CD41a | | 1.77E−01 |

The SEVEN assay was further applied to assess EVs enriched in membrane proteins highly expressed in patients with pancreatic ductal adenocarcinoma (PDAC). In this pilot study, 4 patients with resectable PDAC were assessed (patient characteristics are shown in Table 6).

TABLE 6
PDAC patient characteristics

| | CA 19-9 | | Involved | Tumor |
|---|---|---|---|---|
| Patient | levels | Tumor size | lymph nodes | differentiation |
| P1 | Non-secretor | 6.5 × 4.2 × 4.0 cm | 0/29 | moderate to poor |
| P2 | Normal | 2.1 × 1.7 × 1.5 cm | 2/22 | poor |
| P3 | Elevated | 2.3 × 1.8 × 1.7 cm | 6/30 | moderate |
| P4 | Elevated | 5.5 × 3.2 × 3.0 cm | 7/22 | moderate |

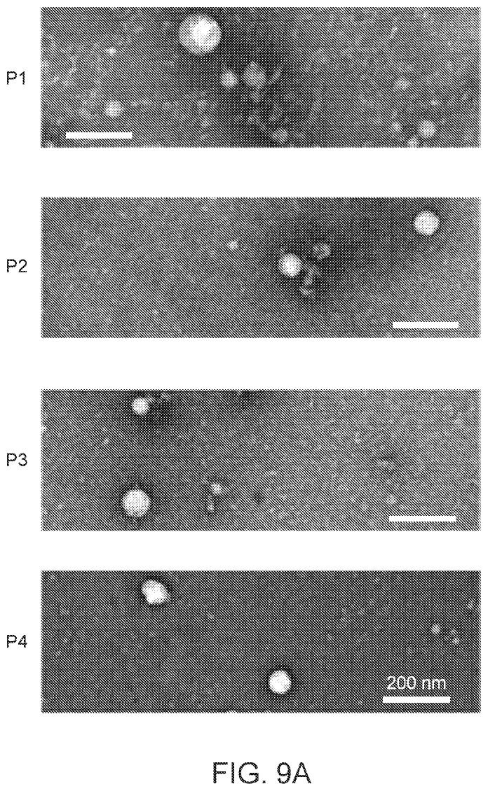

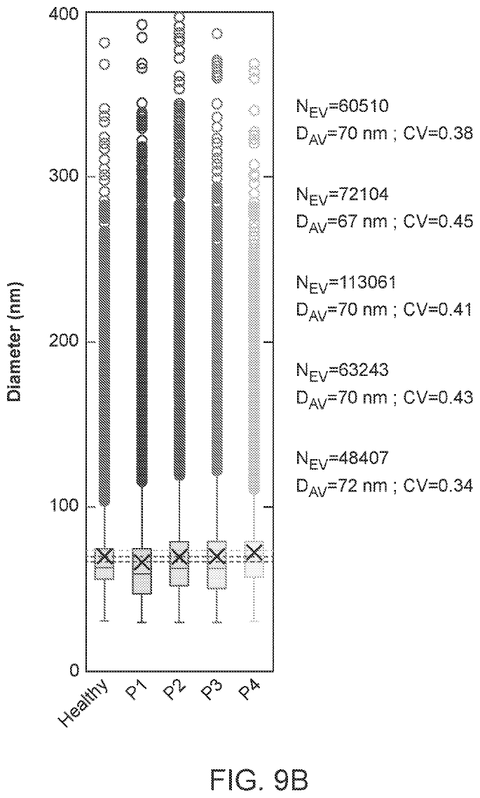

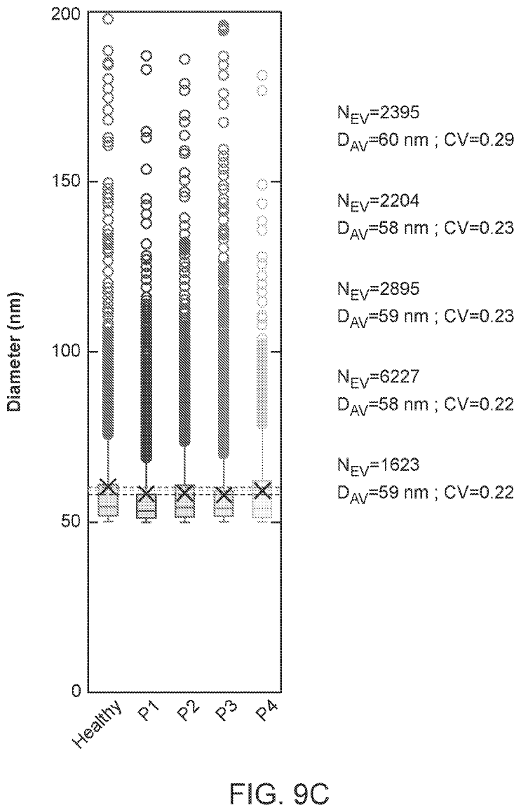

Patient plasma was obtained prior to surgery in EDTA tubes and processed in the same manner (Methods). EVs were first evaluated using ExoView. Images of CD81-enriched EVs that contained tetraspanin markers are shown in FIG. 10; the number of detected EVs (for different subpopulations) in the fluorescence channel is shown in FIGS. 8A-8F. As with pooled healthy plasma, CD41a/CD9 double-positive EVs were detected (indicative of platelet origin), but for each patient, this population was less abundant as compared to CD81-enriched EVs (not associated with platelets). Compared to other patients, patient 3 had more CD81-enriched EVs (and also more CD41a/CD9 double-positive EVs). Next, EVs from PDAC patient plasma were enriched using SEC and combined fraction F1-F5 were evaluated with TEM and microfluidic resistive pulse sensing (MRPS). EVs with intact morphology were observed (FIG. 9A). The average EV diameter obtained with MRPS (FIG. 9B) ranged between 67 and 72 nm for PDAC patients and was 70 nm for pooled healthy plasma (in all cases, EVs were filtered through 200 nm membrane filter before measurements). EV sizes were also evaluated using ExoView (FIG. 9C). The diameter of CD9-enriched EVs ranged between 58 and 59 nm for PDAC patients and it was 60 nm for pooled healthy plasma. Finally, patient samples (directly from crude plasma) were evaluated using the SEVEN assay.

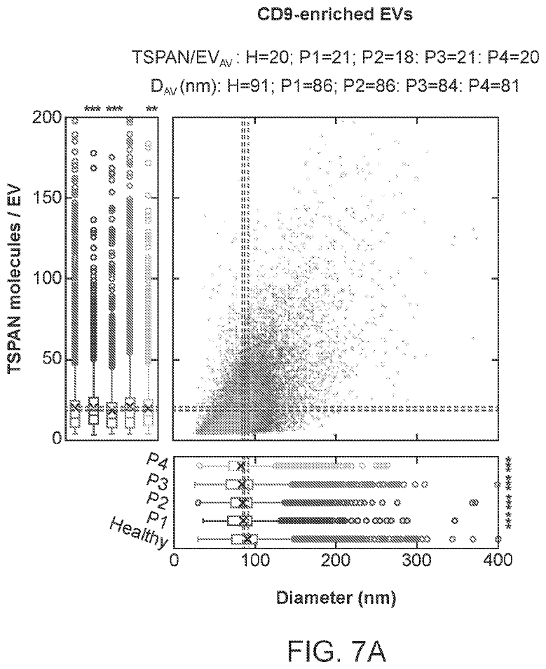

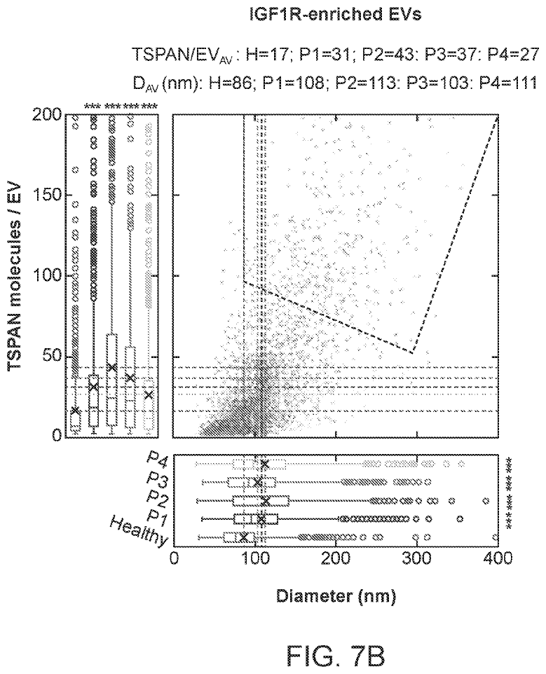

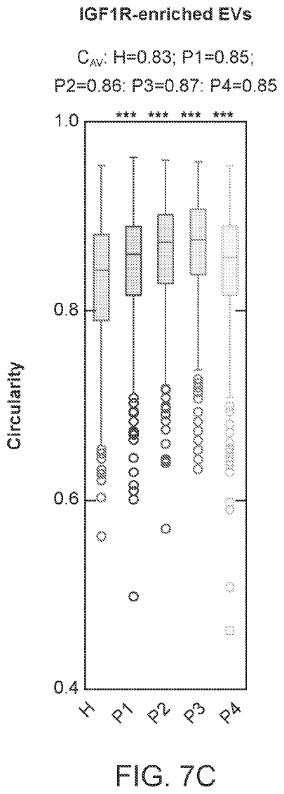

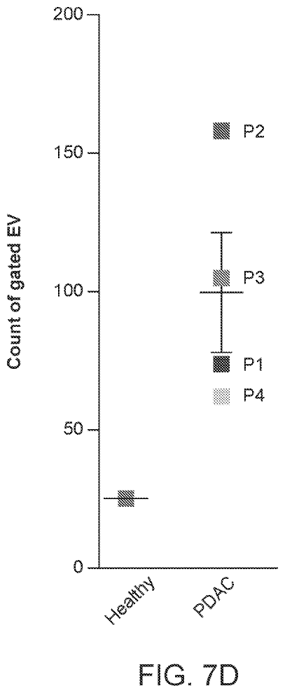

CD9 is generally highly expressed in PDAC microenvironment (stroma), and its abundance is associated with poor prognosis in PDAC patients. Thus, the SEVEN assay was applied to detect differences in CD9-enriched populations in 1:100 diluted healthy pooled plasma vs 1:100 diluted PDAC plasma samples. While the detected number of CD9-enriched EVs varied between all subjects, CD9-enriched EVs from PDAC patients had on average significantly smaller diameter (91 nm for healthy pooled plasma and 81-86 nm for PDAC patients (FIG. 7A and Table 7). Little difference was observed in EV circularity (FIG. 5F, FIG. 4D, and Table 8).

TABLE 7
EV characteristics for CD9- and IGFIR-enriched EVs from healthy
pooled plasma and PDAC patient plasma obtained using SEVEN

| | | | Mean | Median | SEM | CV |
|---|---|---|---|---|---|---|
| CD9-enriched | Healthy | EV count/ROI | 511 | 510 | 16 | 0.123 |
| EVs from | P1 | | 333 | 309 | 26 | 0.305 |
| healthy pooled | P2 | | 364 | 365 | 27 | 0.288 |
| plasma and | P3 | | 540 | 537 | 22 | 0.155 |
| PDAC patient | P4 | | 382 | 414 | 20 | 0.206 |
| plasma | Healthy | Diameter | 91 nm | 85 nm | 0.36 nm | 0.343 |
| | | TSPAN/EV | 20 | 14 | 0.23 | 0.978 |
| | | Circularity | 0.863 | 0.872 | 0.0006 | 0.058 |
| | P1 | Diameter | 86 nm | 83 nm | 0.33 nm | 0.275 |
| | | TSPAN/EV | 21 | 17 | 0.22 | 0.764 |
| | | Circularity | 0.869 | 0.878 | 0.0007 | 0.054 |
| | P2 | Diameter | 86 nm | 83 nm | 0.34 nm | 0.295 |
| | | TSPAN/EV | 18 | 13 | 0.22 | 0.885 |
| | | Circularity | 0.864 | 0.873 | 0.0007 | 0.060 |
| | P3 | Diameter | 84 nm | 80 nm | 0.33 nm | 0.357 |
| | | TSPAN/EV | 21 | 14 | 0.24 | 1.024 |
| | | Circularity | 0.862 | 0.872 | 0.0006 | 0.062 |
| | P4 | Diameter | 81 nm | 79 nm | 0.29 nm | 0.269 |
| | | TSPAN/EV | 20 | 15 | 0.21 | 0.816 |
| | | Circularity | 0.868 | 0.877 | 0.0006 | 0.055 |
| IGFR-enriched | Healthy | EV count/ROI | 55 | 84 | 3.4 | 0.238 |
| EVs from healthy | P1 | | 48 | 40 | 7.6 | 0.617 |
| pooled plasma | P2 | | 58 | 55 | 3.7 | 0.246 |
| and PDAC | P3 | | 53 | 41 | 7.0 | 0.508 |
| patient plasma | P4 | | 46 | 51 | 7.7 | 0.653 |
| | Healthy | Diameter | 86 nm | 77 nm | 1.36 nm | 0.452 |
| | | TSPAN/EV | 17 | 8 | 0.85 | 1.431 |
| | | Circularity | 0.830 | 0.843 | 0.0023 | 0.080 |
| | P1 | Diameter | 108 nm | 96 nm | 1.87 nm | 0.465 |
| | | TSPAN/EV | 31 | 19 | 1.34 | 1.152 |
| | | Circularity | 0.847 | 0.860 | 0.0023 | 0.071 |
| | P2 | Diameter | 113 nm | 100 nm | 1.85 nm | 0.484 |
| | | TSPAN/EV | 43 | 25 | 1.57 | 1.069 |
| | | Circularity | 0.860 | 0.874 | 0.0020 | 0.068 |
| | P3 | Diameter | 103 nm | 92 nm | 1.76 nm | 0.480 |
| | | TSPAN/EV | 37 | 23 | 1.35 | 1.031 |
| | | Circularity | 0.866 | 0.875 | 0.0019 | 0.063 |
| | P4 | Diameter | 111 nm | 98 nm | 2.02 nm | 0.476 |
| | | TSPAN/EV | 27 | 12 | 1.32 | 1.285 |
| | | Circularity | 0.846 | 0.856 | 0.0024 | 0.075 |

TABLE 8
P-values for CD9- and IGF1R-enriched EVs from
healthy pooled plasma and PDAC patient plasma

| | | | | | p-value |
|---|---|---|---|---|---|
| CD9-enriched | Healthy | vs | P1 | Diameter | 2.55E−23 |
| EVs | | | | TSPAN/EV | 1.14E−14 |
| from | | | | Circularity | 1.21E−11 |
| healthy | | vs | P2 | Diameter | 4.76E−25 |
| pooled | | | | TSPAN/EV | 4.95E−06 |
| plasma | | | | Circularity | 1.84E−01 |
| and | | vs | P3 | Diameter | 2.83E−79 |
| PDAC | | | | TSPAN/EV | 9.20E−02 |
| patient | | | | Circularity | 6.69E−01 |
| plasma | | vs | P4 | Diameter | 3.31E−106 |
| | | | | TSPAN/EV | 8.73E−03 |
| | | | | Circularity | 2.75E−11 |
| IGF1R-enriched | Healthy | vs | P1 | Diameter | 2.38E−23 |
| EVs from | | | | TSPAN/EV | 1.47E−29 |
| healthy pooled | | | | Circularity | 6.09E−07 |
| plasma and | | vs | P2 | Diameter | 8.06E−32 |
| PDAC patient | | | | TSPAN/EV | 3.92E−54 |
| plasma | | | | Circularity | 3.44E−24 |
| | | vs | P3 | Diameter | 1.07E−13 |
| | | | | TSPAN/EV | 5.63E−40 |
| | | | | Circularity | 3.08E−32 |
| | | vs | P4 | Diameter | 3.84E−26 |
| | | | | TSPAN/EV | 1.93E−12 |
| | | | | Circularity | 2.64E−06 |

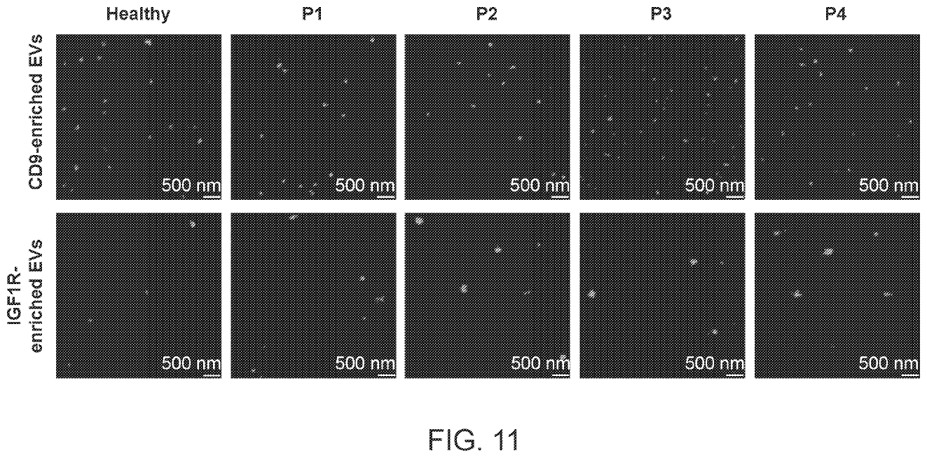

Raw SMLM images of CD9-enriched EVs are shown in FIG. 11, top row. Given that higher expression of IGF1R is associated with worse patient outcomes in resected PDAC, IGF1R-enriched EVs were analyzed. For these experiments, a larger volume and higher concentration of diluted plasma (Methods) was used, as IGF1R-enriched EVs are typically in low abundance as compared to tetraspanin-enriched EVs. For healthy pooled plasma, 0.5% of EVs were IGF1R-enriched (compared to tetraspanin-enriched EVs). Compared to EVs from healthy pooled plasma, PDAC patients had on average larger IGF1R-enriched EVs (86 nm for healthy pooled plasma and 103-113 nm for PDAC patients) with more detected tetraspanin molecules (FIG. 7B and Table 7) and a more rounded shape (FIG. 7C and Table 7). Raw SMLM images of IGF1R-enriched EVs are shown in FIG. 11, bottom row. Based on EV size and molecular tetraspanin content, IGF1R-enriched EVs were gated (polygon, FIG. 7B), and a unique population was observed (FIG. 7C) in which EVs from PDAC patients were more abundant.

The above experiments demonstrate advantageous properties of the disclosed SEVEN assay, including (1) highly sensitive assessment of EV numbers, size, molecular content, shape, and heterogeneity; (2) high signal-to-noise ratio; (3) linearity across very low volume (as low as 0.08 μL) sample volumes and/or samples with low EV abundancy; (4) direct analysis from crude plasma samples; (5) detection of a broader distribution of EV sizes as compared to conventional methods; (6) accurate representations of EV shape; (7) highly sensitive and precise quantitative measurement (i.e., molecular counting) of EV content; and (8) high reproducibility between measurements. Furthermore, SEVEN facilitates comparison of the effects of various EV purification methods (e.g., SEC) on different EV properties, including abundance, tetraspanin content, and shape (e.g., circularity) of EVs. Further still. SEVEN allows for highly-sensitive measurements of EV molecular content/cargo in disease states, e.g., PDAC, thereby providing clinically-relevant applications for early-stage detection of disease pathobiology.

### Example 2: Transcriptomic Profiling of Target Cells Via Analysis of Extracellular Vesicle Cargo

### Methods and Materials

### Study Cohort

Between September 2016 and May 2019, 181 patients with ADHF (discovery, N=14; validation, N=167). ADHF diagnosis required (1) clinical symptoms (including exertional or rest dyspnea, orthopnea, or PND) and biochemical evidence (N-terminal pro-BNP level >1000 μg/ml or BNP >400 μg/ml), or (2) clinical signs: radiographic pulmonary edema or pleural effusions, elevated jugular venous pressure, lower extremity edema, or rales on pulmonary examination, hemodynamic evidence (right atrial pressure >10 mmHg: pulmonary capillary wedge pressure >18 mmHg) and clinical response to intravenous diuretic therapy (as determined by a physician). Additional inclusion criteria were age ≥18 years and an assessment of left ventricular function within the last year or planned during hospital admission. LVEF was acquired non-invasively within one day from admission, based on echocardiography readings. LVEF was acquired by end-diastolic frame minus net counts in the end-systolic frame divided by net counts in end-diastole and confirmed by another clinician using recalculation in 2D mode (DV). Patients with cardiac amyloidosis or active malignancies were excluded. Diuretic therapy was determined by treating providers. As a healthy “control” sample, 33 patients were included who underwent ablation for supraventricular tachycardia (SVT), with blood available at the time of ablation (discovery, N=9; validation, N=24: Table 9 and Table 10) with normal cardiac function by clinical echocardiographic study in the past year. The study protocol was approved by the local Institutional Review Board.

RNA Isolation from EVs

RNA was isolated from EVs from a starting volume of 0.5 mL pooled plasma (control and heart failure samples) for each replicate using the detailed protocol is provided in the Supplementary material. Each fraction was validated for its characteristic markers using a western blot. EV compartment was validated using markers for Alix, CD63, CD81, Syntenin, 58K Golgi protein: lipid fractions by ApoA1 and ApoE, Ago fractions with Ago antibody.

cDNA and qRT-PCR

cDNA was synthesized from 100 ng of total RNA using standard methods. For the validation cohort, 0.1 pg of C. elegans RNA spike-in control was used as a normalizer during cDNA synthesis of 100ng of plasma EV-RNAs. Real-time qPCR analysis was performed to quantify target expression using gene-specific primers generated from coverage analysis and normalized to the spike-in control for calculating the dCt values.

### Single Nuclear RNA Sequencing

To investigate cardiac cell type of origin and expression patterns, the expression of single cardiac nuclear transcriptome from 11 human hearts with dilated cardiomyopathy (DCM), 15 human hearts with end-stage hypertrophic cardiomyopathy (HCM), and 16 non-failing human hearts were analyzed. The snRNAseq data was normalized and scaled. The top-most differentially expressed genes in plasma from HFpEF vs. Control EVs and HFrEF vs. Control EVs were filtered based on FDR (<5%), analyzed using the scanpy function, and then enriched nuclei were overlayed on a Uniform manifold approximation and projection (UMAP) plot from normal heart, DCM and HCM. These genes were also represented by a dot-plot of the different cardiac cell types generated using custom code.

### In Vitro (Cellular) Studies

Human induced pluripotent stem cell-derived cardiomyocytes (iPSC-CMs) were cultured according to known protocols. Human cardiac fibroblasts were cultured using conventional methods. Cell stress was applied using conventional methods. Hypoxia and nutrient deprivation (glucose serum deprivation) were each subjected for 5 hours in iPSC-CMs and 24 hrs for fibroblasts and pericytes. For EV RNA isolation, condition media was collected after the stress treatment, concentrated, and EV was isolated. Cellular RNA was collected from cells using known methods. EV and cellular RNA collected were further proceeded with cDNA synthesis and qRT-PCR.

### Statistical Analysis

Statistical analyses of qPCR and dPCR were performed using known methods, qPCR data are expressed as mean±SEM, and the statistical significance was assessed by Kruskal-Wallis test. All other statistical analysis was performed using custom code.

### Results

### Cohort Characteristics

Baseline clinical characteristics for the described cohorts are shown in Table 9 and Table 10.

TABLE 9
Baseline characteristics of the discovery cohort

| | | Heart Failure | | |
|---|---|---|---|---|
| | Controls | HFrEF | HFpEF | |
| Measures | (n = 9) | (n = 7) | (n = 7) | p-value |
| Age (y), median | 62 (58-68) | 82 (60-83) | 69 (63-90) | 0.5 |
| (IQR) | | | | |
| Sex (male %) | 33 (3/9) | 71 (5/7) | 85 (6/7) | 0.1 |
| Body mas index | 29 (27-33) | 24 (20-58) | 35 (31-43) | <0.01 |
| (kg/m²), median | | | | |
| (IQR) | | | | |
| Tobacco use (%) | 33 (3/9) | 43 (3/7) | 71 (5/7) | 0.6 |
| Diabetes (%) | 11 (1/9) | 43 (3/7) | 71 (5/7) | 0.05 |
| Paroxysmal AF | 0 (0/9) | 29 (2/7) | 85 (6/7) | <0.01 |
| (%) | | | | |
| Coronary Artery | 0 (0/9) | 57 (4/7) | 71 (5/7) | 0.06 |
| Disease (CAD) | | | | |
| (%) | | | | |
| Hypertension (%) | 56 (5/9) | 71 (5/7) | 100 (7/7) | 0.1 |
| Weight change, | na | 1.9 (1.5-3.9) | 2.6 (1.9-9.3) | 0.4 |
| kg, median (IQR) | | | | |
| Creatinine, mg/dL, | 0.8 (0.7-1) | 1.4 (1.2-2.3) | 1 (1-1.5) | 0.2 |
| median (IQR) | | | | |
| Troponin, ng/ml, | na | 41 (0.1-71) | 86 (0.01-400) | 0.7 |
| median (IQR) | | | | |
| NTproBNP, | na | 5239 | 5253 | 0.9 |
| pg/mL, median | | (2425-8399) | (2792-10417) | |
| (IQR) | | | | |
| QRS duration, ms, | 84 (84-88) | 150 (101-164) | 92 (88-120) | 0.04 |
| median (IQR) | | | | |
| NYHA (% > 2) | na | 57 (4/7) | 57 (4/7) | 1 |
| Left ventricular | 61 (60-68) | 24 (11-43) | 63 (54-68) | <0.01 |
| ejection fraction | | | | |
| (%), median (IQR) | | | | |
| Left ventricular | 39 (38-47) | 59 (49-74) | 51 (45-59) | 0.1 |
| end-diastolic | | | | |
| diameter (mm), | | | | |
| median (IQR) | | | | |
| Intraventricular | 10 (9.5-10.5) | 9 (8-10) | 12 (11-14) | 0.02 |
| septum thickness | | | | |
| (mm), median | | | | |
| (IQR) | | | | |
| Left ventricular | 8.5 (8-10.5) | 9 (7-10) | 12 (10-13) | 0.1 |
| posterior wall | | | | |
| thickness (mm), | | | | |
| median (IQR) | | | | |
| Left atrium | 33 (30.5-35) | 46 (42-48) | 47 (43-53) | 0.2 |
| diameter | | | | |
| anteroposterior | | | | |
| (mm), median | | | | |
| (IQR) | | | | |
| Beta blocker (%) | 33 (3/9) | 57 (4/7) | 71 (5/7) | 0.4 |
| ACEi/ARB/ARNI | 33 (3/9) | 43 (3/7) | 29 (2/7) | 1 |
| (%) | | | | |
| Diuretic (%) | 11 (1/9) | 71 (5/7) | 100 (7/7) | <0.01 |
| Calcium channel | 33 (3/9) | 14 (1/7) | 29 (2/7) | 0.8 |
| blocker (%) | | | | |

Abbreviations:
ACEi/ARB/ARNI: Angiotensin Converting Enzyme inhibitors/Angiotensin Receptor Blocker/Angiotensin Receptor blocker neprilysin inhibitor;
AF: atrial fibrillation;
BMI: Body Mass Index;
CAD: coronary artery disease;
CKD: chronic kidney disease;
DM = diabetes mellitus 2;
NYHA: New York Heart Association;
na: not applicable.
Data are shown as n, (%), median (1st, 3rd quartile).
Bold values indicate a statistically significant difference with a p value < 0.05.

TABLE 10
Baseline characteristics of the validation cohort

| | | Heart Failure | | |
|---|---|---|---|---|
| | Controls | HFrEF | HFpEF | |
| Measures | (n = 24) | (n = 79) | (n = 88) | p-value |
| Age (y), median | 41 (32-54) | 64 (56-74) | 74 (63-84) | <0.01 |
| (IQR) | | | | |
| Sex (male %) | 42 (10/24) | 67 (53/79) | 60 (53/88) | 0.3 |
| Body mas index | 28 (22-34) | 28 (23-34) | 33 (28-41) | <0.01 |
| (kg/m²), median | | | | |
| (IQR) | | | | |
| Tobacco use (%) | na | 56 (42/75) | 63 (52/83) | 0.5 |
| Diabetes (%) | na | 42 (33/79 | 47 (39/83) | 0.7 |
| Paroxysmal AF | na | 46 (36/79) | 68 (60/88) | <0.01 |
| (%) | | | | |
| Coronary Artery | na | 58 (46/79) | 52 (46/88) | 0.5 |
| Disease (CAD) | | | | |
| (%) | | | | |
| Hypertension (%) | na | 73 (58/79) | 90 (79/88) | <0.01 |
| Weight change, | na | 4 (2-8) | 4 (1-7) | 0.2 |
| kg, median (IQR) | | | | |
| Creatinine, mg/dL, | 0.9 | 1 (0.9-2) | 1 (1-2) | 0.1 |
| median (IQR) | (0.5-1) | | | |
| Troponin, ng/ml, | na | 27 (0.04-71) | 33.5 | 0.6 |
| median (IQR) | | | (8-76) | |
| NTproBNP, | na | 5245 | 2808 | 0.3 |
| pg/mL, median | | (1896-7361) | (1419-6087) | |
| (IQR) | | | | |
| QRS duration, ms, | na | 110 (96-136) | 102 (88-146) | 0.9 |
| median (IQR) | | | | |
| NYHA (% > 2) | na | 64 (34/53) | 71 (37/52) | 0.5 |
| Left ventricular | 61 (60-66) | 23 (17-35) | 63 (56-68) | <0.01 |
| ejection fraction | | | | |
| (%), median (IQR) | | | | |
| Left ventricular | 50 (42-52) | 61 (56-67) | 47 (43-51) | <0.01 |
| end-diastolic | | | | |
| diameter (mm), | | | | |
| median (IQR) | | | | |
| Intraventricular | 10 (8-10) | 10 (8-12) | 12 (11-14) | <0.01 |
| septum thickness | | | | |
| (mm), median | | | | |
| (IQR) | | | | |
| Left ventricular | 9 (8-10) | 10 (9-12) | 11 (10-13) | <0.01 |
| posterior wall | | | | |
| thickness (mm), | | | | |
| median (IQR) | | | | |
| Left atrium | 35 (24-38) | 48 (42-52) | 45 (41-49) | 0.2 |
| diameter | | | | |
| anteroposterior | | | | |
| (mm), median | | | | |
| (IQR) | | | | |
| Beta blocker (%) | na | 82 (65/79) | 74 (65/88) | 0.2 |
| ACEi/ARB/ARNI | na | 48 (38/79) | 23 (20/88) | <0.01 |
| % | | | | |
| Diuretic (%) | na | 90 (71/79) | 93 (82/88) | 0.6 |
| Calcium channel | na | 8 (6/79) | 28 (25/88) | <0.01 |
| blocker (%) | | | | |

Abbreviations:
ACEi/ARB/ARNI: Angiotensin Converting Enzyme inhibitors/Angiotensin Receptor Blocker/Angiotensin Receptor blocker neprilysin inhibitor;
AF: atrial fibrillation;
BMI: Body Mass Index;
CAD: coronary artery disease;
CKD: chronic kidney disease;
DM = diabetes mellitus 2;
NYHA: New York Heart Association;
na: not applicable.
Data are shown as n, (%), median (1st, 3rd quartile).
Bold values indicate a statistically significant difference with a p value < 0.05.

The median (interquartile range [IQR]) time between admission and initial blood draw was 2 (1-3) days for the discovery cohort and 3 (2-4) days for the validation cohort. The median (IQR) hospital stay was 6 (3-8) days in the discovery cohort and 5 (3-7) days in the validation cohort. The median (IQR) time between the final blood draw and discharge was 1 (1-3) day in both cohorts. In general, individuals with HFpEF had increased body mass index (median BMI [IQR], discovery cohort HFpEF 35 kg/m2, [35-43 kg/m2]; p<0.01), with a higher prevalence of atrial fibrillation (p<0.01). HFpEF patients in the validation cohort were older and had a higher prevalence of hypertension. Sixty-seven percent of HFrEF patients were male, 58% had ischemic cardiac disease and a history of CAD, while 38% had undergone coronary artery bypass graft surgery at the time of admission.

### Transcriptional Profiling of EVs in ADHF Revealed Broad Differences Across HF Subtypes

Long RNA sequencing of plasma extracellular RNA was performed on the discovery cohort, as described herein. The use of this protocol allowed for the discovery of both mRNA and lncRNA transcripts. Of the total reads, an average of 53.4% was uniquely mapped to the human genome. Multi-mapped reads were omitted for the gene counting. On average, 10.4% of uniquely mapped reads were assigned to genes. An average number of 14111 and 16697 mRNA transcripts and 13766 and 15580 lncRNA transcripts were identified (transcripts per million, TPM >=1.0) in control and heart failure patient plasma samples, respectively. An average of 17045 and 16350 mRNA transcripts and 16590 and 14570 lncRNA transcripts were detected at ADHF state (Visit 1 or V1) and after decongestion therapy (V2), respectively.

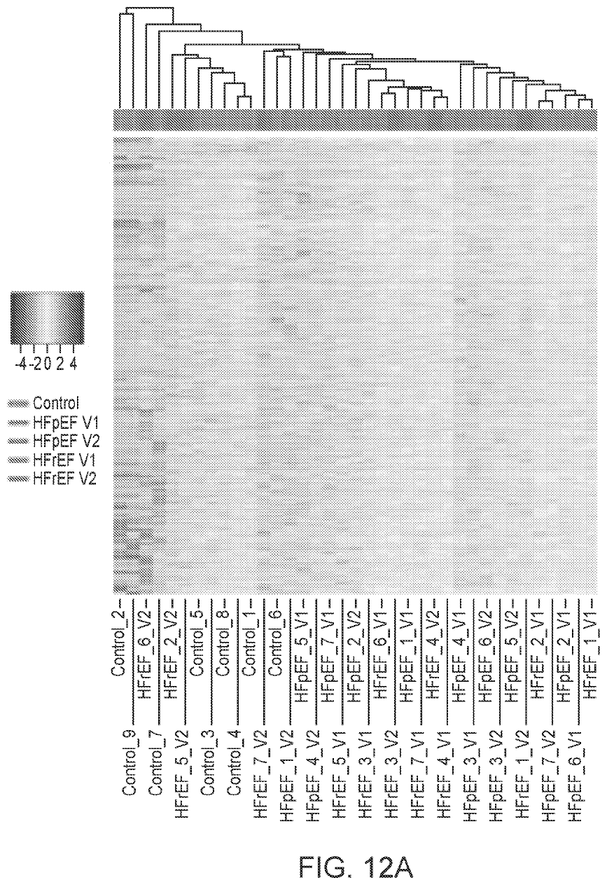

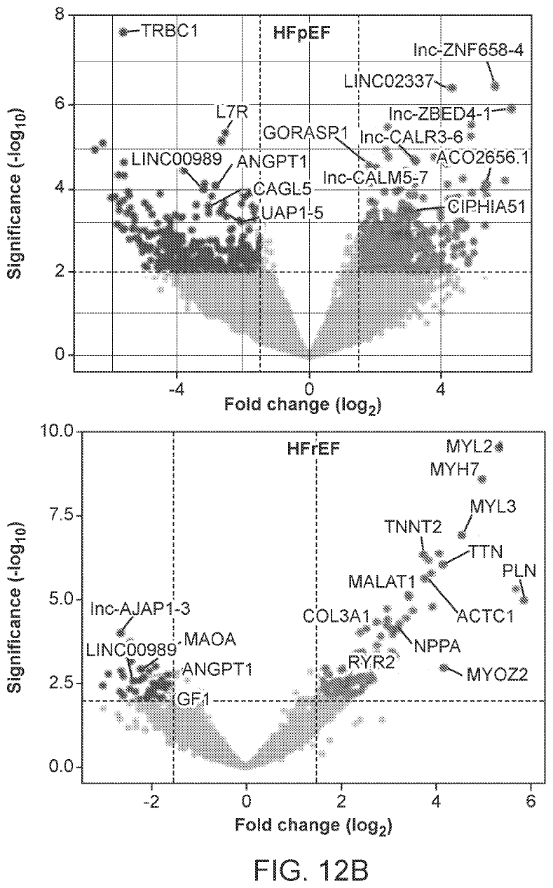

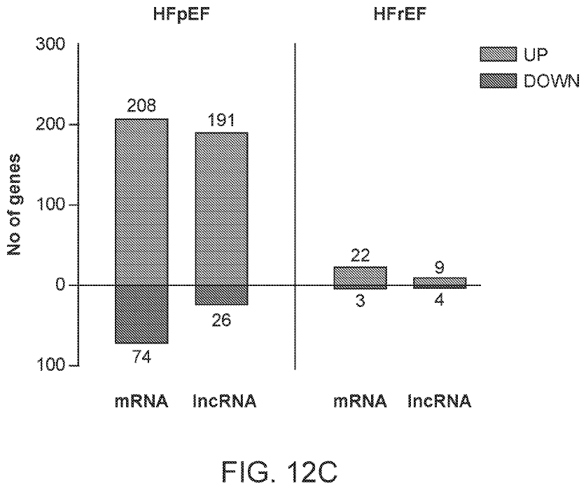

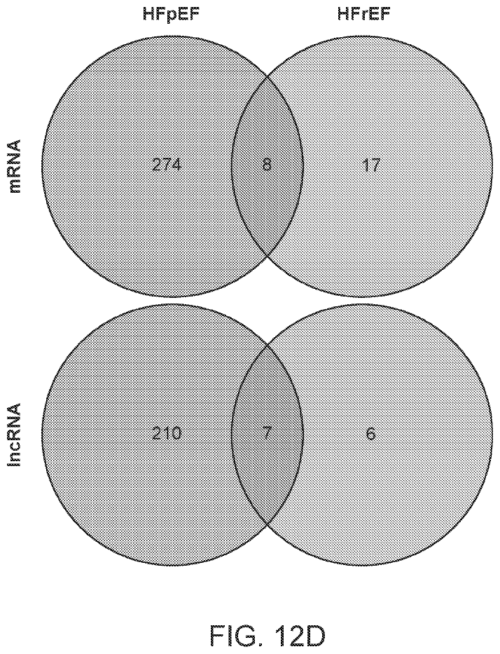

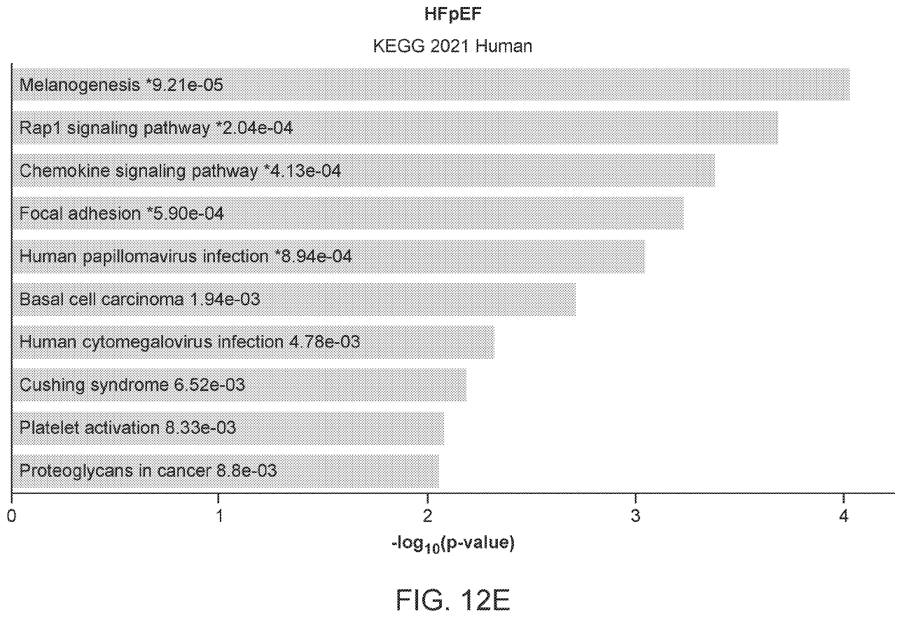

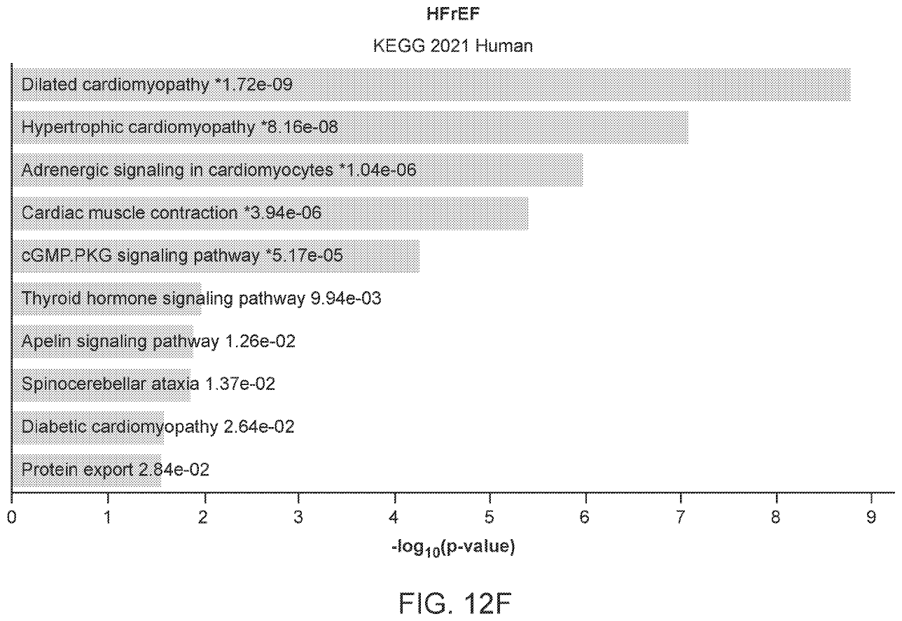

In unsupervised clustering of the transcripts profiled in ADHF (corresponding to V1) (FIG. 12A), control and HF subtypes were grouped separately, with the long non-coding RNA transcriptome demonstrating more consistent differences between the groups. Broad transcriptional differences were observed between ADHF states (both HFpEF and HFrEF) and control patients (FIG. 12B). Notably, much broader differences were observed in the transcriptome of HFpEF, as compared to HFrEF, with a prominent contribution of lncRNAs. A total of 399 RNAs were increased in HFpEF plasma relative to control (208 mRNAs; 191 lncRNAs) and 100 transcripts decreased (74 mRNAs: 26 lncRNAs). In comparison, of 31 RNAs increased in HFrEF patient plasma relative to controls, 22 were mRNAs, and 9 were lncRNAs, while 7 transcripts were decreased (3 mRNAs and 4 lncRNAs) (FIG. 12C). The differential transcriptome during congested ADHF was largely distinct between HFpEF and HFrEF as compared to controls (FIG. 12D), with most transcripts being specific to a given HF subtype. Kyoto Encyclopedia of Genes and Genomes (KEGG) Pathway enrichment analysis showed that the differentially expressed transcripts in HFrEF were primarily associated with the heart, such as dilated cardiomyopathy, hypertrophic cardiomyopathy, adrenergic signaling in cardiomyocytes, etc. By contrast, in HFpEF, implicated pathways were variable and included a broad range of cellular signaling pathways, such as melanogenesis RapT signaling pathway, chemokine signaling pathway, focal adhesion, and pathways involving cancer (FIG. 12E).

### EVs as a Source for the Differential ADHF Transcriptome

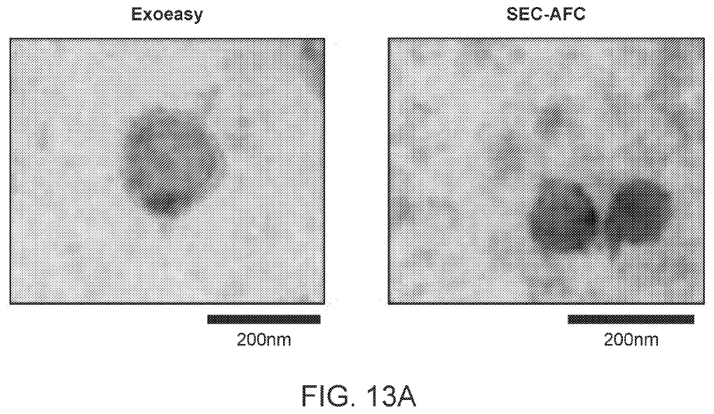

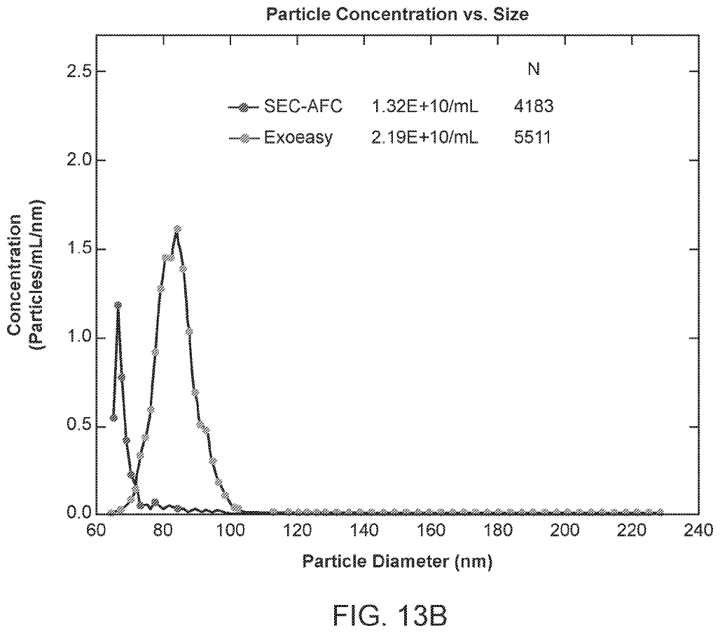

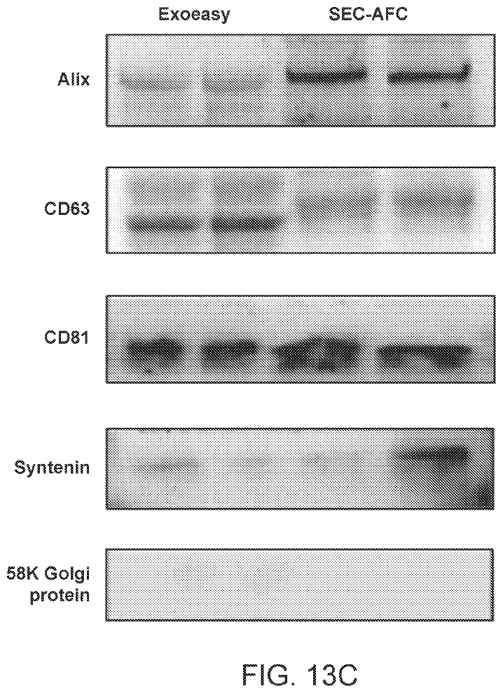

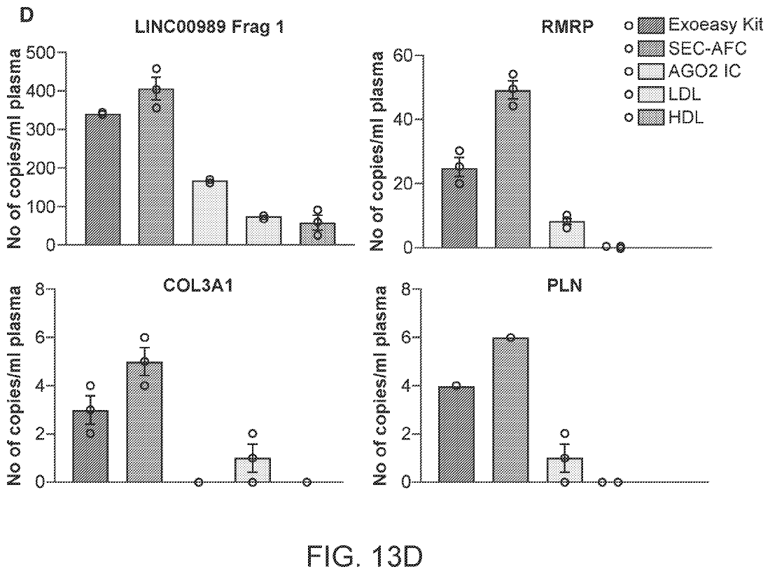

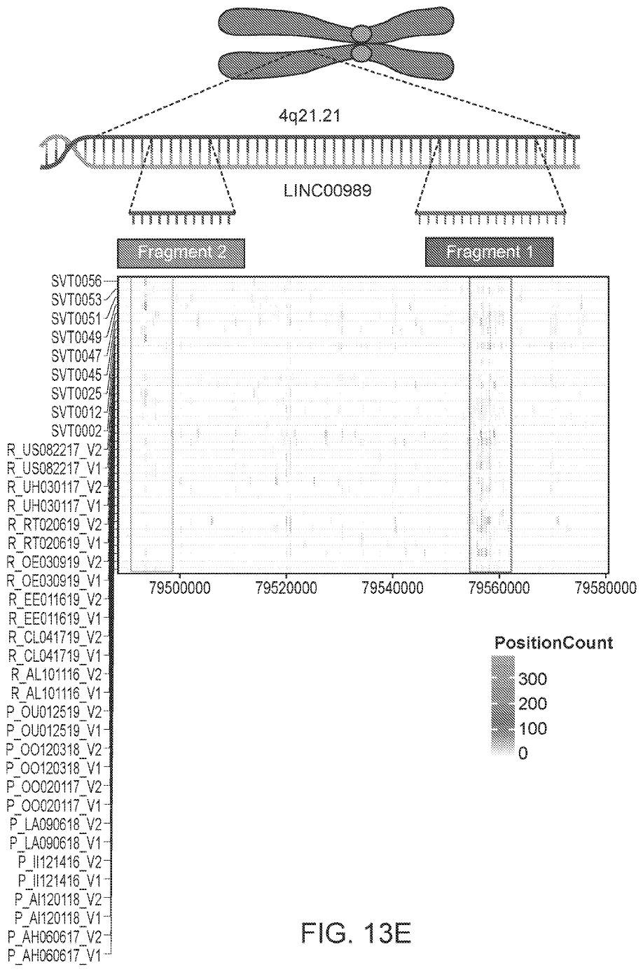

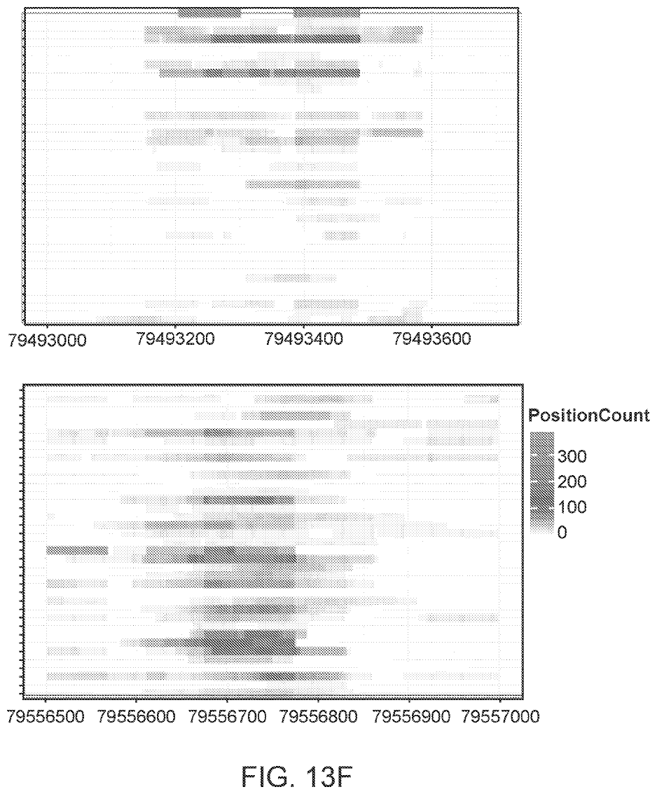

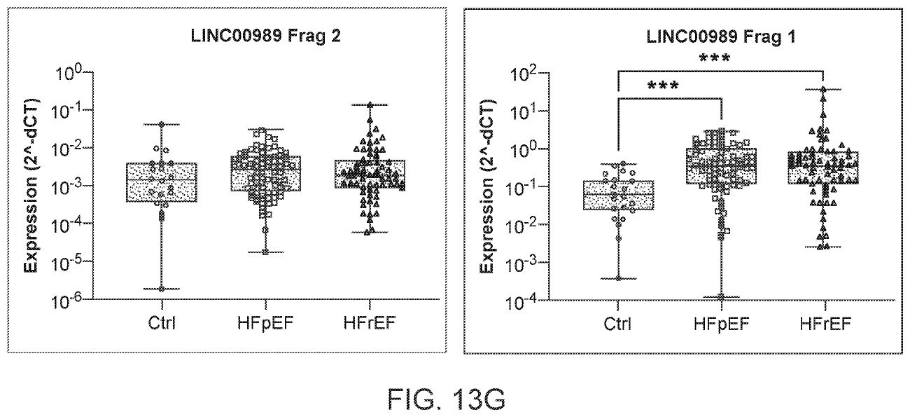

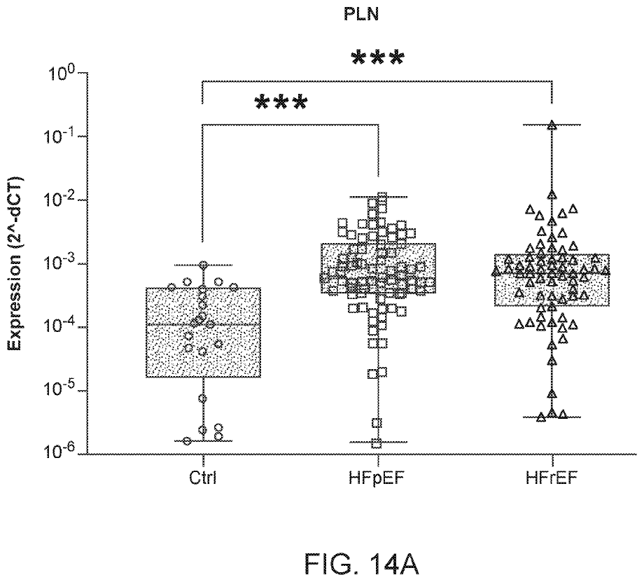

“Unprotected” long RNAs have a short half-life in circulation. To test whether circulating long transcripts were primarily associated with EVs or other RNA carriers, RNA was isolated from different plasma compartments known to stabilize small RNAs in circulation, including argonaut-2 (Ago2)-associated, lipoprotein-associated, and EV compartments in pooled human plasma from both heart failure and control samples. The EV compartment was validated using Minimal Information for Studies of Extracellular Vesicles (MISEV) guidelines (FIGS. 13A-13C). The MISEV guidelines prescribe several criteria that are required to validate presence of EVs, among them: (1) size characterization; and (2) assessment of concentration. These criteria were assessed using Transmission Electron Microscopy (TEM; FIG. 13A), and Microfluidic Resistive Pulse Sensing (MRPS; FIG. 13B). EVs were isolated, purified using size exclusion chromatography (SEC), and measured between 60-100 nm in size with a concentration of around 1010 EV particles/mL of plasma, although the EVs isolated by the SEC method appeared to be smaller in size. The third MISEV criterion requires the characterization of the EV protein content using westem blot with EV markers, specifically, the tetraspanins present on the EV surface (e.g., CD81, CD63 or CD9) and EV cargo proteins (ALIX, TSG101 or SDCBP) and, notably, for small EVs, a negative marker that includes a soluble intracellular compartment (e.g., 58KDa Golgi protein). Therefore, EVs were validated by demonstrating the presence of CD81. CD63, Alix, Syntenin, and the absence of 58KDa Golgi protein (FIG. 13C). Differentially expressed transcripts were assessed between HF and control cohorts using digital PCR in different plasma compartments and identified that the EV compartment exhibited enriched expression (FIG. 13D). Finally, the EV markers and EV concentrations were also assessed in the different patient groups. This analysis did not reveal any significant differences between the control, HFpEF, and HFrEF groups in the baseline characteristics of EVs. Notably, these groups did not differ in total EV concentration, as well as CD81-, CD9-, and CD63-positive EVs. Taken together, the data suggest that plasma concentration of the total EV populations is not significantly altered in HF patients and that long RNA transcripts are primarily present in EVs. Since the exRNA transcripts are primarily enriched in EVs, they will henceforth be referred to as “EV-RNAs.”

To determine the state of the RNA transcripts within the EVs, expression pattern of the differentially expressed EV-derived RNAs was analyzed using coverage plots from RNA-sequencing data. If the full-length transcript were present, uniform coverage of the RNA sequencing reads across the length of the transcript would be expected. Instead, enrichment of selective RNA fragments was observed, suggesting that RNAs were present as fragments within EVs, and that this fragmentation is non-random. For example, while there are two major fragments of LINC00989 detected in the RNAseq data, when the expression was examined using primers designed against each of these fragments, only one fragment was differentially expressed between control and HF plasma (LINC00989 in FIGS. 13E-13G). This finding highlights the importance of post-transcriptional processing in RNA biomarker quantification and the importance of using initial RNAseq data to determine the key fragments to analyze using methodologies such as PCR.

### Validation Ofdifferentially Expressed RNAs in HF Subtypes

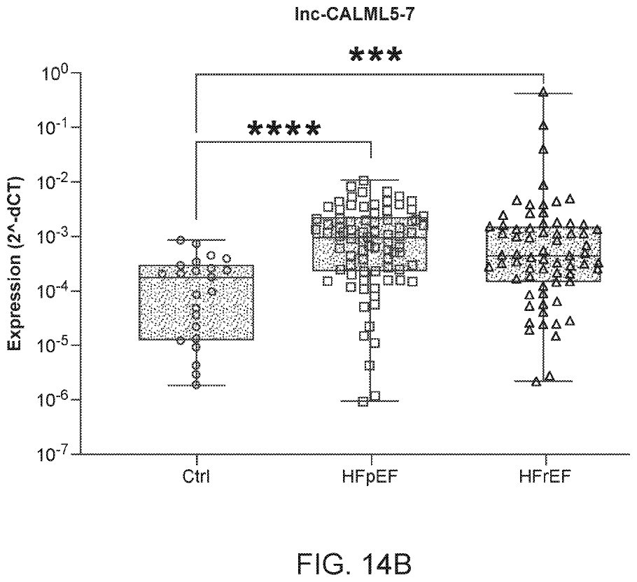

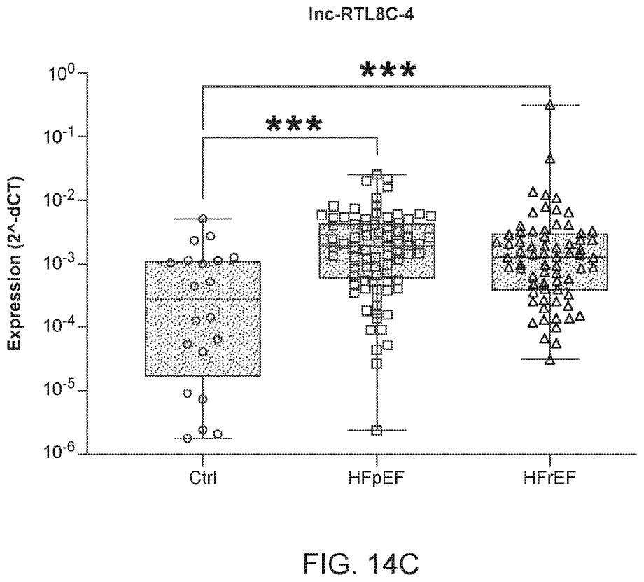

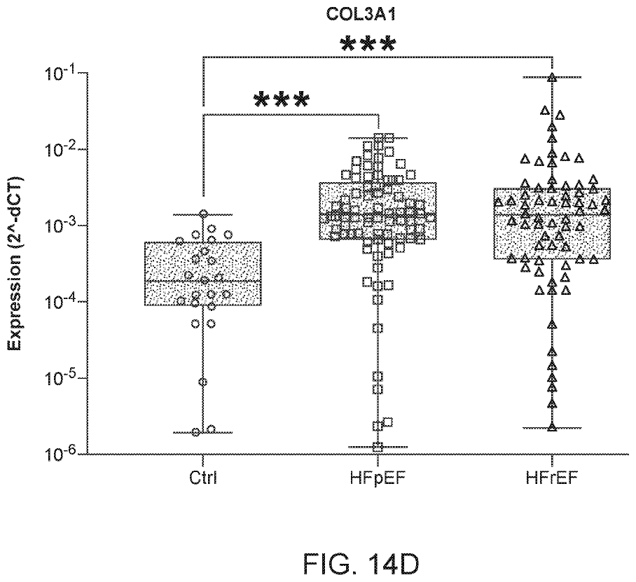

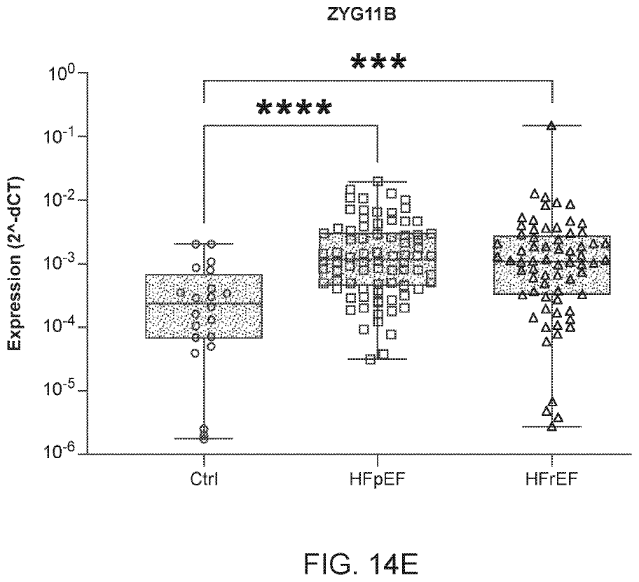

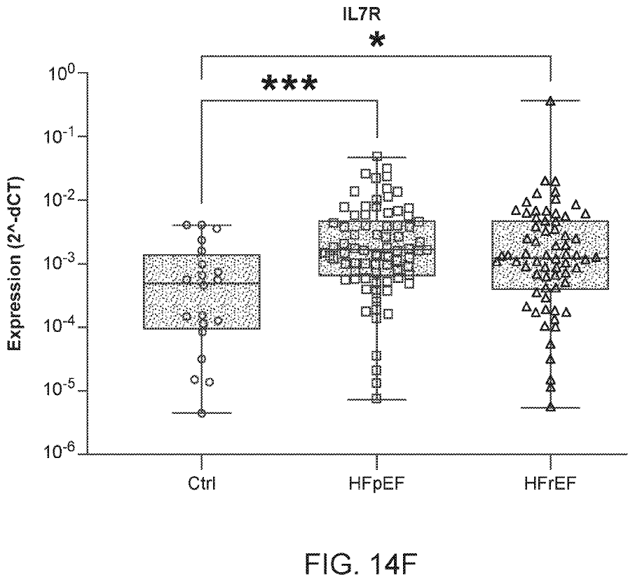

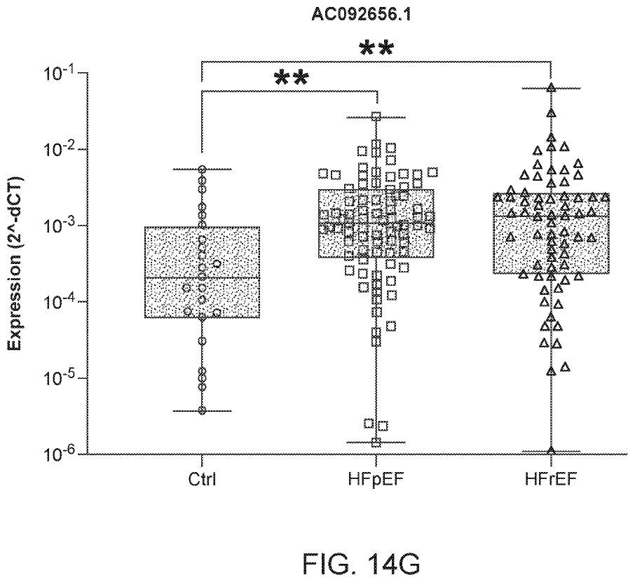

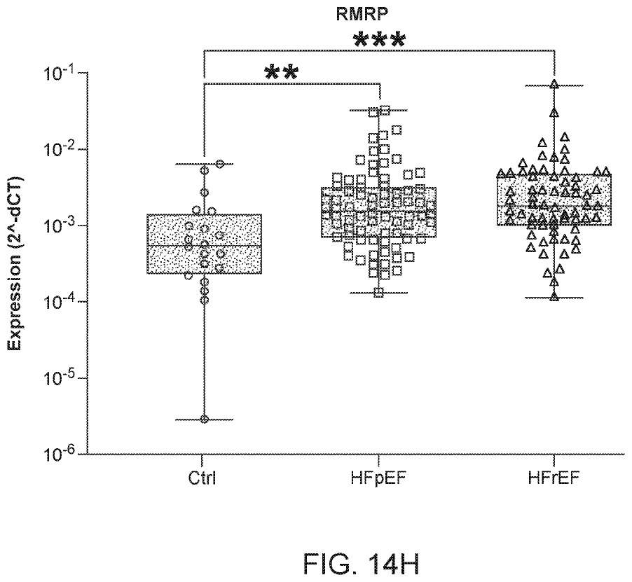

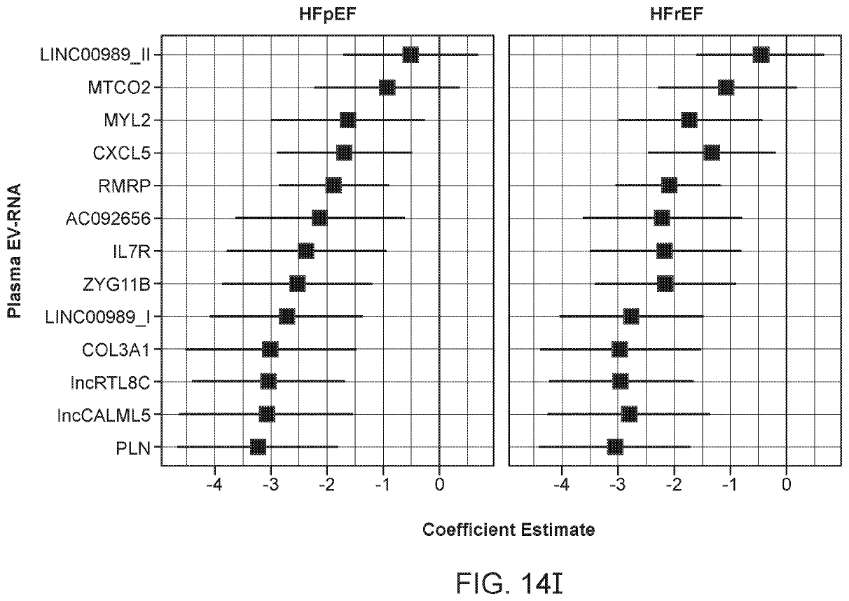

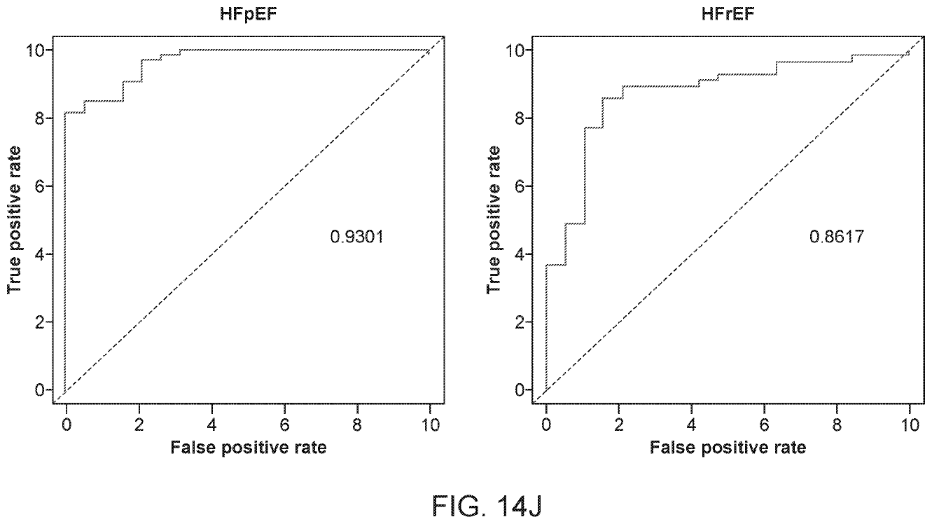

Next, top differentially expressed RNAs were measured using qRT-PCR on plasma-derived EVs obtained at admission from the validation cohort. Expression of 13 targets was examined in 182 patients (24 control; 86 HFpEF: 72 HFrEF), out of which 11 targets (5 lncRNAs and 6 mRNAs) were significantly different between control and ADHF after adjustment for age and sex, stratified by HF subtype (FIGS. 14A-141). Digital PCR was used to confirm the qRT-PCR results. Notably, these EV-RNAs were altered in a similar direction in HFpEF and HFrEF, and receiver-operating characteristic (ROC) analysis performed on the top-five validated targets for each of the HF subtypes after adjustment for age and sex (0.9301 for HFpEF and 0.8617 for HFrEF) improved the C-statistics (FIG. 14J) when compared to age and sex alone (0.8176 for HFpEF and 0.6369 for HFrEF).

Plasma EV lncRNAs Exhibit Dynamic Expression During Decongestion

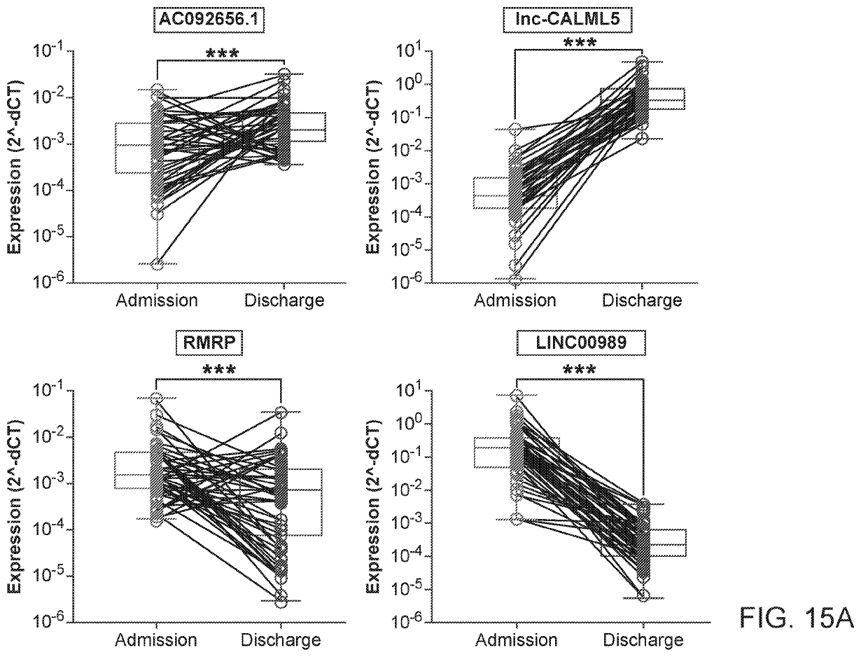

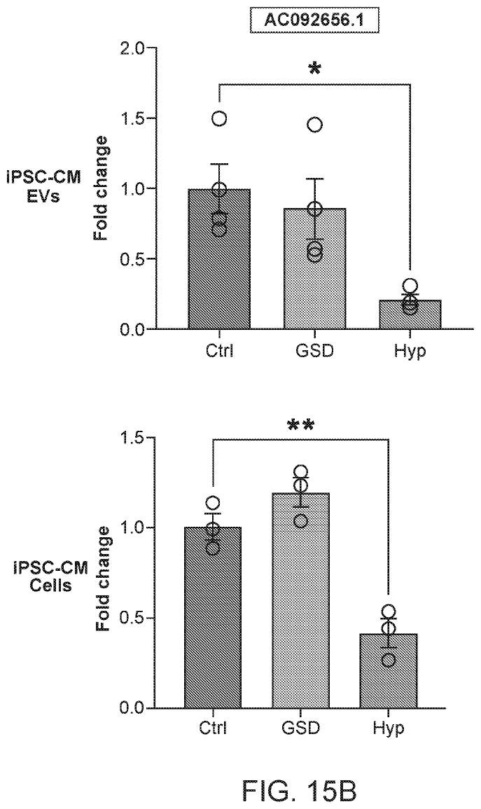

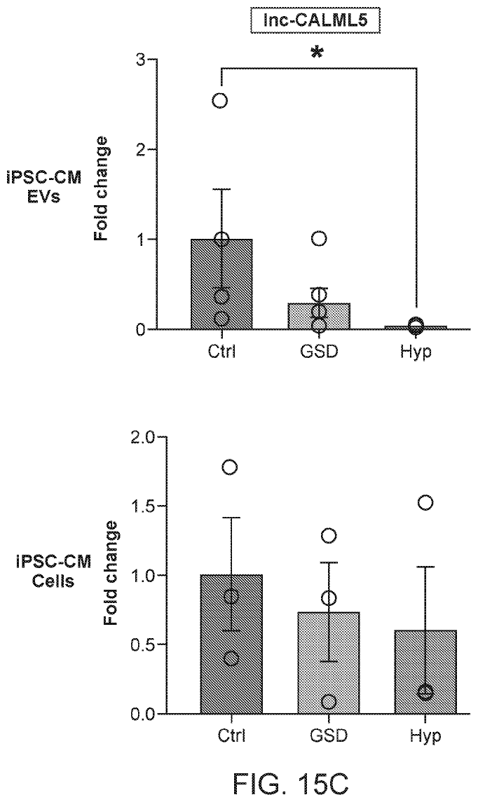

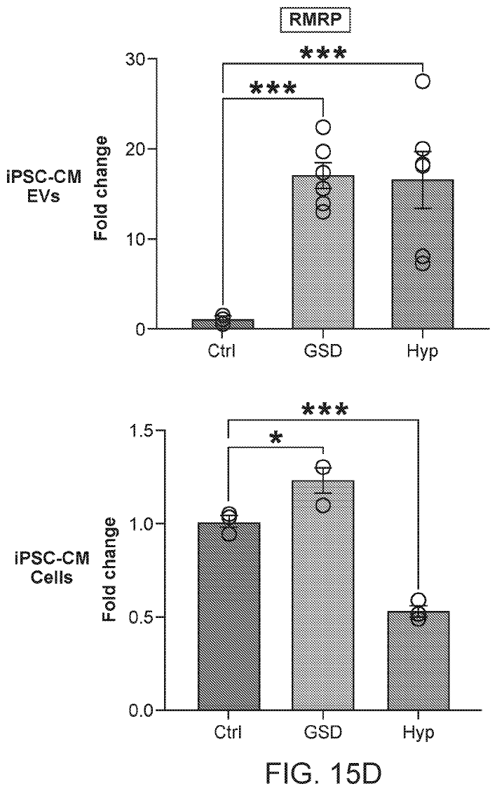

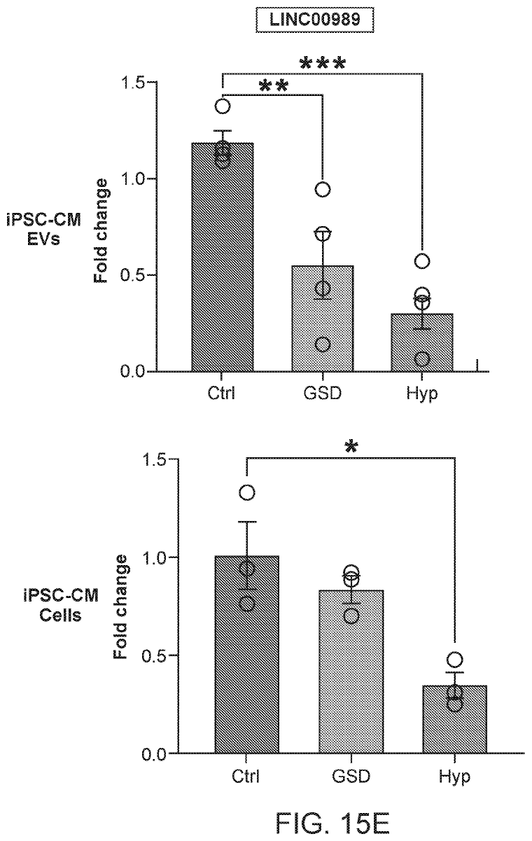

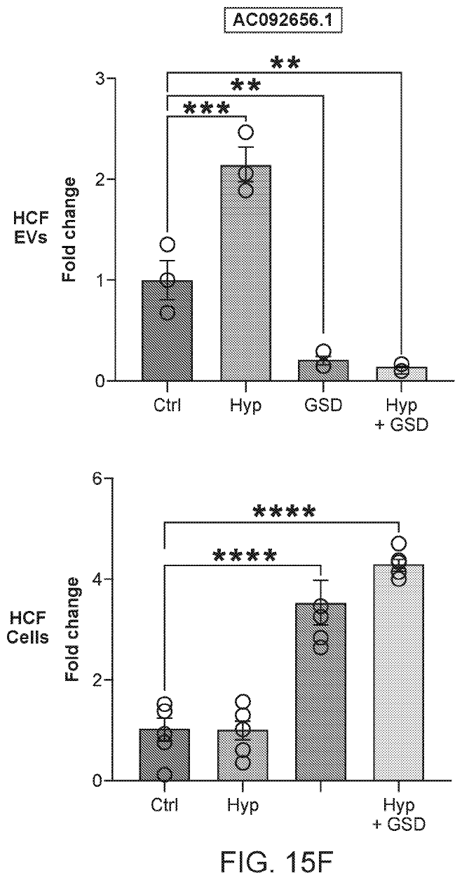

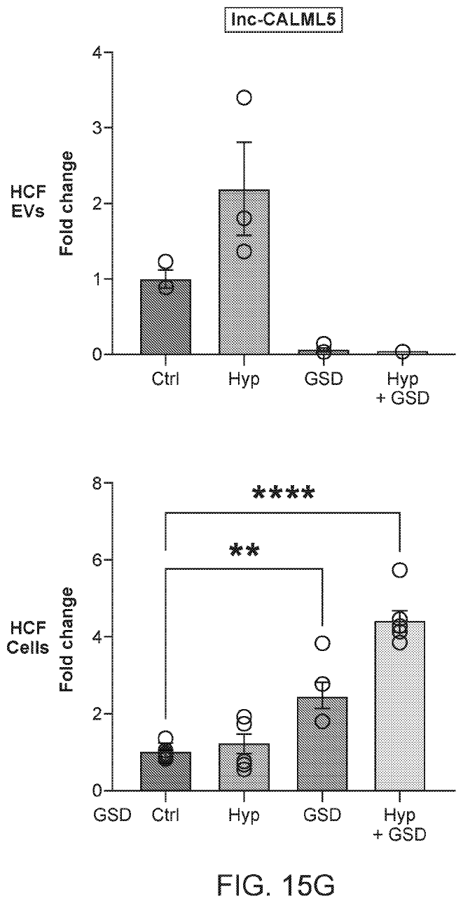

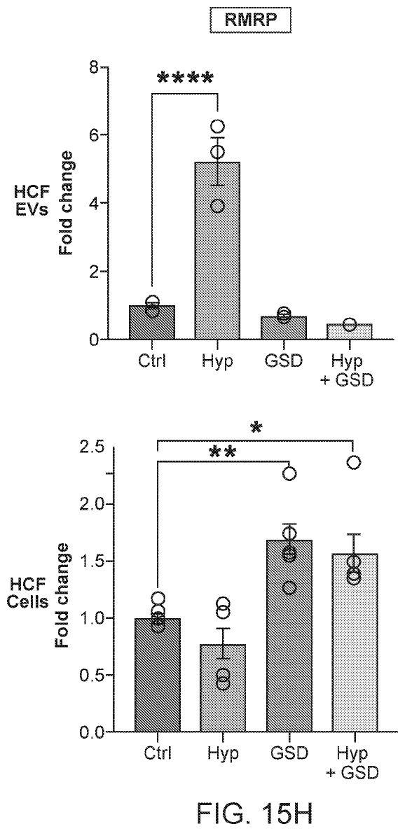

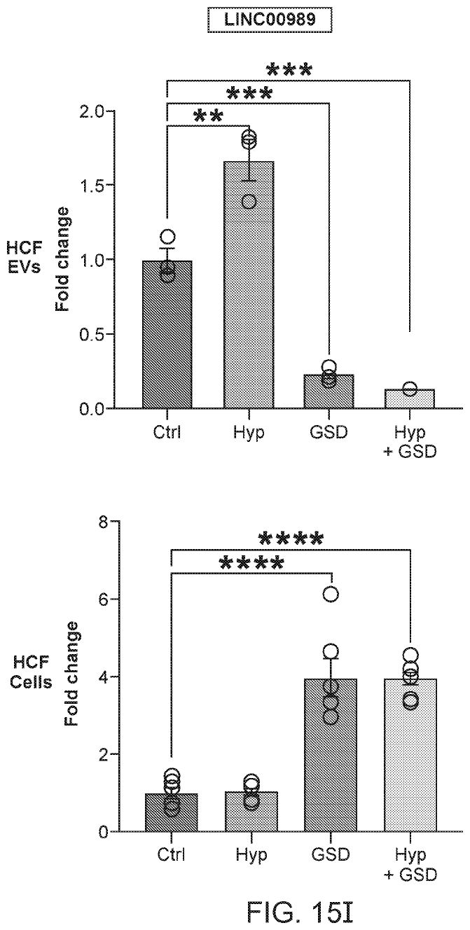

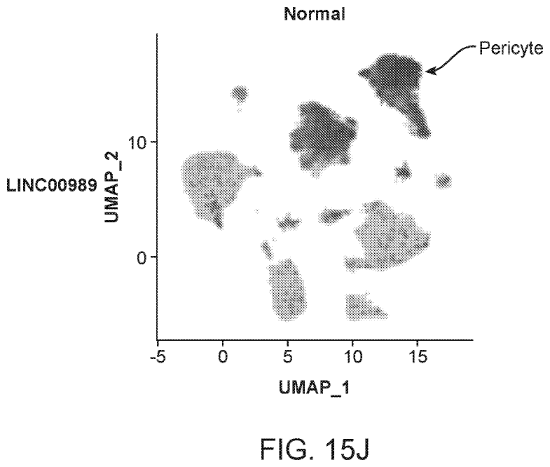

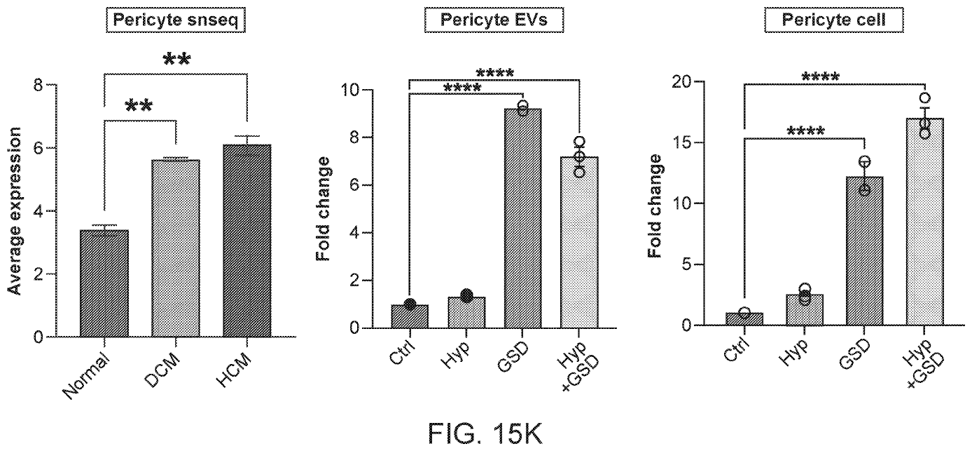

To understand whether the transcripts that validated during the admission of the HF subtypes were a marker of congestion status or reflected other pathophysiological markers during ADHF, changes in expression during decongestion were analyzed. Transcripts that were most differentially expressed in HF patients at the congested (acute) state were prioritized and analyzed for their expression via qRT-PCR after decongestion (diuretic treatment). Of the 11 targets examined, only 4 non-coding RNAs (LINC00989, lnc-CALML5, AC092656.1. RMRP) changed during therapy for ADHF (FIG. 15A). The expression of LINC00989 and RMRP decreased with therapy, and the expression of Inc-CALML5 and AC092656.1 increased.

Dynamic changes in specific lncRNAs with cellular stress in vitro

Given that the aforementioned four lncRNAs dynamically change from the acute state to decongested states, alteration of expression of these lncRNAs was assessed at the cellular level subjected to different stress conditions, such as hypoxia (Hyp) and nutrient deprivation (glucose serum deprivation or GSD). Upon subjecting induced pluripotent stem cells-derived cardiomyocytes (iPSC-CMs) to cellular stressors, the expression of lnc-CALML5-7. AC092656.1, and LINC00989 in EVs and in cells was found to be decreased under hypoxic stress (coordinate with the changes in ADHF for the first two, where plasma EV levels showed lower expression of lnc-CALML5-7 and AC092656.1). In contrast, the expression of RMRP increased in iPSC-CM EVs during stress, similar to the higher levels in plasma EVs of patients with ADHF while its cellular levels showed a decrease (FIGS. 15B-15E). Cellular and EV expression of these lncRNAs were also examined in other cardiac cell-types. When human cardiac fibroblasts (HCF) were subjected to cellular stressors, the expression of all the four lncRNAs in EVs increased upon hypoxia and decreased upon GSD, whereas the converse trend was observed at the cellular level, i.e., expression increased during GSD (FIGS. 15F-15I). Since acute decompensation is a form of acute stress for patients with HF, the trends observed in stressed iPSC-CM EVs for AC09265.18, lnc-CALML5-7, and RMRP, but not for LINC00989, were concordant with those observed in the plasma of HF patients. Notably, the single nuclear transcriptomic analysis revealed that LINC00989 was highly expressed in cardiac pericytes, but not in cardiomyocytes. Thus, the expression of LINC00989 was examined in these cells. When pericytes were subjected to cellular stressors, the expression of LINC00989 was elevated in both cellular and EV compartments. This is in agreement with the increased expression of LINC00989 observed in patient plasma during the acute congested state (FIG. 15J and FIG. 15K).

Combined, the results of this example demonstrate the diagnostic capacity of EVs in HF, as based on: (1) the observation that the HFpEF and HFrEF EV transcriptome is distinct and exhibits organ- and cell-type specificity in the acutely congested state that reflects a divergent underlying pathophysiology; and (2) EV lncRNA transcripts change with decongestion, and are altered with stress in human-derived cells (iPSC-derived cardiomyocytes, and pericytes) in a direction similar to that observed in patients during the acute-to-recompensated transition. These findings further demonstrate the usefulness of target tissue-derived EVs in elucidating disease pathobiology. Specifically, The use of EVs as a “liquid biopsy” across HF subtypes suggested that while the EV transcripts in decompensated HFrEF were more clearly expressed in the heart, specifically in cardiomyocytes, those in decompensated HFpEF exhibited broader systemic origins (non-cardiomyocyte), confirming current clinical conception of HFpEF as a multi-systems disorder. Furthermore, the transcripts prioritized based on patient-oriented EV findings during decongestion demonstrated dynamic in vitro expression, which further underscores the possibility that select lncRNA expression profiles may be specifically implicated in myocardial responses to stress in HF. Together, these results demonstrate dynamic transitions in the EV transcriptome across acute to decongested states that reflect a potential divergent HF pathophysiology, with cellular model evidence that prioritizing EV transcriptome changes during HF therapy may inform discovery efforts across HF sub-types.

### Example 3: Molecular Characterization of EVs from Breast Cancer

HER2-directed therapy, such as monoclonal antibody trastuzumab, has significantly improved the outcome of patients with HER2-positive breast cancer. While current diagnostic methods can identify patients eligible for trastuzumab (HER2-positive), they cannot predict whether these patients would respond to trastuzumab-based therapy. Trastuzumab resistance is a major clinical challenge for which non-invasive diagnostic approaches for monitoring patient response are lacking. EVs secreted from tumor cells represent can serve as a biomarker source, as they carry molecular signatures of their tumor cells of origin. Additionally. EVs can be collected frequently from accessible biofluids.

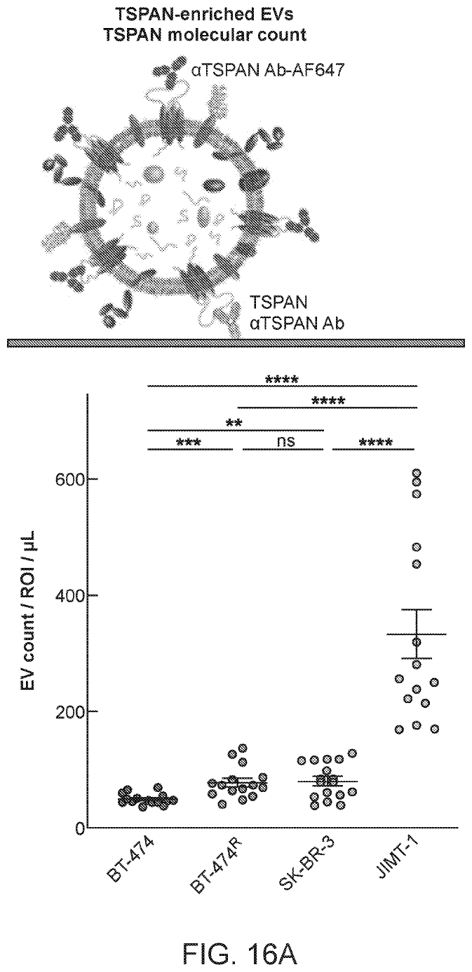

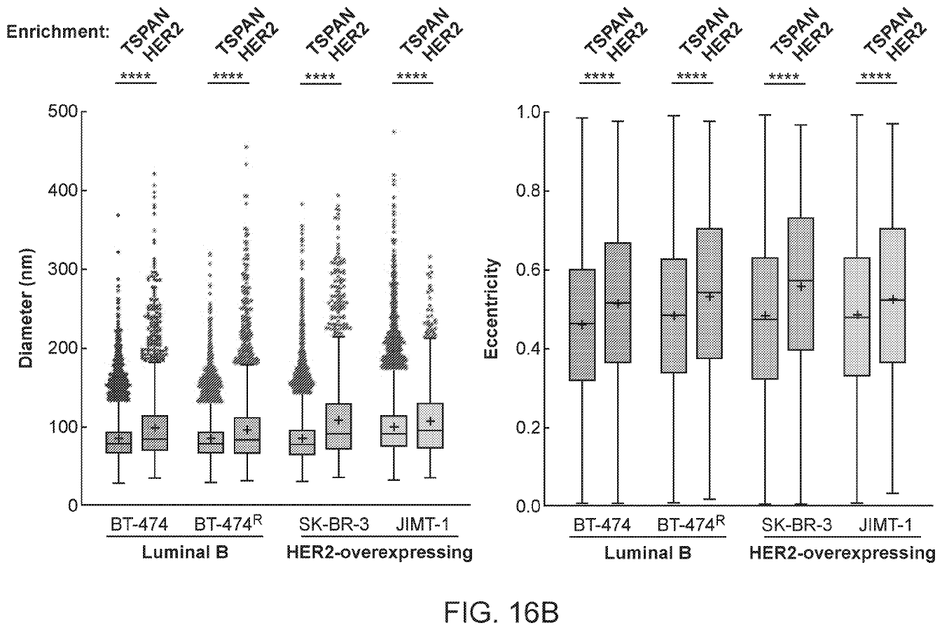

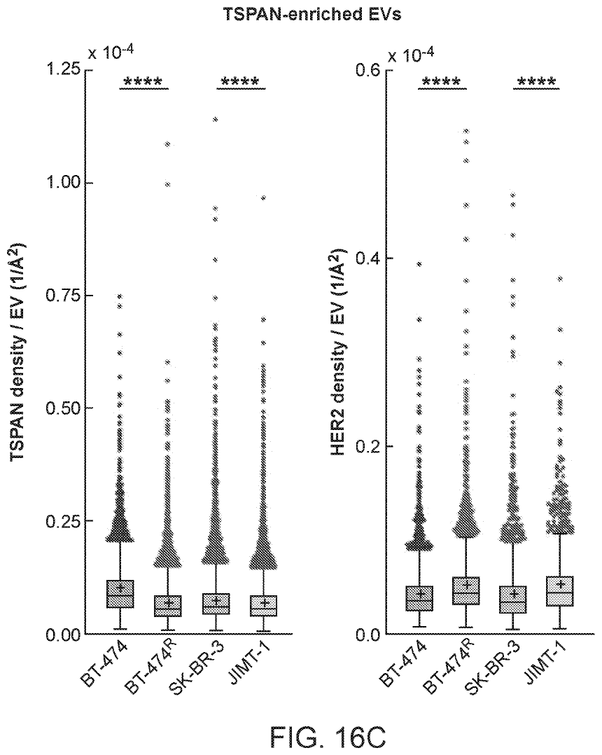

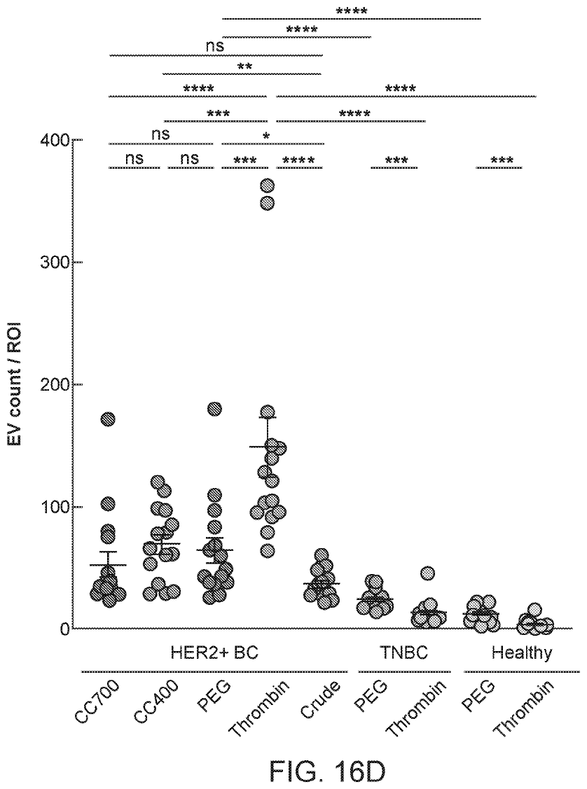

Here, Single Extracellular Vesicle Nanoscopy (SEVEN) was used to comprehensively characterize individual EVs from cultured trastuzumab-sensitive (SK-BR-3 and BT-474) and trastuzumab-resistant (JIMT-1 and BT-474R) breast cancer cell lines. HER2-enriched EVs were isolated on coverslips functionalized with trastuzumab using a method akin to that described in Example 1, above. EV identity was confirmed with AF647-labeled anti-TSPAN antibodies. Imaging and EV analysis were performed according to the method described in Example 1. Moreover, imaging of HER2-enriched EVs from patient plasma was optimized. The number of tetraspanin (CD9, CD63, CD81)-enriched EVs were determined. Trastuzumab-resistant cells were shown to secrete a substantial amount of EVs (FIG. 16A). Next, for HER2- and tetraspanin-enriched EVs, size, shape, and HER2 or tetraspanin molecular density were determined (FIGS. 16B and 16C). HER2-enriched EVs (compared to tetraspanin-enriched EVs from the same cell line) were larger and more elongated (FIG. 16B). Furthermore trastuzumab-resistant cells had a higher density of HER2 molecules and lower density of tetraspanin molecules as compared to corresponding trastuzumab-sensitive cells (FIG. 16C). In a separate experiment, different EV isolation methods were tested for patient plasma and combined with trastuzumab-capture and SEVEN method. A significant number of HER2-enriched EVs were efficiently detected from plasma of HER2-positive breast cancer patient (FIG. 16D). Combined, these results show that SEVEN is a promising method for the assessment of EVs from patient plasma and may have the potential to monitor patient response to therapy.

### Example 4: Characterization of Brain-Enriched Extracellular Vesicles

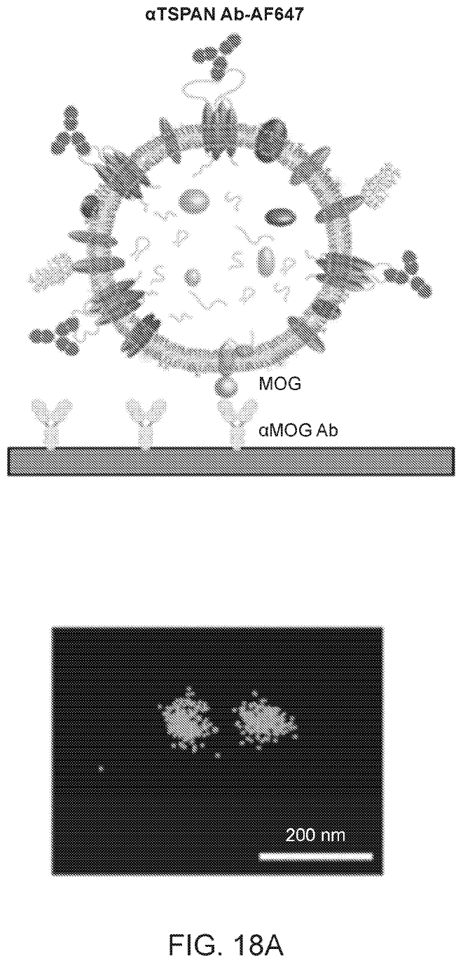

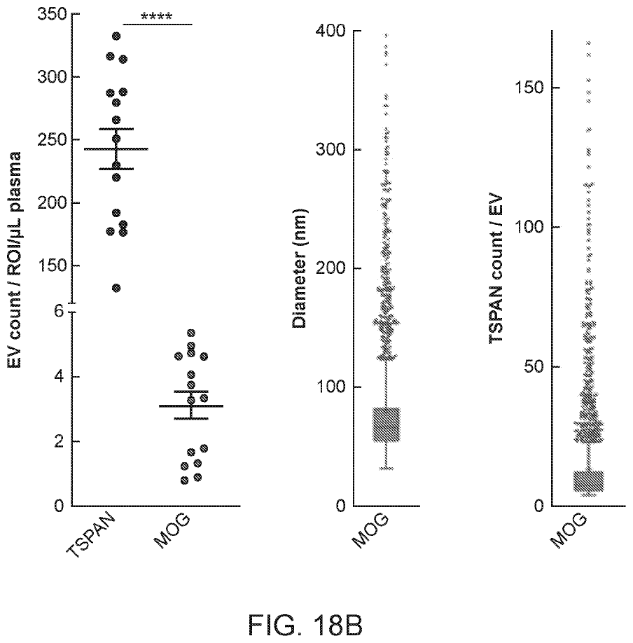

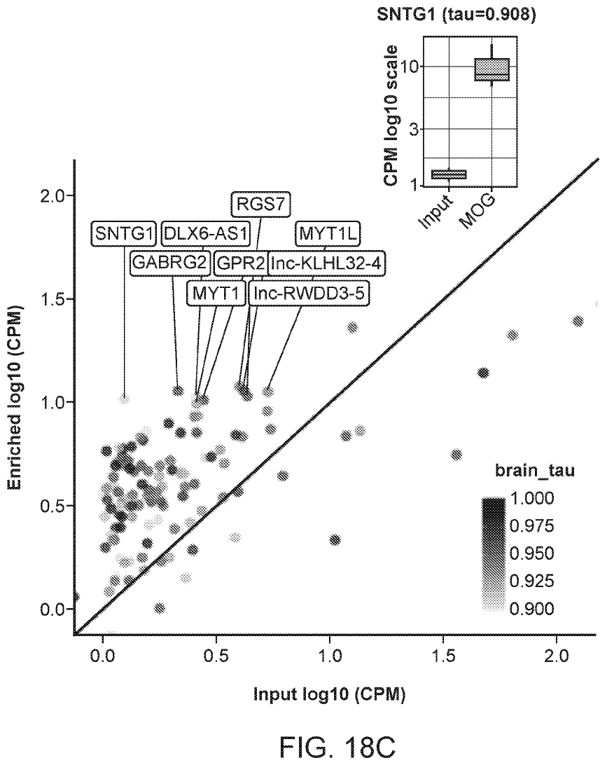

Although brain-derived EVs are generally rare, the following experiments were able to successfully characterize thousands of brain-derived EVs from human pooled plasma using optimized affinity isolation for Myelin Oligodendrocyte Glycoprotein (MOG)-enriched EVs and the SEVEN protocol described above (FIG. 18A). Only about 1% of all TSPAN-enriched EVs were also MOG-enriched. MOG-enriched EVs were characterized as having an average diameter of 80 nm (coefficient of variation=0.3) and an average TSPAN content of 13 molecules per EV (coefficient of variation=0.77) (FIG. 18B). Transcriptomic analysis revealed enrichment of brain-specific transcripts in MOG-enriched EVs, including Syntrophin Gamma 1 (SNTGT). Gamma-Aminobutyric Acid Type A Receptor Subunit Gamma 2 (GABRG2), DLX6 Antisense RNA 1 (DLX6-AS1), Myelin Transcription Factor 1 (MYTT), G-Protein Coupled Receptor 2 (GPR2), Regulator of G Protein Signaling 7 (RGS7), Myelin Transcription Factor 1 μLike (MYTIL), lnc-KLHL32-4, and lnc-RWDD3-6 (FIG. 18C). Enrichment was determined as EVs having an enrichment score tau >0.9. For tau, raw counts were downloaded from GTEx v8, and CPM (counts per million) normalized. The mean expression per tissue was calculated, log 2 transformed, and quantile normalized, and then used to calculate tau.

### Example 5: Characterization of Extracellular Vesicles Using Liver-On-Chip Culture Assay

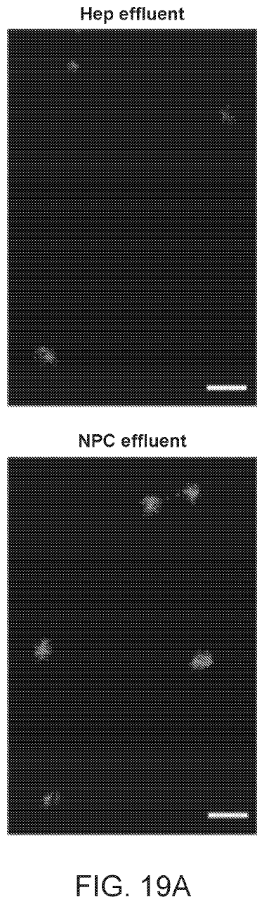

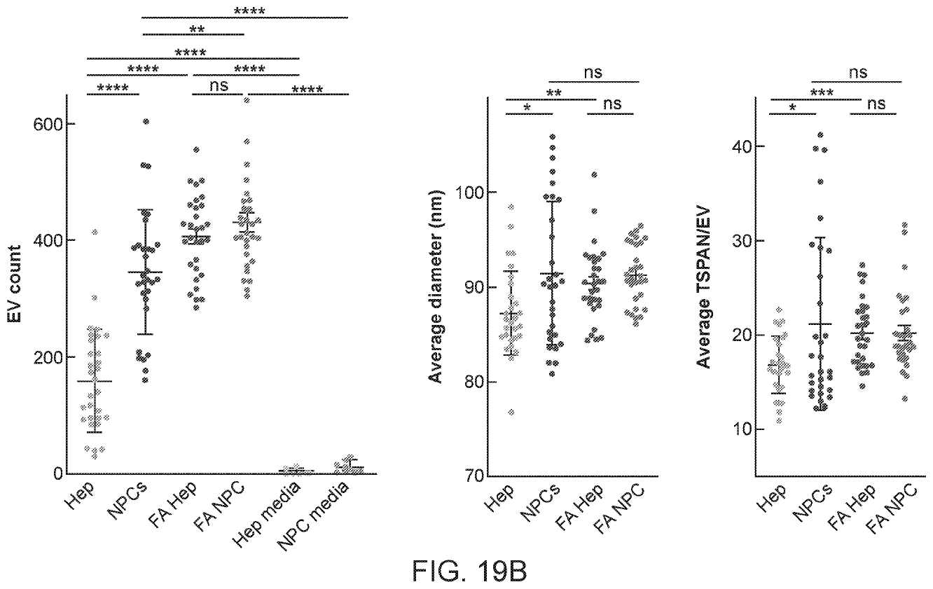

To characterize liver-specific EVs, the SEVEN assay was used in conjunction with a liver-on-chip experimental design—a ‘quad’ culture model with four different cell types under steady state conditions or subjected to treatment with fatty acids. These experiments demonstrated that EVs originating from the hepatocyte channel (Hep) were less abundant, smaller, and contained fewer TSPAN molecules as compared to EVs from the non-parenchymal cells (NPC) channel (FIGS. 19A-19B). Treatment with fatty acids produced an increase in the number of released EVs. EV size, and TSPAN content in the hepatocyte channel (FIG. 19B). In the NPC channel, fatty acid treatment slightly increased the number of EVs, but did not affect their properties (FIG. 19B). No significant differences were observed in the number of EVs, size, and TSPAN content between the hepatocyte and NPC channels after fatty acid treatment (FIG. 19B).

### Example 6: Characterization of Extracellular Vesicles from HER2-Positive Breast Cancer Patients

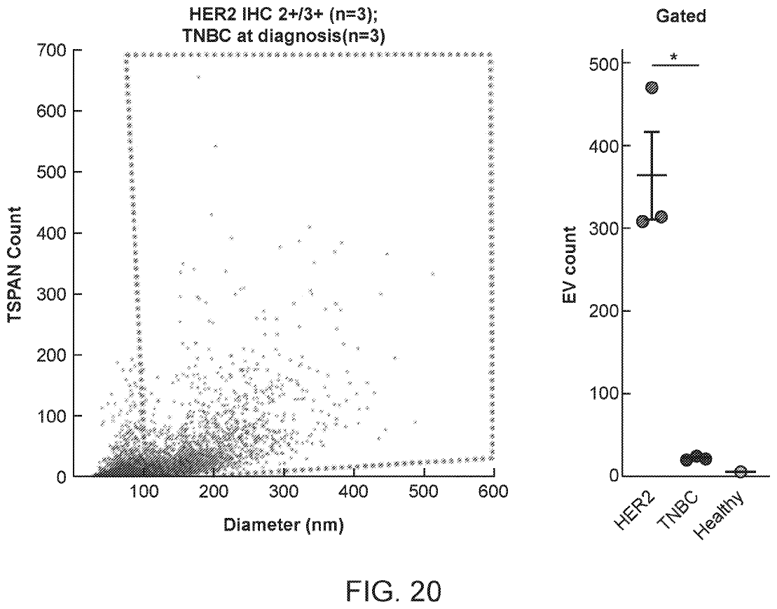

To characterize EVs from HER2-positive breast cancer patients, the SEVEN assay was used to isolate HER2-enriched EVs from plasma samples. Plasma EVs were isolated with thrombin treatment and captured on coverslips coated with trastuzumab (anti-HER2 antibody). HER2-enriched EVs from breast cancer patients with elevated HER2 (IHC2+ or IHC3+status; n=3) were compared against those from patients with triple-negative breast cancer (TNBC: n=3) at diagnosis and from pooled plasma of healthy subjects (healthy; n=1). A significantly higher number of HER2-enriched EVs with unique properties (size and detected tetraspanin content) was observed from plasma of breast cancer patients with elevated HER2 expression (FIG. 20).

### Example 7: Imaging of Extracellular Vesicles Using Super Resolution Radial Fluctuations Microscopy

EVs are heterogeneous in size, origin, and molecular content. Unlike traditional bulk analysis methods, single EV quantification allows for the characterization of individual vesicles, providing a more detailed and accurate representation of the sample. Moreover, they facilitate higher sensitivity and ability to detect rare vesicle populations that may have significant biological roles. Currently, single EV methods require specialized and typically expensive equipment that is not always readily accessible. While many projects still require comprehensive EV assessment with molecular sensitivity and very high resolution (e.g., SEVEN), fast and accessible single EV imaging methods are needed. Super resolution radial fluctuation (SRRF) is ideally poised to fill this gap because of its versatility. SRRF has a resolution of~70 nm, is compatible with a wide range of microscopes, and does not require specialized fluorophores. Here, SRRF imaging was combined with affinity capture and analysis algorithms for the detection of EVs. A correlation was demonstrated between SRRF and SMLM methods, and algorithms were developed to resolve closely spaced vesicles and to quantify data.

### Preliminary Data

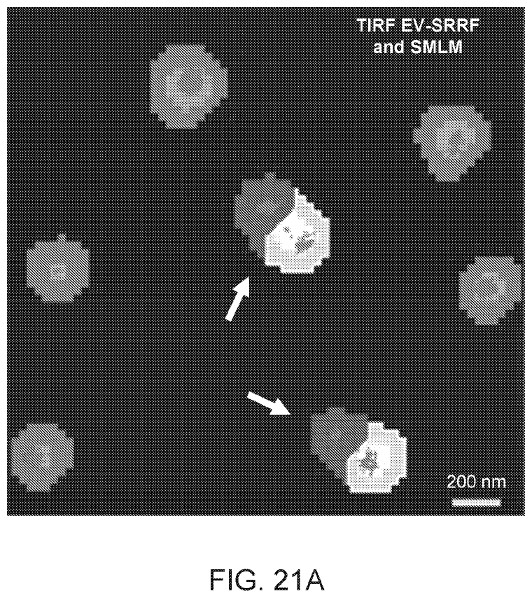

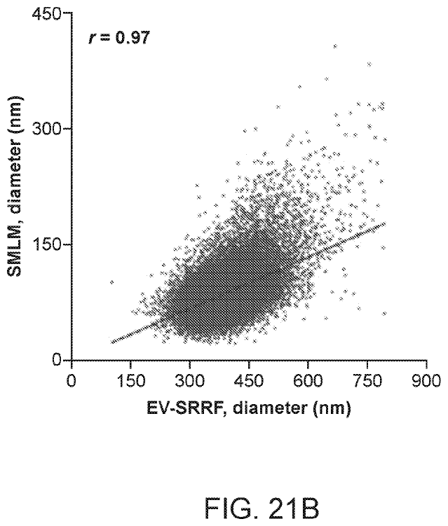

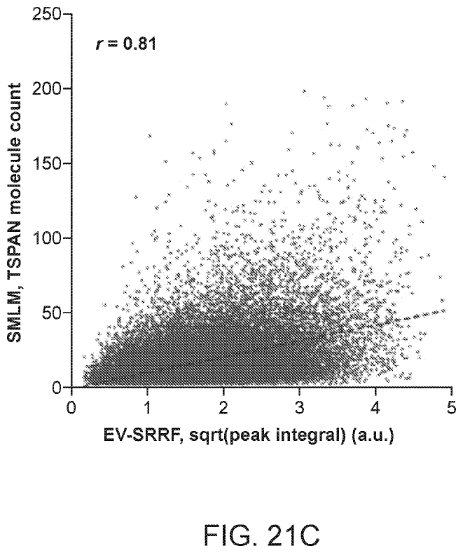

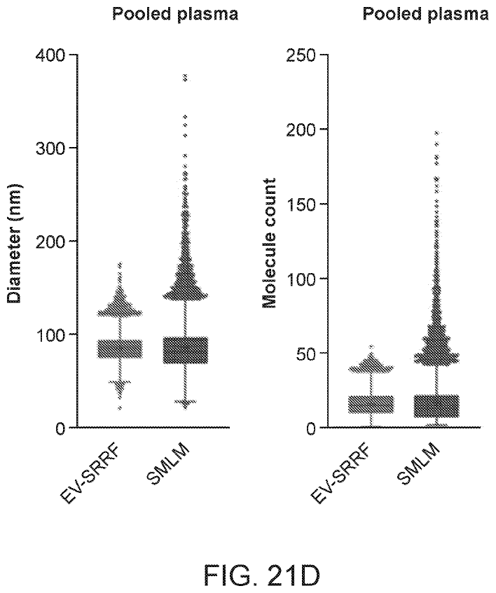

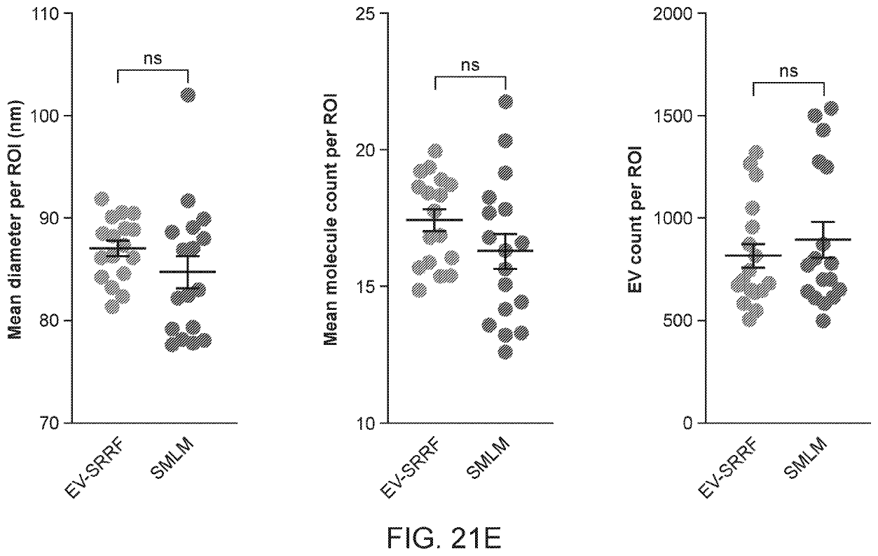

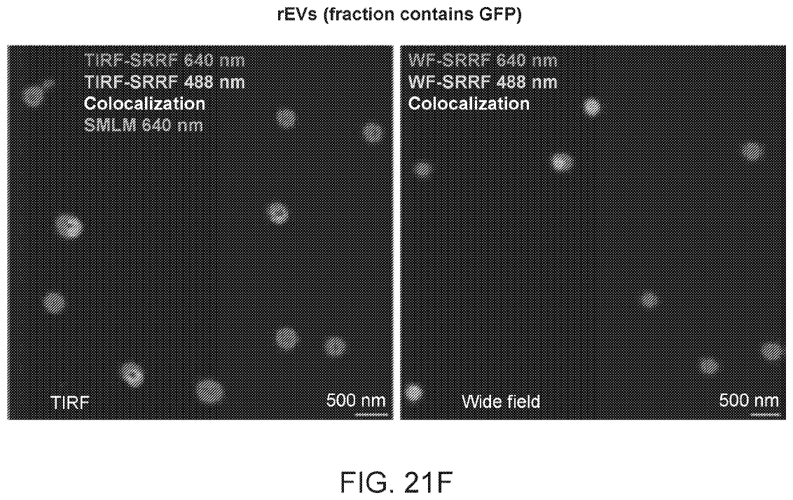

SRRF constructs images from a short diffraction-limited image sequence (few seconds of imaging time) by calculating radial and temporal fluorescence intensity fluctuations. This imaging modality is predicated on the premise that fluorophores correlate in time, whereas noise does not. EVs were first imaged from pooled plasma samples using SRRF in total internal reflection fluorescence (TIRF) illumination mode. To develop and validate the EV-SRRF analysis workflow, correlative single molecule localization microscopy (SMLM)-SRRF analysis was performed and supported with k-means clustering of Laplacian image segments and machine learning-based classification for resolving EVs located in close vicinity (FIG. 21A; EVs designated by white arrows). Conditions were optimized based on ~50,000 plasma EVs as a training data set. Testing the method on an independent data set of ~16.000 plasma EVs, an 86% overlap was obtained between SRRF and SMLM imaging modalities, where 92% of SRRF-identified EVs were accurately resolved as individual EVs. Only 0.9% of SRRF-identified EVs did not show any detected localizations in SMLM. In a subset of images. EV sizes and tetraspanin content were correlated between the two methods. A robust positive correlation was observed in EV diameter (FIG. 21B) and detected TSPAN content/EV (FIG. 21C) between EVs measured using SMLM and SRRF modalities. These correlations were separately confirmed in an independent set of pooled plasma (FIG. 21D), demonstrating a correspondence in the average EV size and detected TSPAN content/EV per ROI between SMLM and EV-SRRF (FIG. 21E). Moreover, EV-SRRF imaging was successfully performed using a wide-field microscope (FIG. 21F).

### SRRF Analysis

SRRF images were processed using routine methods. Segmentation was performed by region-growing-supported thresholding of the normalized image to generate an initial binary image and connected component labeling with equivalence class resolution to label binary segments. To improve the separation of EVs in close vicinity, intensity peak detection was used along with multi-component 2D Gaussian fitting of Laplacian image segments for peak deconvolution of those objects in which up to three peaks were initially detected. Based on binary object features and additional features extracted from the Gaussian fitting, a medium Gaussian support vector machine model was trained to classify objects up to three resolved components. Next, the pixel-based image was converted to a localization-based image by generating a grid of virtual localizations over the foreground pixels of the binary SRRF image with an optimized grid density according to pixel intensities in the Laplacian segments. The virtual localizations were segmented using k-means clustering with number of components determined from peak detection and using the trained classification model. The developed analysis workflow was tested and validated by evaluating overlay images of corresponding SRRF and SMLM images, where the SMLM image was used as the ground truth. Segmented pixels containing polygons (SRRF) were overlapped with polygons obtained from Voronoi tessellation-based clustering of SMLM localizations.

### Example 8: Characterization of Heart-Enriched Extracellular Vesicles

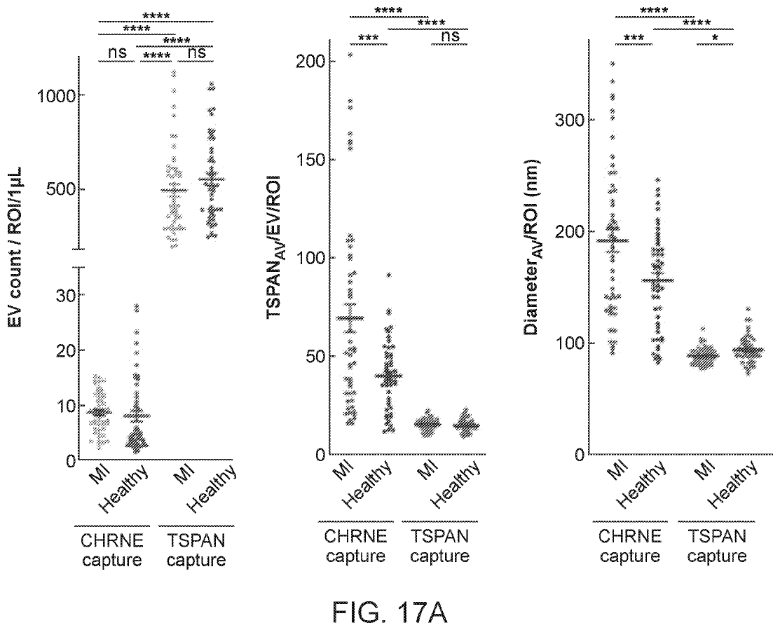

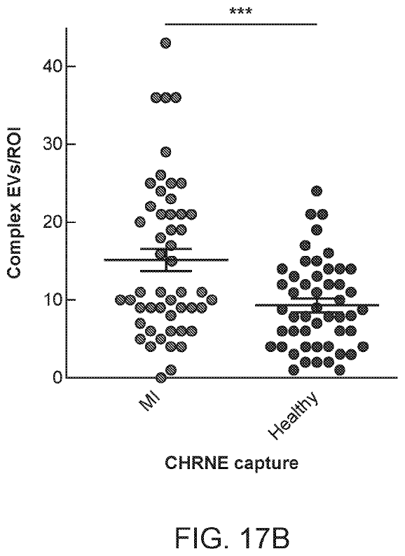

To identify potential differences in the molecular profile of heart-enriched EVs between healthy and disease states, Tetraspanin (TSPAN)- and Nicotinic Acetylcholine Receptor, Epsilon Subunit (CHRNE)-enriched EVs from five healthy patients and five myocardial infarction (MI) patients were isolated and characterized according to the SEVEN assay described above. The CHRNE subunit of acetylcholine receptors has a recognized expression preferentially within cardiac tissue. For each patient, 10 regions of interest (ROI) were obtained in two independent experiments for both CHRNE and TSPAN captures. While no or very small changes were observed in the number, TSPAN molecular content, and diameter of TSPAN-enriched EVs between the healthy and MI patient groups, CHRNE-enriched EVs from MI patients were significantly larger and contained more detected TSPAN molecules per EV (FIG. 17A). Thus, quantification of complex EVs was performed —complex EVs include donut-like shapes, elongated or odd shapes, and clumping/budding shapes. For CHRNE capture, a significantly higher number of CHRNE-enriched complex EVs per ROI was observed for MI patients over healthy patients (FIG. 17B).

### Example 9: Discovery and Analysis of Cardiovesicles as Biomarkers of Cardiovascular Disease

### INTRODUCTION

The present study sought to discover heart-specific antigens on the membrane of cardiomyocyte-derived EVs (“Cardiovesicles”) by implementing the disclosed immunoaffinity-based techniques to capture and characterize these heart-derived EVs in plasma. To this end, a comprehensive pipeline for the discovery and validation of Cardiovesicles was employed, beginning with the isolation of EVs from induced pluripotent stem cell-derived cardiomyocytes (iPSC-CMs) via size-exclusion chromatography (SEC). The cardiomyocyte-derived EV proteome was then defined using Liquid Chromatography-Mass Spectrometry (LC/MS). Candidates for heart-specific EVs were prioritized using bioinformatic methods mining data from the Genotype-Tissue Expression (GTEx) database and the Human Protein Atlas. For the specific capture of Cardiovesicles from plasma, an immunoaffinity capture method was developed using biotinylated antibodies and streptavidin-coated paramagnetic beads (Exocapture-MSB). The enrichment of heart tissue-derived proteins in Cardiovesicles was confirmed through Westem Blot assays on immunocaptured EVs from pooled plasma samples of healthy volunteers. Heart tissue-enriched genes were identified via long RNA sequencing. Finally, the isolation method was validated with plasma samples from patient cohorts with cardiovascular diseases.

### Methods

### Size Exclusion Chromatography (SEC)

SEC was employed for the enrichment of EVs from plasma samples. Initially, a 1 mL aliquot of plasma underwent a precleaning process, which involved centrifugation at 12,000 g for 12 minutes at 4° C. Subsequently, size exclusion columns (35 nm) were utilized for the isolation procedure. The columns, kept at 4° C., were initially prepared by allowing the 20% ethanol storage solution to drain completely. These columns were then conditioned with 20 mL of particle-free Phosphate-Buffered Saline (PBS). The pre-cleaned plasma was carefully overlaid onto the size exclusion column, followed by elution with particle-free PBS. The elution process was controlled to collect flow-through in 500 μL fractions. Fractions 7 through 10 were specifically pooled and concentrated down to 1 mL. Post-procedure, the columns were thoroughly cleaned using 50 mL of particle-free PBS and subsequently stored in a 20% ethanol solution. This meticulous protocol ensured optimal isolation and enrichment of EVs from plasma samples.

### Liquid Chromatography Mass Spectrometry (LC-MS/MS)

EVs were enriched from cardiomyocyte culture media via SEC as outlined in the earlier protocol and subsequently processed for shotgun proteomics. SEC fractions 7-10, containing the EVs, were pooled and the buffer was exchanged to 50 mM ammonium bicarbonate using a filter with a specified molecular weight cut-off (MWCO). Each retentate was then lysed in a 50 mM HEPES buffer, which contained 2% sodium deoxycholate (DOC) and a 1× protease and phosphatase inhibitor cocktail. This was achieved via sonication. The clarified lysates were quantified using the BCA protein assay. Subsequent to this, the samples were incubated with dithiothreitol (final concentration 10 mM) for 30 minutes, followed by incubation with iodoacetamide (final concentration 20 mM) in darkness for an additional 30 minutes. Thereafter, the samples were diluted with 50 mM HEPES to reduce the DOC concentration to 1%. Overnight protein digestion was then performed using trypsin endoproteinase at an enzyme to substrate ratio of 1:50. Upon digestion, the proteins were acidified and DOC was removed using 96-well filter plates (0.45 μM) coupled with a positive pressure system. Tryptic peptides were subsequently desalted through C18 solid-phase extraction and the resulting peptides were vacuum centrifuged to dryness. The dried peptides were reconstituted in LC-MS grade water supplemented with 2% acetonitrile and 0.1% formic acid and then quantified. An aliquot of 500 ng of peptides was loaded onto a 50 cm C18 column and analyzed using a 2-hour LC-MS/MS method. Utilizing a duty cycle of 1 second for each of the 3 FAIMS CVs (−40/−60/−80), precursor scans were conducted in the Orbitrap at a resolution of 120,000. The most abundant precursor ions with a charge state between 2-6 in the survey scan underwent fragmentation by high energy collision dissociation, followed by measurement in the ion-trap.

Raw mass spectrometry data were matched against a human protein database (Swissprot/UniprotKB, 2020). The search parameters were configured to allow for fully tryptic peptides only, accounting for up to 2 missed cleavages and 10 ppm and 0.6 Da mass tolerance for the precursor and fragment ions, respectively. A false discovery rate of 1% was applied in the Percolator node for stringent data analysis.

### RNA Extraction

Extracellular RNA (ExRNA) was isolated from either raw plasma samples or immunocaptured EV samples using routine methods. Initially, samples underwent a centrifugation process at 12,500 g for 10 minutes at 4° C. to remove any debris or cells. Following centrifugation, a 200 μL aliquot of the supernatant was transferred to a fresh 1.5 mL tube. Subsequently, 1 mL of lysis reagent was added to the tube and thoroughly mixed to ensure a homogenous mixture. This mixture was then transferred to 2 mL tubes, followed by the addition of 200 μL of chloroform. The mixture was vigorously vortexed and then centrifuged at 13,000 g for 15 minutes at 4° C. to facilitate phase separation. Following centrifugation, the aqueous phase containing RNA was carefully transferred into a new tube, with the addition of 2 μL of glycogen to facilitate RNA precipitation during the subsequent steps. The samples were then processed further to successfully isolate extracellular RNA. This method ensures effective extraction of RNA, making it suitable for downstream applications such as RNA sequencing.

### ExRNA Sequencing and Analysis

Sequencing libraries were generated from RNA using conventional methods. Single stranded DNA was generated with their template switching mechanism without the need for adapter ligation and integration of unique molecular identifiers. cDNA derived from rRNA was removed prior to an additional PCR amplification (16 cycles). Libraries were characterized using high sensitivity screen tapes. Libraries were pooled together using output reads and then normalized equally to create equimolar pools. The equimolar pools were quantified using qRT-PCR and sequenced using conventional methods.

### ExoCapture: Biotin-Streptavidin Magnetic Vesicle Isolation

This study employed a method termed “ExoCapture.” This procedure is founded on the affinity of biotin and streptavidin and uses streptavidin magnetic beads to isolate extracellular vesicles. The ExoCapture method was initiated by determining the requisite amount of magnetic beads according to their binding capacity. Approximately 80 μLL of these beads, which were chemically pre-blocked, were used for every 12 μg of biotinylated peptide.

Prior to use, the beads were washed thrice to eliminate preservatives or blocking buffer. To ensure effective washing, 80 μL of magnetic beads were put into a 1.5 mL tube, followed by the addition of 1 mL of Binding/Wash buffer. After gentle vortexing, the tube was placed into a magnetic stand to collect the beads against the side of the tube. The supernatant was removed by aspiration, and the beads were resuspended with the washing buffer. This procedure was performed three times. Subsequently, 200 μL of the sample was diluted with 200 μL of C5 Binding/Wash Buffer. Afterward, 12 μg of biotinylated antibody was added to the diluted sample and incubated on a rotating mixer at 4° C. overnight. The pre-washed magnetic beads were then added to the diluted sample, incubated at room temperature on a rotating mixer for one hour, and subsequently separated using the magnetic stand. Lastly, the beads were resuspended in 300 μL of C5 Binding/Wash Buffer and vortexed. After placing the tube back on the magnetic stand, the flowthrough was discarded. This washing and resuspension process was repeated twice for a total of three washes. Overall, the ExoCapture protocol provides a robust and reproducible methodology for isolating extracellular vesicles from various samples using magnetic streptavidin-coated beads and biotinylated antibodies.

### Western Blot Analysis

The protein content in the samples was first quantified using standard methods. Subsequently, the protein extract was normalized with PBS to the desired concentration, and an equal volume of buffer was added, ensuring a 1:1 volume ratio of normalized protein to buffer. The buffer (60 mM Tris-HCl pH 6.8; 20% glycerol; 2% SDS; 4% beta-mercaptoethanol: 0.01% bromophenol blue) was employed due to its unique reagents, which facilitate SDS-PAGE. The samples were then heated at 95° C. for 10 minutes to further denature the proteins, ensuring that the proteins underwent electrophoresis according to their monomeric weight.

Subsequently, 5 g of total protein per lane was resolved on a sodium dodecyl sulfate-polyacrylamide gel electrophoresis (SDS-PAGE) system. The separated proteins were then transferred onto a polyvinylidene difluoride (PVDF) membrane. The PVDF membrane was then blocked with 5% bovine serum albumin (BSA) in Tris-buffered saline with Tween 20 (TBST) to prevent non-specific antibody binding. The membrane was subsequently incubated with primary antibodies in the same blocking buffer, allowing specific binding to their respective target proteins. Following the primary antibody incubation, the membrane was exposed to horseradish peroxidase (HRP)-conjugated secondary antibodies. The blots were developed using a highly sensitive enhanced chemiluminescent (ECL) substrate, enabling the detection of low-abundance proteins.

### Microfluidic Resistive Pulse Sensing

Microfluidic resistive pulse sensing (MRPS) provides precise measurements of particle sizes within a specified range. This permitted the measurement of particle sizes between 65 nm and 400 nm. To perform MRPS measurements, the microfluidic system was primed using a 0.1% (v/v) Tween 20 solution in phosphate-buffered saline (PBST 0.1%). This ensured a clean, low-surface-tension environment within the system, aiding the flow of samples. Instrument parameters, including pressure at each cartridge port and voltage at each bias electrode, were automatically determined by the device. Diluent was filtered through syringe filters with a pore size of 0.02 mm to remove any particles that could contribute to false-positive counts. For the MRPS measurements, samples were diluted in PBST 0.10% at a ratio of 1:100. A total of 7 μL of this diluted sample was then applied to the cartridge. To obtain reliable data, all measurements were carried out until the standard error reached less than 2%. Experiments were conducted in triplicate for reliability and repeatability. Upon completion of the measurements, data from the replicates were combined. Peak filters were applied and background subtraction was performed. This resulted in accurate particle size distributions for the EV samples.

### Statistical Analysis

Given the nature of our dataset and research design, we selected two specific non-parametric tests for comparing our groups. For comparisons involving three groups, the Kruskal-Wallis test was used. For comparisons between two groups, the Mann-Whitney U test was employed. A significance level of 0.05 was established for all the statistical tests. Thus, any test resulting in a p-value of less than 0.05 was considered statistically significant.

### Human Induced Pluripotent Stem Cell-Derived Cardiomyocyte Differentiation

The protocol for differentiating induced pluripotent stem cells (iPSCs) into cardiomyocytes involved multiple stages over a period of several weeks. On day −1, plates were prepared by coating with a basement membrane matrix that provides an ideal growth environment for the iPSCs. The matrix was diluted in sterile 1× DPBS and added to each well, then the plates are incubated overnight at 37° C. On day 0, iPSCs were revived from storage in liquid nitrogen. Essential 8 medium, supplemented with Essential 8 Supplement and Penicillin-Streptomycin, was prepared and warmed, iPSCs were quickly thawed and added to the warmed medium. The cell suspension was centrifuged to form a pellet, which was then resuspended in complete medium with supplement and Y27632, an RHO/ROCK pathway inhibitor. This cell suspension was added to the matrix-coated wells and incubated at 37° C. On days 1 and 2, the medium was changed, with the addition of Y27632 if cell growth appeared slower than expected. Extra matrix-coated plates were prepared on day 2 to allow for the expansion of the iPSC population. On day 3, the cells were split and also cryopreserved for future use. For splitting, cells were washed and incubated with a digestion buffer (0.5 mM EDTA in 1× DPBS). After incubation, cells were resuspended in the medium with the RHO/ROCK pathway inhibitor and distributed across the matrix-coated plates. For cryopreservation, cells were resuspended in cold PSC Cryopreservation media and stored in cryovials. The differentiation phase of the protocol began after a certain number of passages. Specialized media was prepared according to the stage of differentiation and the medium was changed regularly. Cells were treated with a series of signaling molecules such as CHIR99021 and IWP4 to direct the differentiation towards cardiomyocyte lineage. From day 18, the medium was changed to one containing DMEM, high glucose, pyruvate media, Fetal Bovine Serum, and Penicillin-Streptomycin. At this point, the differentiated cells were exhibiting the characteristics of cardiomyocytes. They were kept in this medium until ready for use in experiments. Throughout this process, steps were taken to ensure sterility and prevent contamination, including the use of sterile equipment and solutions, filtration of media, and appropriate storage of reagents. The state of the cells was monitored regularly through visual inspection and appropriate measures are taken to address any issues, such as slow growth.

### Results

Proteomic Profiling of iPSC-Cardiomyocyte-Derived EVs and Identification of Cardiac-Specific Markers

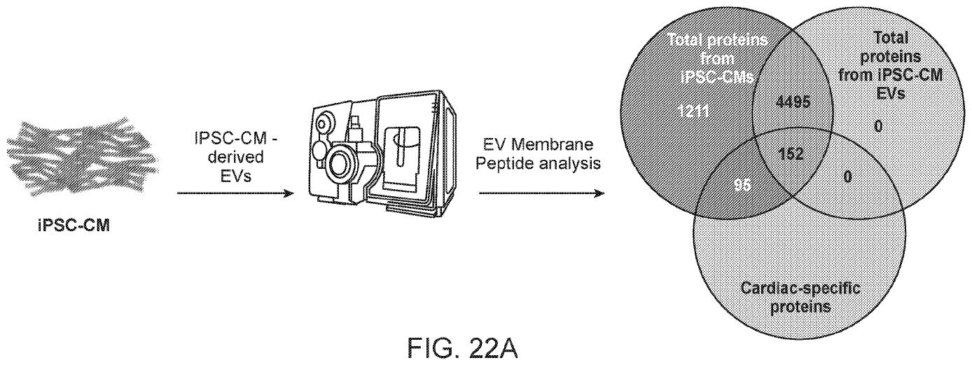

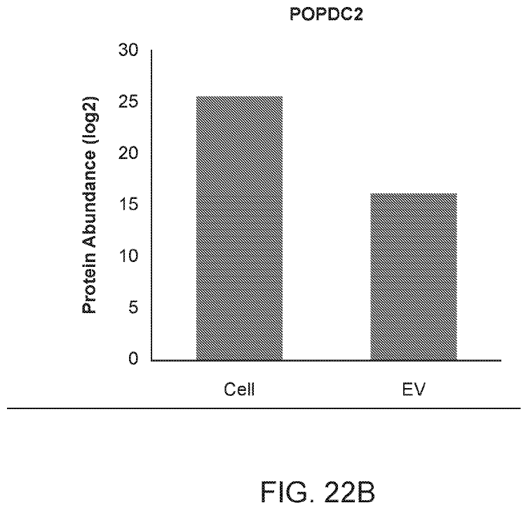

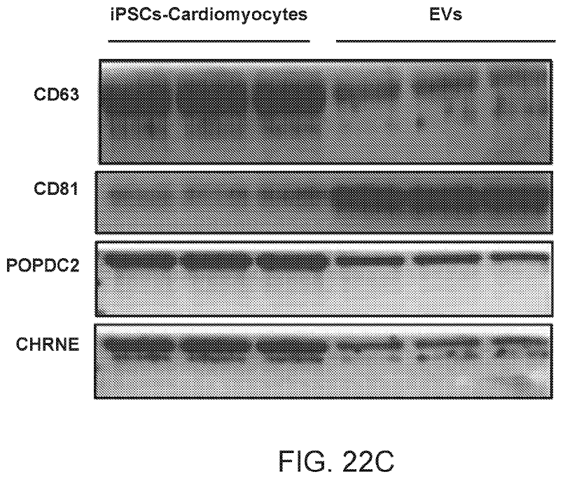

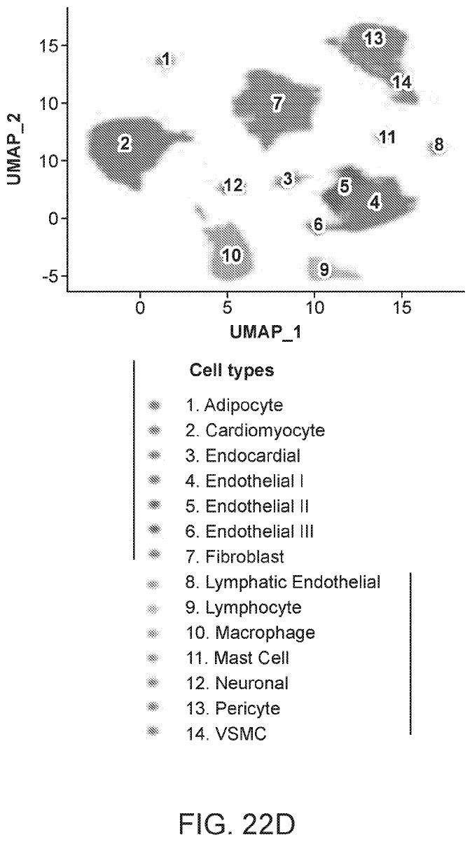

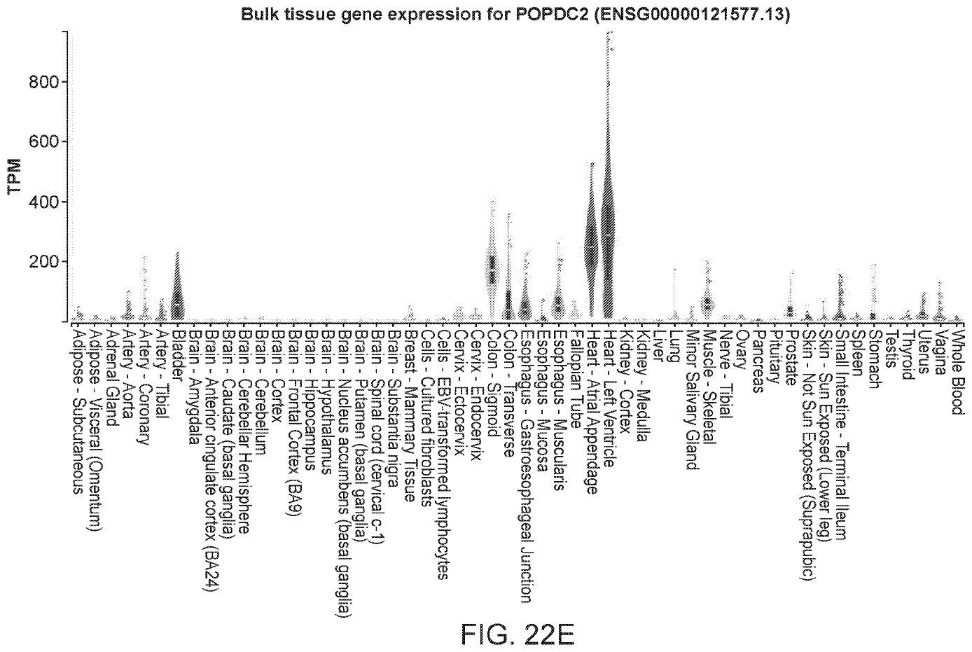

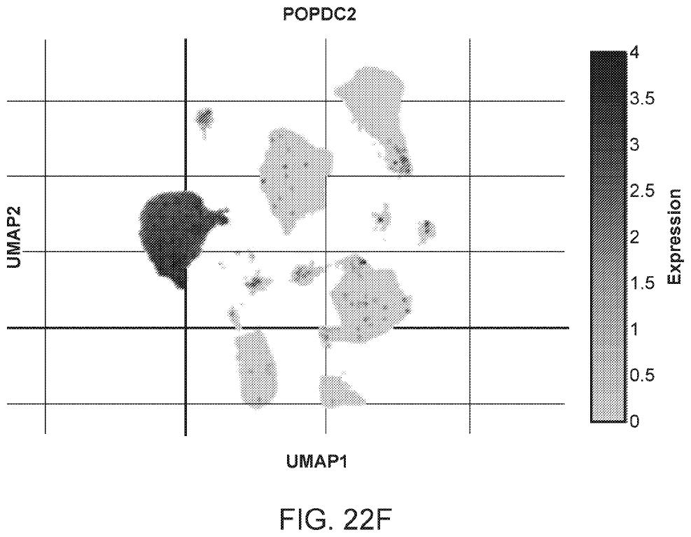

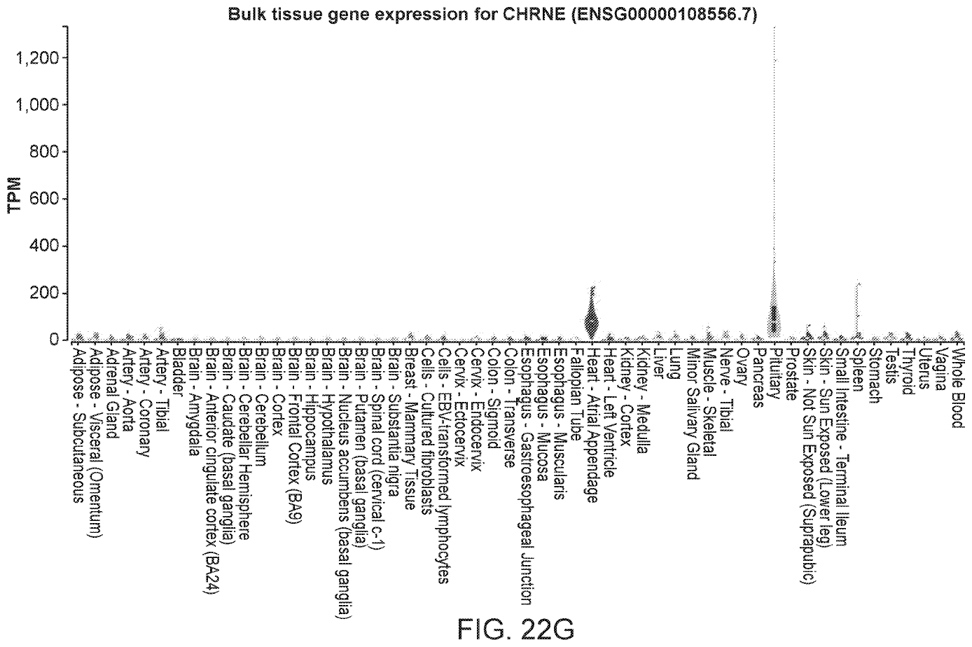

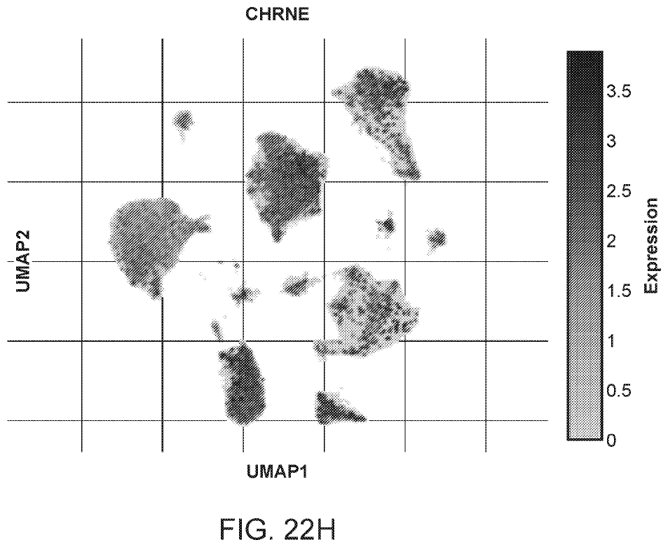

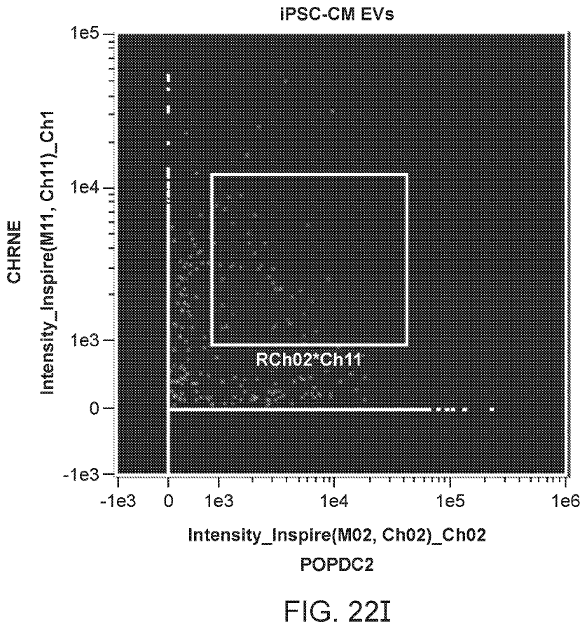

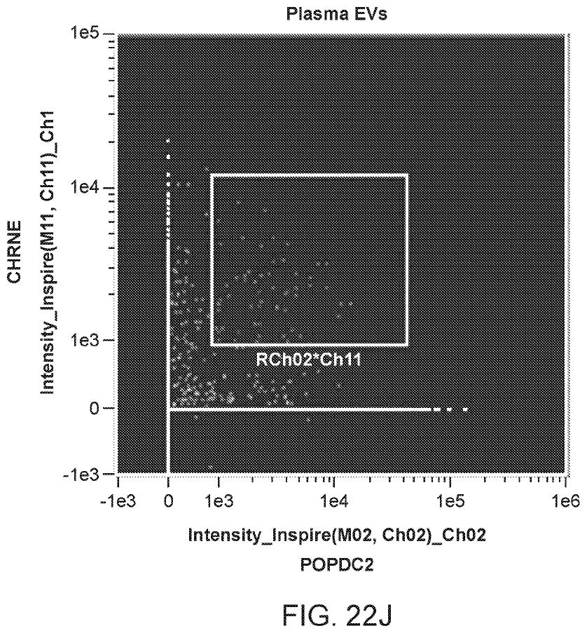

This study identified and characterized Cardiovesicles using a comprehensive analysis performed on induced iPSC-CMs and their released EVs using LC-MS analysis, yielding 5,397 and 4,647 proteins, respectively (FIG. 22A). By cross-referencing the list of identified proteins with the Human Protein Atlas database, 245 proteins were identified in iPSC-CMs that were predominantly enriched in heart tissue. Of these, 150 proteins were also identified in iPSC-CM-derived EVs. In addition, proteomics measurements also identified two heart enriched proteins, RILP and OTOGL exclusively in the EV sample. The LC/MS analysis of the iPSC-CM and the CM-derived EVs confirmed the presence of POPDC2, a candidate cardiomyocyte marker. Further bioinformatics analysis was performed to identify membrane proteins with high gene expression in the heart tissue. Candidates with a plasma membrane localization score >3 that are also elevated in heart tissue were selected. This analysis identified two new proteins, CHRNE and TMEM182 as putative marker proteins of cardiomyocytes EVs. Notably. CHRNE was observed in a subsequent proteomics analysis that allowed for inclusion of proteins identified with a low confidence threshold. The decision to focus on these proteins for immuno-isolation of EVs was guided by this combined bioinformatics and proteomic approach. Within this context, both POPDC2 and CHRNE were particularly emphasized due to their distinct bioinformatics attributes and direct validation through proteomic analysis. An exemplary comparison between the relative quantification levels of POPDC2 obtained from iPSC-CMs and their derived EVs from proteomic analysis is shown in FIG. 22B. The presence of these cardiac-specific proteins was further validated through Western blot assays (FIG. 22C). The Uniform Manifold Approximation and Projection (UMAP) and tissue-wise enrichment data obtained from the GTEx database underscored the cardiac specificity of POPDC2 and CHRNE, illustrating their predominant expression in heart tissue at cellular levels (FIGS. 22D-22H). Additionally, AMNIS flow cytometry confirmed the co-localization of these markers in double-positive populations of iPSC-CM- and human plasma-derived EVs, confirming their cardiac origin and specificity (FIGS. 221 and 22J).

### Validation of Cardiac-Specificity Using Cardiac-Specific Cre-Driven EXOMAP Mouse Model

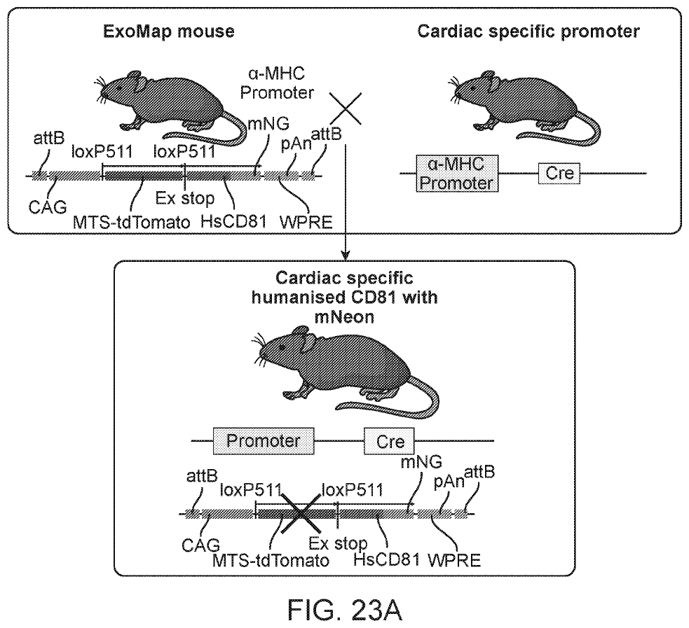

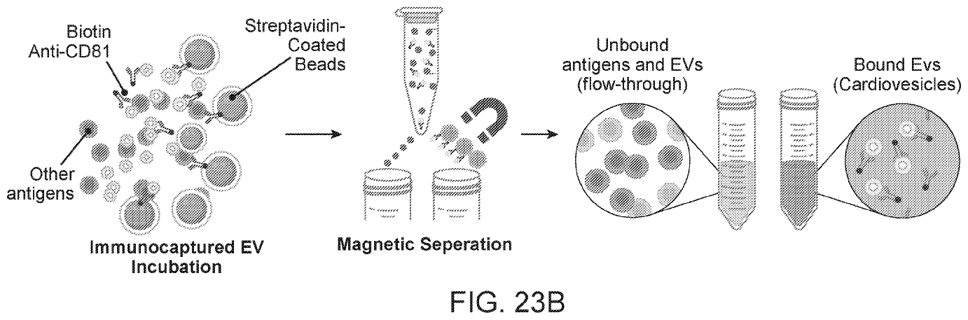

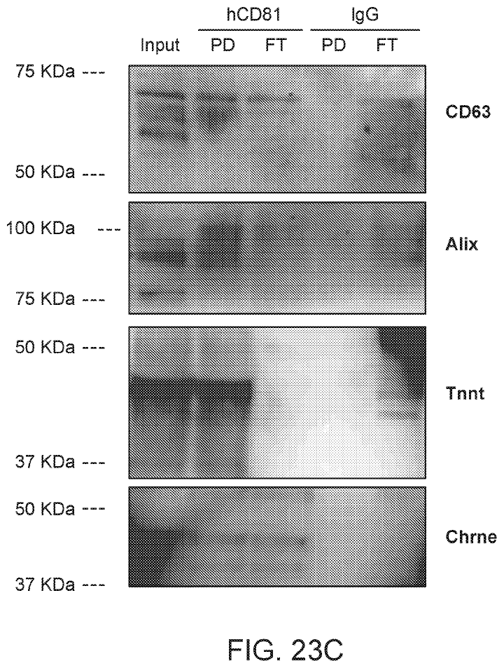

The EXOMAP mouse model, specifically the cardiac-specific Cre-driven variant, was used to verify the cardiac specificity of certain proteins by using a genetically engineered system that produces exosomes identifiable by a fusion protein. This allowed for the precise tracing and analysis of cardiac-derived EVs, facilitating the study of cardiac biology and heart-related conditions. This mouse model expresses a humanized version of CD81 tagged with mNeonGreen (HsCD8ImNG) specifically in cardiac cells (FIG. 23A), which allowed tracking and confirming the cardiac origin of the EVs. In particular, this model confirmed the cardiac-specificity of POPDC2 and CHRNE. Western blot assays confirmed the enrichment of CHRNE and cardiac troponin in HsCD81-positive EVs isolated from these mice using the disclosed ExoCapture-MSB method (FIG. 23B), thereby validating the cardiac origin of the isolated EVs (FIG. 23C).

### Cardiovesicle Transcriptomics Revealed Presence Cardiac-Specific Transcripts

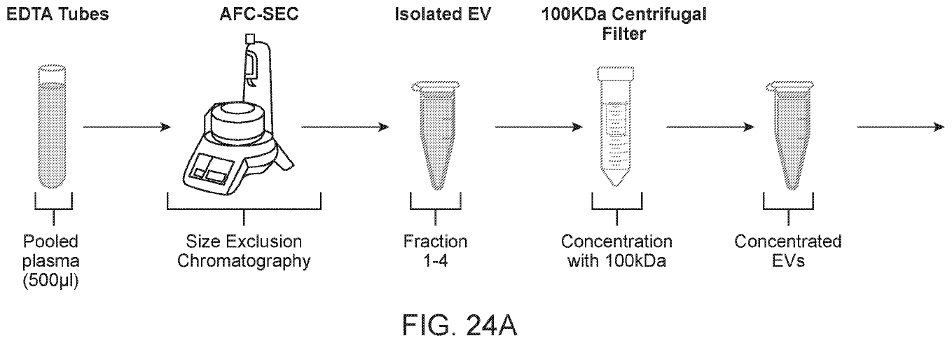

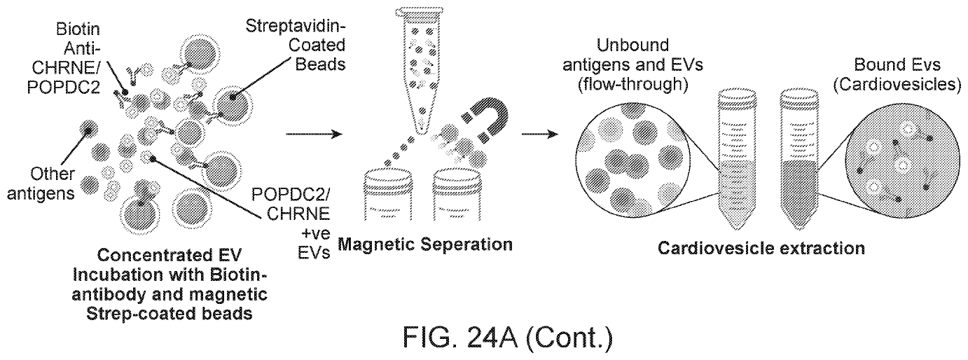

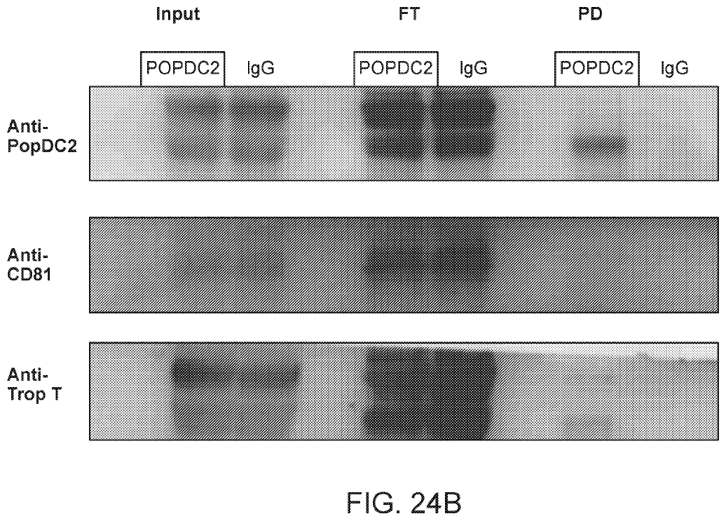

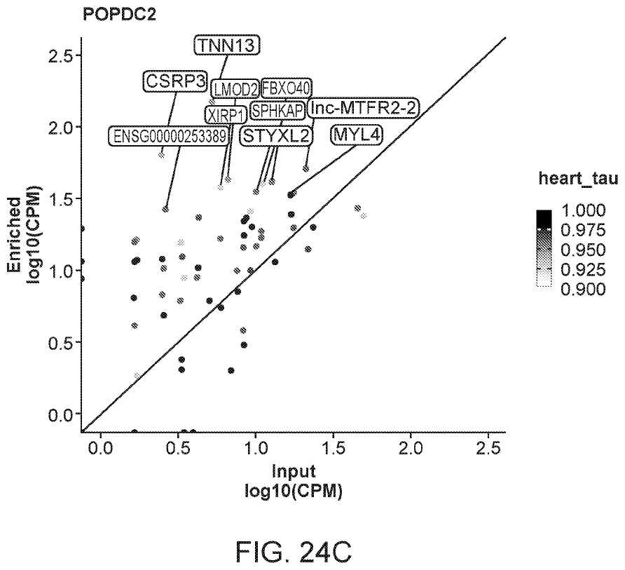

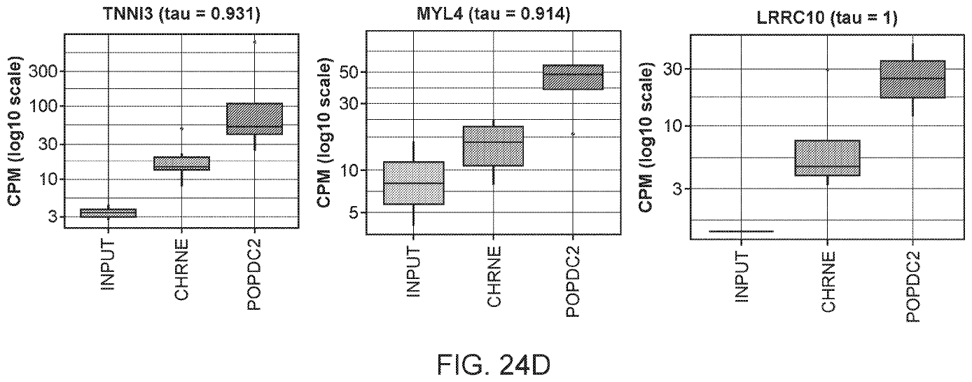

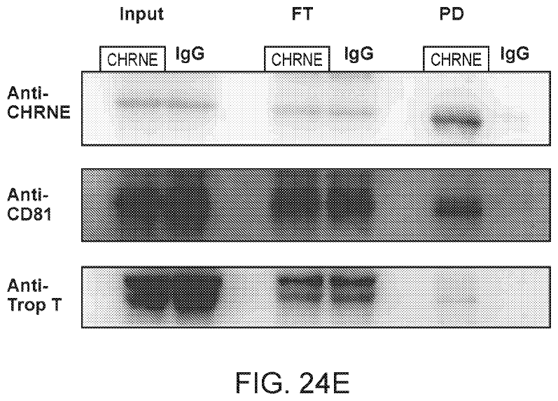

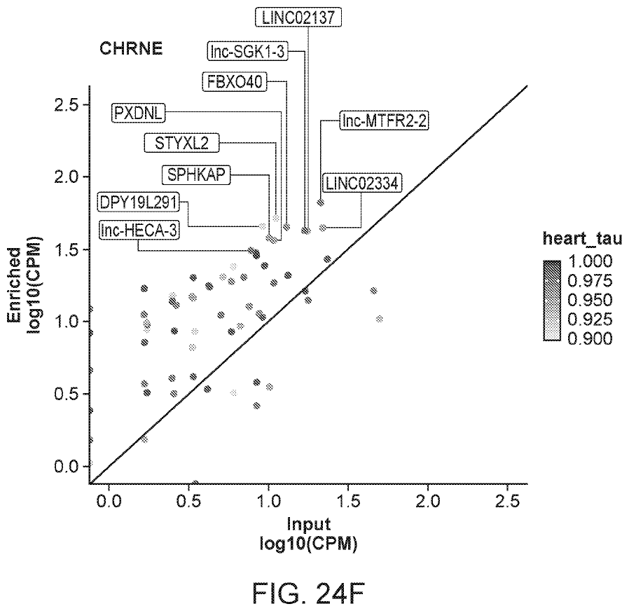

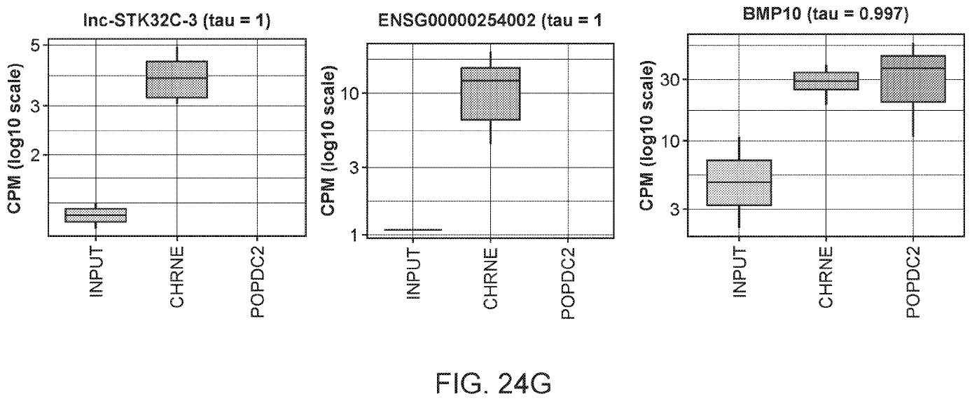

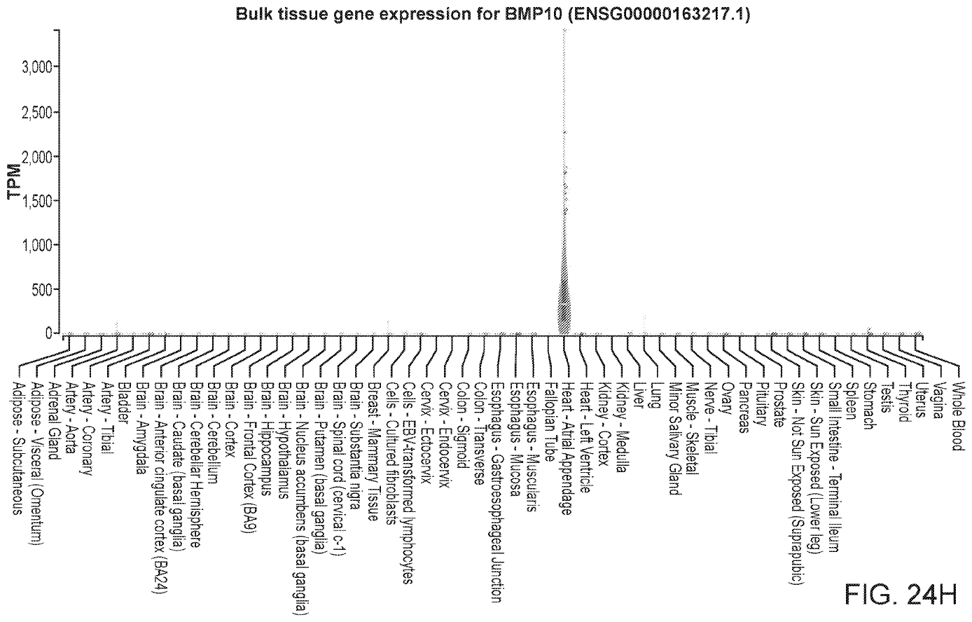

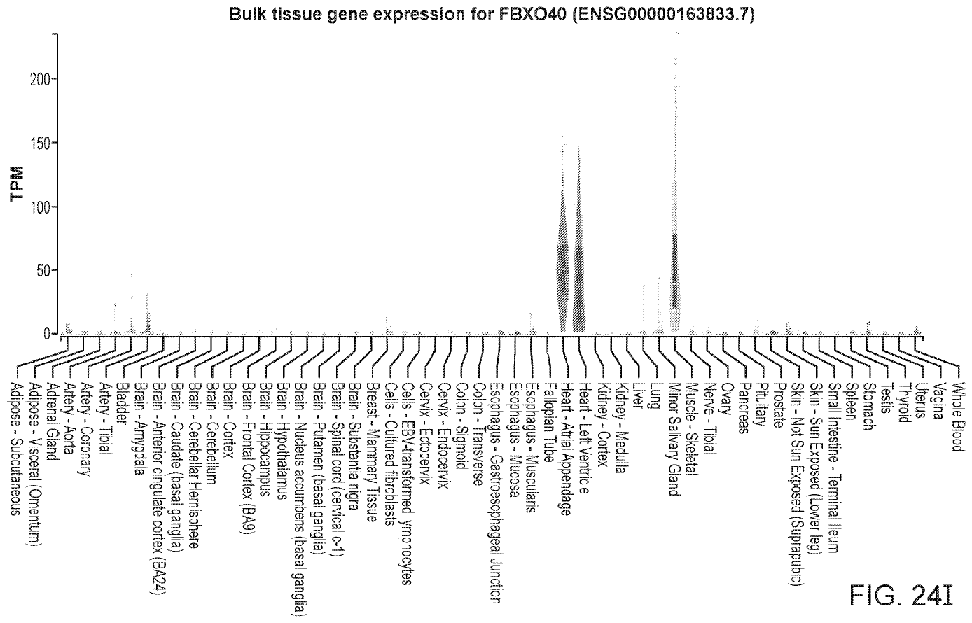

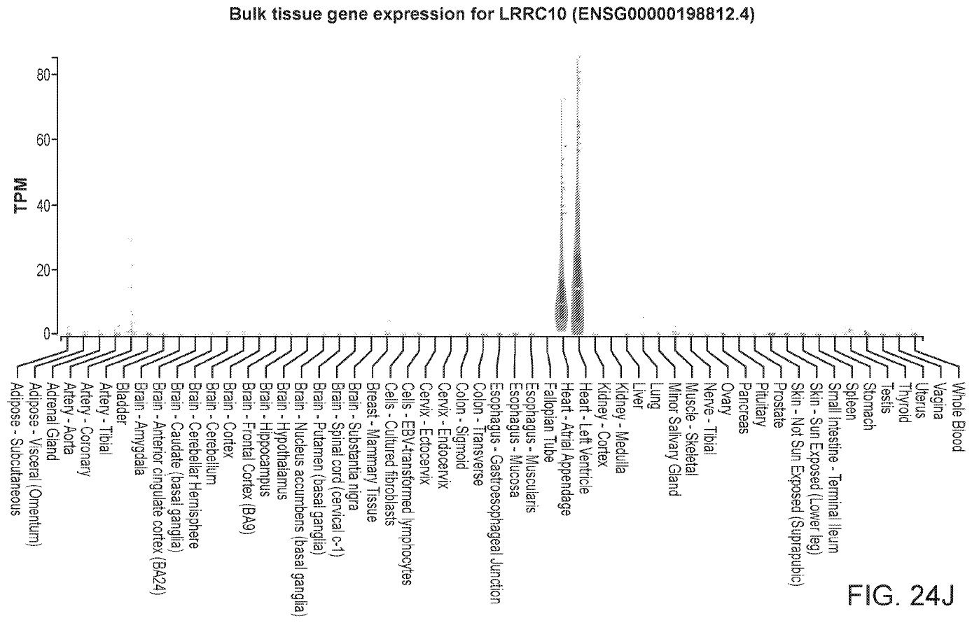

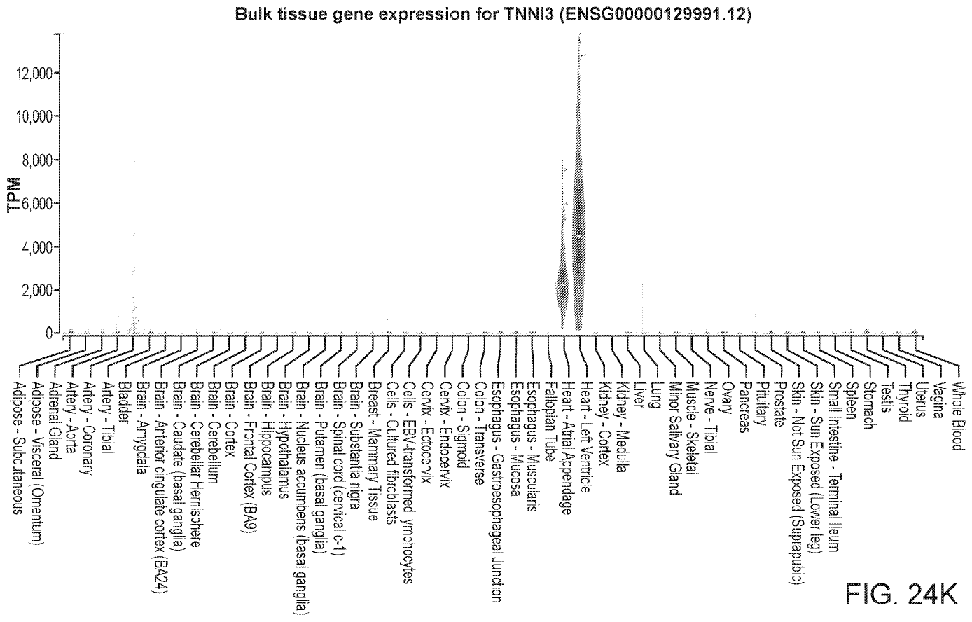

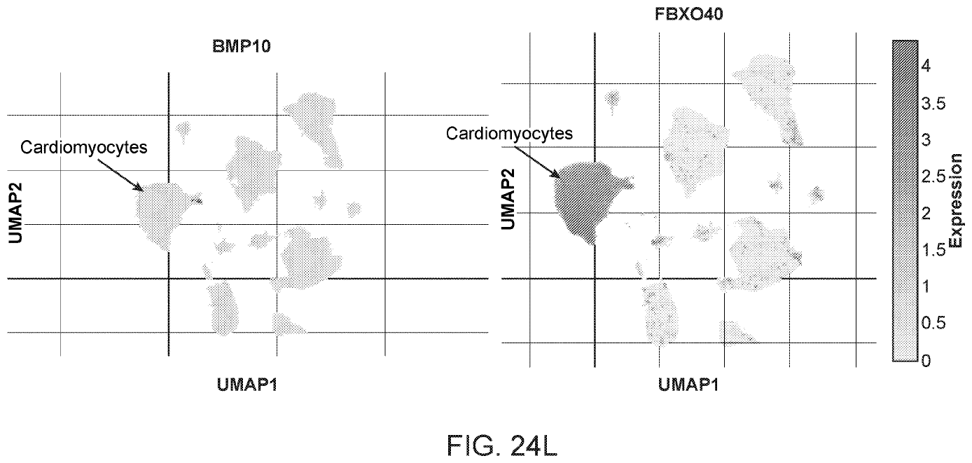

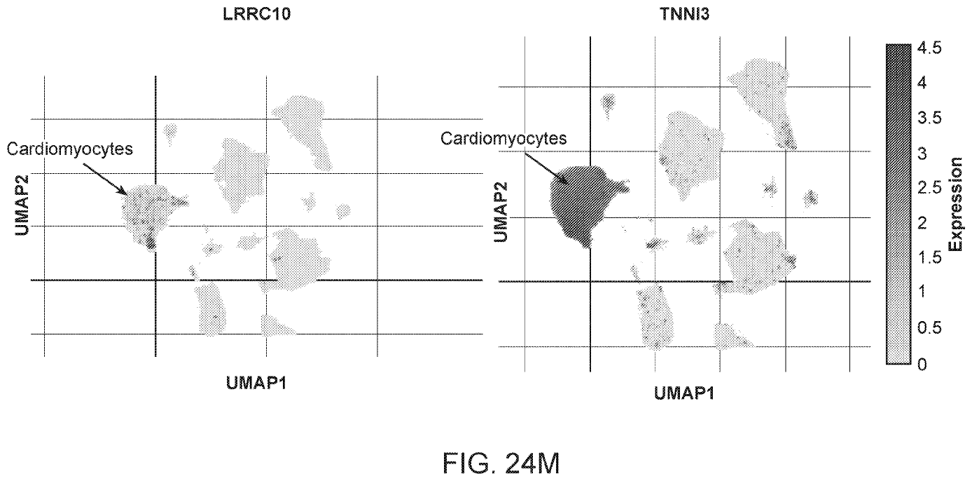

The ExoCapture-MSB method for the isolation and analysis of cardiac-derived EVs from human plasma is outlined in FIG. 24A. This method harnesses the affinity of biotin-streptavidin, thereby allowing for the precise isolation of Cardiovesicles. Western blot analyses revealed significant enrichment for cardiac proteins, such as Troponin, within Cardiovesicles captured using antibodies against POPDC2 and CHRNE, (FIGS. 24B and 24E). Transcriptomic analyses further confirmed the cardiac-specificity, showing an enrichment of heart-specific transcripts prioritized by tau score (cardiac-specificity score) in these vesicles (FIGS. 24C and 24F). For tau, raw counts were downloaded from GTEx v8, and CPM (counts per million) normalized. The mean expression per tissue was calculated, log 2 transformed, and quantile normalized, and then used to calculate tau. Notably, the box plots in FIGS. 24D and 24G underscore the clear transcriptomic enrichment for cardiac transcripts captured by POPDC2 and CHRNE antibodies. Additionally, tissue enrichment and UMAP analysis confirmed the high cardiac-enrichment of transcripts such as BMP10, TNNL3, LRRC10, and FBXO40 within Cardiovesicles (FIGS. 24H-24M), confirming their cardiac origin and supporting the specificity of the ExoCapture-MSB method for cardiac-derived EV capture.

Cardiovesicle Transcript Profiling and snRNA Mapping in Cardiovascular Conditions

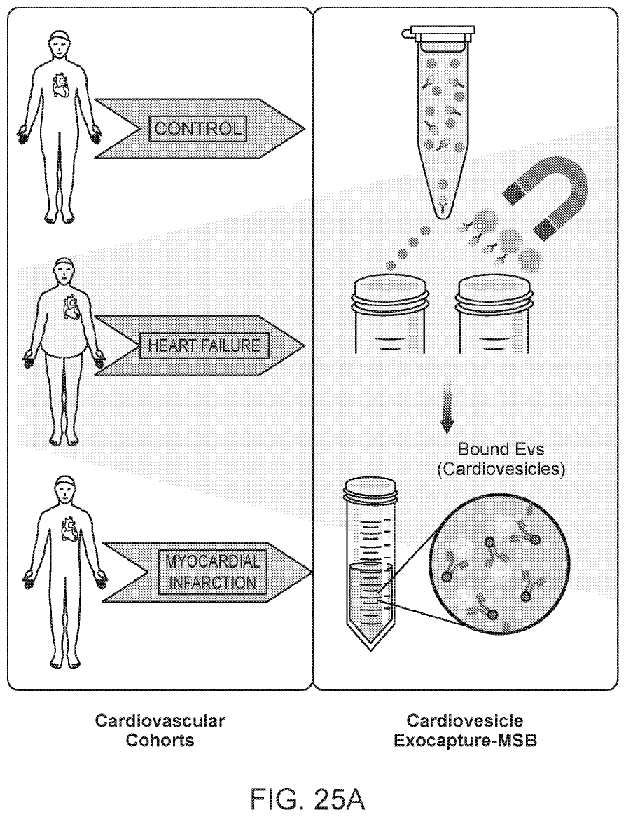

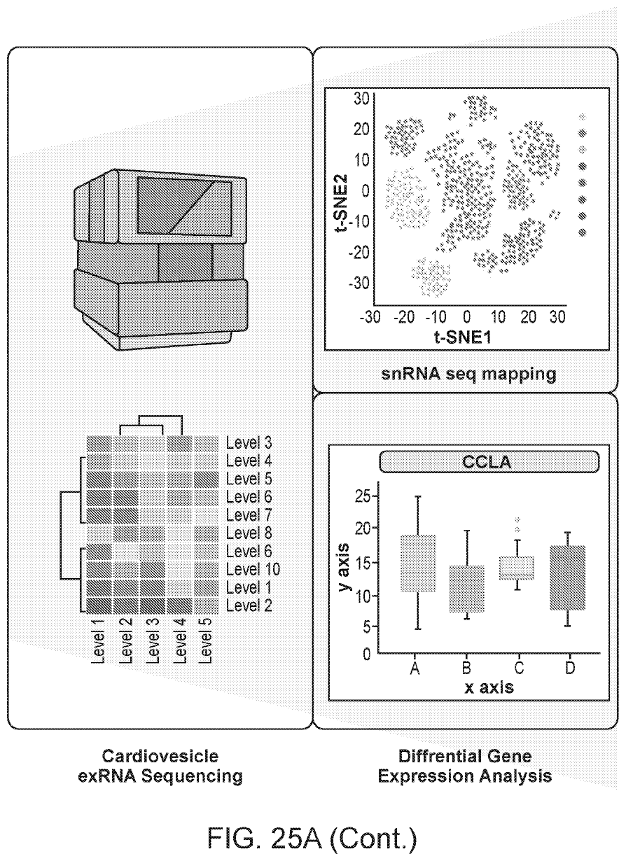

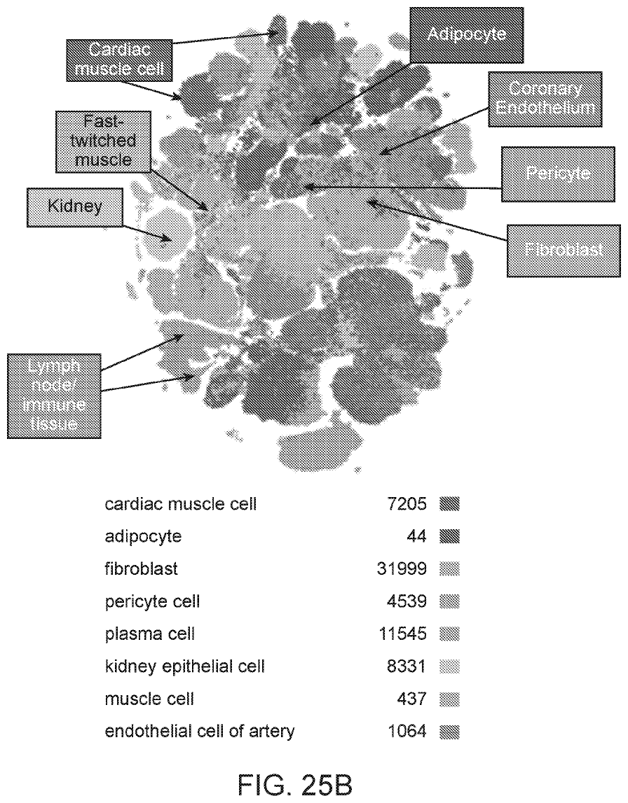

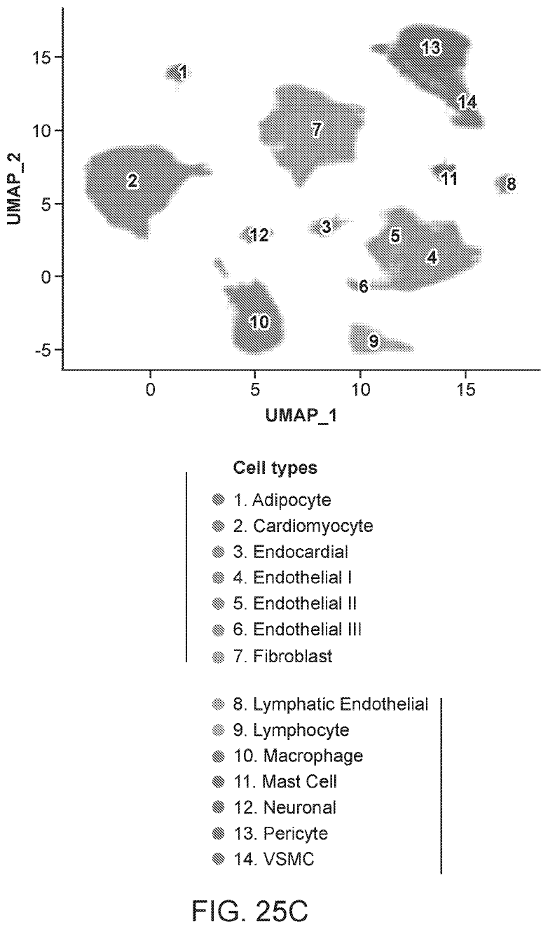

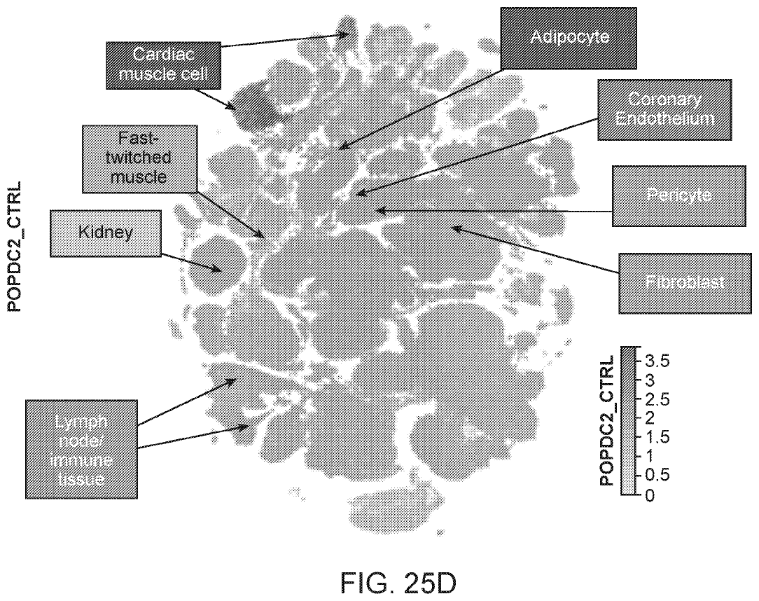

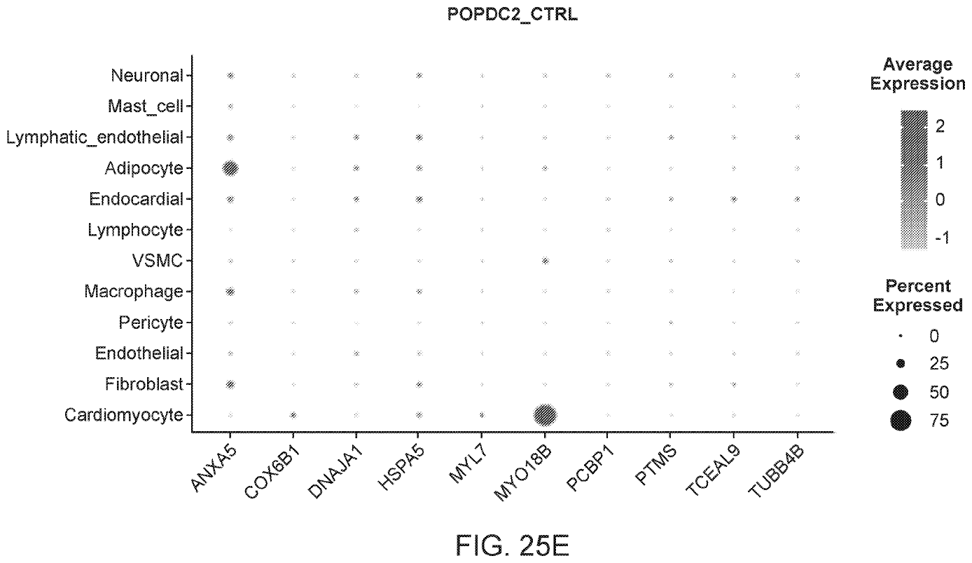

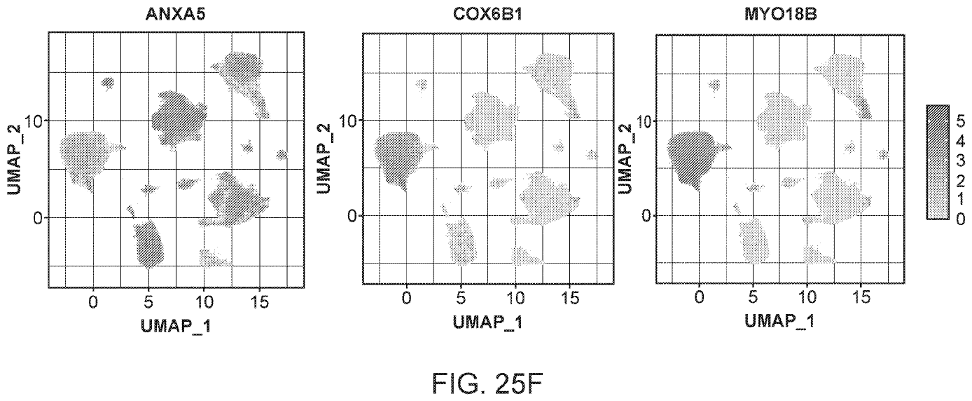

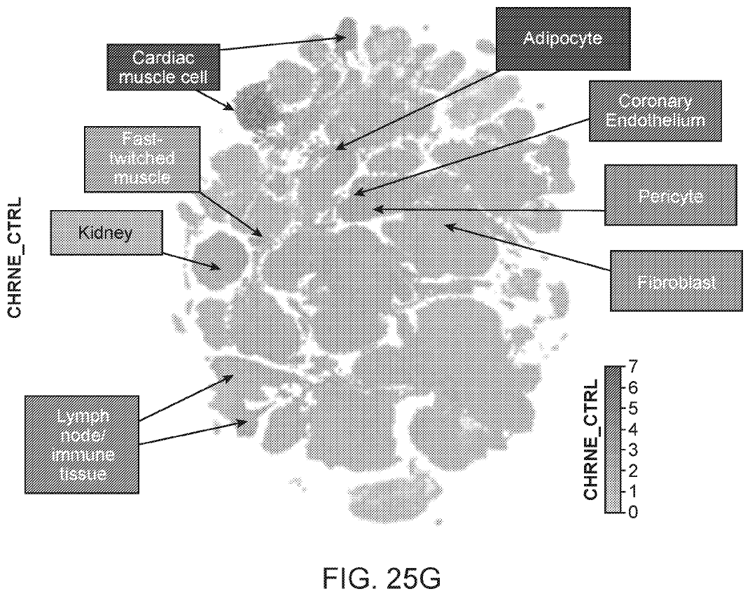

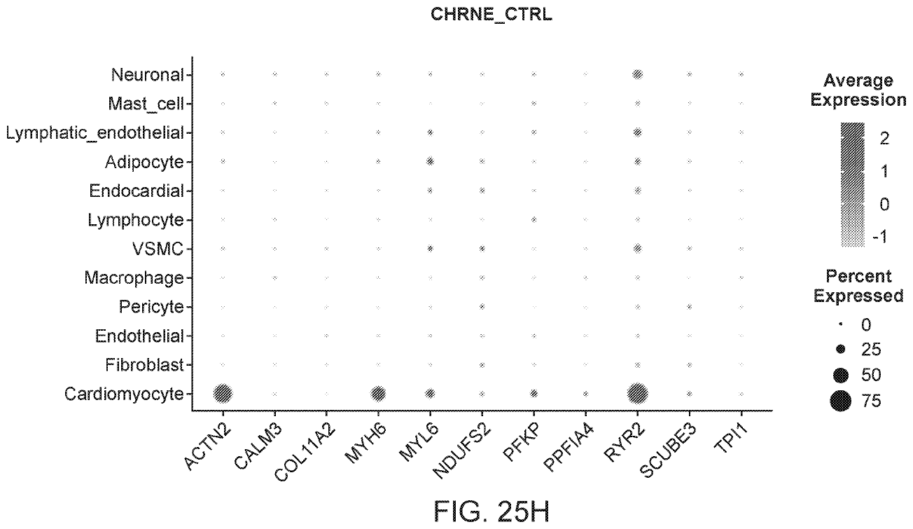

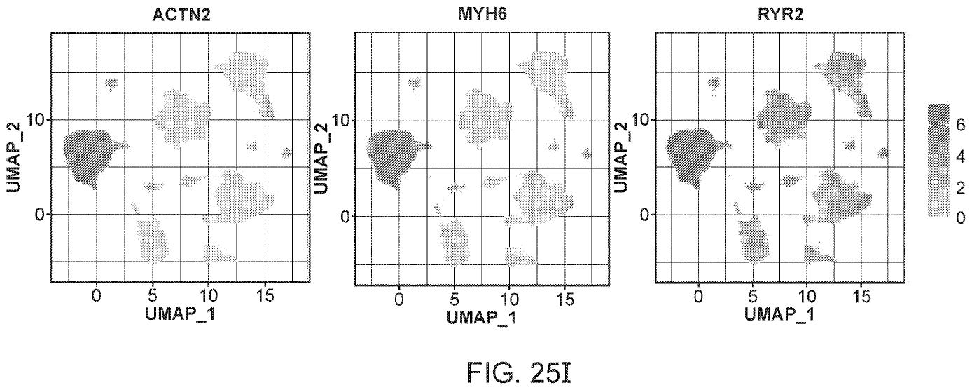

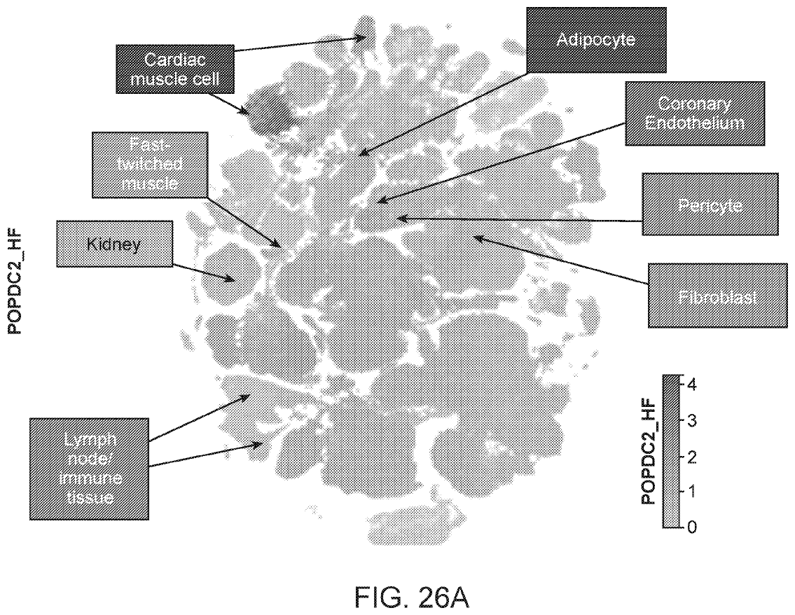

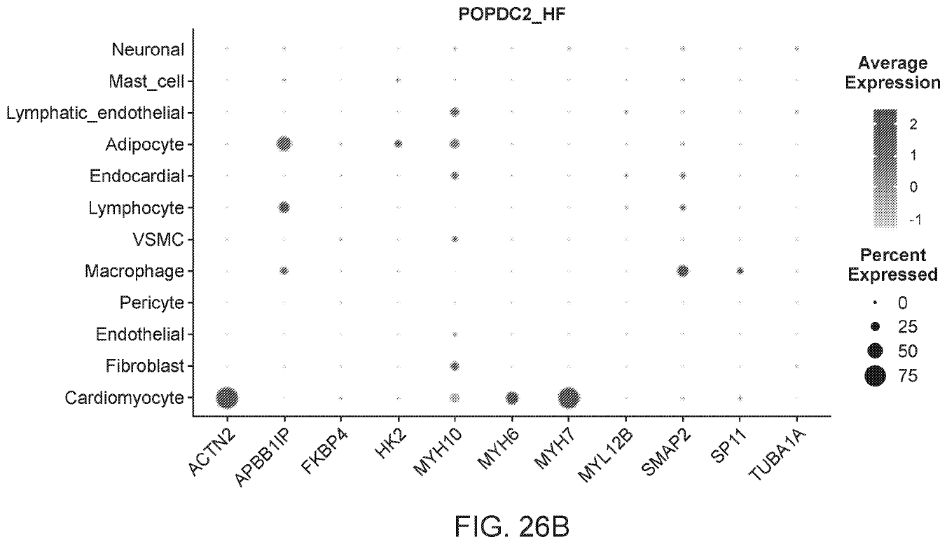

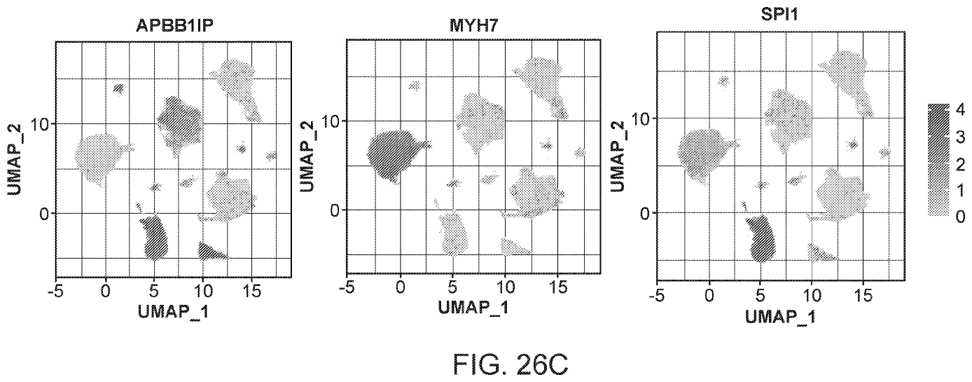

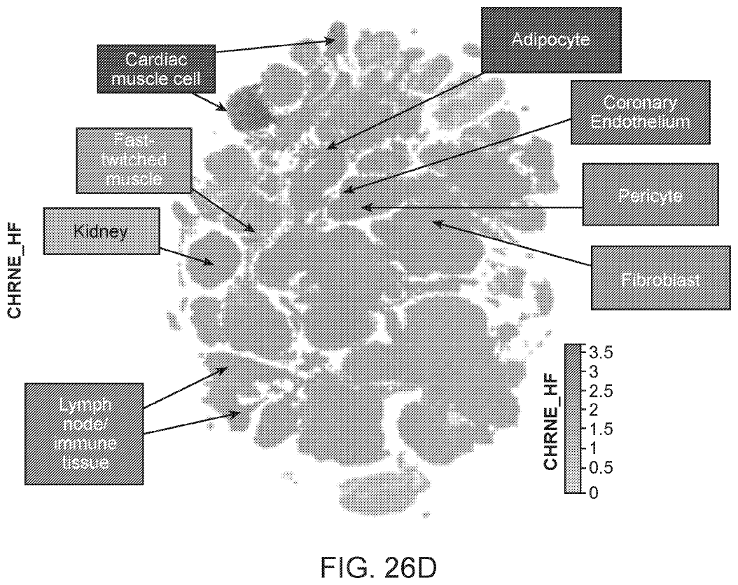

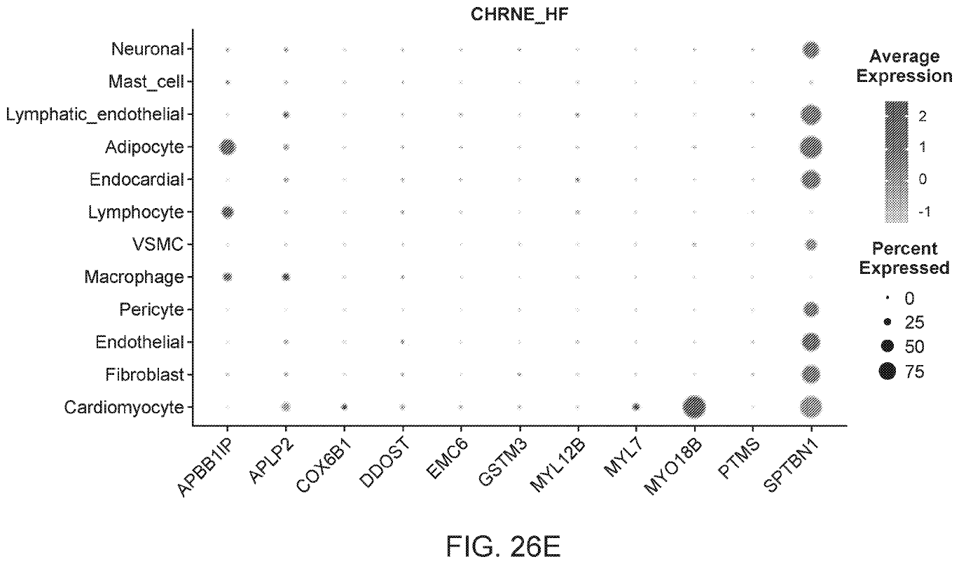

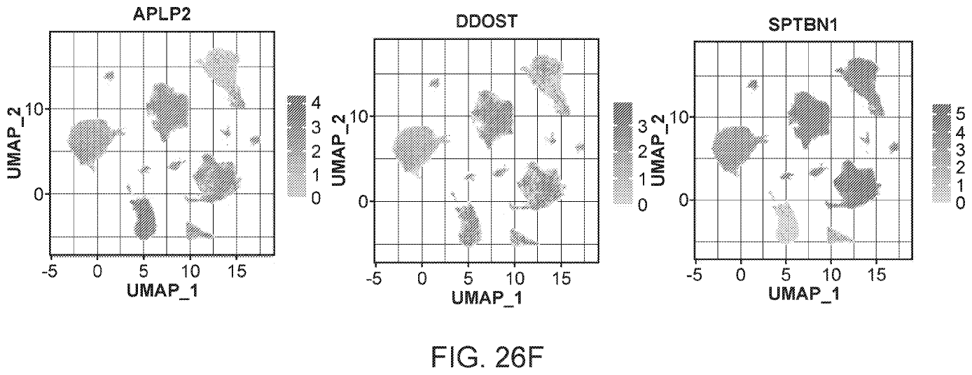

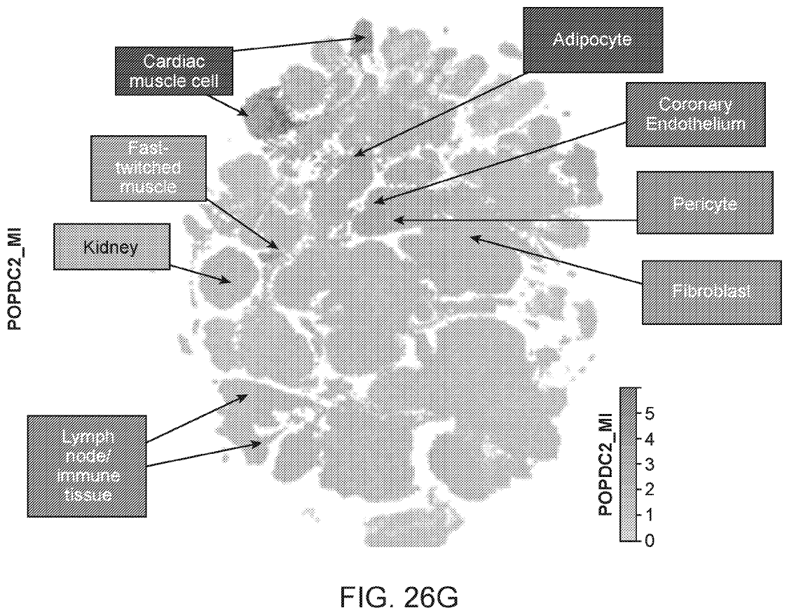

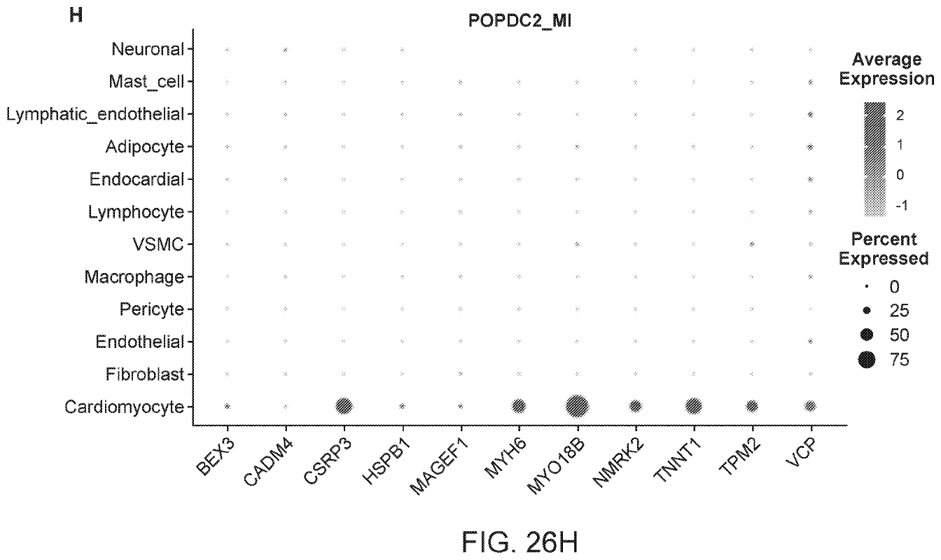

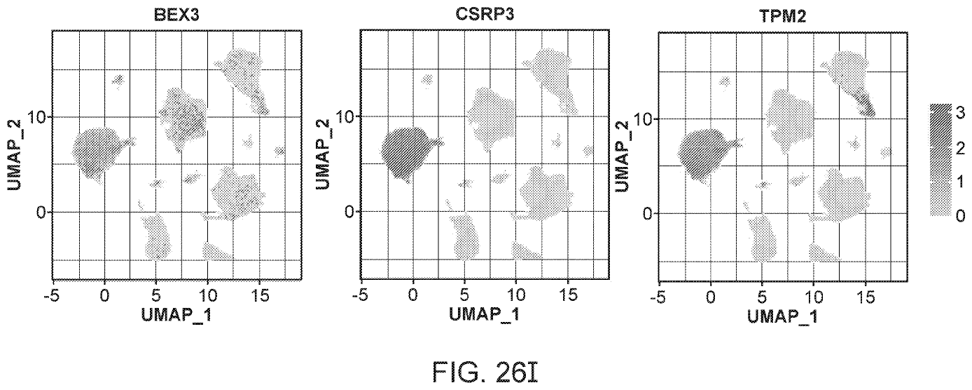

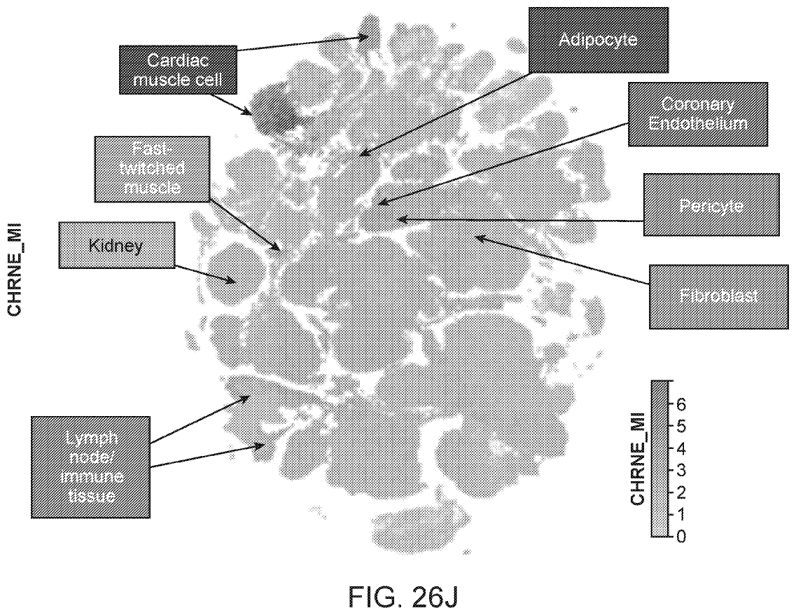

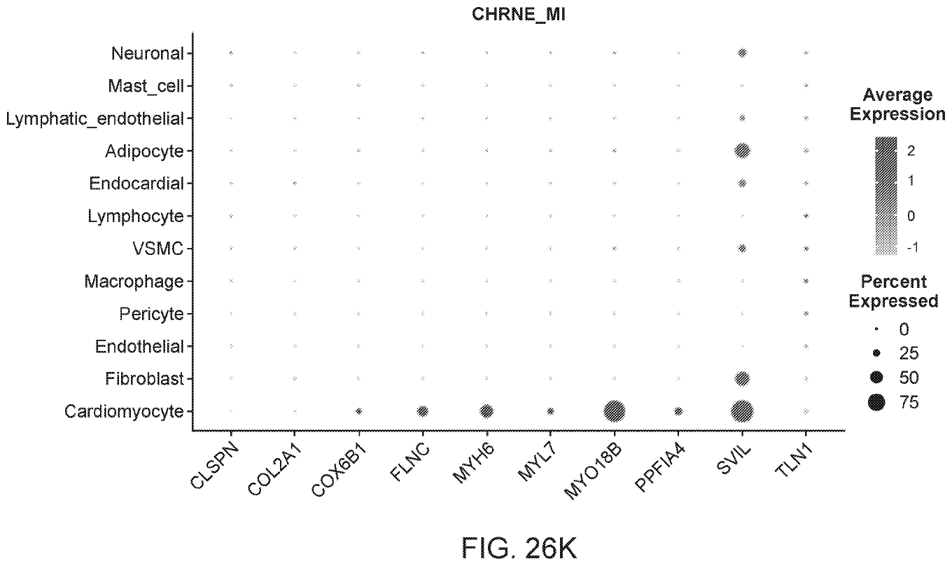

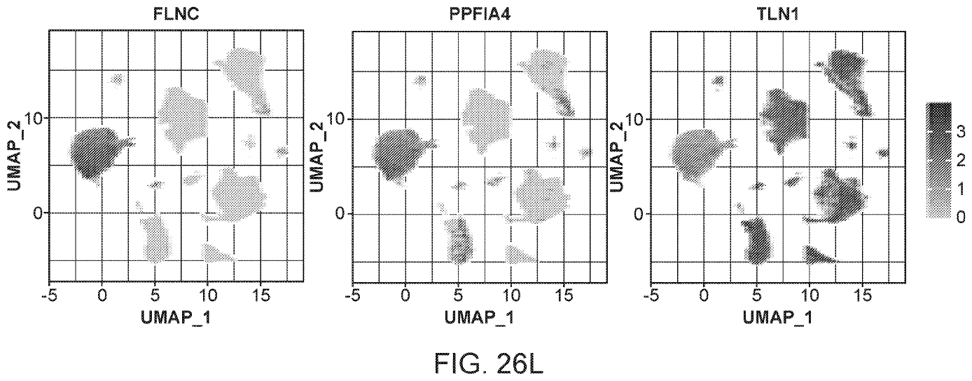

A systematic approach was employed to isolate and analyze Cardiovesicles from cardiovascular cohorts (FIG. 25A). ExoCapture-MSB facilitated the isolation of EVs from patients with heart failure (HF), myocardial infarction (MI), and control groups. Subsequent sequencing and analysis of the exRNA from these vesicles revealed that the most consistently abundant transcripts, when immunocaptured using POPDC2 and CHRNE from controls, displayed high cardiac specificity, as confirmed by mapping onto the multi-organ single-cell transcriptomic atlas dataset, Tabula Sapiens, as well as onto a single-nuclear dataset of human dilated and hypertrophic cardiomyopathy (FIGS. 25B-D and 25G). Detailed UMAP analyses, as shown in FIGS. 25F and 25I, alongside summary dotplots of different cardiac cellular populations (FIGS. 25E and 25H), confirmed the cardiac-specific nature of the transcripts within Cardiovesicles. Consistently abundant transcripts from HF and MI patients were mapped onto various cellular populations (Tabula Sapiens dataset) and the snRNA dataset of human cardiomyopathy, as shown in FIGS. 26A, 26D, 26G, and 26J for the overview and FIGS. 26B, 26E, 26H, and 26K for summary dotplots. The individual target UMAPs in FIGS. 26C, 26F, 26I, and 26L further illustrate the cardiac-specific nature of Cardiovesicle content, which remained consistent across different disease states, underscoring the diagnostic value of these markers in cardiovascular diseases.

Differential Gene Expression in Cardiovesicles from Heart Failure and Myocardial Infarction Patients

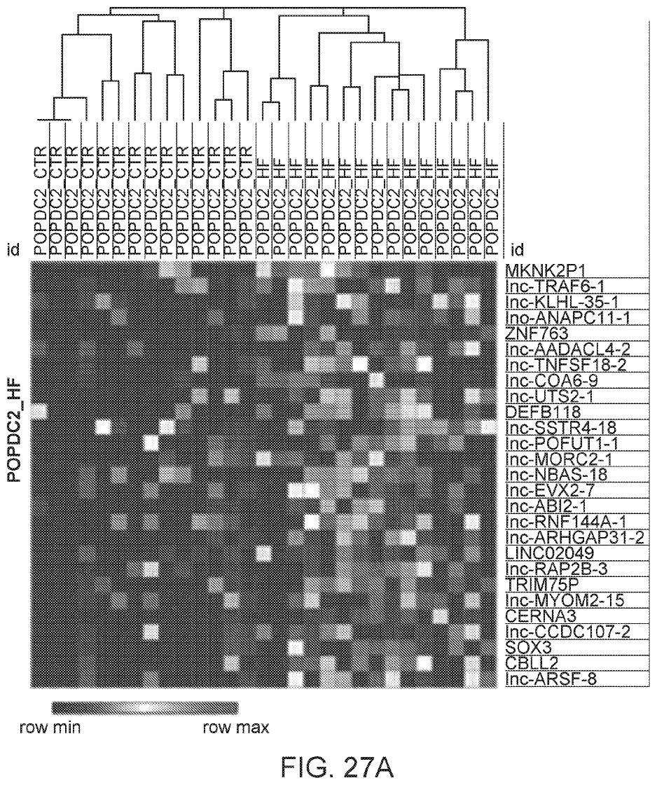

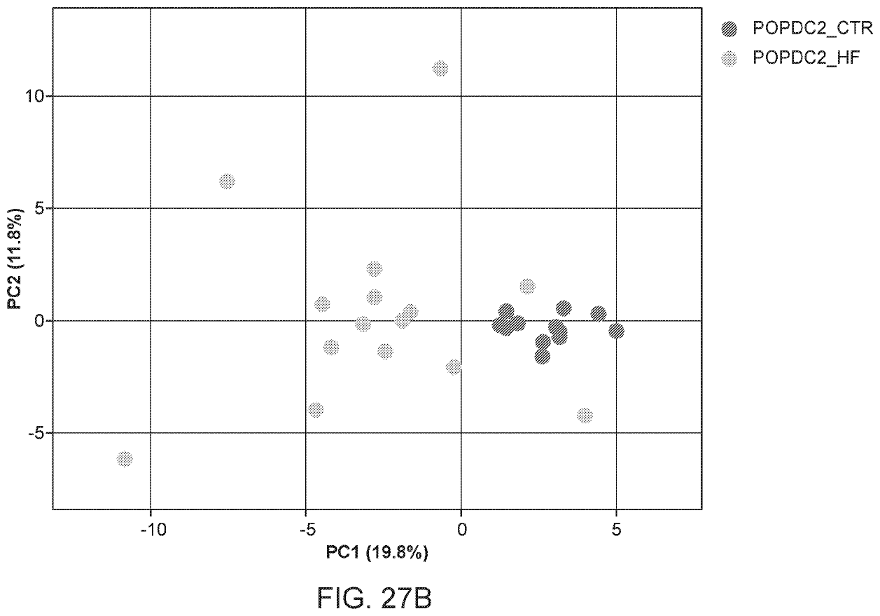

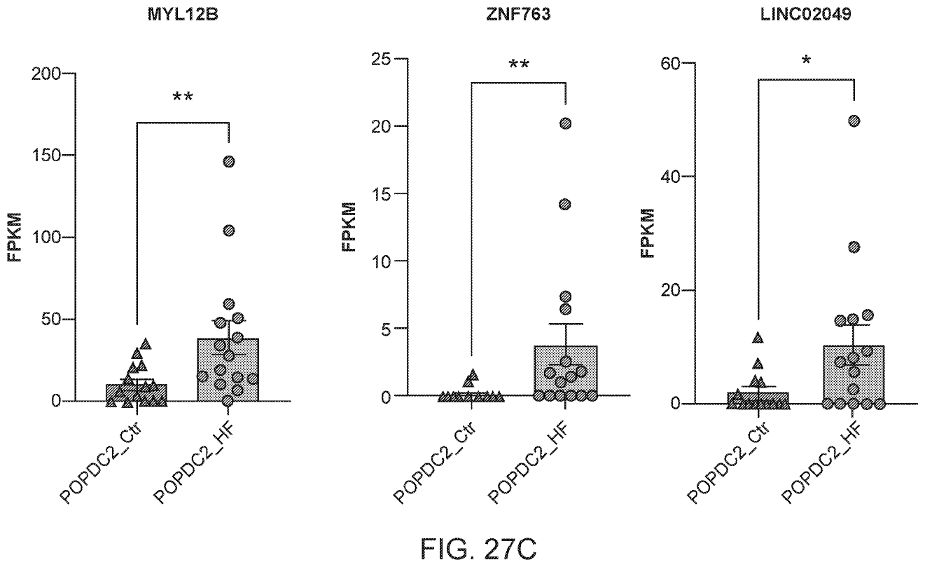

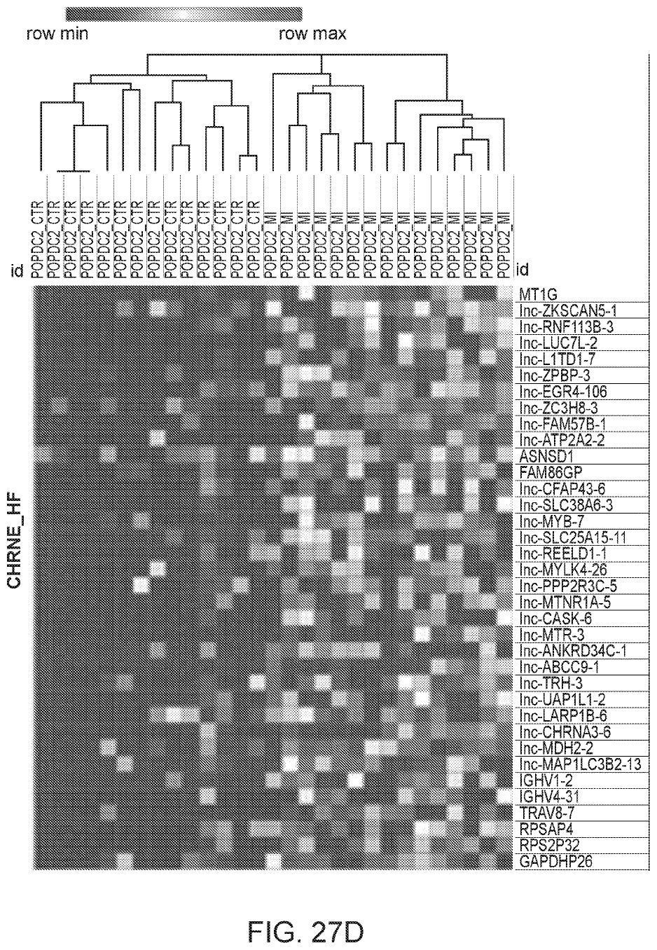

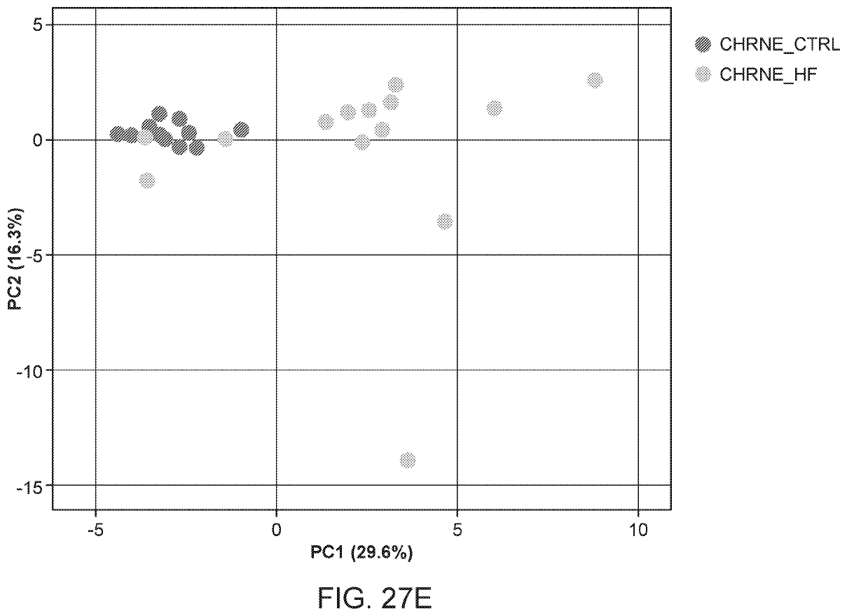

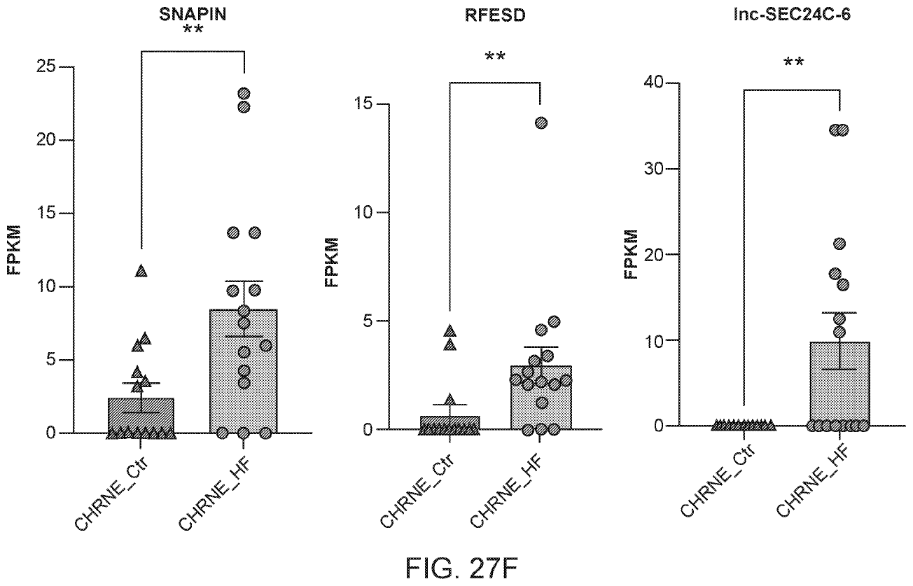

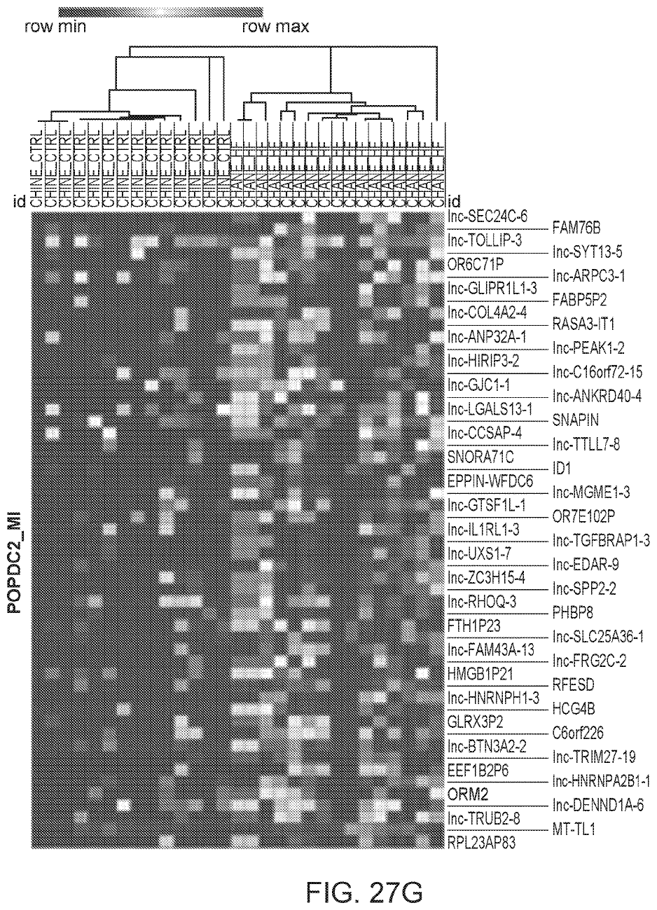

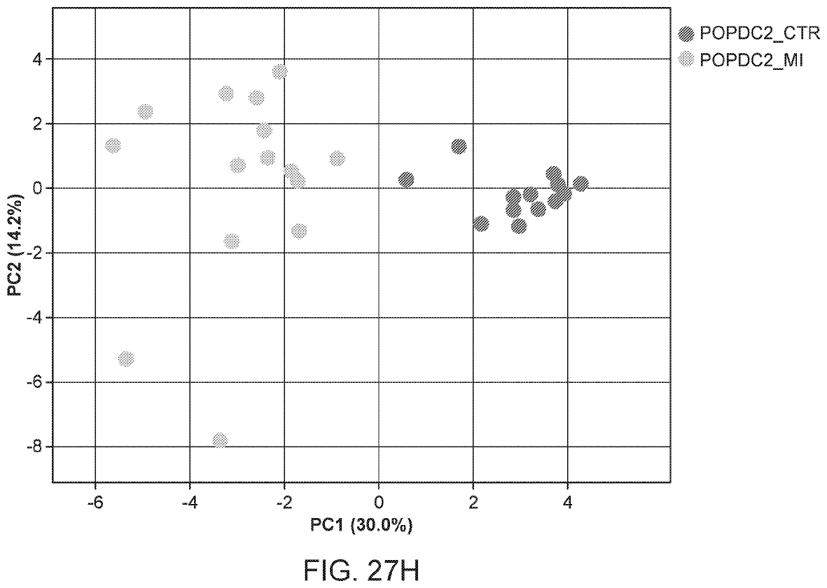

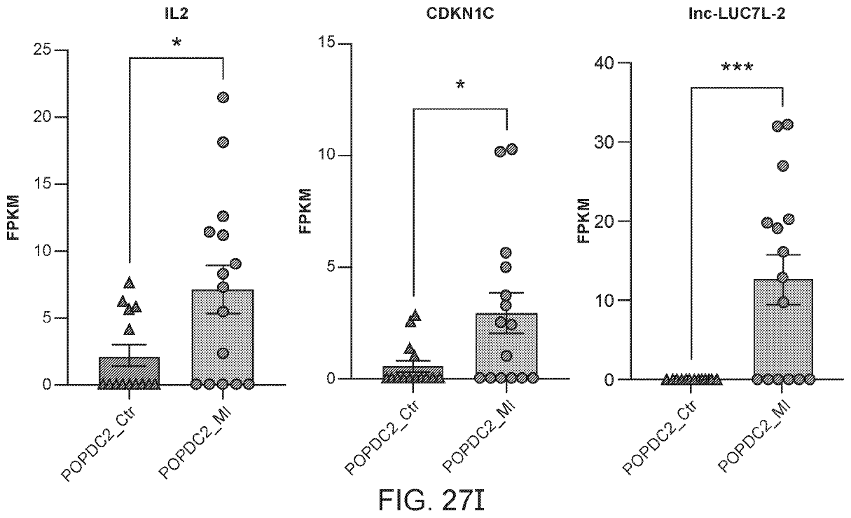

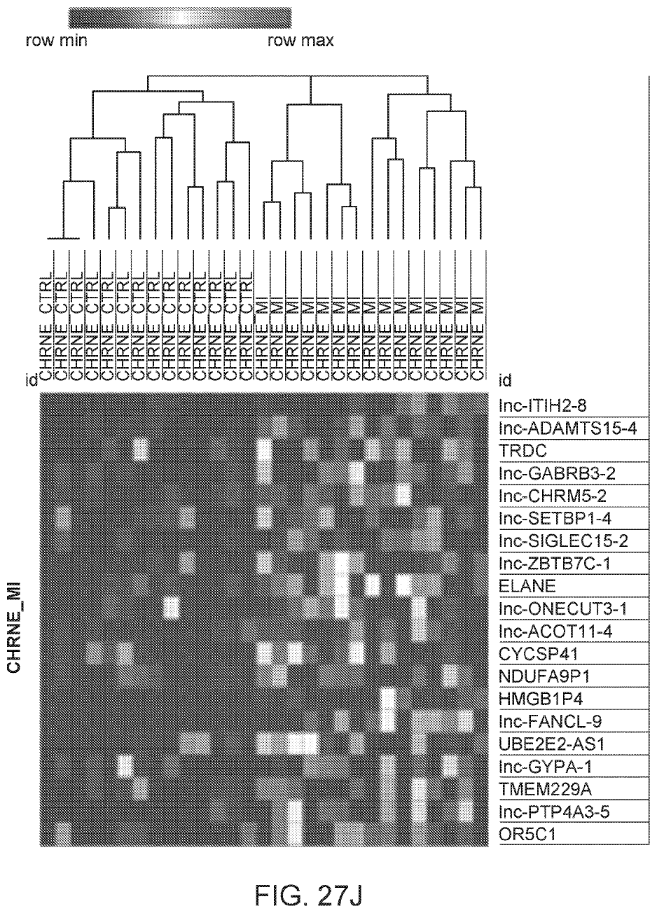

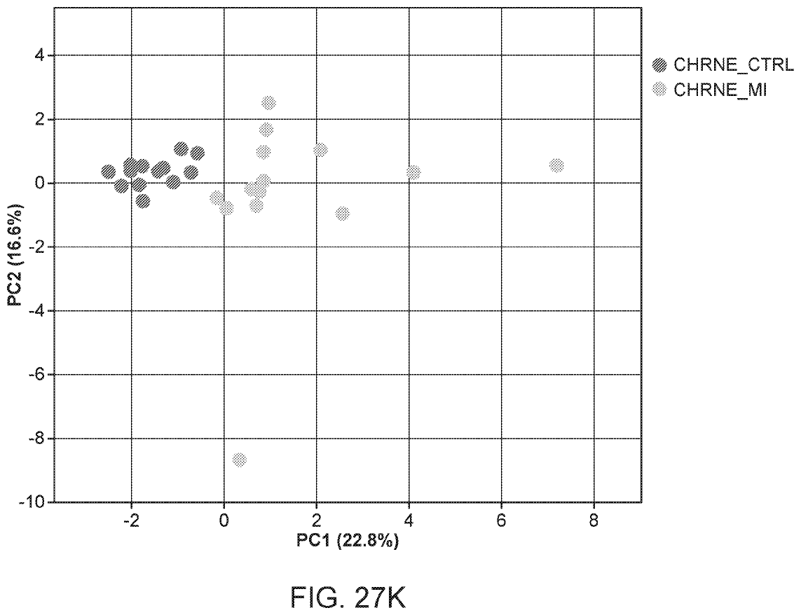

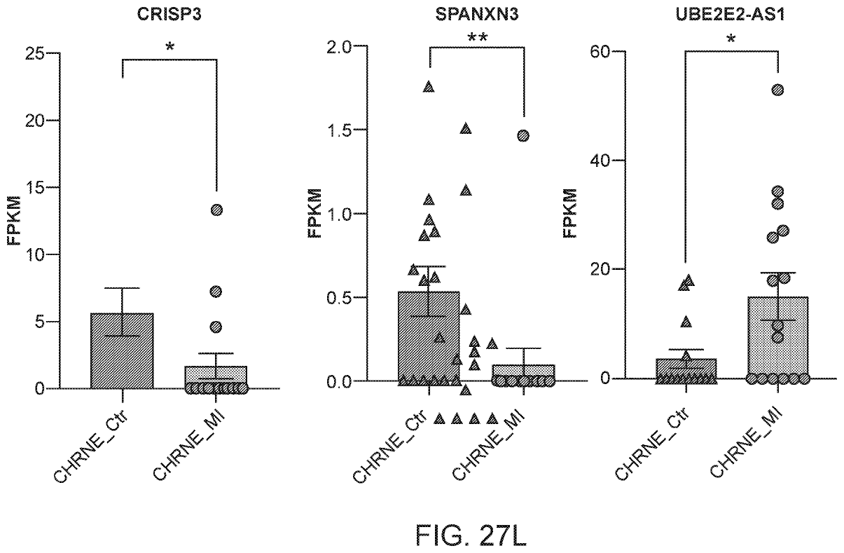

Differential gene expression in cardiovesicles from HF and MI patients was performed and compared against healthy controls. Heatmap (FIGS. 27A, 27D, 27G, and 27J) and principal component analyses (PCA) (FIGS. 27B, 27E, 27H, and 27K) were performed to delineate the variations in gene expression levels among different patient groups and controls. Boxplots in FIGS. 27C, 27F, 27I, and 27L illustrate differentially expressed transcripts in POPDC2 (FIGS. 27C and 271) and CHRNE (FIGS. 27F and 27L) Cardiovesicles from HF (FIGS. 27C and 27F) and MI patients (FIGS. 271 and 27L). These findings indicate significant alterations in the cardiovesicular transcriptome, reflecting the underlying cardiac conditions. The differentially expressed genes identified were further validated via qPCR in an additional cohort, confirming their potential as biomarkers for myocardial infarction and heart failure.

### CONCLUSION

The analysis pipeline described above represents a robust approach to the discovery and validation of tissue-specific EVs. The experiments described herein demonstrate the benefit the disclosed methods in the context of a “liquid biopsy” in cardiovascular health and provide an avenue to deploy these methods to reshape diagnostic and therapeutic strategies for patients suffering from cardiovascular disease. At the same time, these methods are applicable to tissue-specific EV discovery and immunocapturing methodology to other tissues, providing an advancement in the development of more accurate and non-invasive diagnostic tests, a better understanding of the pathophysiology of several diseases, and paving the way towards targeted therapeutic interventions.

### Additional Embodiments

Further embodiments contemplated by the present disclosure are enumerated below.
E1. A multiplexed method for characterizing a population of tissue-specific extracellular vesicles (TSEVs) in a biofluid and/or tissue sample, comprising a mixed population of EVs (MEVs), comprising:
   (a) immobilizing the population of TSEVs in the sample on a surface functionalized with an affinity capture agent that specifically binds to a biomarker present on the plasma membrane of TSEVs; and
   (b) analyzing individual TSEVs within the population TSEVs using super-resolution microscopy to produce a profile of the population of TSEVs;
   wherein the profile of the population of TSEVs includes information on at least:
   (i) membrane protein composition of individual TSEVs; and
      (ii) intra-vesicular cargo composition of individual TSEVs.
E2. A multiplexed method for characterizing a population of tissue-specific extracellular vesicles (TSEVs) in a biofluid and/or tissue sample, comprising a mixed population of EVs (MEVs), comprising:
   (a) immobilizing the population of TSEVs in the sample on a surface functionalized with an affinity capture agent that specifically binds to a biomarker present on the plasma membrane of TSEVs; and
   (b) analyzing individual TSEVs within the population TSEVs using super-resolution microscopy;
   (c) producing a profile of the population of TSEVs, wherein the profile of the population of TSEVs includes information on at least:
      (i) membrane protein composition of individual TSEVs; and
      (ii) intra-vesicular cargo composition of individual TSEVs.
E3. The method of E1 or E2, wherein the method further comprises, prior to step (a), identifying a biomarker as a tissue-specific biomarker in the population of TSEVs on the basis that the biomarker:
   (a) is a plasma membrane protein;
   (b) exhibits greater than 5 transcripts per million (TPM) RNA expression in a tissue of origin; and
   (c) exhibits less than 0.1 TPM RNA expression in tissues other than the tissue of origin.
E4. The method of any one of E1-E3, wherein the method further comprises, prior to step (a), identifying a biomarker as a tissue-specific biomarker in the population of TSEVs on the basis of a tissue enrichment score, tau (i).
E5. The method of E4, wherein z is calculated according to the following formula:

$$τ = \frac{{\sum }_{i = 1}^{n} ( 1 - {\hat{x}}_{i} )}{n - 1} ; {\hat{x}}_{i} = \frac{{x}_{i}}{{max}_{1 \leq i \leq n} ( {x}_{i} )}$$

wherein n is the number of tissues: xᵢ corresponds to expression of the gene in a given tissue; and \hat{x}ᵢ is the expression profile component normalized by the maximal component value.
E6. The method of E5, wherein τ is greater than or equal to 0.9.
E7. The method of E5, wherein τ is greater than 0.9.
E8. The method of any one of E1-E7, wherein the method further comprises, prior to step (a), isolating the population of MEVs from the sample, thereby producing an enriched population of MEVs.
E9. The method of E8, further comprising isolating the population of TSEVs from the enriched population of MEVs, wherein the isolated population of TSEVs is used for immobilizing in step (a).
E10. The method of E8 or E9, wherein isolating the population of MEVs from the sample is performed via affinity capture, size exclusion chromatography (SEC), ultracentrifugation, ultrafiltration, flow field-flow fractionation, hydrostatic filtration dialysis, enzyme-linked immunosorbent assay (ELISA) density gradient, immunoprecipitation, polyethylene glycol (PEG) precipitation, PEG/dextran aqueous two phase system (ATPS) isolation, lectin-induced agglutination, acoustic nanofilter, and/or a microfluidic separation.
E11. The method of E9 or E10, wherein isolating the population of MEVs is performed using an affinity capture method selected from the group consisting of affinity chromatography, flow-based affinity immunoassay, affinity pulldown, affinity bead capture, affinity resin capture, microfluidic affinity capture, ELISA, magneto-immunoprecipitation, mixed-mode chromatography (MMC), and membrane-affinity spin column (MASC).
E12. The method of E10 or E11, wherein isolating the population of MEVs is performed using an affinity capture agent that specifically binds to one or more EV-specific biomarkers selected from the group consisting of CD9, CD63, and CD81.
E13. The method of any one of E1-E7, wherein the method does not comprise, prior to step (a), isolating the population of MEVs from the sample.
E14. The method of any one of E1-E13, wherein the biomarker present on the TSEVs of step (a) is a tissue-specific biomarker or an EV-specific biomarker.
E15. The method of E14, wherein EV-specific biomarker is a protein selected from the group consisting of CD9, CD63, and CD81.
E16. The method of any one of E1-E15, wherein the MEVs and TSEVs do not exhibit substantial expression of a negative selection marker selected from the group consisting of Apolipoprotein A1 (ApoA1), Apolipoprotein A2 (ApoA2), Apolipoprotein B (ApoB), albumin (ALB), cytochrome C (CYC), fibronectin (FN), and nuclear RNA (nRNA).
E17. The method of any one of E1-E16, wherein the super-resolution microscopy comprises Single Extracellular Vesicle Nanoscopy (SEVEN).
E18. The method of E17, SEVEN comprises use of single-molecule localization microscopy (SMLM).
E19. The method of E18, wherein the SMLM is quantitative SMLM (qSMLM).
E20. The method of E18, wherein the qSMLM comprises use of a surface assay for molecular isolation (SAMI-qSMLM).
E21. The method of E17, wherein the SMLM is photoactivated localization microscopy (PALM).
E22. The method of E17, wherein the SMLM is stochastic optical reconstruction microscopy (STORM).
E23. The method of E21, wherein the STORM is direct STORM (dSTORM).
E24. The method of E17, wherein the SMLM is point accumulation in nanoscale topography (PAINT).
E25. The method of any one of E1-E17, wherein the super-resolution microscopy comprises super-resolution radial fluctuations (SRRF) imaging.
E26. The method of any one of E1-E17 and E25, wherein the super-resolution microscopy comprises total internal reflection fluorescence (TIRF) illumination.
E27. The method of any one of E1-E17, E25, and E26, wherein the super-resolution microscopy comprises SRRF imaging and TIRF illumination.
E28. The method of any one of E1-E17, and E25 wherein the super-resolution microscopy comprises wide field illumination.
E29. The method of any one of E1-E17, E25, and E28, wherein the super-resolution microscopy comprises SRRF imaging and wide field illumination.
E30. The method of any one of ET-E17 and E25, wherein the super-resolution microscopy comprises confocal illumination.
E31. The method of any one of E1-E17, E25, and E30, wherein the super-resolution microscopy comprises SRRF imaging and confocal illumination.
E32. The method of any one of ET-E31, wherein the profile of the population of TSEVs further includes information on one or more of the following:
   (i) size of TSEVs;
   (ii) shape of TSEVs;
   (ii) quantity or concentration of TSEVs; and
   (iii) heterogeneity of TSEVs.
E33. The method of E32, wherein the information on the shape of TSEVs comprises information on the circularity and/or eccentricity of the TSEVs.
E34. The method of any one of E14-E33, wherein the tissue-specific biomarker is from a tissue selected from the group consisting of cardiac tissue, neural tissue, pancreatic tissue, immune tissue, and cancer tissue.
E35. The method of any one of E14-E34, wherein the EV-specific biomarker and/or the tissue-specific biomarker is a membrane protein, cytoplasmic protein, glycan, nucleic acid, lipid, or a combination thereof.
E36. The method of any one of E1-E35, wherein the affinity capture agent of step (a) is a protein, peptide, aptamer, carbohydrate, or a combination thereof.
E37. The method of E36, wherein the carbohydrate is a lectin.
E38. The method of E35, wherein the protein or peptide is selected from the group consisting of a primary antibody, secondary antibody. Fab, F(ab′), F(ab′)2, single chain variable fragment (scFv), Fd, minibody, variable heavy domain, variable light domain, variable NAR domain, single chain binding polypeptide, dAb fragment, nanobody. VHH, and toxin.
E39. The method of any one of E1-E38, further comprising labeling the TSEVs with a labeling agent.
E40. The method of E39, wherein the labeling agent a fluorescent reporter or a binding agent conjugated to a fluorescent reporter.
E41. The method of E40, wherein the fluorescent reporter is a photoswitchable fluorescent reporter, photoactivatable fluorescent reporter, photoconvertible fluorescent reporter, spontaneously blinking fluorescent reporter, or temporarily binding fluorescent reporter.
E42. The method of E40 or E41, wherein the fluorescent reporter is selected from the group consisting of AF532, AF488, AF532, AF555, AF568, AF594, AF647, AF680, AF700, AF750, Atto488, Atto532, Atto647N, Atto680, Atto700, CF532, CF555, CF568, CF647, CF660C, CF680, CF750, CF488A, CF583R, CF597R, CF680R, CF535ST, Cy3, Cy3b, Cy5, DY-634, DyLight650, Dronpa, JF549. JF646, JFX549, JFX554, JFX646. JFX650, mlrisFP, mMaple, mMaple 3. PAGFP, PAmCherry, PATagRFP. PAmKate, PS-CFP2, and quantum dots.
E43. The method of any one of E1-E42, wherein the functionalized surface of step (a) is a coverslip.
E44. The method of any one of E1-E43, wherein the functionalized surface of step (a) is coated with a coupling agent.
E45. The method of E44, wherein the coupling agent is attached to the functionalized surface by way of a linker moiety.
E46. The method of E44 or E45, wherein the coupling agent is selected from the group consisting of MCP2, MCP4, p-aminophenyltrimethoxysilane (APTMS), and aminotrimethoxy silane (ATMS).
E47. The method of any one of E1-E46, wherein the intra-vesicular cargo comprises a protein, nucleic acid, lipid, or carbohydrate.
E48. The method of E47, wherein the nucleic acid is an RNA or a DNA.
E49. The method of E48, wherein the RNA is a messenger RNA (mRNA), microRNA (miRNA), long non-coding RNA (lncRNA), transfer RNA (tRNA), tRNA-derived small RNA (tsRNA), ribosomal RNA (rRNA), small rDNA-derived RNA (srRNA), small nucleolar RNA (snoRNA). Piwi-interacting RNA (piRNA).
E50. The method of any one of E1-E49, wherein the membrane protein composition and/or the intra-vesicular cargo composition of individual TSEVs is further assayed using proteomic analysis to generate a proteomic profile of the population of TSEVs.
E51. The method of E50, wherein the proteomic analysis comprises an immunoassay, mass spectrometry (MS), high performance liquid chromatography (HPLC), reversed-phase chromatography, dot blot analysis, two-dimensional gel electrophoresis, Edman sequencing, protein microarray analysis, structural proteomic analysis, functional proteomic analysis, protein-protein interaction analysis, proteome mining, and post-translational modification analysis.
E52. The method of any one of E1-E51, wherein the intra-vesicular cargo composition of individual TSEVs is further analyzed using transcriptomic analysis to generate a transcriptomic profile of the population of TSEVs.
E53. The method of E52, wherein the transcriptomic analysis comprises RNA sequencing (RNA-seq).
E54. The method of E52 or E53, wherein the transcriptomic analysis comprises analysis of lncRNAs.
E55. The method of any one of E1-E54, wherein the sample is: (1) a biofluid or tissue sample obtained from a subject; or (2) a cell culture medium.
E56. The method of E55, wherein the biofluid sample obtained from a subject is selected from the group consisting of whole blood, plasma, serum, cerebrospinal fluid, saliva, sputum, nasal secretion, ocular secretion, cystic fluid, synovial fluid, bronchoalveolar lavage fluid, amniotic fluid, bone marrow aspirate, bile, milk, stool, swab, smear, semen, or urine.
E57. The method of E55 or E56, wherein the biofluid sample has a volume between 0.08 μL and 400 μL.
E58. The method of E57, wherein the biofluid sample has a volume no greater than 1 μL.
E59. The method of any one of E55-E58, wherein the tissue sample is a formalin-fixed paraffin-embedded (FFPE) tissue block, fixed tissue, fresh tissue, or frozen tissue.
E60. The method of any one of E1-E59, wherein the method is performed in accord with Minimal Information for Studies of Extracellular Vesicles (MISEV) guidelines.
E61. The method of any one of E1-E60, wherein the method identifies one or more biomarkers present on the plasma membrane or in the lumen of TSEVs as being associated with a disease or disorder.
E62. A method of identifying one or more biomarkers associated with a disease or disorder from a population of TSEVs in a sample, comprising:
   (i) performing the method of any one of E1-E61, thereby obtaining a level of expression of one or more tissue-specific biomarkers from an enriched population of disease-associated TSEVs in the sample;
   (ii) calculating a difference score for the one or more tissue-specific biomarkers by comparing a level of expression of the one or more tissue-specific biomarkers from the population of disease-associated TSEVs to a level of expression of the one or more tissue-specific biomarkers from a reference population of TSEVs;
wherein a difference score above a cutoff value indicates that the one more tissue-specific biomarkers are associated with a disease or disorder.
E63. The method of E62, wherein the cutoff value is at a 50^{th} percentile, 60^{th} percentile, 70^{th} percentile, 80^{th} percentile, 90^{th} percentile, or greater of the difference score in the reference population of TSEVs.
E64. The method of E62 or E63, wherein the sample is: (1) a fluid sample obtained from a subject having the disease or disorder: or (2) a cell culture medium comprising a population of cells that model the disease or disorder.
E65. A method of diagnosing a subject as having or at risk of developing a disease or disorder, comprising:
   (i) performing the method of any one of E1-E61 on a sample obtained from the subject, thereby obtaining a level of expression of one or more tissue-specific biomarkers associated with the disease or disorder;
   (ii) calculating a difference score for the subject by comparing a level of expression of the one or more tissue-specific biomarkers in the subject to a level of expression of the one or more tissue-specific biomarkers from a reference population of subjects without the disease or disorder:
wherein a difference score above a cutoff value identifies the subject as having or at risk of developing the disease or disorder.
E66. The method of E65, wherein the cutoff value is at a 50^{th} percentile, 60^{th} percentile, 70^{th} percentile, 80^{th} percentile, 90^{th} percentile, or greater of the difference score in the reference population of subjects without the disease or disorder.
E67. A method of assessing therapeutic efficacy of a therapeutic agent in a subject having or at risk of developing a disease or disorder, comprising:
   (i) performing the method of any one of E1-E61 on a sample obtained from the subject prior to treatment with the therapeutic agent thereby obtaining a pre-treatment level of expression of one or more tissue-specific biomarkers characterized as having altered expression associated with the disease or disorder;
   (ii) administering an amount of the therapeutic agent to the subject;
   (iii) performing the method of any one of E1-E61 on a sample obtained from the subject following treatment with the therapeutic agent thereby obtaining a post-treatment level of expression of the one or more tissue-specific biomarkers;
   (iv) calculating a difference score for the therapeutic agent by comparing the pre-treatment level of expression of the one or more tissue-specific biomarkers to the post-treatment level of expression of the one or more tissue-specific biomarkers; wherein a difference score above a cutoff value indicates that the therapeutic agent is effective at treating the disease or disorder.
E68. The method of E67, wherein the cutoff value is at a 50t percentile, 60^{th} percentile, 70^{th} percentile, 80^{th} percentile, 90^{th} percentile, or greater of the difference score in the pretreatment level of expression of the one or more tissue-specific biomarkers.
E69. The method of E67 or E68, the method comprising characterizing the one or more tissue-specific biomarkers as having altered expression associated with the disease or disorder prior to performing step (i).
E70. A method of selecting a therapeutic agent for the treatment of a subject having or at risk of developing a disease or disorder, comprising:
   (i) performing the method of any one of E1-E61, thereby obtaining a level of expression of one or more tissue-specific biomarkers from an enriched population of disease-associated TSEVs:
   (ii) calculating a difference score for the one or more tissue-specific biomarkers by comparing a level of expression of the one or more tissue-specific biomarkers from the population of disease-associated TSEVs to a level of expression of the one or more tissue-specific biomarkers from a reference population of TSEVs, wherein a difference score above a cutoff value indicates that the one more tissue-specific biomarkers are associated with a disease or disorder; and
   (iii) identifying a common biological signaling pathway associated with the one or more disease-associated tissue-specific biomarkers; and
   (iv) selecting a therapeutic agent that modulates activity of the common biological signaling pathway.
E71. The method of E70, further comprising (v) administering an effective amount of the therapeutic agent to the subject having or at risk of developing the disease or disorder.
E72. The method of E70 or E71, wherein the cutoff value is at a 50^{th} percentile, 60^{th} percentile, 70^{th} percentile, 80^{th} percentile, 90^{th} percentile, or greater of the difference score in the reference population of TSEVs.
E73. The method of any one of E62-E72, wherein the level of expression is a mean or median level of expression.
E74. The method of any one of E65-E73, the method further comprising obtaining the sample from the subject having or at risk of developing the disease or disorder prior to performing step (i).
E75. The method of any one of E65-E73, the method further comprising having obtained the sample from the subject having or at risk of developing the disease or disorder prior to performing step (i).
E76. The method of any one of E62-E75, wherein the disease or disorder is selected from the group consisting of a cancer, cardiovascular disease or disorder, neurological disease or disorder, and autoimmune disease or disorder.
E77. The method of any one of E62-E76, wherein the subject is a human, non-human primate, or rodent.

### OTHER EMBODIMENTS

Various modifications and variations of the described disclosure will be apparent to those skilled in the art without departing from the scope and spirit of the disclosure. Although the disclosure has been described in connection with specific embodiments, it should be understood that the disclosure as claimed should not be unduly limited to such specific embodiments. Indeed, various modifications of the described modes for carrying out the disclosure that are obvious to those skilled in the art are intended to be within the scope of the disclosure. Other embodiments are in the claims.

1. A multiplexed method for characterizing a population of tissue-specific extracellular vesicles (TSEVs) in a biofluid and/or tissue sample, comprising a mixed population of EVs (MEVs), comprising:
(a) immobilizing the population of TSEVs in the sample on a surface functionalized with an affinity capture agent that specifically binds to a biomarker present on the plasma membrane of TSEVs; and
(b) analyzing individual TSEVs within the population TSEVs using super-resolution microscopy to produce a profile of the population of TSEVs;
wherein the profile of the population of TSEVs includes information on at least:
(i) membrane protein composition of individual TSEVs; and
(ii) intra-vesicular cargo composition of individual TSEVs.

2. The method of claim 1, wherein the method further comprises, prior to step (a), identifying a biomarker as a tissue-specific biomarker in the population of TSEVs on the basis that the biomarker:
(a) is a plasma membrane protein;
(b) exhibits greater than 5 transcripts per million (TPM) RNA expression in a tissue of origin; and
(c) exhibits less than 0.1 TPM RNA expression in tissues other than the tissue of origin.

3. The method of claim 1 or 2, wherein the method further comprises, prior to step (a), isolating the population of MEVs from the sample, thereby producing an enriched population of MEVs.

4. The method of claim 3, further comprising isolating the population of TSEVs from the enriched population of MEVs, wherein the isolated population of TSEVs is used for immobilizing in step (a).

5. The method of claim 3 or 4, wherein isolating the population of MEVs from the sample is performed via affinity capture, size exclusion chromatography (SEC), ultracentrifugation, ultrafiltration, flow field-flow fractionation, hydrostatic filtration dialysis, enzyme-linked immunosorbent assay (ELISA) density gradient, immunoprecipitation, polyethylene glycol (PEG) precipitation, PEG/dextran aqueous two phase system (ATPS) isolation, lectin-induced agglutination, acoustic nanofilter, and/or a microfluidic separation.

6. The method of claim 4 or 5, wherein isolating the population of MEVs is performed using an affinity capture method selected from the group consisting of affinity chromatography, flow-based affinity immunoassay, affinity pulldown, affinity bead capture, affinity resin capture, microfluidic affinity capture, ELISA, magneto-immunoprecipitation, mixed-mode chromatography (MMC), and membrane-affinity spin column (MASC).

7. The method of claim 5 or 6, wherein isolating the population of MEVs is performed using an affinity capture agent that specifically binds to one or more EV-specific biomarkers selected from the group consisting of CD9, CD63, and CD81.

8. The method of claim 1 or 2, wherein the method does not comprise, prior to step (a), isolating the population of MEVs from the sample.

9. The method of any one of claims 1-8, wherein the biomarker present on the TSEVs of step (a) is a tissue-specific biomarker or an EV-specific biomarker.

10. The method of claim 9, wherein EV-specific biomarker is a protein selected from the group consisting of CD9, CD63, and CD81.

11. The method of any one of claims 1-10, wherein the MEVs and TSEVs do not exhibit substantial expression of a negative selection marker selected from the group consisting of Apolipoprotein A1 (ApoA1), Apolipoprotein A2 (ApoA2), Apolipoprotein B (ApoB), albumin (ALB), cytochrome C (CYC), fibronectin (FN), and nuclear RNA (nRNA).

12. The method of any one of claims 1-11, wherein the super-resolution microscopy comprises Single Extracellular Vesicle Nanoscopy (SEVEN).

13. The method of claim 12, SEVEN comprises use of single-molecule localization microscopy (SMLM).

14. The method of claim 13, wherein the SMLM is quantitative SMLM (qSMLM).

15. The method of claim 13, wherein the qSMLM comprises use of a surface assay for molecular isolation (SAMI-qSMLM).

16. The method of claim 12, wherein the SMLM is photoactivated localization microscopy (PALM).

17. The method of claim 12, wherein the SMLM is stochastic optical reconstruction microscopy (STORM).

18. The method of claim 16, wherein the STORM is direct STORM (dSTORM).

19. The method of claim 12, wherein the SMLM is point accumulation in nanoscale topography (PAINT).

20. The method of any one of claims 1-12, wherein the super-resolution microscopy comprises super-resolution radial fluctuations (SRRF) imaging.

21. The method of any one of claims 1-12 and 20, wherein the super-resolution microscopy comprises total internal reflection fluorescence (TIRF) illumination.

22. The method of any one of claims 1-12, 20, and 21, wherein the super-resolution microscopy comprises SRRF imaging and TIRF illumination.

23. The method of any one of claims 1-12, and 20 wherein the super-resolution microscopy comprises wide field illumination.

24. The method of any one of claims 1-12, 20, and 23, wherein the super-resolution microscopy comprises SRRF imaging and wide field illumination.

25. The method of any one of claims 1-12 and 20, wherein the super-resolution microscopy comprises confocal illumination.

26. The method of any one of claims 1-12, 20, and 25, wherein the super-resolution microscopy comprises SRRF imaging and confocal illumination.

27. The method of any one of claims 1-26, wherein the profile of the population of TSEVs further includes information on one or more of the following:
(i) size of TSEVs;
(ii) shape of TSEVs;
(ii) quantity or concentration of TSEVs; and
(iii) heterogeneity of TSEVs.

28. The method of claim 27, wherein the information on the shape of TSEVs comprises information on the circularity and/or eccentricity of the TSEVs.

29. The method of any one of claims 9-28, wherein the tissue-specific biomarker is from a tissue selected from the group consisting of cardiac tissue, neural tissue, pancreatic tissue, immune tissue, and cancer tissue.

30. The method of any one of claims 9-29, wherein the EV-specific biomarker and/or the tissue-specific biomarker is a membrane protein, cytoplasmic protein, glycan, nucleic acid, lipid, or a combination thereof.

31. The method of any one of claims 1-30, wherein the affinity capture agent of step (a) is a protein, peptide, aptamer, carbohydrate, or a combination thereof.

32. The method of claim 31, wherein the carbohydrate is a lectin.

33. The method of claim 30, wherein the protein or peptide is selected from the group consisting of a primary antibody, secondary antibody, Fab, F(ab′), F(ab′)2, single chain variable fragment (scFv), Fd, minibody, variable heavy domain, variable light domain, variable NAR domain, single chain binding polypeptide, dAb fragment, nanobody, VHH, and toxin.

34. The method of any one of claims 1-33, further comprising labeling the TSEVs with a labeling agent.

35. The method of claim 34, wherein the labeling agent a fluorescent reporter or a binding agent conjugated to a fluorescent reporter.

36. The method of claim 35, wherein the fluorescent reporter is a photoswitchable fluorescent reporter, photoactivatable fluorescent reporter, photoconvertible fluorescent reporter, spontaneously blinking fluorescent reporter, or temporarily binding fluorescent reporter.

37. The method of claim 35 or 36, wherein the fluorescent reporter is selected from the group consisting of AF532, AF488, AF532, AF555, AF568, AF594, AF647, AF680, AF700, AF750, Atto488, Atto532, Atto647N, Atto680, Atto700, CF532, CF555, CF568, CF647, CF660C, CF680, CF750, CF488A, CF583R, CF597R, CF680R, CF535ST, Cy3, Cy3b, Cy5, DY-634, DyLight650, Dronpa, JF549, JF646, JFX549, JFX554, JFX646, JFX650, mIrisFP, mMaple, mMaple 3, PAGFP, PAmCherry, PATagRFP, PAmKate, PS-CFP2, and quantum dots.

38. The method of any one of claims 1-37, wherein the functionalized surface of step (a) is a coverslip.

39. The method of any one of claims 1-38, wherein the functionalized surface of step (a) is coated with a coupling agent.

40. The method of claim 39, wherein the coupling agent is attached to the functionalized surface by way of a linker moiety.

41. The method of claim 39 or 40, wherein the coupling agent is selected from the group consisting of MCP2, MCP4, p-aminophenyltrimethoxysilane (APTMS), and aminotrimethoxy silane (ATMS).

42. The method of any one of claims 1-41, wherein the intra-vesicular cargo comprises a protein, nucleic acid, lipid, or carbohydrate.

43. The method of claim 42, wherein the nucleic acid is an RNA or a DNA.

44. The method of claim 43, wherein the RNA is a messenger RNA (mRNA), microRNA (miRNA), long non-coding RNA (lncRNA), transfer RNA (tRNA), tRNA-derived small RNA (tsRNA), ribosomal RNA (rRNA), small rDNA-derived RNA (srRNA), small nucleolar RNA (snoRNA), Piwi-interacting RNA (piRNA).

45. The method of any one of claims 1-44, wherein the membrane protein composition and/or the intra-vesicular cargo composition of individual TSEVs is further assayed using proteomic analysis to generate a proteomic profile of the population of TSEVs.

46. The method of claim 45, wherein the proteomic analysis comprises an immunoassay, mass spectrometry (MS), high performance liquid chromatography (HPLC), reversed-phase chromatography, dot blot analysis, two-dimensional gel electrophoresis, Edman sequencing, protein microarray analysis, structural proteomic analysis, functional proteomic analysis, protein-protein interaction analysis, proteome mining, and post-translational modification analysis.

47. The method of any one of claims 1-46, wherein the intra-vesicular cargo composition of individual TSEVs is further analyzed using transcriptomic analysis to generate a transcriptomic profile of the population of TSEVs.

48. The method of claim 47, wherein the transcriptomic analysis comprises RNA sequencing (RNA-seq).

49. The method of claim 47 or 48, wherein the transcriptomic analysis comprises analysis of lncRNAs.

50. The method of any one of claims 1-49, wherein the sample is: (1) a biofluid or tissue sample obtained from a subject; or (2) a cell culture medium.

51. The method of claim 50, wherein the biofluid sample obtained from a subject is selected from the group consisting of whole blood, plasma, serum, cerebrospinal fluid, saliva, sputum, nasal secretion, ocular secretion, cystic fluid, synovial fluid, bronchoalveolar lavage fluid, amniotic fluid, bone marrow aspirate, bile, milk, stool, swab, smear, semen, or urine.

52. The method of claim 50 or 51, wherein the biofluid sample has a volume between 0.08 μL and 400 μL.

53. The method of claim 52, wherein the biofluid sample has a volume no greater than 1 μL.

54. The method of any one of claims 50-53, wherein the tissue sample is a formalin-fixed paraffin-embedded (FFPE) tissue block, fixed tissue, fresh tissue, or frozen tissue.

55. The method of any one of claims 1-54, wherein the method is performed in accord with Minimal Information for Studies of Extracellular Vesicles (MISEV) guidelines.

56. The method of any one of claims 1-55, wherein the method identifies one or more biomarkers present on the plasma membrane or in the lumen of TSEVs as being associated with a disease or disorder.

57. A method of identifying one or more biomarkers associated with a disease or disorder from a population of TSEVs in a sample, comprising:
(i) performing the method of any one of claims 1-56, thereby obtaining a level of expression of one or more tissue-specific biomarkers from an enriched population of disease-associated TSEVs in the sample;
(ii) calculating a difference score for the one or more tissue-specific biomarkers by comparing a level of expression of the one or more tissue-specific biomarkers from the population of disease-associated TSEVs to a level of expression of the one or more tissue-specific biomarkers from a reference population of TSEVs;
wherein a difference score above a cutoff value indicates that the one more tissue-specific biomarkers are associated with a disease or disorder.

58. The method of claim 57, wherein the cutoff value is at a 50^{th} percentile, 60^{th} percentile, 70^{th} percentile, 80^{th} percentile, 90^{th} percentile, or greater of the difference score in the reference population of TSEVs.

59. The method of claim 57 or 58, wherein the sample is: (1) a fluid sample obtained from a subject having the disease or disorder; or (2) a cell culture medium comprising a population of cells that model the disease or disorder.

60. A method of diagnosing a subject as having or at risk of developing a disease or disorder, comprising:
(i) performing the method of any one of claims 1-56 on a sample obtained from the subject, thereby obtaining a level of expression of one or more tissue-specific biomarkers associated with the disease or disorder;
(ii) calculating a difference score for the subject by comparing a level of expression of the one or more tissue-specific biomarkers in the subject to a level of expression of the one or more tissue-specific biomarkers from a reference population of subjects without the disease or disorder;
wherein a difference score above a cutoff value identifies the subject as having or at risk of developing the disease or disorder.

61. The method of claim 60, wherein the cutoff value is at a 50^{th} percentile, 60^{th} percentile, 70^{th} percentile, 80^{th} percentile, 90^{th} percentile, or greater of the difference score in the reference population of subjects without the disease or disorder.

62. A method of assessing therapeutic efficacy of a therapeutic agent in a subject having or at risk of developing a disease or disorder, comprising:
(i) performing the method of any one of claims 1-56 on a sample obtained from the subject prior to treatment with the therapeutic agent thereby obtaining a pre-treatment level of expression of one or more tissue-specific biomarkers characterized as having altered expression associated with the disease or disorder;
(ii) administering an amount of the therapeutic agent to the subject;
(iii) performing the method of any one of claims 1-56 on a sample obtained from the subject following treatment with the therapeutic agent thereby obtaining a post-treatment level of expression of the one or more tissue-specific biomarkers;
(iv) calculating a difference score for the therapeutic agent by comparing the pre-treatment level of expression of the one or more tissue-specific biomarkers to the post-treatment level of expression of the one or more tissue-specific biomarkers;
wherein a difference score above a cutoff value indicates that the therapeutic agent is effective at treating the disease or disorder.

63. The method of claim 62, wherein the cutoff value is at a 50^{th} percentile, 60^{th} percentile, 70^{th} percentile, 80^{th} percentile, 90^{th} percentile, or greater of the difference score in the pretreatment level of expression of the one or more tissue-specific biomarkers.

64. The method of claim 62 or 63, the method comprising characterizing the one or more tissue-specific biomarkers as having altered expression associated with the disease or disorder prior to performing step (i).

65. A method of selecting a therapeutic agent for the treatment of a subject having or at risk of developing a disease or disorder, comprising:
(i) performing the method of any one of claims 1-56, thereby obtaining a level of expression of one or more tissue-specific biomarkers from an enriched population of disease-associated TSEVs;
(ii) calculating a difference score for the one or more tissue-specific biomarkers by comparing a level of expression of the one or more tissue-specific biomarkers from the population of disease-associated TSEVs to a level of expression of the one or more tissue-specific biomarkers from a reference population of TSEVs, wherein a difference score above a cutoff value indicates that the one more tissue-specific biomarkers are associated with a disease or disorder; and
(iii) identifying a common biological signaling pathway associated with the one or more disease-associated tissue-specific biomarkers; and
(iv) selecting a therapeutic agent that modulates activity of the common biological signaling pathway.

66. The method of claim 65, further comprising (v) administering an effective amount of the therapeutic agent to the subject having or at risk of developing the disease or disorder.

67. The method of claim 65 or 66, wherein the cutoff value is at a 50^{th} percentile, 60^{th} percentile, 70^{th} percentile, 80^{th} percentile, 90^{th} percentile, or greater of the difference score in the reference population of TSEVs.

68. The method of any one of claims 57-67, wherein the level of expression is a mean or median level of expression.

69. The method of any one of claims 60-68, the method further comprising obtaining the sample from the subject having or at risk of developing the disease or disorder prior to performing step (i).

70. The method of any one of claims 60-68, the method further comprising having obtained the sample from the subject having or at risk of developing the disease or disorder prior to performing step (i).

71. The method of any one of claims 57-70, wherein the disease or disorder is selected from the group consisting of a cancer, cardiovascular disease or disorder, neurological disease or disorder, and autoimmune disease or disorder.

72. The method of any one of claims 57-71, wherein the subject is a human, non-human primate, or rodent.
